(19) United States
(12) Patent Application Publication
(10) Pub. No.: US 2025/0082771 A1
(43) Pub. Date: Mar. 13, 2025

(54) DOSAGE REGIMEN OF AN ANTI-CDH6 ANTIBODY-DRUG CONJUGATE

(71) Applicant: DAIICHI SANKYO COMPANY, LIMITED, Tokyo (JP)

(72) Inventors: Robert Mcleod, Basking Ridge, NJ (US); Yusuke Myobatake, Basking Ridge, NJ (US); Tomomichi Ishizaka, Tokyo (JP); Yumi Nishiya, Tokyo (JP); Chiemi Saito, Tokyo (JP); Hirokazu Suzuki, Tokyo (JP); Shotaro Nagase, Tokyo (JP); Thuy Vu Craveiro, Basking Ridge, NJ (US); Kulandayan Subramanian, Basking Ridge, NJ (US); Jie Lin, Basking Ridge, NJ (US); Daigo Asano, Tokyo (JP); Felipe Kellermann Hurtado, Basking Ridge, NJ (US)

(73) Assignee: DAIICHI SANKYO COMPANY, LIMITED, Tokyo (JP)

(21) Appl. No.: 18/957,554

(22) Filed: Nov. 22, 2024

Related U.S. Application Data
(63) Continuation of application No. PCT/IB2023/055316, filed on May 23, 2023.
(60) Provisional application No. 63/345,286, filed on May 24, 2022.

(51) Int. Cl. A61K 47/68; A61P 35/00

(52) U.S. Cl. CPC A61K 47/68037; A61K 47/6849; A61K 47/6889; A61P 35/00

(57) ABSTRACT

The present disclosure relates to the field of pharmaceutical preparations, dosage regimens, and administration of an antibody-drug conjugate (ADC). More specifically, the ADC is composed of an anti-cadherin-6 (CDH6) antibody connected via a linker to an anticancer agent, such as topoisomerase I inhibitor.

### CROSS-REFERENCE TO RELATED APPLICATIONS

The present application is a U.S. Bypass Continuation Application of International Patent Application No. PCT/IB2023/055316, filed May 23, 2023, which claims priority to and the benefit of U.S. Provisional Application No. 63/345,286, filed May 24, 2022. The contents of these applications are hereby incorporated by reference in their entireties.

### STATEMENT REGARDING SEQUENCE LISTING

The instant application contains a Sequence Listing which has been submitted electronically in XML format and is hereby incorporated by reference in its entirety. Said XML copy, created on May 23, 2023, is named 122622-0192.xml and is 118 kb in size.

### TECHNICAL FIELD

The present disclosure relates to the field of pharmaceutical preparations, dosage regimens, and administration of an antibody-drug conjugate (ADC). More specifically, the ADC is composed of an anti-cadherin-6 (CDH6) antibody connected via a linker to a topoisomerase I inhibitor, such as a derivative of exatecan.

### BACKGROUND

Cadherins are glycoproteins present on the surface of cell membranes and function as cell-cell adhesion molecules through the calcium ion-dependent binding of their N-terminal extracellular domains, or as signal molecules responsible for cell-cell interaction. Classic cadherins are in the cadherin superfamily and are single-pass transmembrane proteins composed of five extracellular domains (EC domains), one transmembrane region, and an intracellular domain. The classic cadherins are classified into the type I family typified by E-cadherin and N-cadherin, and the type II family according to the homologies of their amino acid sequences.

Cadherin-6 (CDH6) is a single-pass transmembrane protein composed of 790 amino acids, which is classified into the type II cadherin family, and this protein has N-terminal extracellular and C-terminal intracellular domains. The human CDH6 gene was cloned for the first time in 1995 (Non Patent Literature 1), and its sequence can be referred to under, for example, accession Nos. NM 004932 and NP 004923 (NCBI).

CDH6 is specifically expressed in the brain or the kidney at the stage of development and has been reported to play an important role in the circuit formation of the central nervous system (Non Patent Literature 2 and 3) and nephron development in the kidney (Non Patent Literature 4 and 5). The expression of CDH6 in the normal tissues of adult humans is localized to the tubules of the kidney, bile duct epithelial cells, and the like.

Meanwhile, it is known that CDH6 is specifically overexpressed at tumor sites in some types of human adult cancers. The correlation of CDH6 expression with poor prognosis and its applicability as a tumor marker has been reported with respect to human renal cell carcinoma, particularly, renal clear cell carcinoma (Non Patent Literature 6 and 7). The high expression of CDH6 has also been reported with respect to human ovarian cancer (Non Patent Literature 8). It has also been reported that CDH6 is involved in the epithelial-mesenchymal transition of human thyroid cancer (Non Patent Literature 9). Furthermore, it has been reported that CDH6 is also expressed in human bile duct cancer and human small-cell lung cancer (Non Patent Literature 12 and 13).

Cancers rank high in causes of death. Although the number of cancer patients is expected to increase with aging of the population, treatment needs have not yet been sufficiently satisfied. The problems of conventional chemotherapeutics are that: due to their low selectivity, these chemotherapeutics are toxic not only to tumor cells but also to normal cells and thereby have adverse reactions; and the chemotherapeutics cannot be administered in sufficient amounts and thus cannot produce their effects sufficiently. Hence, in recent years, more highly selective molecular target drugs or antibody drugs have been developed, which target molecules that exhibit mutations or a high expression characteristic in cancer cells, or specific molecules involved in malignant transformation of cells.

Antibodies are highly stable in blood, and specifically bind to their target antigens. For these reasons, a reduction in adverse reaction is expected, and a large number of antibody drugs have been developed for molecules highly expressed on the surface of cancer cells. One of the techniques relying on the antigen-specific binding ability of antibodies is to use an antibody-drug conjugate (ADC). ADC is a conjugate in which an antibody that binds to an antigen expressed on the surface of cancer cells and can internalize the antigen into the cell through the binding is conjugated to a drug having cytotoxic activity. ADC can efficiently deliver the drug to cancer cells, and can thereby be expected to kill the cancer cells by accumulating the drug in the cancer cells (Non Patent Literature 10 and Patent Literature 1 and 2). With regard to ADC, for example, Adcetris™ (brentuximab vedotin) comprising an anti-CD30 monoclonal antibody conjugated to monomethyl auristatin E has been approved as a therapeutic drug for Hodgkin's lymphoma and anaplastic large cell lymphoma. Also, Kadcyla™ (trastuzumab emtansine) comprising an anti-HER2 monoclonal antibody conjugated to emtansine is used in the treatment of HER2-positive progressive or recurrent breast cancer.

The features of a target antigen suitable for ADC as an antitumor drug are that: the antigen is specifically highly expressed on the surface of cancer cells but has low expression or is not expressed in normal cells; the antigen can be internalized into cells; the antigen is not secreted from the cell surface; etc. The internalization ability of the antibody depends on the properties of both the target antigen and the antibody. It is difficult to predict an antigen-binding site suitable for internalization from the molecular structure of a target or to predict an antibody having high internalization ability from binding strength, physical properties, and the like of the antibody. Hence, an important challenge in developing ADC having high efficacy is obtaining an antibody having high internalization ability against the target antigen (Non Patent Literature 11).

ADC comprising DM4 conjugated to an anti-CDH6 antibody specifically binding to EC domain 5 (EC5) of CDH6 are known as ADC targeting CDH6 (Patent Literature 3, Non Patent Literature 14 and 15). ADC comprising a derivative of exatecan conjugated to an anti-CDH6 antibody specifically binding to EC domain 3 (EC3) of CDH6 is known (Patent Literature 4 and 5).

### CITATION LIST

### Patent Literature

Patent Literature 1: WO2014/057687
Patent Literature 2: US2016/0297890
Patent Literature 3: WO2016/024195
Patent Literature 4: WO2018/212136
Patent Literature 5: WO2023/042097

### Non Patent Literature

Non Patent Literature 1: Shimoyama Y, et al., Cancer Research, 2206-2211, 55, May 15, 1995
Non Patent Literature 2: Inoue T, et al., Developmental Biology, 183-194, 1997
Non Patent Literature 3: Osterhout J A, et al., Neuron, 632-639, 71, Aug. 25, 2011
Non Patent Literature 4: Cho E A, et al., Development, 803-812, 125, 1998
Non Patent Literature 5: Mah S P, et al., Developmental Biology, 38-53, 223, 2000
Non Patent Literature 6: Paul R, et al., Cancer Research, 2741-2748 July 1, 57, 1997
Non Patent Literature 7: Shimazui T, et al., Cancer, 963-968, 101 (5), Sep. 1, 2004
Non Patent Literature 8: Koebel M, et al., PLOS Medicine, 1749-1760, 5 (12), e232, December 2008
Non Patent Literature 9: Gugnoni M, et al., Oncogene, 667-677, 36, 2017
Non Patent Literature 10: Polakis P., Pharmacological Reviews, 3-19, 68, 2016
Non Patent Literature 11: Peters C, et al., Bioscience Reports, 1-20, 35, 2015
Non Patent Literature 12: Goeppert B, et al., Epigenetics, 780-790, 11 (11), 2016
Non Patent Literature 13: Yokoi S, et al., American Journal of Pathology, 207-216, 161, 1, 2002
Non Patent Literature 14: Bialucha et al., Cancer Discovery, 1030-1045, 7, 9. 2017.
Non Patent Literature 15: Schöffski et al., Oncology Research and Treatment, 547-556, 44, 10, 2021.

### SUMMARY

The present disclosure provides a method for treating a cancer in a subject in need thereof, comprising administering a specific anti-CDH6 antibody-drug conjugate to the subject according to a specific dosage regimen and a pharmaceutical composition for use in treating a cancer according to the dosage regimen. The present disclosure also provides a method for treating renal cell carcinoma or ovarian cancer in a subject in need thereof, comprising administering a specific anti-CDH6 antibody-drug conjugate to the subject and a pharmaceutical composition for use in treating renal cell carcinoma or ovarian cancer, comprising the anti-CDH6 antibody-drug conjugate.

The present disclosure includes the following aspects:
[1] An anti-CDH6 antibody-drug conjugate for use in treating or preventing cancer, the antibody-drug conjugate comprising an anti-CDH6 antibody and an antitumor compound connected by a linker, wherein the linker and the antitumor compound are represented by the following formula:

-(Succinimid-3-yl-N)—CH₂CH₂CH₂CH₂CH₂—C(═O)-GGFG-NH—CH₂—O—CH₂—C(═O)—(NH-DX)
wherein -(Succinimid-3-yl-N)— has a structure represented by the following formula:

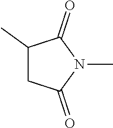

which is connected to the antibody at position 3 thereof and is connected to a methylene group in the linker structure containing this structure on the nitrogen atom at position 1, and (NH-DX) represents a group represented by the following formula:

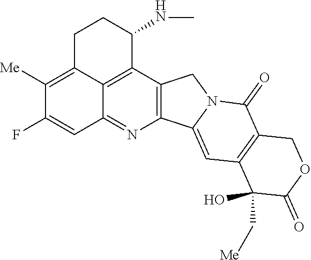 [SMILES: CC[C@@]1(O)C(=O)OCc2c1cc1n(c2=O)Cc2c-1nc1cc(F)c(C)c3c1c2[C@@H](NC)CC3]

wherein the nitrogen atom of the amino group at position 1 is the connecting position, wherein the anti-CDH6 antibody comprises CDRH1 consisting of the amino acid sequence of SEQ ID NO: 17, CDRH2 consisting of the amino acid sequence of SEQ ID NO: 60 and CDRH3 consisting of the amino acid sequence of SEQ ID NO: 19 in its heavy chain variable region and CDRL1 consisting of the amino acid sequence of SEQ ID NO: 12, CDRL2 consisting of the amino acid sequence of SEQ ID NO: 13 and CDRL3 consisting of the amino acid sequence of SEQ ID NO: 14 in its light chain variable region.
[2] The antibody-drug conjugate according to [1], wherein the antibody comprises a heavy chain variable region comprising amino acids sequence of SEQ ID NO: 71 and a light chain variable region comprising amino acids sequence of SEQ ID NO: 63.
[3] The antibody-drug conjugate according to [1] or [2], which has a structure represented by the following formula:

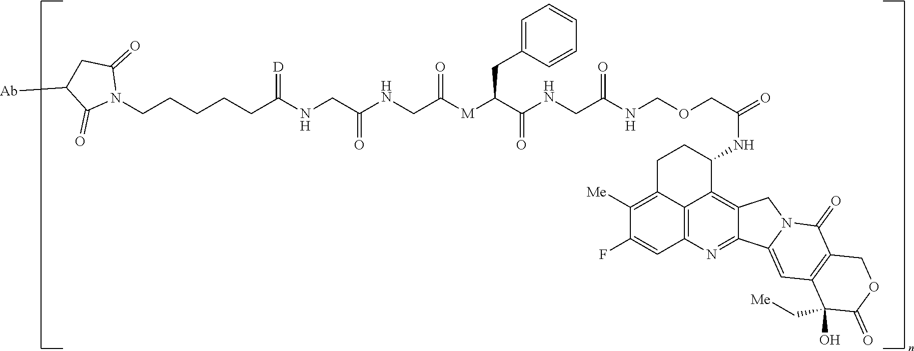

wherein Ab is an antibody comprising a heavy chain variable region comprising SEQ ID NO: 71 and a light chain variable region comprising SEQ ID NO: 63, and n represents the average number of units of the drug-linker structure conjugated to the antibody per antibody, wherein the average number of units of the selected drug-linker structure conjugated per antibody is in the range of from 1 to 10.
[4] The antibody-drug conjugate according to any one of [1] to [3], wherein the antibody comprises a heavy chain comprising the amino acid sequence at positions 20 to 471 in SEQ ID NO: 69 and a light chain comprising the amino acid sequence at positions 21 to 233 in SEQ ID NO: 61.
[5] The antibody-drug conjugate according to any one of [1] to [4], wherein the heavy chain or the light chain has undergone one or two or more modifications selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, conversion of N-terminal glutamine or N-terminal glutamic acid to pyroglutamic acid, and a deletion of one or two amino acids from the carboxyl terminus.
[6] The antibody-drug conjugate according to any one of [1] to [5], wherein the anti-CDH6 antibody lacks a lysine residue at the carboxyl terminus of the heavy chain.
[7] The antibody-drug conjugate according to any one of [1] to [6], wherein an average number of units of the antitumor compound conjugated per antibody is in a range of from 2 to 8.
[8] The antibody-drug conjugate according to any one of [1] to [7], wherein an average number of units of the antitumor compound conjugated per antibody is 7 to 8.
[9] The antibody-drug conjugate according to any one of [1] to [8], wherein a dose of the antibody-drug conjugate in a range of 1.6 mg/kg to 9.6 mg/kg is administered to a subject with cancer.
[10 ] The antibody-drug conjugate according to any one of [1] to [8], wherein a dose of the antibody-drug conjugate in a range of 3.2 mg/kg to 9.6 mg/kg is administered to a subject with cancer.
[11 ] The antibody-drug conjugate according to any one of [1] to [8], wherein a dose of the antibody-drug conjugate in a range of 4.8 mg/kg to 9.6 mg/kg is administered to a subject with cancer.
[12 ] The antibody-drug conjugate according to any one of [1] to [8], wherein a dose of the antibody-drug conjugate in a range of 3.2 mg/kg to 8.0 mg/kg is administered to a subject with cancer.
[13 ] The antibody-drug conjugate according to any one of [1] to [8], wherein a dose of the antibody-drug conjugate in a range of 3.2 mg/kg to 6.4 mg/kg is administered to a subject with cancer.
[14 ] The antibody-drug conjugate according to any one of [1] to [8], wherein a dose of the antibody-drug conjugate in a range of 4.8 mg/kg to 8.0 mg/kg is administered to a subject with cancer.
[15 ] The antibody-drug conjugate according to any one of [1] to [8], wherein a dose of the antibody-drug conjugate in a range of 4.8 mg/kg to 6.4 mg/kg is administered to a subject with cancer.
[16 ] The antibody-drug conjugate according to any one of [1] to [8], wherein a dose of the antibody-drug conjugate of about 3.2 mg/kg, about 4.8 mg/kg, about 5.4 mg/kg, about 5.6 mg/kg, about 6.4 mg/kg or about 8.0 mg/kg is administered to a subject with cancer.
[17 ] The antibody-drug conjugate according to any one of [1] to [8], wherein a dose of the antibody-drug conjugate of about 3.2 mg/kg is administered to a subject with cancer.
[18 ] The antibody-drug conjugate according to any one of [1] to [8], wherein a dose of the antibody-drug conjugate of about 4.8 mg/kg is administered to a subject with cancer.
[19 ] The antibody-drug conjugate according to any one of [1] to [8], wherein a dose of the antibody-drug conjugate of about 5.4 mg/kg is administered to a subject with cancer.
[20 ] The antibody-drug conjugate according to any one of [1] to [8], wherein a dose of the antibody-drug conjugate of about 5.6 mg/kg is administered to a subject with cancer.
[21 ] The antibody-drug conjugate according to any one of [1] to [8], wherein a dose of the antibody-drug conjugate of about 6.4 mg/kg is administered to a subject with cancer.
[22 ] The antibody-drug conjugate according to any one of [1] to [8], wherein a dose of the antibody-drug conjugate of about 8.0 mg/kg is administered to a subject with cancer.
[23 ] The antibody-drug conjugate according to any one of [1] to [8], wherein a dose of the antibody-drug conjugate of 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg or 6.4 mg/kg is administered to a subject with cancer.
[24 ] The antibody-drug conjugate according to any one of [1] to [23], wherein the antibody-drug conjugate is administered by intravenous administration.
[25 ] The antibody-drug conjugate according to any one of [1] to [24], wherein the antibody-drug conjugate is administered once every 3 weeks.
[26 ] The antibody-drug conjugate according to any one of [1] to [25], wherein the cancer is selected from the group consisting of renal cell carcinoma, renal clear cell carcinoma, papillary renal cell carcinoma, ovarian cancer, ovarian serous adenocarcinoma, clear cell carcinoma of ovary, endometrioid carcinoma of ovary, ovarian mucinous tumor, thyroid cancer, bile duct cancer, lung cancer, non-small cell lung cancer, cervix cancer, brain tumor, head and neck cancer, sarcoma, osteosarcoma, small cell lung cancer, glioblastoma, mesothelioma, uterine cancer, pancreatic cancer, Wilms' tumor, neuroblastoma, colorectal cancer, gastric cancer, endometrial cancer, nasopharyngeal cancer, prostate cancer or cancers associated with von Hippel-Lindau disease.
[27 ] The antibody-drug conjugate according to [26], wherein the cancer is renal cell carcinoma, renal clear cell carcinoma or papillary renal cell carcinoma.
[28 ] The antibody-drug conjugate according to [26], wherein the cancer is ovarian cancer.
[29 ] The antibody-drug conjugate according to [28], wherein the ovarian cancer is selected from the group consisting of epithelial ovarian cancer, fallopian tube cancer, or primary peritoneal cancer.
[30 ] The antibody-drug conjugate according to any one of [1] to [29], wherein the cancer is metastatic.
[31 ] The antibody-drug conjugate according to any one of [1] to [30], wherein the cancer is resistant or refractory.
[32 ] The antibody-drug conjugate according to [31], wherein the resistance or refractoriness is resistance or refractoriness acquired by the cancer due to treatment with an anticancer drug.
[33 ] The antibody-drug conjugate according to [32], wherein the anticancer drug is a platinum-based chemotherapeutic, a chemotherapeutic, a PARP inhibitor, an immune checkpoint inhibitor, an angiogenic inhibitor or a VEGFR-TKI.
[34 ] The antibody-drug conjugate according to any one of [1] to [33], wherein the subject has a history of treatment with one or two or more anticancer drugs selected from the group consisting of a platinum-based chemotherapeutic, a chemotherapeutic, a PARP inhibitor, an immune checkpoint inhibitor, an angiogenic inhibitor and a VEGFR-TKI.
[35 ] The antibody-drug conjugate according to [32], wherein the anticancer drug is a platinum-based chemotherapeutic.
[36 ] The antibody-drug conjugate according to [35], wherein the platinum-based chemotherapeutic comprises a platinum-based drug and a taxane.
[37 ] The antibody-drug conjugate according to any one of [1] to [36], wherein the cancer is a CDH6-expressing cancer.
[38 ] The antibody-drug conjugate according to [37], wherein the CDH6-expressing cancer is CDH6-overexpressing cancer.
[39 ] The antibody-drug conjugate according to any one of [1] to [38], wherein the cancer is an inoperable or recurrent cancer.
[40 ] The antibody-drug conjugate according to any one of [1] to [39], wherein the subject exhibits a recurrence of the cancer prior to administration of the antibody-drug conjugate.
[41 ] The antibody-drug conjugate according to [40], wherein the recurrence of the cancer occurs in less than or within about six months of completion of a chemotherapy regimen comprising a platinum-based drug.
[42 ] The antibody-drug conjugate according to [40], wherein the recurrence of the cancer occurs in less than or within about six months of completion of a chemotherapy regimen comprising a platinum-based drug and a taxane.
[43 ] The antibody-drug conjugate according to [40], wherein the recurrence of the cancer occurs in or after about six months of completion of a chemotherapy regimen comprising a platinum-based drug.
[44 ] The antibody-drug conjugate according to [40], wherein the recurrence of the cancer occurs in or after about six months of completion of a chemotherapy regimen comprising a platinum-based drug and a taxane.
[45 ] The antibody-drug conjugate according to any one of [1] to [44], wherein the objective response rate of a subject is at least about 20%.
[46 ] The antibody-drug conjugate according to any one of [1] to [44], wherein the objective response rate of a subject is at least about 30%.
[47 ] The antibody-drug conjugate according to any one of [1] to [46], wherein the progression free survival of a subject is at least 5 months after administration of the antibody-drug conjugate.
[48 ] The antibody-drug conjugate according to any one of [1] to [46], wherein the progression free survival of a subject is at least 5.5 months after administration of the antibody-drug conjugate.
[49 ] The antibody-drug conjugate according to any one of [1] to [48], wherein the antibody-drug conjugate is administered as monotherapy.
[50 ] The antibody-drug conjugate according to any one of [1] to [48], wherein a subject is administered the antibody-drug conjugate with a second drug.
[51 ] The antibody-drug conjugate according to [50], wherein the antibody-drug conjugate is administered prior to, after, or concurrently with the second drug.
[52 ] The antibody-drug conjugate according to any one of [1] to [44], wherein the antibody-drug conjugate is administered as maintenance therapy.
[53 ] The antibody-drug conjugate according to any one of [1] to [44], wherein the antibody-drug conjugate is administered as adjuvant therapy.
[54 ] The antibody-drug conjugate according to any one of [1] to [44], wherein the antibody-drug conjugate is administered after surgical resection of the tumor.
[55 ] The antibody-drug conjugate according to any one of [1] to [44], wherein the antibody-drug conjugate is administered as neoadjuvant therapy.
[56 ] The antibody-drug conjugate according to any one of [1] to [44], wherein the antibody-drug conjugate is administered prior to surgical resection of the tumor.
[57 ] An anti-CDH6 antibody-drug conjugate for use in treating or preventing cancer, the anti-CDH6 antibody-drug conjugate having the structure represented by the following formula:

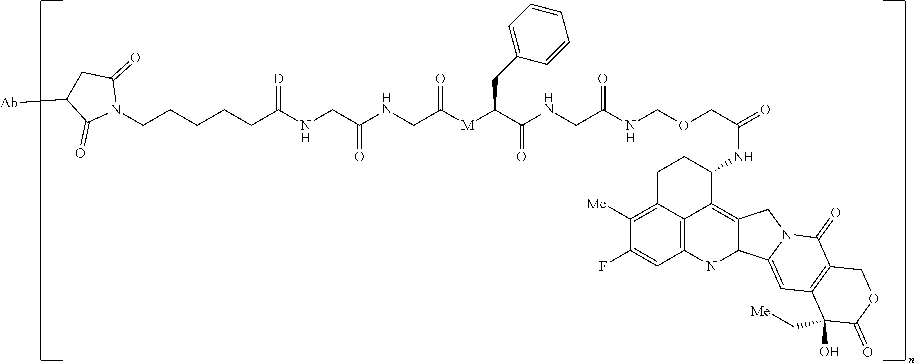

wherein AB represents an anti-CDH6 antibody or the functional fragment of the antibody, n represents the average number of units of the drug-linker structure conjugated to the antibody per antibody, and the antibody is connected to the linker via a sulfhydryl group derived from the antibody; and wherein the anti-CDH6 antibody-drug conjugate is a salt thereof or a hydrate of the anti-CDH6 antibody-drug conjugate or the salt, wherein the average number of units of the drug-linker structure conjugated per antibody is 7 to 8, wherein the antibody comprises: the heavy chain amino acid sequence represented by SEQ ID NO: 87 or an amino acid sequence derived from the amino acid sequence represented by SEQ ID NO: 87 in which one or two amino acids are deleted from the carboxyl terminus thereof; and the light chain amino acid sequence represented by SEQ ID NO: 88, wherein a dose of the antibody-drug conjugate is in a range of 1.6 mg/kg to 9.6 mg/kg, wherein the antibody-drug conjugate is administered by intravenous administration, wherein the antibody-drug conjugate is administered once every 3 weeks.
[58 ] A pharmaceutical composition containing the antibody-drug conjugate according to any one of [1] to [57 ] or a salt thereof as an active component, and a pharmaceutically acceptable formulation component.
[59 ] A method of treating or preventing cancer in a subject, comprising administering to a subject with cancer an anti-CDH6 antibody-drug conjugate comprising an anti-CDH6 antibody and an antitumor compound connected by a linker, wherein the linker and the antitumor compound are represented by the following formula:

-(Succinimid-3-yl-N)—CH₂CH₂CH₂CH₂CH₂—C(═O)-GGFG-NH—CH₂—O—CH₂—C(═O)—(NH-DX)
wherein -(Succinimid-3-yl-N)— has a structure represented by the following formula:

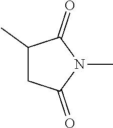

which is connected to the antibody at position 3 thereof and is connected to a methylene group in the linker structure containing this structure on the nitrogen atom at position 1, and (NH-DX) represents a group represented by the following formula:

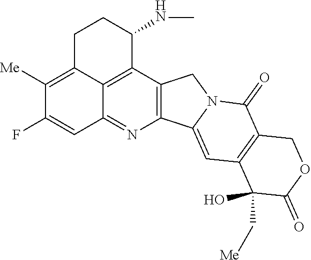 [SMILES: CC[C@@]1(O)C(=O)OCc2c1cc1n(c2=O)Cc2c-1nc1cc(F)c(C)c3c1c2[C@@H](NC)CC3]

wherein the nitrogen atom of the amino group at position 1 is the connecting position, wherein the anti-CDH6 antibody comprises CDRH1 consisting of the amino acid sequence of SEQ ID NO: 17, CDRH2 consisting of the amino acid sequence of SEQ ID NO: 60 and CDRH3 consisting of the amino acid sequence of SEQ ID NO: 19 in its heavy chain variable region and CDRL1 consisting of the amino acid sequence of SEQ ID NO: 12, CDRL2 consisting of the amino acid sequence of SEQ ID NO: 13 and CDRL3 consisting of the amino acid sequence of SEQ ID NO: 14 in its light chain variable region.
[60 ] The method according to [59], wherein the antibody comprises a heavy chain variable region comprising amino acids sequence of SEQ ID NO: 71 and a light chain variable region comprising amino acids sequence of SEQ ID NO: 63.
[61 ] The method according to [59 ] or [60], wherein the antibody-drug conjugate has a structure represented by the following formula:

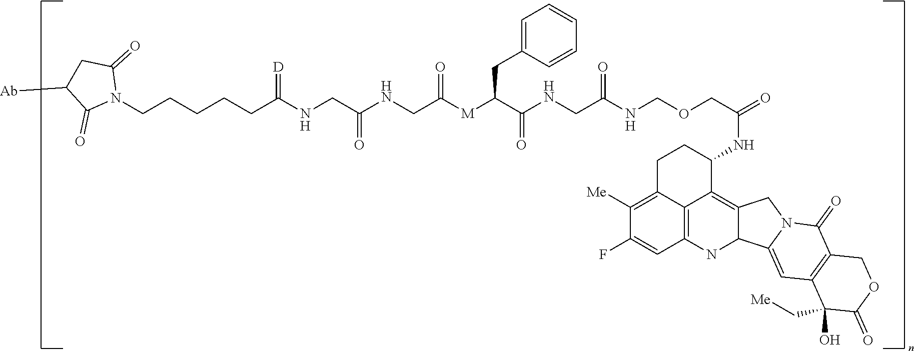

wherein Ab is an antibody comprising a heavy chain variable region comprising SEQ ID NO: 71 and a light chain variable region comprising SEQ ID NO: 63, and n represents the average number of units of the drug-linker structure conjugated to the antibody per antibody, wherein the average number of units of the selected drug-linker structure conjugated per antibody is in the range of from 1 to 10.
[62 ] The method according to any one of [59 ] to [61], wherein the antibody comprises a heavy chain comprising the amino acid sequence at positions 20 to 471 in SEQ ID NO: 69 and a light chain comprising the amino acid sequence at positions 21 to 233 in SEQ ID NO: 61.
[63 ] The method according to any one of [59 ] to [62], wherein the heavy chain or the light chain has undergone one or two or more modifications selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, conversion of N-terminal glutamine or N-terminal glutamic acid to pyroglutamic acid, and a deletion of one or two amino acids from the carboxyl terminus.
[64 ] The method according to any one of [59 ] to [63], wherein the anti-CDH6 antibody lacks a lysine residue at the carboxyl terminus of the heavy chain.
[65 ] The method according to any one of [59 ] to [64], wherein an average number of units of the antitumor compound conjugated per antibody is in a range of [59 ] from 2 to 8.
[66 ] The method according to any one of to [65], wherein an average number of units of the antitumor compound conjugated per antibody is 7 to 8.
[67 ] The method according to any one of [59 ] to [66], wherein a dose of the antibody-drug conjugate in a range of 1.6 mg/kg to 9.6 mg/kg is administered to a subject with cancer.
[68 ] The method according to any one of [59 ] to [66], wherein a dose of the antibody-drug conjugate in a range of 3.2 mg/kg to 9.6 mg/kg is administered to a subject with cancer.
[69 ] The method according to any one of [59 ] to [66], wherein a dose of the antibody-drug conjugate in a range of 4.8 mg/kg to 9.6 mg/kg is administered to a subject with cancer.
[70 ] The method according to any one of [59 ] to [66], wherein a dose of the antibody-drug conjugate in a range of 3.2 mg/kg to 8.0 mg/kg is administered to a subject with cancer.
[71 ] The method according to any one of [59 ] to [66], wherein a dose of the antibody-drug conjugate in a range of 3.2 mg/kg to 6.4 mg/kg is administered to a subject with cancer.
[72 ] The method according to any one of [60 ] to [66], wherein a dose of the antibody-drug conjugate in a range of 4.8 mg/kg to 8.0 mg/kg is administered to a subject with cancer.
[73 ] The method according to any one of [59 ] to [66], wherein a dose of the antibody-drug conjugate in a range of 4.8 mg/kg to 6.4 mg/kg is administered to a subject with cancer.
[74 ] The method according to any one of [59 ] to [66], wherein a dose of the antibody-drug conjugate of about 3.2 mg/kg, about 4.8 mg/kg, about 5.4 mg/kg, about 5.6 mg/kg, about 6.4 mg/kg or about 8.0 mg/kg is administered to a subject with cancer.
[75 ] The method according to any one of [59 ] to [66], wherein a dose of the antibody-drug conjugate of about 3.2 mg/kg is administered to a subject with cancer.
[76 ] The method according to any one of [59 ] to [66], wherein a dose of the antibody-drug conjugate of about 4.8 mg/kg is administered to a subject with cancer.
[77 ] The method according to any one of [59 ] to [66], wherein a dose of the antibody-drug conjugate of about 5.4 mg/kg is administered to a subject with cancer.
[78 ] The method according to any one of [59 ] to [66], wherein a dose of the antibody-drug conjugate of about 5.6 mg/kg is administered to a subject with cancer.
[79 ] The method according to any one of [59 ] to [66], wherein a dose of the antibody-drug conjugate of about 6.4 mg/kg is administered to a subject with cancer.
[80 ] The method according to any one of [59 ] to [66], wherein a dose of the antibody-drug conjugate of about 8.0 mg/kg is administered to a subject with cancer.
[81 ] The method according to any one of [59 ] to [66], wherein a dose of the antibody-drug conjugate of 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg or 6.4 mg/kg is administered to a subject with cancer.
[82 ] The method according to any one of [59 ] to [81], wherein the antibody-drug conjugate is administered by intravenous administration.
[83 ] The method according to any one of [59 ] to [82], wherein the antibody-drug conjugate is administered once every 3 weeks.
[84 ] The method according to any one of [59 ] to [83], wherein the cancer is selected from the group consisting of renal cell carcinoma, renal clear cell carcinoma, papillary renal cell carcinoma, ovarian cancer, ovarian serous adenocarcinoma, clear cell carcinoma of ovary, endometrioid carcinoma of ovary, ovarian mucinous tumor, thyroid cancer, bile duct cancer, lung cancer, non-small cell lung cancer, cervix cancer, brain tumor, head and neck cancer, sarcoma, osteosarcoma, small cell lung cancer, glioblastoma, mesothelioma, uterine cancer, pancreatic cancer, Wilms' tumor, neuroblastoma, colorectal cancer, gastric cancer, endometrial cancer, nasopharyngeal cancer, prostate cancer or cancers associated with von Hippel-Lindau disease.
[85 ] The method according to [84], wherein the cancer is renal cell carcinoma, renal clear cell carcinoma or papillary renal cell carcinoma.
[86 ] The method according to [84], wherein the cancer is ovarian cancer.
[87 ] The method according to [86], wherein the ovarian cancer is selected from the group consisting of epithelial ovarian cancer, fallopian tube cancer, or primary peritoneal cancer.
[88 ] The antibody-drug conjugate according to any one of [59 ] to [87], wherein the cancer is metastatic.
[89 ] The method according to any one of [59 ] to [88], wherein the cancer is resistant or refractory.
[90 ] The method according to [89], wherein the resistance or refractoriness is resistance or refractoriness acquired by the cancer due to treatment with an anticancer drug.
[91 ] The method according to [90], wherein the anticancer drug is a platinum-based chemotherapeutic, a chemotherapeutic, a PARP inhibitor, an immune checkpoint inhibitor, an angiogenic inhibitor or a VEGFR-TKI.
[92 ] The method according to any one of [59 ] to [91], wherein the subject has a history of treatment with one or two or more anticancer drugs selected from the group consisting of a platinum-based chemotherapeutic, a chemotherapeutic, a PARP inhibitor, an immune checkpoint inhibitor, an angiogenic inhibitor and a VEGFR-TKI.
[93 ] The method according to [90], wherein the anticancer drug is a platinum-based chemotherapeutic.
[94 ] The method according to [93], wherein the platinum-based chemotherapeutic comprises a platinum-based drug and a taxane.
[95 ] The method according to any one of [59 ] to [94], wherein the cancer is a CDH6-expressing caner.
[96 ] The method according to [95], wherein the CDH6-expressing cancer is CDH6-overexpressing cancer.
[97 ] The method according to any one of [59 ] to [96], wherein the cancer is an inoperable or recurrent cancer.
[98 ] The method according to any one of [59 ] to [97], wherein the subject exhibits a recurrence of the cancer prior to administration of the antibody-drug conjugate.
[99 ] The method according to [98], wherein the recurrence of the cancer occurs in less than or within about six months of completion of a chemotherapy regimen comprising a platinum-based drug.
[100 ] The method according to [98], wherein the recurrence of the cancer occurs in less than or within about six months of completion of a chemotherapy regimen comprising a platinum-based drug and a taxane.
[101 ] The method according to [98], wherein the recurrence of the cancer occurs in or after about six months of completion of a chemotherapy regimen comprising a platinum-based drug.
[102 ] The method according to [98], wherein the recurrence of the cancer occurs in or after about six months of completion of a chemotherapy regimen comprising a platinum-based drug and a taxane.
[103 ] The method according to any one of [59 ] to [102], wherein the objective response rate of a subject is at least about 20%.
[104 ] The method according to any one of [59 ] to [102], wherein the objective response rate of a subject is at least about 30%.
[105 ] The method according to any one of [59 ] to [104], wherein the progression free survival of a subject is at least 5 months after administration of the antibody-drug conjugate.
[106 ] The method according to any one of [59 ] to [104], wherein the progression free survival of a subject is at least 5.5 months after administration of the antibody-drug conjugate.
[107 ] The method according to any one of [59 ] to [106], wherein the antibody-drug conjugate is administered as monotherapy.
[108 ] The method according to any one of [59 ] to [106], wherein a subject is administered the antibody-drug conjugate with a second drug.
[109 ] The method according to [108], wherein the antibody-drug conjugate is administered prior to, after, or concurrently with the second drug.
[110 ] The method according to any one of [59 ] to [102], wherein the antibody-drug conjugate is administered as maintenance therapy.
[111 ] The method according to any one of [59 ] to [102], wherein the antibody-drug conjugate is administered as adjuvant therapy.
[112 ] The method according to any one of [59 ] to [102], wherein the antibody-drug conjugate is administered after surgical resection of the tumor.
[113 ] The method according to any one of [59 ] to [102], wherein the antibody-drug conjugate is administered as neoadjuvant therapy.
[114 ] The method according to any one of [59 ] to [102], wherein the antibody-drug conjugate is administered prior to surgical resection of the tumor.
[115 ] A method of treating or preventing cancer in a subject, comprising administering to a subject with cancer an anti-CDH6 antibody-drug conjugate having the structure represented by the following formula:

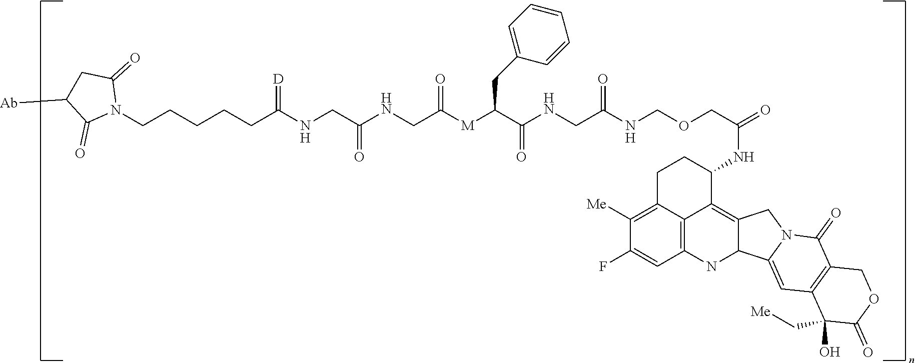

wherein AB represents an anti-CDH6 antibody or the functional fragment of the antibody, n represents the average number of units of the drug-linker structure conjugated to the antibody per antibody, and the antibody is connected to the linker via a sulfhydryl group derived from the antibody; and wherein the anti-CDH6 antibody-drug conjugate is a salt thereof or a hydrate of the anti-CDH6 antibody-drug conjugate or the salt, wherein the average number of units of the drug-linker structure conjugated per antibody is 7 to 8, wherein the antibody comprises: the heavy chain amino acid sequence represented by SEQ ID NO: 87 or an amino acid sequence derived from the amino acid sequence represented by SEQ ID NO: 87 in which one or two amino acids are deleted from the carboxyl terminus thereof; and the light chain amino acid sequence represented by SEQ ID NO: 88, wherein a dose of the antibody-drug conjugate is in a range of 1.6 mg/kg to 9.6 mg/kg, wherein the antibody-drug conjugate is administered by intravenous administration, wherein the antibody-drug conjugate is administered once every 3 weeks.
[116 ] The method according to any one of [59 ] to [115], wherein the antibody-drug conjugate is administered in a pharmaceutical composition comprising at least one pharmaceutically acceptable formulation component.
[117 ] Use of an anti-CDH6 antibody-drug conjugate in the manufacture of a medicament for treating or preventing cancer, the antibody-drug conjugate comprising an anti-CDH6 antibody and an antitumor compound connected by a linker, wherein the linker and the antitumor compound are represented by the following formula:

-(Succinimid-3-yl-N)—CH₂CH₂CH₂CH₂CH₂—C(═O)-GGFG-NH—CH₂—O—CH₂—C(═O)—(NH-DX)
wherein -(Succinimid-3-yl-N)— has a structure represented by the following formula:

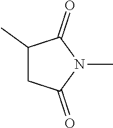

which is connected to the antibody at position 3 thereof and is connected to a methylene group in the linker structure containing this structure on the nitrogen atom at position 1, and (NH-DX) represents a group represented by the following formula:

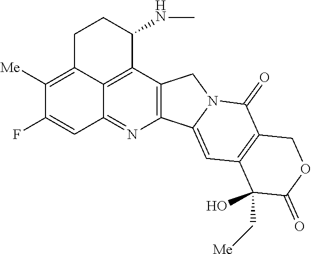 [SMILES: CC[C@@]1(O)C(=O)OCc2c1cc1n(c2=O)Cc2c-1nc1cc(F)c(C)c3c1c2[C@@H](NC)CC3]

wherein the nitrogen atom of the amino group at position 1 is the connecting position, wherein the anti-CDH6 antibody comprises CDRH1 consisting of the amino acid sequence of SEQ ID NO: 17, CDRH2 consisting of the amino acid sequence of SEQ ID NO: 60 and CDRH3 consisting of the amino acid sequence of SEQ ID NO: 19 in its heavy chain variable region and CDRL1 consisting of the amino acid sequence of SEQ ID NO: 12, CDRL2 consisting of the amino acid sequence of SEQ ID NO: 13 and CDRL3 consisting of the amino acid sequence of SEQ ID NO: 14 in its light chain variable region.
[118 ] The use according to [117], wherein the antibody comprises a heavy chain variable region comprising amino acids sequence of SEQ ID NO: 71 and a light chain variable region comprising amino acids sequence of SEQ ID NO: 63.
[119 ] The use according to [117 ] or [118], wherein the antibody-drug conjugate has a structure represented by the following formula:

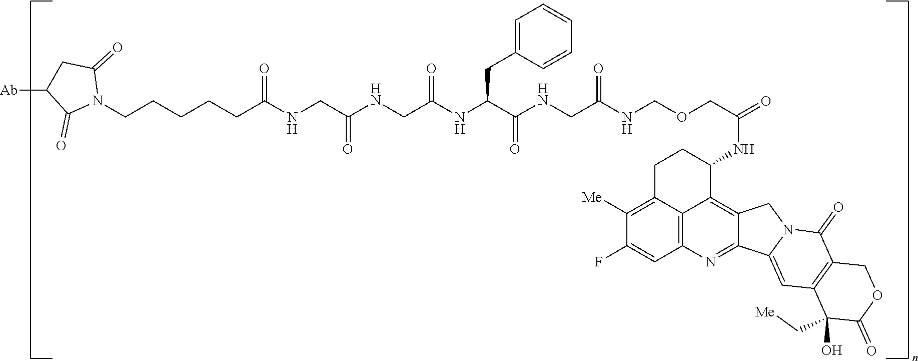

wherein Ab is an antibody comprising a heavy chain variable region comprising SEQ ID NO: 71 and a light chain variable region comprising SEQ ID NO: 63, and n represents the average number of units of the drug-linker structure conjugated to the antibody per antibody, wherein the average number of units of the selected drug-linker structure conjugated per antibody is in the range of from 1 to 10.
[120 ] The use according to any one of [117 ] to [119], wherein the antibody comprises a heavy chain comprising the amino acid sequence at positions 20 to 471 in SEQ ID NO: 69 and a light chain comprising the amino acid sequence at positions 21 to 233 in SEQ ID NO: 61.
[121 ] The use according to any one of [117 ] to [120], wherein the heavy chain or the light chain has undergone one or two or more modifications selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, conversion of N-terminal glutamine or N-terminal glutamic acid to pyroglutamic acid, and a deletion of one or two amino acids from the carboxyl terminus.
[122 ] The use according to any one of [117 ] to [121], wherein the anti-CDH6 antibody lacks a lysine residue at the carboxyl terminus of the heavy chain.
[123 ] The use according to any one of [117 ] to [122], wherein an average number of units of the antitumor compound conjugated per antibody is in a range of from 2 to 8.
[124 ] The use according to any one of [117 ] to [123], wherein an average number of units of the antitumor compound conjugated per antibody is 7 to 8.
[125 ] The use according to any one of [117 ] to [124], wherein a dose of the antibody-drug conjugate in a range of 1.6 mg/kg to 9.6 mg/kg is administered to a subject with cancer.
[126 ] The use according to any one of [117 ] to [124], wherein a dose of the antibody-drug conjugate in a range of 3.2 mg/kg to 9.6 mg/kg is administered to a subject with cancer.
[127 ] The use according to any one of [117 ] to [124], wherein a dose of the antibody-drug conjugate in a range of 4.8 mg/kg to 9.6 mg/kg is administered to a subject with cancer.
[128 ] The use according to any one of [117 ] to [124], wherein a dose of the antibody-drug conjugate in a range of 3.2 mg/kg to 8.0 mg/kg is administered to a subject with cancer.
[129 ] The use according to any one of [117 ] to [124], wherein a dose of the antibody-drug conjugate in a range of 3.2 mg/kg to 6.4 mg/kg is administered to a subject with cancer.
[130 ] The use according to any one of [117 ] to [124], wherein a dose of the antibody-drug conjugate in a range of 4.8 mg/kg to 8.0 mg/kg is administered to a subject with cancer.
[131 ] The use according to any one of [117 ] to [124], wherein a dose of the antibody-drug conjugate in a range of 4.8 mg/kg to 6.4 mg/kg is administered to a subject with cancer.
[132 ] The use according to any one of [117 ] to [124], wherein a dose of the antibody-drug conjugate of about 3.2 mg/kg, about 4.8 mg/kg, about 5.4 mg/kg, about 5.6 mg/kg, about 6.4 mg/kg or about 8.0 mg/kg is administered to a subject with cancer.
[133 ] The use according to any one of [117 ] to [124], wherein a dose of the antibody-drug conjugate of about 3.2 mg/kg is administered to a subject with cancer.
[134 ] The use according to any one of [117 ] to [124], wherein a dose of the antibody-drug conjugate of about 4.8 mg/kg is administered to a subject with cancer.
[135 ] The use according to any one of [117 ] to [124], wherein a dose of the antibody-drug conjugate of about 5.4 mg/kg is administered to a subject with cancer.
[136 ] The use according to any one of [117 ] to [124], wherein a dose of the antibody-drug conjugate of about 5.6 mg/kg is administered to a subject with cancer.
[137 ] The use according to any one of [117 ] to [124], wherein a dose of the antibody-drug conjugate of about 6.4 mg/kg is administered to a subject with cancer.
[138 ] The use according to any one of [117 ] to [124], wherein a dose of the antibody-drug conjugate of about 8.0 mg/kg is administered to a subject with cancer.
[139 ] The use according to any one of [117 ] to [124], wherein a dose of the antibody-drug conjugate of 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg or 6.4 mg/kg is administered to a subject with cancer.
[140 ] The use according to any one of [117 ] to [139], wherein the antibody-drug conjugate is administered by intravenous administration.
[141 ] The use according to any one of [117 ] to [140], wherein the antibody-drug conjugate is administered once every 3 weeks.
[142 ] The use according to any one of [117 ] to [141], wherein the cancer is selected from the group consisting of renal cell carcinoma, renal clear cell carcinoma, papillary renal cell carcinoma, ovarian cancer, ovarian serous adenocarcinoma, clear cell carcinoma of ovary, endometrioid carcinoma of ovary, ovarian mucinous tumor, thyroid cancer, bile duct cancer, lung cancer, non-small cell lung cancer, cervix cancer, brain tumor, head and neck cancer, sarcoma, osteosarcoma, small cell lung cancer, glioblastoma, mesothelioma, uterine cancer, pancreatic cancer, Wilms' tumor, neuroblastoma, colorectal cancer, gastric cancer, endometrial cancer, nasopharyngeal cancer, prostate cancer or cancers associated with von Hippel-Lindau disease.
[143 ] The use according to [142], wherein the cancer is renal cell carcinoma, renal clear cell carcinoma or papillary renal cell carcinoma.
[144 ] The use according to [142], wherein the cancer is ovarian cancer.
[145 ] The use according to [144], wherein the ovarian cancer is selected from the group consisting of epithelial ovarian cancer, fallopian tube cancer, or primary peritoneal cancer.
[146 ] The use according to any one of [117 ] to [145], wherein the cancer is metastatic.
[147 ] The use according to any one of [117 ] to [146], wherein the cancer is resistant or refractory.
[148 ] The use according to [147], wherein the resistance or refractoriness is resistance or refractoriness acquired by the cancer due to treatment with an anticancer drug.
[149 ] The use according to [148], wherein the anticancer drug is a platinum-based chemotherapeutic, a chemotherapeutic, a PARP inhibitor, an immune checkpoint inhibitor, an angiogenic inhibitor or a VEGFR-TKI.
[150 ] The use according to any one of [117 ] to [149], wherein the subject has a history of treatment with one or two or more anticancer drugs selected from the group consisting of a platinum-based chemotherapeutic, a chemotherapeutic, a PARP inhibitor, an immune checkpoint inhibitor, an angiogenic inhibitor and a VEGFR-TKI.
[151 ] The use according to [148], wherein the anticancer drug is a platinum-based chemotherapeutic.
[152 ] The use according to [151], wherein the platinum-based chemotherapeutic comprises a platinum-based drug and a taxane.
[153 ] The use according to any one of [117 ] to [152], wherein the cancer is a CDH6-expressing caner.
[154 ] The use according to [153], wherein the CDH6-expressing cancer is CDH6-overexpressing cancer.
[155 ] The use according to any one of [117 ] to [154], wherein the cancer is an inoperable or recurrent cancer.
[156 ] The use according to any one of [117 ] to [155], wherein the subject exhibits a recurrence of the cancer prior to administration of the antibody-drug conjugate.
[157 ] The use according to [156], wherein the recurrence of the cancer occurs in less than or within about six months of completion of a chemotherapy regimen comprising a platinum-based drug.
[158 ] The use according to [156], wherein the recurrence of the cancer occurs in less than or within about six months of completion of a chemotherapy regimen comprising a platinum-based drug and a taxane.
[159 ] The use according to [156], wherein the recurrence of the cancer occurs in or after about six months of completion of a chemotherapy regimen comprising a platinum-based drug.
[160 ] The use according to [156], wherein the recurrence of the cancer occurs in or after about six months of completion of a chemotherapy regimen comprising a platinum-based drug and a taxane.
[161 ] The use according to any one of [117 ] to [160], wherein the objective response rate of a subject is at least about 20%.
[162 ] The use according to any one of [117 ] to [160], wherein the objective response rate of a subject is at least about 30%.
[163 ] The use according to any one of [117 ] to [162], wherein the progression free survival of a subject is at least 5 months after administration of the antibody-drug conjugate.
[164 ] The use according to any one of [117 ] to [162], wherein the progression free survival of a subject is at least 5.5 months after administration of the antibody-drug conjugate.
[165 ] The use according to any one of [117 ] to [164], wherein the antibody-drug conjugate is administered as monotherapy.
[166 ] The use according to any one of [117 ] to [164], wherein a subject is administered the antibody-drug conjugate with a second drug.
[167 ] The use according to [166], wherein the antibody-drug conjugate is administered prior to, after, or concurrently with the second drug.
[158 ] The use according to any one of [117 ] to [160], wherein the antibody-drug conjugate is administered as maintenance therapy.
[159 ] The use according to any one of [117 ] to [160], wherein the antibody-drug conjugate is administered as adjuvant therapy.
[170 ] The use according to any one of [117 ] to [160], wherein the antibody-drug conjugate is administered after surgical resection of the tumor.
[171 ] The use according to any one of [117 ] to [160], wherein the antibody-drug conjugate is administered as neoadjuvant therapy.
[172 ] The use according to any one of [117 ] to [160], wherein the antibody-drug conjugate is administered prior to surgical resection of the tumor.
[173 ] Use of an anti-CDH6 antibody-drug conjugate in the manufacture of a medicament for treating or preventing cancer, the anti-CDH6 antibody-drug conjugate having the structure represented by the following formula:

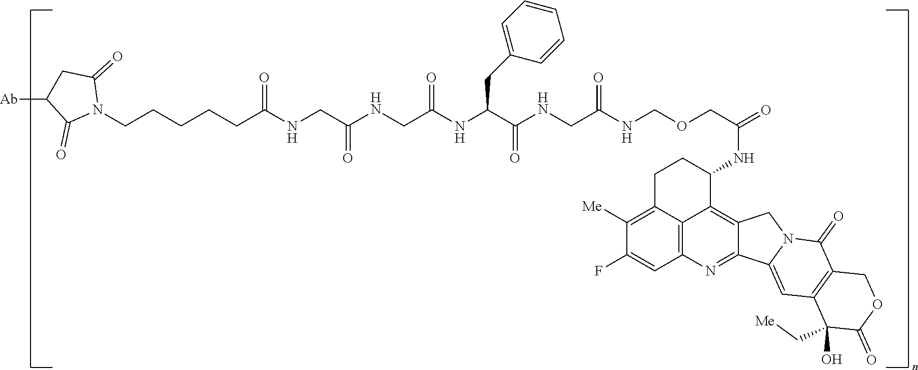

wherein AB represents an anti-CDH6 antibody or the functional fragment of the antibody, n represents the average number of units of the drug-linker structure conjugated to the antibody per antibody, and the antibody is connected to the linker via a sulfhydryl group derived from the antibody; and wherein the anti-CDH6 antibody-drug conjugate is a salt thereof or a hydrate of the anti-CDH6 antibody-drug conjugate or the salt, wherein the average number of units of the drug-linker structure conjugated per antibody is 7 to 8, wherein the antibody comprises: the heavy chain amino acid sequence represented by SEQ ID NO: 87 or an amino acid sequence derived from the amino acid sequence represented by SEQ ID NO: 87 in which one or two amino acids are deleted from the carboxyl terminus thereof; and the light chain amino acid sequence represented by SEQ ID NO: 88, wherein a dose of the antibody-drug conjugate is in a range of 1.6 mg/kg to 9.6 mg/kg, wherein the antibody-drug conjugate is administered by intravenous administration, wherein the antibody-drug conjugate is administered once every 3 weeks.
[174 ] The use according to any one of [117 ] to [173], wherein the antibody-drug conjugate is administered in a pharmaceutical composition comprising at least one pharmaceutically acceptable formulation component.

The present invention also includes the following aspects of the invention:
[1A] An anti-CDH6 antibody-drug conjugate for use in treating or preventing cancer, the antibody-drug conjugate comprising an anti-CDH6 antibody and an antitumor compound connected by a linker, wherein the linker and the antitumor compound are represented by the following formula:

-(Succinimid-3-yl-N)—CH₂CH₂CH₂CH₂CH₂—C(═O)-GGFG-NH—CH₂—O—CH₂—C(═O)—(NH-DX)
wherein -(Succinimid-3-yl-N)— has a structure represented by the following formula:

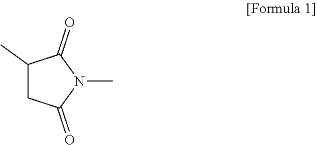

which is connected to the antibody at position 3 thereof and is connected to a methylene group in the linker structure containing this structure on the nitrogen atom at position 1, and (NH-DX) represents a group represented by the following formula:

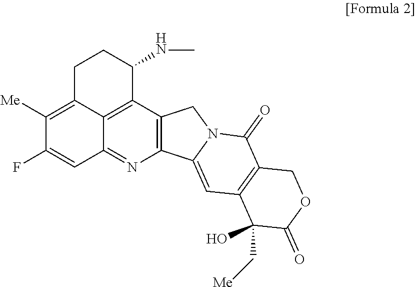 [SMILES: CC[C@@]1(O)C(=O)OCc2c1cc1n(c2=O)Cc2c-1nc1cc(F)c(C)c3c1c2[C@@H](NC)CC3]

wherein the nitrogen atom of the amino group at position 1 is the connecting position, wherein the anti-CDH6 antibody comprises CDRH1 consisting of the amino acid sequence of SEQ ID NO: 17, CDRH2 consisting of the amino acid sequence of SEQ ID NO: 60 and CDRH3 consisting of the amino acid sequence of SEQ ID NO: 19 in its heavy chain variable region and CDRL1 consisting of the amino acid sequence of SEQ ID NO: 12, CDRL2 consisting of the amino acid sequence of SEQ ID NO: 13 and CDRL3 consisting of the amino acid sequence of SEQ ID NO: 14 in its light chain variable region.
[2A] The antibody-drug conjugate according to [1A], wherein the antibody comprises a heavy chain variable region comprising amino acids sequence of SEQ ID NO: 71 and a light chain variable region comprising amino acids sequence of SEQ ID NO: 63.
[3A] The antibody-drug conjugate according to [1A] or [2A], which has a structure represented by the following formula:

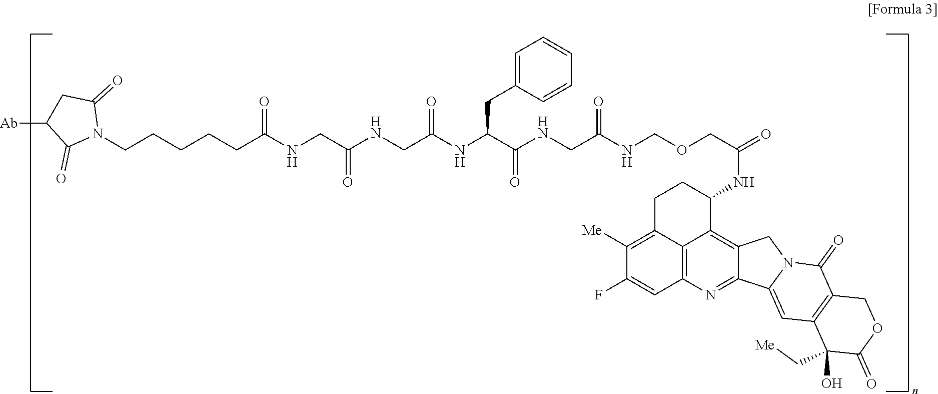

wherein Ab is an antibody comprising a heavy chain variable region comprising SEQ ID NO: 71 and a light chain variable region comprising SEQ ID NO: 63, and n represents the average number of units of the drug-linker structure conjugated to the antibody per antibody, wherein the average number of units of the selected drug-linker structure conjugated per antibody is in the range of from 1 to 10.
[4A] The antibody-drug conjugate according to any one of [1A] to [3A], wherein the antibody comprises a heavy chain comprising the amino acid sequence at positions 20 to 471 in SEQ ID NO: 69 and a light chain comprising the amino acid sequence at positions 21 to 233 in SEQ ID NO: 61.
[5A] The antibody-drug conjugate according to any one of [1A] to [4A], wherein the heavy chain or the light chain has undergone one or two or more modifications selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, conversion of N-terminal glutamine or N-terminal glutamic acid to pyroglutamic acid, and a deletion of one or two amino acids from the carboxyl terminus.
[6A] The antibody-drug conjugate according to any one of [1A] to [5A], wherein the anti-CDH6 antibody lacks a lysine residue at the carboxyl terminus of the heavy chain.
[7A] The antibody-drug conjugate according to any one of [1A] to [6A], wherein an average number of units of the antitumor compound conjugated per antibody is in a range of from 2 to 8.
[8A] The antibody-drug conjugate according to any one of [1A] to [7A], wherein an average number of units of the antitumor compound conjugated per antibody is 7 to 8.
[9A] The antibody-drug conjugate according to any one of [1A] to [8A], wherein a dose of the antibody-drug conjugate is in a range of 1.6 mg/kg to 9.6 mg/kg is administered to a subject with cancer.
[10A] The antibody-drug conjugate according to any one of [1A] to [9A], wherein a dose of the antibody-drug conjugate of about 3.2 mg/kg is administered to a subject with cancer.
[11A] The antibody-drug conjugate according to any one of [1A] to [9A], wherein a dose of the antibody-drug conjugate of about 4.8 mg/kg is administered to a subject with cancer.
[12A] The antibody-drug conjugate according to any one of [1A] to [9A], wherein a dose of the antibody-drug conjugate of about 6.4 mg/kg is administered to a subject with cancer.
[13A] The antibody-drug conjugate according to any one of [1A] to [9A], wherein a dose of the antibody-drug conjugate of about 8.0 mg/kg is administered to a subject with cancer.
[14A] The antibody-drug conjugate according to any one of [1A] to [13A], wherein the antibody-drug conjugate is administered by intravenous administration.
[15A] The antibody-drug conjugate according to any one of [1A] to [14A], wherein the antibody-drug conjugate is administered once every 3 weeks.
[16A] The antibody-drug conjugate according to any one of [1A] to [15A], wherein the cancer is selected from the group consisting of renal cell carcinoma, renal clear cell carcinoma, papillary renal cell carcinoma, ovarian cancer, ovarian serous adenocarcinoma, thyroid cancer, bile duct cancer, lung cancer, small-cell lung cancer, glioblastoma, mesothelioma, uterine cancer, pancreatic cancer, Wilms' tumor or neuroblastoma.
[17A] The antibody-drug conjugate according to [16A], wherein the cancer is renal cell carcinoma.
[18A] The antibody-drug conjugate according to [16A], wherein the cancer is ovarian cancer.
[19A] The antibody-drug conjugate according to any one of [1A] to [18A], wherein the cancer is resistant or refractory.
[20A] The antibody-drug conjugate according to [19A], wherein the resistance or refractoriness is resistance or refractoriness acquired by the cancer due to treatment with an anticancer drug.
[21A] The antibody-drug conjugate according to [20A], wherein the anticancer drug is a platinum-based chemotherapeutic, a chemotherapy, a PARP inhibitor, an immune checkpoint inhibitor, an angiogenic inhibitor or a VEGFR-TKI.
[22A] The antibody-drug conjugate according to [20A], wherein the anticancer drug is a platinum-based chemotherapeutic.
[23A] The antibody-drug conjugate according to [22A], wherein the platinum-based chemotherapeutic comprises a platinum-based drug and a taxane.
[24A] The antibody-drug conjugate according to any one of [1A] to [23A], wherein the cancer is a CDH6-expressing cancer.
[25A] The antibody-drug conjugate according to [24A], wherein the CDH6-expressing cancer is CDH6-overexpressing cancer.
[26A] The antibody-drug conjugate according to any one of [1A] to [25A], wherein the cancer is an inoperable or recurrent cancer.
[27A] A pharmaceutical composition containing the antibody-drug conjugate according to any one of [1A] to [26A] or a salt thereof as an active component, and a pharmaceutically acceptable formulation component.
[28A] A method of treating or preventing cancer in a subject, comprising administering to a subject with cancer an anti-CDH6 antibody-drug conjugate comprising an anti-CDH6 antibody and an antitumor compound connected by a linker, wherein the linker and the antitumor compound are represented by the following formula:

-(Succinimid-3-yl-N)—CH2CH2CH2CH2CH2-C(═O)-GGFG-NH—CH2-O—CH2-C(═O)—(NH-DX)
wherein -(Succinimid-3-yl-N)— has a structure represented by the following formula:

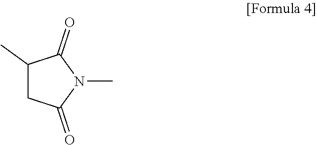

which is connected to the antibody at position 3 thereof and is connected to a methylene group in the linker structure containing this structure on the nitrogen atom at position 1, and (NH-DX) represents a group represented by the following formula:

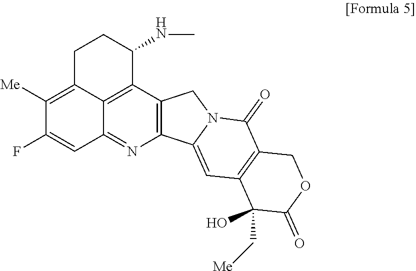 [SMILES: CC[C@@]1(O)C(=O)OCc2c1cc1n(c2=O)Cc2c-1nc1cc(F)c(C)c3c1c2[C@@H](NC)CC3]

wherein the nitrogen atom of the amino group at position 1 is the connecting position, wherein the anti-CDH6 antibody comprises CDRH1 consisting of the amino acid sequence of SEQ ID NO: 17, CDRH2 consisting of the amino acid sequence of SEQ ID NO: 60 and CDRH3 consisting of the amino acid sequence of SEQ ID NO: 19 in its heavy chain variable region and CDRL1 consisting of the amino acid sequence of SEQ ID NO: 12, CDRL2 consisting of the amino acid sequence of SEQ ID NO: 13 and CDRL3 consisting of the amino acid sequence of SEQ ID NO: 14 in its light chain variable region.
[29A] The method according to [28A], wherein the antibody comprises a heavy chain variable region comprising amino acids sequence of SEQ ID NO: 71 and a light chain variable region comprising amino acids sequence of SEQ ID NO: 63.
[30A] The method according to [28A] or [29A], wherein the antibody-drug conjugate has a structure represented by the following formula:

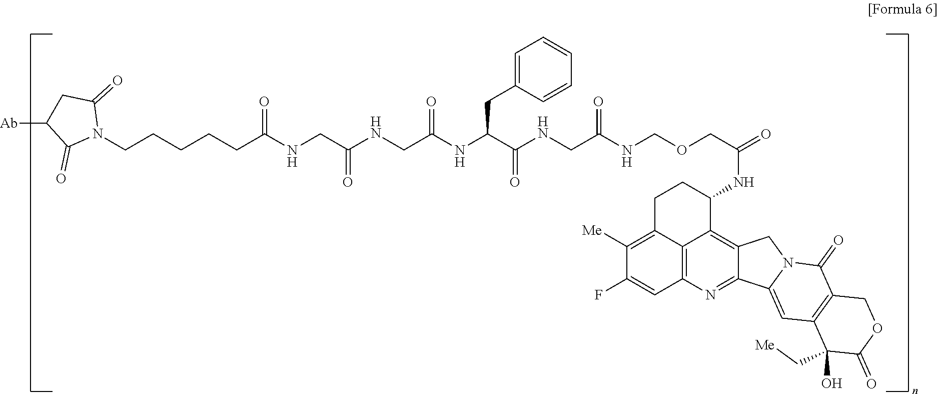

wherein Ab is an antibody comprising a heavy chain variable region comprising SEQ ID NO: 71 and a light chain variable region comprising SEQ ID NO: 63, and n represents the average number of units of the drug-linker structure conjugated to the antibody per antibody, wherein the average number of units of the selected drug-linker structure conjugated per antibody is in the range of from 1 to 10.
[31A] The method according to any one of [28A] to [30A], wherein the antibody comprises a heavy chain comprising the amino acid sequence at positions 20 to 471 in SEQ ID NO: 69 and a light chain comprising the amino acid sequence at positions 21 to 233 in SEQ ID NO: 61.
[32A] The method according to any one of [28A] to [31A], wherein the heavy chain or the light chain has undergone one or two or more modifications selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, conversion of N-terminal glutamine or N-terminal glutamic acid to pyroglutamic acid, and a deletion of one or two amino acids from the carboxyl terminus.
[33A] The method according to any one of [28A] to [32A], wherein the anti-CDH6 antibody lacks a lysine residue at the carboxyl terminus of the heavy chain.
[34A] The method according to any one of [28A] to [33A], wherein an average number of units of the antitumor compound conjugated per antibody is in a range of from 2 to 8.
[35A] The method according to any one of [28A] to [33A], wherein an average number of units of the antitumor compound conjugated per antibody is 7 to 8.
[36A] The method according to any one of [28A] to [35A], wherein a dose of the antibody-drug conjugate is in a range of 1.6 mg/kg to 9.6 mg/kg is administered to a subject with cancer.
[37A] The method according to any one of [28A] to [35A], wherein a dose of the antibody-drug conjugate of about 3.2 mg/kg is administered to a subject with cancer.
[38A] The method according to any one of [28A] to [35A], wherein a dose of the antibody-drug conjugate of about 4.8 mg/kg is administered to a subject with cancer.
[39A] The method according to any one of [28A] to [35A], wherein a dose of the antibody-drug conjugate of about 6.4 mg/kg is administered to a subject with cancer.
[40A] The method according to any one of [28A] to [35A], wherein a dose of the antibody-drug conjugate of about 8.0 mg/kg is administered to a subject with cancer.
[41A] The method according to any one of [28A] to [40A], wherein the antibody-drug conjugate is administered by intravenous administration.
[42A] The method according to any one of [28A] to [41A], wherein the antibody-drug conjugate is administered once every 3 weeks.
[43A] The method according to any one of [28A] to [42A], wherein the cancer is selected from the group consisting of renal cell carcinoma, renal clear cell carcinoma, papillary renal cell carcinoma, ovarian cancer, ovarian serous adenocarcinoma, thyroid cancer, bile duct cancer, lung cancer, small-cell lung cancer, glioblastoma, mesothelioma, uterine cancer, pancreatic cancer, Wilms' tumor or neuroblastoma.
[44A] The method according to [43A], wherein the cancer is renal cell carcinoma.
[45A] The method according to [43A], wherein the cancer is ovarian cancer.
[46A] The method according to any one of [28A] to [45A], wherein the cancer is resistant or refractory.
[47A] The method according to [46A], wherein the resistance or refractoriness is resistance or refractoriness acquired by the cancer due to treatment with an anticancer drug.
[48A] The method according to [47A], wherein the anticancer drug is a platinum-based chemotherapeutic, a chemotherapy, a PARP inhibitor, an immune checkpoint inhibitor, an angiogenic inhibitor or a VEGFR-TKI.
[49A] The method according to [47A], wherein the anticancer drug is a platinum-based chemotherapeutic.
[50A] The method according to [49A], wherein the platinum-based chemotherapeutic comprises a platinum-based drug and a taxane.
[51A] The method according to any one of [28A] to [50A], wherein the cancer is a CDH6-expressing caner.
[52A] The method according to [51A], wherein the CDH6-expressing cancer is CDH6-overexpressing cancer.
[53A] The method according to any one of [28A] to [52A], wherein the cancer is an inoperable or recurrent cancer.
[54A] The method according to any one of [28A] to [53A], wherein the antibody-drug conjugate is administered in a pharmaceutical composition comprising at least one pharmaceutically acceptable formulation component.
[55A] Use of an anti-CDH6 antibody-drug conjugate in the manufacture of a medicament for treating or preventing cancer, the antibody-drug conjugate comprising an anti-CDH6 antibody and an antitumor compound connected by a linker, wherein the linker and the antitumor compound are represented by the following formula:

-(Succinimid-3-yl-N)—CH2CH2CH2CH2CH2-C(═O)-GGFG-NH—CH2-O—CH2-C(═O)—(NH-DX)
wherein -(Succinimid-3-yl-N)— has a structure represented by the following formula:

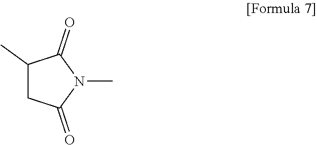

which is connected to the antibody at position 3 thereof and is connected to a methylene group in the linker structure containing this structure on the nitrogen atom at position 1, and (NH-DX) represents a group represented by the following formula:

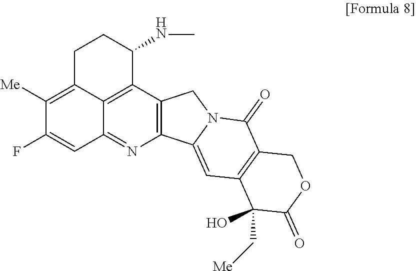 [SMILES: CC[C@@]1(O)C(=O)OCc2c1cc1n(c2=O)Cc2c-1nc1cc(F)c(C)c3c1c2[C@@H](NC)CC3]

wherein the nitrogen atom of the amino group at position 1 is the connecting position, wherein the anti-CDH6 antibody comprises CDRH1 consisting of the amino acid sequence of SEQ ID NO: 17, CDRH2 consisting of the amino acid sequence of SEQ ID NO: 60 and CDRH3 consisting of the amino acid sequence of SEQ ID NO: 19 in its heavy chain variable region and CDRL1 consisting of the amino acid sequence of SEQ ID NO: 12, CDRL2 consisting of the amino acid sequence of SEQ ID NO: 13 and CDRL3 consisting of the amino acid sequence of SEQ ID NO: 14 in its light chain variable region.
[56A] The use according to [55A], wherein the antibody comprises a heavy chain variable region comprising amino acids sequence of SEQ ID NO: 71 and a light chain variable region comprising amino acids sequence of SEQ ID NO: 63.
[57A] The use according to [55A] or [56A], wherein the antibody-drug conjugate has a structure represented by the following formula:

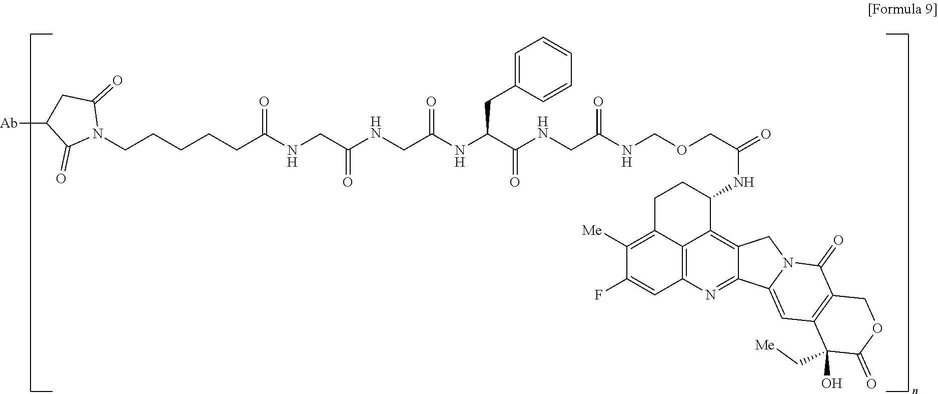

wherein Ab is an antibody comprising a heavy chain variable region comprising SEQ ID NO: 71 and a light chain variable region comprising SEQ ID NO: 63, and n represents the average number of units of the drug-linker structure conjugated to the antibody per antibody, wherein the average number of units of the selected drug-linker structure conjugated per antibody is in the range of from 1 to 10.
[58A] The use according to any one of [55A] to [57A], wherein the antibody comprises a heavy chain comprising the amino acid sequence at positions 20 to 471 in SEQ ID NO: 69 and a light chain comprising the amino acid sequence at positions 21 to 233 in SEQ ID NO: 61.
[59A] The use according to any one of [55A] to [58A], wherein the heavy chain or the light chain has undergone one or two or more modifications selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, conversion of N-terminal glutamine or N-terminal glutamic acid to pyroglutamic acid, and a deletion of one or two amino acids from the carboxyl terminus.
[60A] The use according to any one of [55A] to [59A], wherein the anti-CDH6 antibody lacks a lysine residue at the carboxyl terminus of the heavy chain.
[61A] The use according to any one of [55A] to [60A], wherein an average number of units of the antitumor compound conjugated per antibody is in a range of from 2 to 8.
[62A] The use according to any one of [55A] to [61A], wherein an average number of units of the antitumor compound conjugated per antibody is 7 to 8.
[63A] The use according to any one of [55A] to [62A], wherein a dose of the antibody-drug conjugate is in a range of 1.6 mg/kg to 9.6 mg/kg is administered to a subject with cancer.
[64A] The use according to any one of [55A] to [63A], wherein a dose of the antibody-drug conjugate of about 3.2 mg/kg is administered to a subject with cancer.
[65A] The use according to any one of [55A] to [63A], wherein a dose of the antibody-drug conjugate of about 4.8 mg/kg is administered to a subject with cancer.
[66A] The use according to any one of [55A] to [63A], wherein a dose of the antibody-drug conjugate of about 6.4 mg/kg is administered to a subject with cancer.
[67A] The use according to any one of [55A] to [63A], wherein a dose of the antibody-drug conjugate of about 8.0 mg/kg is administered to a subject with cancer.
[68A] The use according to any one of [55A] to [67A], wherein the antibody-drug conjugate is administered by intravenous administration.
[69A] The use according to any one of [55A] to [68A], wherein the antibody-drug conjugate is administered once every 3 weeks.
[70A] The use according to any one of [55A] to [69A], wherein the cancer is selected from the group consisting of renal cell carcinoma, renal clear cell carcinoma, papillary renal cell carcinoma, ovarian cancer, ovarian serous adenocarcinoma, thyroid cancer, bile duct cancer, lung cancer, small-cell lung cancer, glioblastoma, mesothelioma, uterine cancer, pancreatic cancer, Wilms' tumor or neuroblastoma.
[71A] The use according to [70A], wherein the cancer is renal cell carcinoma.
[72A] The antibody-drug conjugate according to [70A], wherein the cancer is ovarian cancer.
[73A] The use according to any one of [55A] to [72A], wherein the cancer is resistant or refractory.
[74A] The use according to [73A], wherein the resistance or refractoriness is resistance or refractoriness acquired by the cancer due to treatment with an anticancer drug.
[75A] The use according to [74A], wherein the anticancer drug is a platinum-based chemotherapeutic, a chemotherapy, a PARP inhibitor, an immune checkpoint inhibitor, an angiogenic inhibitor or a VEGFR-TKI.
[76A] The use according to [74A], wherein the anticancer drug is a platinum-based chemotherapeutic.
[77A] The use according to [76A], wherein the platinum-based chemotherapeutic comprises a platinum-based drug and a taxane.
[78A] The use according to any one of [55A] to [77A], wherein the cancer is a CDH6-expressing caner.
[79A] The use according to [78A], wherein the CDH6-expressing cancer is CDH6-overexpressing cancer.
[80A] The use according to any one of [55A] to [79A], wherein the cancer is an inoperable or recurrent cancer.
[81A] The use according to any one of [55A] to [80A], wherein the antibody-drug conjugate is administered in a pharmaceutical composition comprising at least one pharmaceutically acceptable formulation component.

In some aspects, the present disclosure provides an anti-CDH6 antibody-drug conjugate for use in treating or preventing cancer, the antibody-drug conjugate comprising an anti-CDH6 antibody and an antitumor compound connected by a linker.

In some aspects, the present disclosure provides a method of treating or preventing cancer in a subject, comprising administering to a subject with cancer an anti-CDH6 antibody-drug conjugate comprising an anti-CDH6 antibody and an antitumor compound connected by a linker.

In some aspect, the present disclosure provides a use of an anti-CDH6 antibody-drug conjugate in the manufacture of a medicament for treating or preventing cancer, the antibody-drug conjugate comprising an anti-CDH6 antibody and an antitumor compound connected by a linker.

In some aspect, the present disclosure provides a pharmaceutical composition for treating or preventing cancer, comprising an anti-CDH6 antibody-drug conjugate as an active ingredient, the antibody-drug conjugate comprising an anti-CDH6 antibody and an antitumor compound connected by a linker.

In some embodiments, the linker and the antitumor compound are represented by the following formula:

-(Succinimid-3-yl-N)—CH₂CH₂CH₂CH₂CH₂—C(═O)-GGFG-NH—CH₂—O—CH₂—C(═O)—(NH-DX)

wherein -(Succinimid-3-yl-N)— has a structure represented by the following formula:

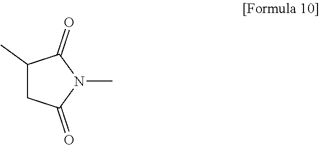

which is connected to the antibody at position 3 thereof and is connected to a methylene group in the linker structure containing this structure on the nitrogen atom at position 1, and (NH-DX) represents a group represented by the following formula:

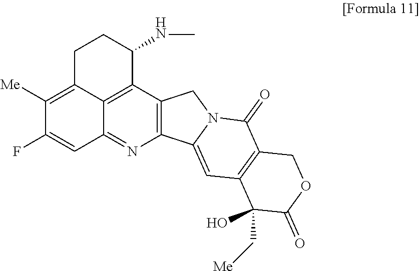 [SMILES: CC[C@@]1(O)C(=O)OCc2c1cc1n(c2=O)Cc2c-1nc1cc(F)c(C)c3c1c2[C@@H](NC)CC3]

wherein the nitrogen atom of the amino group at position 1 is the connecting position.

In some aspects, the anti-CDH6 antibody comprises CDRH1 consisting of the amino acid sequence of SEQ ID NO: 17, CDRH2 consisting of the amino acid sequence of SEQ ID NO: 60 and CDRH3 consisting of the amino acid sequence of SEQ ID NO: 19 in its heavy chain variable region and CDRL1 consisting of the amino acid sequence of SEQ ID NO: 12, CDRL2 consisting of the amino acid sequence of SEQ ID NO: 13 and CDRL3 consisting of the amino acid sequence of SEQ ID NO: 14 in its light chain variable region.

In some aspects, the anti-CDH6 antibody comprises a heavy chain variable region comprising amino acids sequence of SEQ ID NO: 71 and a light chain variable region comprising amino acids sequence of SEQ ID NO: 63.

In some aspects, the antibody-drug conjugate has a structure represented by the following formula:

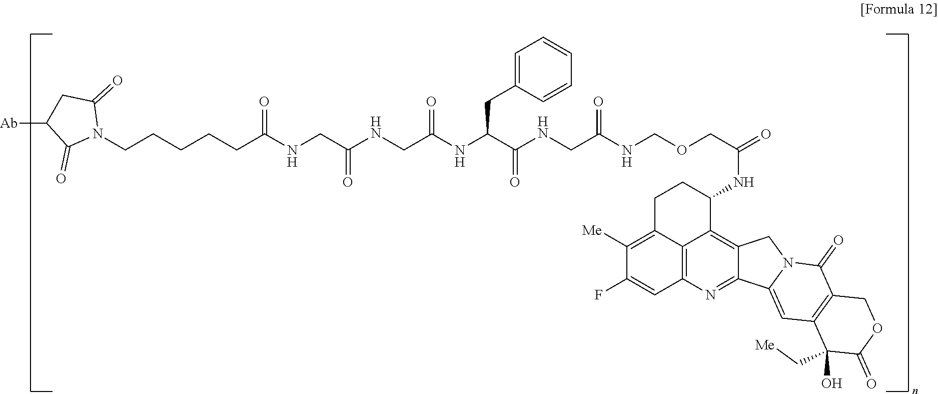

wherein Ab is an antibody comprising a heavy chain variable region comprising SEQ ID NO: 71 and a light chain variable region comprising SEQ ID NO: 63, and n represents the average number of units of the drug-linker structure conjugated to the antibody per antibody, wherein the average number of units of the selected drug-linker structure conjugated per antibody is in the range of from 1 to 10.

In some aspects, the anti-CDH6 antibody comprises a heavy chain comprising the amino acid sequence at positions 20 to 471 in SEQ ID NO: 69 and a light chain comprising the amino acid sequence at positions 21 to 233 in SEQ ID NO: 61.

In some aspects, the heavy chain or the light chain has undergone two or more modifications selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, conversion of N-terminal glutamine or N-terminal glutamic acid to pyroglutamic acid, and a deletion of one or two amino acids from the carboxyl terminus.

In some aspects, the anti-CDH6 antibody lacks a lysine residue at the carboxyl terminus of the heavy chain.

In some aspects, the average number of units of the selected drug-linker structure conjugated per antibody is in the range of from 1 to 10. In some aspects, the average number of units of the selected drug-linker structure conjugated per antibody is in the range of from 2 to 8. In some aspects, the average number of units of the selected drug-linker structure conjugated per antibody is in the range of from 5 to 8. In some aspects, the average number of units of the selected drug-linker structure conjugated per antibody is in the range of from 7 to 8.

In some aspects, a dose of the antibody-drug conjugate in a range of 1.6 mg/kg to 9.6 mg/kg is administered to a subject with cancer. In some aspects, a dose of the antibody-drug conjugate in a range of 3.2 mg/kg to 9.6 mg/kg is administered to a subject with cancer. In some aspects, a dose of the antibody-drug conjugate in a range of 4.8 mg/kg to 9.6 mg/kg is administered to a subject with cancer. In some aspects, a dose of the antibody-drug conjugate in a range of 3.2 mg/kg to 8.0 mg/kg is administered to a subject with cancer. In some aspects, a dose of the antibody-drug conjugate in a range of 3.2 mg/kg to 6.4 mg/kg is administered to a subject with cancer. In some aspects, a dose of the antibody-drug conjugate in a range of 4.8 mg/kg to 8.0 mg/kg is administered to a subject with cancer. In some aspects, a dose of the antibody-drug conjugate in a range of 4.8 mg/kg to 6.4 mg/kg is administered to a subject with cancer. In some aspects, a dose of the antibody-drug conjugate of about 3.2 mg/kg is administered to a subject with cancer. In some aspects, a dose of the antibody-drug conjugate of about 4.8 mg/kg is administered to a subject with cancer. In some aspects, a dose of the antibody-drug conjugate of about 5.4 mg/kg is administered to a subject with cancer. In some aspects, a dose of the antibody-drug conjugate of about 5.6 mg/kg is administered to a subject with cancer. In some aspects, a dose of the antibody-drug conjugate of about 6.4 mg/kg is administered to a subject with cancer. In some aspects, a dose of the antibody-drug conjugate of about 8.0 mg/kg is administered to a subject with cancer. In some aspects, a dose of the antibody-drug conjugate of about 9.6 mg/kg is administered to a subject with cancer.

In some aspects, a dose of the antibody-drug conjugate of 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg or 6.4 mg/kg is administered to a subject with cancer.

In some aspects, the antibody-drug conjugate is administered by intravenous administration.

In some aspects, the antibody-drug conjugate is administered once every 3 weeks.

In some aspects, the cancer is selected from the group consisting of renal cell carcinoma, renal clear cell carcinoma, papillary renal cell carcinoma, ovarian cancer, ovarian serous adenocarcinoma, clear cell carcinoma of ovary, endometrioid carcinoma of ovary, ovarian mucinous tumor, thyroid cancer, bile duct cancer, lung cancer, non-small cell lung cancer, cervix cancer, brain tumor, head and neck cancer, sarcoma, osteosarcoma, small cell lung cancer, glioblastoma, mesothelioma, uterine cancer, pancreatic cancer, Wilms' tumor, neuroblastoma, colorectal cancer, gastric cancer, endometrial cancer, nasopharyngeal cancer, prostate cancer or cancers associated with von Hippel-Lindau disease. In some aspects, the cancer is selected from the group consisting of renal cell carcinoma, renal clear cell carcinoma, papillary renal cell carcinoma, ovarian cancer, ovarian serous adenocarcinoma, thyroid cancer, bile duct cancer, lung cancer, small-cell lung cancer, glioblastoma, mesothelioma, uterine cancer, pancreatic cancer, Wilms' tumor and neuroblastoma. In some aspects, the cancer is ovarian cancer. In some aspects, the ovarian cancer is epithelial ovarian cancer, fallopian tube cancer, or primary peritoneal cancer. In some aspects, the cancer is metastatic. In some aspects, the cancer is renal cell carcinoma, renal clear cell carcinoma or papillary renal cell carcinoma. In some aspects, the cancer is renal cell carcinoma.

In some aspects, the cancer is resistant or refractory. In some aspects, the resistance or refractoriness is resistance or refractoriness acquired by the cancer due to treatment with an anticancer drug. In some aspects, the anticancer drug is a platinum-based chemotherapeutic, a chemotherapeutic, a poly ADP-ribose polymerase (PARP) inhibitor, an immune checkpoint inhibitor (ICI), an angiogenic inhibitor or a vascular endothelial growth factor-tyrosine kinase inhibitor (VEGFR-TKI). In some aspects, the anticancer drug is a platinum-based chemotherapeutic. In some aspects, the platinum-based chemotherapeutic comprises a platinum-based drug and a taxane.

In some aspects, the subject has a cancer that is resistant to platinum-based chemotherapy. In some aspects, the subject exhibits a recurrence of the cancer prior to administration of the ADC. In some aspects, the subject has a cancer that is resistant or refractory to a chemotherapeutic regimen comprising a platinum-based drug and a taxane.

In some aspects, the recurrence of the cancer occurs in less than or within about six months of completion of a chemotherapy regimen comprising a platinum-based drug. In some aspects, the recurrence of the cancer occurs in less than about six months of completion of a chemotherapy regimen comprising a platinum-based drug. In some aspects, the recurrence of the cancer occurs within about six months of completion of a chemotherapy regimen comprising a platinum-based drug.

In some aspects, the recurrence of the cancer occurs in less than or within about six months of completion of a chemotherapy regimen comprising a platinum-based drug and a taxane. In some aspects, the recurrence of the cancer occurs in less than about six months of completion of a chemotherapy regimen comprising a platinum-based drug and a taxane. In some aspects, the recurrence of the cancer occurs within about six months of completion of a chemotherapy regimen comprising a platinum-based drug and a taxane.

In some aspects, the recurrence of the cancer occurs in or after about six months of completion of a chemotherapy regimen comprising a platinum-based drug. In some aspects, the recurrence of the cancer occurs in about six months of completion of a chemotherapy regimen comprising a platinum-based drug. In some aspects, the recurrence of the cancer occurs after about six months of completion of a chemotherapy regimen comprising a platinum-based drug.

In some aspects, the recurrence of the cancer occurs in or after about six months of completion of a chemotherapy regimen comprising a platinum-based drug and a taxane. In some aspects, the recurrence of the cancer occurs in about six months of completion of a chemotherapy regimen comprising a platinum-based drug and a taxane. In some aspects, the recurrence of the cancer occurs after about six months of completion of a chemotherapy regimen comprising a platinum-based drug and a taxane.

In some aspects, the recurrence of the cancer occurs in less than or within six months of completion of a chemotherapy regimen comprising a platinum-based drug. In some aspects, the recurrence of the cancer occurs in less than six months of completion of a chemotherapy regimen comprising a platinum-based drug. In some aspects, the recurrence of the cancer occurs within six months of completion of a chemotherapy regimen comprising a platinum-based drug.

In some aspects, the recurrence of the cancer occurs in less than or within six months of completion of a chemotherapy regimen comprising a platinum-based drug and a taxane. In some aspects, the recurrence of the cancer occurs in less than six months of completion of a chemotherapy regimen comprising a platinum-based drug and a taxane. In some aspects, the recurrence of the cancer occurs within six months of completion of a chemotherapy regimen comprising a platinum-based drug and a taxane.

In some aspects, the recurrence of the cancer occurs in or after six months of completion of a chemotherapy regimen comprising a platinum-based drug. In some aspects, the recurrence of the cancer occurs in six months of completion of a chemotherapy regimen comprising a platinum-based drug. In some aspects, the recurrence of the cancer occurs after six months of completion of a chemotherapy regimen comprising a platinum-based drug.

In some aspects, the recurrence of the cancer occurs in or after six months of completion of a chemotherapy regimen comprising a platinum-based drug and a taxane. In some aspects, the recurrence of the cancer occurs in six months of completion of a chemotherapy regimen comprising a platinum-based drug and a taxane. In some aspects, the recurrence of the cancer occurs after six months of completion of a chemotherapy regimen comprising a platinum-based drug and a taxane.

In some aspects, the subject has a cancer that is resistant or refractory to poly ADP-ribose polymerase (PARP) inhibitors. In some aspects, the subject has a cancer that is resistant or refractory to immune checkpoint inhibitors (ICIs). In some aspects, the subject has a cancer that is resistant or refractory to angiogenic inhibitors. In some aspects, the subject has a cancer that is resistant or refractory to vascular endothelial growth factor-tyrosine kinase inhibitors (VEGFR-TKIs).

In some aspects, the subject has a history of treatment with one or two or more anticancer drugs selected from the group consisting of a platinum-based chemotherapeutic, a chemotherapeutic, a PARP inhibitor, an immune checkpoint inhibitor, an angiogenic inhibitor and a VEGFR-TKI.

In some aspects, the cancer comprises one or more tumors expressing CDH6. In some aspects, the CDH6-expressing cancer is CDH6-overexpressing cancer. In some aspects, the CDH6-overexpressing cancer is cancer given a high score for the expression of CDH6 in an immunohistochemical method. In some aspects, the CDH6-overexpressing cancer is cancer given a high score for the expression of CDH6 in an in situ hybridization method. In some aspects, the CDH6-overexpressing cancer is cancer given a high score for the expression of CDH6 in a next generation sequencing method.

In some aspects, the cancer is an inoperable or recurrent cancer.

In some aspects, the objective response rate of a subject administered a therapeutically effective amount of the ADC is at least about 20%. In some aspects, the objective response rate of a subject administered a therapeutically effective amount of the ADC is at least about 30%. In some aspects, the objective response rate of a subject administered a therapeutically effective amount of the ADC is at least about 40%. In some aspects, the objective response rate of a subject administered a therapeutically effective amount of the ADC is at least about 50%. In some aspects, the objective response rate of a subject administered a therapeutically effective amount of the ADC is at least about 60%.

In some aspects, the progression free survival of a subject administered a therapeutically effective amount of the ADC is at least about 5 months. In some aspects, the progression free survival of a subject administered a therapeutically effective amount of the ADC is at least about 5.5 months. In some aspects, the progression free survival of a subject administered a therapeutically effective amount of the ADC is at least about 5.6 months. In some aspects, the progression free survival of a subject administered a therapeutically effective amount of the ADC is at least about 5.8 months. In some aspects, the progression free survival of a subject administered a therapeutically effective amount of the ADC is at least about 6.0 months. In some aspects, the progression free survival of a subject administered a therapeutically effective amount of the ADC is at least about 7.0 months. In some aspects, the progression free survival of a subject administered a therapeutically effective amount of the ADC is at least about 7.9 months. In some aspects, the progression free survival of a subject administered a therapeutically effective amount of the ADC is at least about 8 months. In some aspects, the progression free survival of a subject administered a therapeutically effective amount of the ADC is at least about 9 months. In some aspects, the progression free survival of a subject administered a therapeutically effective amount of the ADC is at least about 10 months. In some aspects, the progression free survival of a subject administered a therapeutically effective amount of the ADC is at least about 11 months. In some aspects, the progression free survival of a subject administered a therapeutically effective amount of the ADC is at least about 12 months. In some aspects, the progression free survival of a subject administered a therapeutically effective amount of the ADC is at least about 13 months. In some aspects, the progression free survival of a subject administered a therapeutically effective amount of the ADC is at least about 13.9 months.

In some aspects, the median progression free survival of a subject administered a therapeutically effective amount of the ADC is at least about 5 months. In some aspects, the median progression free survival of a subject administered a therapeutically effective amount of the ADC is at least about 5.5 months. In some aspects, the median progression free survival of a subject administered a therapeutically effective amount of the ADC is at least about 5.6 months. In some aspects, the median progression free survival of a subject administered a therapeutically effective amount of the ADC is at least about 5.8 months. In some aspects, the median progression free survival of a subject administered a therapeutically effective amount of the ADC is at least about 6.0 months. In some aspects, the median progression free survival of a subject administered a therapeutically effective amount of the ADC is at least about 7.0 months. In some aspects, the median progression free survival of a subject administered a therapeutically effective amount of the ADC is at least about 7.9 months. In some aspects, the median progression free survival of a subject administered a therapeutically effective amount of the ADC is at least about 8 months. In some aspects, the median progression free survival of a subject administered a therapeutically effective amount of the ADC is at least about 9 months. In some aspects, the median progression free survival of a subject administered a therapeutically effective amount of the ADC is at least about 10 months. In some aspects, the median progression free survival of a subject administered a therapeutically effective amount of the ADC is at least about 11 months. In some aspects, the median progression free survival of a subject administered a therapeutically effective amount of the ADC is at least about 12 months. In some aspects, the median progression free survival of a subject administered a therapeutically effective amount of the ADC is at least about 13 months. In some aspects, the median progression free survival of a subject administered a therapeutically effective amount of the ADC is at least about 13.9 months.

In some aspects, the ADC is administered to the subject as monotherapy.

In some aspects, the subject is administered the ADC with a second drug. In some aspects, the ADC is administered prior to the second drug. In some aspects, the ADC is administered after the second drug. In some aspects, the ADC is administered concurrently with the second drug.

In some aspects, the ADC is administered to the subject as a maintenance therapy.

In some aspects, the ADC is administered to the subject as adjuvant therapy. In some aspects, the ADC is administered to the subject after surgical resection of the tumor.

In some aspects, the ADC is administered to the subject as neoadjuvant therapy. In some aspects, the ADC is administered to the subject prior to surgical resection of the tumor.

In some aspects, the disclosure is generally drawn to an anti-CDH6 antibody-drug conjugate (ADC) for use in treating or preventing cancer, the ADC having the structure represented by the following formula:

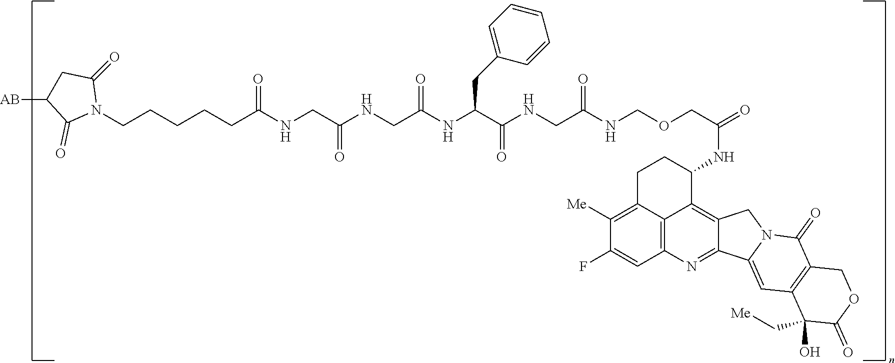

wherein AB represents an anti-CDH6 antibody or the functional fragment of the antibody, n represents the average number of units of the drug-linker structure conjugated to the antibody per antibody, and the antibody is connected to the linker via a sulfhydryl group derived from the antibody; and wherein the ADC is a salt thereof or a hydrate of the ADC or the salt, wherein the average number of units of the drug-linker structure conjugated per antibody is 7 to 8, wherein the antibody comprises: the heavy chain amino acid sequence represented by SEQ ID NO: 87 or an amino acid sequence derived from the amino acid sequence represented by SEQ ID NO: 87 in which one or two amino acids are deleted from the carboxyl terminus thereof; and the light chain amino acid sequence represented by SEQ ID NO: 88, wherein a dose of the antibody-drug conjugate is in a range of 1.6 mg/kg to 8.0 mg/kg, wherein the antibody-drug conjugate is administered by intravenous administration, wherein the antibody-drug conjugate is administered once every 3 weeks.

Also provided herein are pharmaceutical compositions containing the antibody-drug conjugate according to any one of the foregoing aspects or embodiments or a salt thereof as an active component, and a pharmaceutically acceptable formulation component.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF DRAWINGS

FIG. 1 shows a Phase 1 study design for treating renal cell carcinoma (RCC) and ovarian cancer (OVC).

FIG. 2 shows patient demographics and baseline characteristics for the dose escalation part of the Phase 1 study.

FIG. 3 shows the number of patients in the dose escalation part of the Phase 1 study with treatment-emergent adverse events (TEAEs), which occurred in 210% of patients, regardless of causality.

FIG. 4 shows the number of patients in the dose escalation part of the Phase 1 study with treatment-emergent adverse events (TEAEs), which occurred in 210% of patients, related to DS-6000a.

FIG. 5 shows the tumor response for the subjects (n=24) in the dose escalation part of the Phase 1 study.

FIG. 6 shows the percent change in sum of target lesion from baseline in the dose escalation part of the Phase 1 study. The top panel shows a spider plot including all dosing group. The bottom panels show a spider plot divided into each dosing group.

FIG. 7 shows CA-125 response for the OVC subjects (n=17) in the dose escalation part of the Phase 1 study.

FIG. 8 shows the percent change of CA-125 from baseline in the dose escalation part of the Phase 1 study. The top panel shows a spider plot including all dosing group. The bottom panels show a spider plot divided into each dosing group.

FIG. 9 shows a reduction in the size of target lesions in RCC patient treated with 3.2 mg/kg of DS-6000a.

FIG. 10 shows a reduction in the size of target lesions in OVC patient treated with 6.4 mg/kg of DS-6000a.

FIG. 11 shows the best percent change in sum of longest dimensions from baseline in the dose escalation part of the Phase 1 study.

FIG. 12 shows the updated Phase 1 study design for treating RCC and OVC.

FIG. 13 shows patient demographics and base line characteristics in dose escalation and expansion parts of the Phase 1 study.

FIG. 14 shows the number of patients in the dose escalation and expansion parts of the Phase 1 study with TEAE, which occurred in 215% of patients, regardless of causality.

FIG. 15 shows the best percent change in sum of longest dimensions from baseline for OVC subjects in dose escalation and expansion parts of the Phase 1 study.

FIG. 16 shows the percent change in sum of target lesion from baseline for OVC subjects in the dose escalation and expansion parts of the Phase 1 study. The top panel shows a spider plot including all dosing group. The bottom panels show a spider plot divided into each dosing group.

FIG. 17 shows the best overall tumor response for the OVC subjects (n=44) in the dose escalation and expansion parts of the Phase 1 study.

FIG. 18 shows the percent change of CA-125 from baseline for OVC subjects in the dose escalation and expansion parts of the Phase 1 study. The top panel shows a spider plot including all dosing group. The bottom panels show a spider plot divided into each dosing group.

FIG. 19 shows the best percent change in CA-125 from baseline for OVC subjects in the dose escalation and expansion parts of the Phase 1 study.

FIG. 20 shows the progression-free survival for OVC subjects in the dose escalation and expansion parts of the Phase 1 study.

FIG. 21 shows the objective response rate for OVC subjects in the dose escalation and expansion parts of the Phase 1 study.

### DESCRIPTION OF EMBODIMENTS

Hereinafter, the preferred embodiments for carrying out the disclosed methods and uses will be described with reference to the drawings. It is to be noted that the embodiments described below merely illustrate the representative embodiments, and the scope of the present disclosure shall not be narrowly interpreted due to these examples.

It is to be understood that methods are not limited to the particular embodiments described, and as such may vary. It is also to be understood that the terminology used herein is for the purpose of describing particular embodiments only, and is not intended to be limiting. The scope of the present technology will be limited only by the appended claims.

Unless defined otherwise, all technical and scientific terms used herein have the same meaning as commonly understood by one of ordinary skill in the art. Although any methods and materials similar or equivalent to those described herein can also be used in the practice or testing of the disclosed methods and uses, representative illustrative methods and materials are now described.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As used in the specification and claims, the singular form “a,” “an” and “the” include singular and plural references unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10% as well as the specified number. For example, “about 10” should be understood as both “10” and “9-11.”

As used herein, “optional” or “optionally” means that the subsequently described event or circumstance may or may not occur, and that the description includes instances where said event or circumstance occurs and instances where it does not.

The terms “individual,” “subject,” and “patient” are used interchangeably herein, and refer to any individual mammal, e.g., bovine, canine, feline, equine, simian, porcine, camelid, bat, or human, being treated according to the disclosed methods or uses. In preferred embodiments, the subject is a human.

As used herein, the phrases “effective amount,” “therapeutically effective amount,” and “therapeutic level” mean the dosage or concentration in a subject that provides the specific pharmacological effect for which the ADC is administered in a subject in need of such treatment, i.e. to treat or prevent a cancer (e.g., renal cell carcinoma, a ovarian cancer, CDH6-expressing cancer, or a resistant or refractory cancer). It is emphasized that a therapeutically effective amount or therapeutic level of an ADC will not always be effective in treating the cancers described herein, even though such dosage is deemed to be a therapeutically effective amount by those of skill in the art. For convenience only, exemplary dosages, drug delivery amounts, therapeutically effective amounts, and therapeutic levels are provided below. Those skilled in the art can adjust such the amount in accordance with standard practices as needed to treat a specific subject and/or condition. The therapeutically effective amount may vary based on the route of administration and dosage form, the age and weight of the subject, and/or the subject's condition, including the type and severity of the cancer.

The terms “treatment” or “treating” as used herein with reference to a cancer refer to reducing, suppressing, or eliminating the cancer; reducing, suppressing, or eliminating cancer cell growth; reducing, suppressing, or eliminating spread of the cancer; or causing a tumor or metastasis to regress or die. Treatment and treating may also, optionally, mean improving quality of life or overall survival of a subject, even if cancer cell growth is not inhibited and/or the cancer does not die.

The terms “prevent” or “preventing” as used herein with reference to a cancer refer to delaying or preventing the occurrence of metastasis (i.e., growth of cancer in secondary sites where the cancer is not present at the commencement of treatment), as well as delaying or preventing recurrence of a cancer if a subject achieves remission or a cancer/tumor is completely destroyed or killed.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, for example, Martin, Remington's Pharmaceutical Sciences, 15th Ed., Mack Publ. Co., Easton, PA.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

In the present description, the term “cancer” is used to have the same meaning as that of the term “tumor”.

In the present description, the term “gene” is used to include not only DNA but also its mRNA and cDNA, and cRNA thereof.

In the present description, the term “polynucleotide” or “nucleotide” is used to have the same meaning as that of a nucleic acid, and also includes DNA, RNA, a probe, an oligonucleotide, and a primer. In the present description, the terms “polynucleotide” and “nucleotide” can be used interchangeably with each other unless otherwise specified.

In the present description, the terms “polypeptide” and “protein” can be used interchangeably with each other.

In the present description, the term “cell” includes cells in an individual animal, and cultured cells.

In the present description, the term “CDH6” can be used to have the same meaning as that of the CDH6 protein. In the present description, human CDH6 is also referred to as “hCDH6”.

In the present description, the term “cytotoxic activity” is used to mean that a pathologic change is caused to cells in any given way. The term not only means a direct trauma, but also means all types of structural or functional damage caused to cells, such as DNA cleavage, formation of a base dimer, chromosomal cleavage, damage to cell mitotic apparatus, and a reduction in the activities of various types of enzymes.

In the present description, the phrase “exerting toxicity in cells” is used to mean that toxicity is exhibited in cells in any given way. The term not only means a direct trauma, but also means all types of structural, functional, or metabolic influences caused to cells, such as DNA cleavage, formation of a base dimer, chromosomal cleavage, damage to cell mitotic apparatus, a reduction in the activities of various types of enzymes, and suppression of effects of cell growth factors.

In the present description, the term “functional fragment of an antibody”, also called “antigen-binding fragment of an antibody”, is used to mean a partial fragment of the antibody having binding activity against an antigen, and includes Fab, F(ab′)2, scFv, a diabody, a linear antibody and a multispecific antibody formed from antibody fragments, and the like. Fab′, which is a monovalent fragment of antibody variable regions obtained by treating F(ab′)2 under reducing conditions, is also included in the antigen-binding fragment of an antibody. However, the antigen-binding fragment of an antibody is not limited to these molecules, as long as the antigen-binding fragment has antigen-binding ability. These antigen-binding fragments include not only those obtained by treating a full-length molecule of an antibody protein with an appropriate enzyme, but proteins produced in appropriate host cells using a genetically engineered antibody gene.

In the present description, the term “epitope” is used to mean the partial peptide or partial three-dimensional structure of CDH6, to which a specific anti-CDH6 antibody binds. Such an epitope, which is the above-described partial peptide of CDH6, can be determined by a method well known to a person skilled in the art, such as an immunoassay. First, various partial structures of an antigen are produced. As regards production of such partial structures, a known oligopeptide synthesis technique can be applied. For example, a series of polypeptides, in which CDH6 has been successively truncated at an appropriate length from the C-terminus or N-terminus thereof, are produced by a genetic recombination technique well known to a person skilled in the art. Thereafter, the reactivity of an antibody to such polypeptides is studied, and recognition sites are roughly determined. Thereafter, further shorter peptides are synthesized, and the reactivity thereof to these peptides can then be studied, so as to determine an epitope. When an antibody binding to a membrane protein having a plurality of extracellular domains is directed to a three-dimensional structure composed of a plurality of domains as an epitope, the domain to which the antibody binds can be determined by modifying the amino acid sequence of a specific extracellular domain, and thereby modifying the three-dimensional structure. The epitope, which is a partial three-dimensional structure of an antigen that binds to a specific antibody, can also be determined by specifying the amino acid residues of an antigen adjacent to the antibody by X-ray structural analysis.

In the present description, the phrase “antibodies binding to the same epitope” is used to mean antibodies that bind to a common epitope. If a second antibody binds to a partial peptide or a partial three-dimensional structure to which a first antibody binds, it can be determined that the first antibody and the second antibody bind to the same epitope. Alternatively, by confirming that a second antibody competes with a first antibody for the binding of the first antibody to an antigen (i.e., a second antibody interferes with the binding of a first antibody to an antigen), it can be determined that the first antibody and the second antibody bind to the same epitope, even if the specific sequence or structure of the epitope has not been determined. In the present description, the phrase “binding to the same epitope” refers to the case where it is determined that the first antibody and the second antibody bind to a common epitope by any one or both of these determination methods. When a first antibody and a second antibody bind to the same epitope and further, the first antibody has special effects such as antitumor activity or internalization activity, the second antibody can be expected to have the same activity as that of the first antibody.

In the present description, the term “CDR” is used to mean a complementarity determining region. It is known that the heavy chain and light chain of an antibody molecule each have three CDRs. Such a CDR is also referred to as a hypervariable region, and is located in the variable regions of the heavy chain and light chain of an antibody. These regions have a particularly highly variable primary structure and are separated into three sites on the primary structure of the polypeptide chain in each of the heavy chain and light chain. In the present description, with regard to the CDR of an antibody, the CDRs of a heavy chain are referred to as CDRH1, CDRH2 and CDRH3, respectively, from the amino-terminal side of the amino acid sequence of the heavy chain, whereas the CDRs of a light chain are referred to as CDRL1, CDRL2 and CDRL3, respectively, from the amino-terminal side of the amino acid sequence of the light chain. These sites are located close to one another on the three-dimensional structure, and determine the specificity of the antibody to an antigen to which the antibody binds.

In the present disclosure, the phrase “hybridizing under stringent conditions” is used to mean that hybridization is carried out in the commercially available hybridization solution ExpressHyb Hybridization Solution (manufactured by Clontech Laboratories, Inc.) at 68° C., or that hybridization is carried out under conditions in which hybridization is carried out using a DNA-immobilized filter in the presence of 0.7 to 1.0 M NaCl at 68° C., and the resultant is then washed at 68° C. with a 0.1- to 2-fold concentration of SSC solution (wherein 1-fold concentration of SSC consists of 150 mM NaCl and 15 mM sodium citrate) for identification, or conditions equivalent thereto.

In the present description, the term “one to several” is used to mean 1 to 10, 1 to 9, 1 to 8, 1 to 7, 1 to 6, 1 to 5, 1 to 4, 1 to 3, or 1 or 2.

In the present description, the term “anticancer drug” is used to mean a drug for the purpose of suppressing, preventing, inhibiting and/or slowing the proliferation, the growth and/or the spread of cancer cells or tumor cells. In the present description, the term “anticancer drug” have the same meaning as that of the term “anticancer agent”, “antitumor agent” or “antitumor drug”.

In the present description, the term “resistant” is used to mean having non-response to treatment with an anticancer drug. The term can also be expressed as “refractory”, “non-responsive”, or “unresponsive”. Furthermore, the term can also be expressed as “intolerant” because tumor growth cannot be prevented due to the non-responsive property. In the present description, the term “resistant” may also be used when cancer of a subject exhibits low sensitivity to treatment with an anticancer drug, cancer cells do not disappear or shrink, CR or PR is not achieved, and/or cancer cells progressed earlier (for example, in less than or within 6 months regarding ovarian cancer) after treatment with the anticancer drug.

In the present description, the term “resistant” may be “having resistance acquired by the cancer due to treatment with an anticancer drug” or may be “having resistance intrinsic to the cancer independently of treatment with an anticancer drug”.

In the present description, the term “chemotherapy” is used to mean a therapy using one or more chemotherapeutics used to treat cancer.

In the present description, the term “chemotherapeutic” is used to mean a chemotherapeutic agent used to treat cancer. Chemotherapeutic includes but are not limited to: alkylating agents (for example, mechlorethamine, cyclophosphamide, ifosfamide, melphalan, chlorambucil, hexamethylmelamine, thiotepa, busulfan, carmustine, lomustine, semustine, streptozocin, dacarbazine), antimetabolites (for example, gemcitabine, methotrexate, fluorouracil, doxifluridine, capecitabine, floxuridine, cytarabine, mercaptopurine, thioguanine, pentostatin), vinca alkaloids (for example, vinblastine, vincristine), epipodophyllotoxins (for example, etoposide, teniposide), antibiotics (for example, dactinomycin, daunorubicin, doxorubicin, bleomycin, plicamycin, mitomycin), platinum complexes (for example, cisplatin, carboplatin, oxaliplatin), taxanes (for example, paclitaxel, docetaxel), anthracenediones (for example, mitoxantrone), substituted ureas (for example, hydroxyurea), methylhydrazines (for example, procarbazine hydrochloride), vitamin A metabolite (for example, tretinoin), and pharmacologically acceptable salts thereof.

In the present description, the term “platinum-based chemotherapy” is used to mean a cancer therapy using one or more platinum-based drug with/without other one or more chemotherapeutics.

In the present description, the term “platinum-based chemotherapeutic” is used to mean a platinum-based drug or a set of drugs used in platinum-based chemotherapy.

In the present description, the term “platinum-based drug” is used to mean platinum complexes used to treat cancer. Platinum-based drug includes but are not limited to: cisplatin, carboplatin and oxaliplatin.

In the present description, the term “PARP inhibitor” is used to mean a drug that has the function of inhibiting PARP (poly[adenosine-5′-diphosphate (ADP)-ribose]polymerase), and thus preventing single-strand break repair (Benafif S, et al., Onco. Targets Ther. (2015) 8, 519-528) (Fong P C, et al., N. Engl. J. Med. (2009) 361, 123-134) (Gelmon K A, et al., Lancet Oncol. (2011) 12, 852-861). PARP includes multiple subtypes, but the PARP inhibitor in the present invention preferably inhibits PARP-1 and PARP-2. The PARP inhibitor includes but are not limited to: olaparib (Menear K A, et al., J. Med. Chem. (2008) 51, 6581-6591), rucaparib (Gillmore A T, et al., Org. Process Res. Dev. (2012) 16, 1897-1904), niraparib (Jones P, et al., J. Med. Chem. (2009) 52, 7170-7185), talazoparib (Shen Y, et al., Clin. Cancer Res. (2013) 19 (18), 5003-5015), veliparib, pamiparib, and fluzoparib, and pharmacologically acceptable salts thereof. Olaparib, rucaparib, niraparib, talazoparib, and veliparib, and pharmacologically acceptable salts thereof can be preferably exemplified.

In the present description, the term “immune checkpoint inhibitor” is used to mean an agent which inhibits the immune suppression system to activate tumor immunity. The immune checkpoint inhibitor includes but are not limited to: an anti-PD-1 antibody, an anti-PD-L1 antibody, and an anti-CTLA-4 antibody. An anti-PD-1 antibody and an anti-PD-L1 antibody can be preferably exemplified.

In the present description, the term “anti-PD-1 antibody” is used to mean to an antibody which specifically binds to PD-1 (Programmed cell death-1; CD279; PDCD1), or a functional fragment of the antibody, wherein the antibody has an activity of reducing, inhibiting, and/or interfering with signal transduction caused by interaction between PD-1 and PD-L1 or PD-L2 as a binding partner. The anti-PD-1 antibody includes but are not limited to: nivolumab (International Publication No. WO 2006/121168, etc.), pembrolizumab (International Publication No. WO 2008/156712, etc.), tislelizumab, sintilimab, spartalizumab and cemiplimab.

In the present description, the term “anti-PD-L1 antibody” is used to mean an antibody which specifically binds to PD-L1 (Programmed cell death ligand 1; CD274; B7-H1), or a functional fragment of the antibody, wherein the antibody has an activity of reducing, inhibiting, and/or interfering with signal transduction caused by interaction between PD-L1 and PD-1 or B7.1 (CD80) as a binding partner. The anti-PD-L1 antibody includes but not are limited: atezolizumab (International Publication No. WO 2010/077634, etc.), durvalumab (International Publication No. WO 2011/066389, etc.), and avelumab (International Publication No. WO 2013/079174, etc.).

In the present description, the term “anti-CTLA-4 antibody” is used to mean an antibody which specifically binds to CTLA-4 (Cytotoxic T-lymphocyte-associated protein 4; CD152), or a functional fragment of the antibody, wherein the antibody has an activity of reducing, inhibiting, and/or interfering with signal transduction caused by interaction between CTLA-4 and B7.1 (CD80) or B7.2 (CD86) as a binding partner. The anti-CTLA-4 antibody includes but not are limited to: ipilimumab (International Publication No. WO 2001/014424, etc.) and tremelimumab (International Publication No. WO 2000/037504, etc.).

In the present description, the term “angiogenic inhibitor” is used to mean an agent which inhibits the formation and/or growth of new blood vessels, either directly or indirectly, regardless of mechanism. A VEGF (Vascular Endothelial Growth Factor) inhibitor can be preferably exemplified.

In the present description, the term “VEGF inhibitor” is used to mean an agent which inhibits an interaction between VEGF and VEGF receptor. The VEGF inhibitor includes but not limited to: an anti-VEGF antibody, an anti-VEGF receptor antibody, a fusion protein comprising an extracellular domain of a VEGF receptor and a multispecific antibody having a binding specificity for VEGF.

In the present description, the term “an anti-VEGF antibody” is used to mean an antibody which specifically binds to VEGF, or a functional fragment of the antibody. The anti-VEGF antibody includes but are not limited to: bevacizumab and sevacizumab. Bevacizumab can be preferably exemplified.

In the present description, the term “an anti-VEGF receptor antibody” is used to mean an antibody which specifically binds to VEGF receptor, or a functional fragment of the antibody. The anti-VEGF receptor antibody includes but are not limited to: ramucirumab.

In the present description, the term “a fusion protein comprising an extracellular domain of a VEGF receptor” is used to mean a fusion protein comprising a binding domain to VEGF, wherein the binding domain is derived from an extracellular domain of a VEGF receptor. The fusion protein comprising an extracellular domain of a VEGF receptor includes but not limited to: aflibercept.

In the present description, the term “a multispecific antibody having a binding specificity for VEGF” is used to mean an antibody comprising binding specificities for at least two different sites, wherein one of the binding specificities is for VEGF and the other or others is/are for any other antigen. The multispecific antibody having a binding specificity for VEGF includes but are not limited to: ivonescimab (Esfandiari et al., Nat. Rev. Drug Discov. (2022) 21 (6): 411-412), CTX-009 (also referred as ABL-001, Xu et al., Cancer Lett. (2022) 538:215699), BI836880 (Clin. Exp. Metastasis (2020) 37 (6): 637-648), navicixizumab (Esfandiari et al., Nat. Rev. Drug Discov. (2022) 21 (6): 411-412) and vanucizumab (Xu et al., Cancer Lett. (2022) 538:215699).

In the present description, the term “VEGFR-TKI” is used to mean an agent that inhibits tyrosine kinase of a vascular endothelial growth factor receptor (VEGFR). The VEGFR-TKI may have an effect of inhibiting a kinase other than VEGFR tyrosine kinase.

The VEGFR-TKI includes but not are limited: regorafenib, sorafenib, vandetanib (U.S. Pat. No. 7,173,038), sunitinib (U.S. Pat. No. 6,573,293), axitinib (U.S. Pat. No. 6,534,524), pazopanib (U.S. Pat. No. 7,105,530), lenvatinib (U.S. Pat. No. 7,253,286), nintedanib (hereinafter, also referred to as “BIBF1120”) (U.S. Pat. No. 6,762,180), cabozantinib (U.S. Pat. No. 7,579,473), tivozanib (International Publication No. WO 2002/088110), brivanib (International Publication No. WO 2004/009784), linifanib (International Publication No. WO 2014/022975), lucitanib (International Publication No. WO 2008/112408), ilorasertib, and ENMD-2076, and pharmacologically acceptable salts thereof.

In the present description, the term “recurrence of the cancer” is used to mean coming back of the cancer to the same place as the primary tumor or to another place in the body, after a period of time during which the cancer could not be detected. The term is defined on the basis of “recurrence” in the following reference.

NCI Dictionaries, “recurrence”, NCI Dictionary of Cancer Terms [online]. National Cancer Institute [retrieved on 2022 Sep. 6]. Retrieved from <cancer.gov/publications/dictionaries/cancer-terms/def/recurrence>.

In the present description, the term “a chemotherapy regimen” is used to mean a treatment plan for chemotherapy which defines drug(s), dosage, frequency, and so on.

In the present description, the term “complete response (CR)” is used to mean that all signs of cancer disappeared in response to treatment. “complete response (CR)” does not always mean the cancer has been cured. The term can also be expressed as “complete remission”. The term is defined on the basis of “complete response” in the following reference. NCI Dictionaries, “complete response”, NCI Dictionary of Cancer Terms [online]. National Cancer Institute [retrieved on 2022 Sep. 6]. Retrieved from <cancer.gov/publications/dictionaries/cancer-terms/def/complete-response>.

In the present description, the term “partial response (PR)” is used to mean that the size of a tumor or the extent of cancer in the body decreases in response to treatment. The term can also be expressed as “partial remission”. The term is defined on the basis of “partial response” in the following reference.

NCI Dictionaries, “partial response”, NCI Dictionary of Cancer Terms [online]. National Cancer Institute [retrieved on 2022 Sep. 6]. Retrieved from <cancer.gov/publications/dictionaries/cancer-terms/def/partial-response>.

In the present description, the term “stable disease (SD)” is used to mean that cancer is neither decreasing nor increasing in extent or severity. The term is defined on the basis of “stable disease” in the following reference.

NCI Dictionaries, “stable disease”, NCI Dictionary of Cancer Terms [online]. National Cancer Institute [retrieved on 2022 Sep. 6]. Retrieved from <cancer.gov/publications/dictionaries/cancer-terms/def/stable-disease>.

In the present description, the term “progressive disease” or “PD” is used to mean cancer that is growing, spreading, or getting worse. The term is defined on the basis of “progressive disease” in the following reference.

NCI Dictionaries, “progressive disease”, NCI Dictionary of Cancer Terms [online]. National Cancer Institute [retrieved on 2023 Apr. 2]. Retrieved from <cancer.gov/publications/dictionaries/cancer-terms/def/progressive-disease>.

In the present description, the term “objective response rate” or “ORR” is used to mean the percentage of people in a study or treatment group who have a partial response (PR) or complete response (CR) to the treatment within a certain period of time. The term is defined on the basis of “ORR” in the following reference.

NCI Dictionaries, “ORR”, NCI Dictionary of Cancer Terms [online]. National Cancer Institute [retrieved on 2023 Apr. 2]. Retrieved from <cancer.gov/publications/dictionaries/cancer-terms/def/orr>.

In the present description, the term “progression free survival” or “PFS” is used to mean the length of time during and after the treatment of a disease, such as cancer, that a patient lives with the disease but it does not get worse. The term is defined on the basis of “PFS” in the following reference.

NCI Dictionaries, “PFS”, NCI Dictionary of Cancer Terms [online]. National Cancer Institute [retrieved on 2023 Apr. 2]. Retrieved from <cancer.gov/publications/dictionaries/cancer-terms/def/pfs>.

In the present description, the term “maintenance therapy” is used to mean treatment that is given to help keep cancer from coming back after it has disappeared following the initial therapy. The term is defined on the basis of “maintenance therapy” in the following reference.

NCI Dictionaries, “maintenance therapy”, NCI Dictionary of Cancer Terms [online]. National Cancer Institute [retrieved on 2023 Apr. 10]. Retrieved from <cancer.gov/publications/dictionaries/cancer-terms/def/maintenance-therapy>.

In the present description, the term “adjuvant therapy” is used to mean additional cancer treatment given after the primary treatment to lower the risk that the cancer will come back. The term is defined on the basis of “adjuvant therapy” in the following reference.

NCI Dictionaries, “adjuvant therapy”, NCI Dictionary of Cancer Terms [online]. National Cancer Institute [retrieved on 2023 Apr. 10]. Retrieved from <cancer.gov/publications/dictionaries/cancer-terms/def/adjuvant-therapy>.

In the present description, the term “neoadjuvant therapy” is used to mean treatment given as a first step to shrink a tumor before the main treatment such as surgery is given. The term is defined on the basis of “neoadjuvant therapy” in the following reference.

NCI Dictionaries, “neoadjuvant therapy”, NCI Dictionary of Cancer Terms [online]. National Cancer Institute [retrieved on 2023 Apr. 10]. Retrieved from <cancer.gov/publications/dictionaries/cancer-terms/def/neoadjuvant-therapy>.

As the amino acid substitution in this specification, a conservative amino acid substitution is preferred. The conservative amino acid substitution refers to a substitution occurring within a group of amino acids related to amino acid side chains. Preferred amino acid groups are as follows: an acidic group (aspartic acid and glutamic acid); a basic group (lysine, arginine, and histidine); a non-polar group (alanine, valine, leucine, isoleucine, proline, phenylalanine, methionine, and tryptophan); and an uncharged polar family (glycine, asparagine, glutamine, cysteine, serine, threonine, and tyrosine). More preferred amino acid groups are as follows: an aliphatic hydroxyl group (serine and threonine); an amide-containing group (asparagine and glutamine); an aliphatic group (alanine, valine, leucine, and isoleucine); and an aromatic group (phenylalanine, tryptophan, and tyrosine). Such an amino acid substitution is preferably performed within a range which does not impair the properties of a substance having the original amino acid sequence.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

### 1. CDH6

Cadherins are glycoproteins present on the surface of cell membranes and function as cell-cell adhesion molecules through the calcium ion-dependent binding of their N-terminal extracellular domains, or as signal molecules responsible for cell-cell interaction. Classic cadherins are in the cadherin superfamily and are single-pass transmembrane proteins composed of five extracellular domains (EC domains), one transmembrane region, and an intracellular domain.

CDH6 (cadherin-6) is a single-pass transmembrane protein composed of 790 amino acids, which is classified into the type II cadherin family, and this protein has N-terminal extracellular and C-terminal intracellular domains. The human CDH6 gene was cloned for the first time in 1995 (Non Patent Literature 1), and its sequence can be referred to under, for example, accession Nos. NM 004932 and NP_004923 (NCBI).

The CDH6 protein used in the present disclosure can be directly purified from the CDH6-expressing cells of a human or a non-human mammal (e.g., a rat, a mouse or a monkey) and can then be used, or a cell membrane fraction of the aforementioned cells can be prepared and can be used as the CDH6 protein. Alternatively, CDH6 can also be obtained by synthesizing it in vitro, or by allowing host cells to produce CDH6 by genetic manipulation. According to such genetic manipulation, the CDH6 protein can be obtained, specifically, by incorporating CDH6 CDNA into a vector capable of expressing the CDH6 CDNA, and then synthesizing CDH6 in a solution containing enzymes, substrate and energetic materials necessary for transcription and translation, or by transforming the host cells of other prokaryotes or eukaryotes, so as to allow them to express CDH6. Also, CDH6-expressing cells based on the above-described genetic manipulation, or a cell line expressing CDH6 may be used to present the CDH6 protein. Alternatively, the expression vector into which CDH6 CDNA has been incorporated can be directly administered to an animal to be immunized, and CDH6 can be expressed in the body of the animal thus immunized.

Moreover, a protein which consists of an amino acid sequence comprising a substitution, deletion and/or addition of one or several amino acids in the above-described amino acid sequence of CDH6, and has a biological activity equivalent to that of the CDH6 protein, is also included within the term “CDH6”.

The human CDH6 protein has the amino acid sequence shown in SEQ ID NO: 1. The extracellular region of the human CDH6 protein is composed of extracellular domain 1 (in the present description, also referred to as EC1) having the amino acid sequence at positions 54 to 159 in the amino acid sequence shown in SEQ ID NO: 1, extracellular domain 2 (in the present description, also referred to as EC2) having the amino acid sequence at positions 160 to 268 in the amino acid sequence shown in SEQ ID NO: 1, extracellular domain 3 (in the present description, also referred to as EC3) having the amino acid sequence at positions 269 to 383 in the amino acid sequence shown in SEQ ID NO: 1, extracellular domain 4 (in the present description, also referred to as EC4) having the amino acid sequence at positions 384 to 486 in the amino acid sequence shown in SEQ ID NO: 1, and extracellular domain 5 (in the present description, also referred to as EC5) having the amino acid sequence at positions 487 to 608 in the amino acid sequence shown in SEQ ID NO: 1. The amino acid sequences of EC1 to EC5 are shown in SEQ ID NOs: 2 to 6, respectively (Table 1).

### 2. Production of Anti-CDH6 Antibody

One example of the anti-CDH6 antibody of the present disclosure can include an anti-CDH6 antibody which recognizes an amino acid sequence comprising the amino acid sequence shown in SEQ ID NO: 4, and has internalization activity. One example of the anti-CDH6 antibody of the present disclosure can include an anti-CDH6 antibody which specifically recognizes an amino acid sequence comprising the amino acid sequence shown in SEQ ID NO: 4, and has internalization activity. One example of the anti-CDH6 antibody of the present disclosure can include an anti-CDH6 antibody which recognizes an amino acid sequence consisting of the amino acid sequence shown in SEQ ID NO: 4, and has internalization activity. One example of the anti-CDH6 antibody of the present disclosure can include an anti-CDH6 antibody which specifically recognizes an amino acid sequence consisting of the amino acid sequence shown in SEQ ID NO: 4, and has internalization activity. The phrase “specifically recognize an amino acid sequence comprising the amino acid sequence shown in SEQ ID NO: 4” or “specifically recognize an EC3 domain” as applied to an antibody is used to mean that the antibody strongly recognizes or strongly binds to the EC3 domain of CDH6 compared with the other extracellular domains of CDH6.

The anti-CDH6 antibody of the present disclosure may be derived from any species. Preferred examples of the species can include humans, monkeys, rats, mice and rabbits. When the anti-CDH6 antibody of the present disclosure is derived from a species other than humans, it is preferred to chimerize or humanize the anti-CDH6 antibody by a well-known technique. The antibody of the present disclosure may be a polyclonal antibody or may be a monoclonal antibody, and a monoclonal antibody is preferred.

The anti-CDH6 antibody of the present disclosure is an antibody that can target tumor cells. Specifically, the anti-CDH6 antibody of the present disclosure possesses the property of being able to recognize tumor cells, the property of being able to bind to tumor cells, and/or the property of being internalized into tumor cells by cellular uptake, and the like. Accordingly, the anti-CDH6 antibody of the present disclosure can be conjugated to a compound having antitumor activity via a linker to prepare an antibody-drug conjugate.

The binding activity of an antibody against tumor cells can be confirmed by flow cytometry. The uptake of an antibody into tumor cells can be confirmed by (1) an assay of visualizing a cellularly taken-up antibody under a fluorescent microscope using a secondary antibody (fluorescently labeled) binding to the antibody (Cell Death and Differentiation, 2008, 15, 751-761), (2) an assay of measuring the amount of cellularly taken-up fluorescence using a secondary antibody (fluorescently labeled) binding to the antibody (Molecular Biology of the Cell Vol. 15, 5268-5282 December 2004) or (3) a Mab-ZAP assay using an immunotoxin binding to the antibody, wherein the toxin is released upon cellular uptake, so as to suppress cell growth (Bio Techniques 28:162-165, January 2000). A recombinant conjugated protein of a catalytic region of diphtheria toxin and protein G may be used as the immunotoxin.

In the present description, the term “high internalization ability” is used to mean that the survival rate (which is indicated by a ratio relative to a cell survival rate without antibody addition defined as 100%) of CDH6-expressing cells to which the aforementioned antibody and a saporin-labeled anti-rat IgG antibody have been administered is preferably 70% or less, and more preferably 60% or less.

The antitumor antibody-drug conjugate of the present disclosure comprises a conjugated compound exerting an antitumor effect. Therefore, it is preferred, but not essential, that the antibody itself should have an antitumor effect. For the purpose of specifically and/or selectively exerting the cytotoxicity of the antitumor compound in tumor cells, it is important and preferred that the antibody should have a property of being internalized and transferred into tumor cells.

The anti-CDH6 antibody can be obtained by immunizing an animal with a polypeptide serving as an antigen by a method usually performed in this field, and then collecting and purifying an antibody produced in a living body thereof. It is preferred to use CDH6 retaining a three-dimensional structure as an antigen. Examples of such a method can include a DNA immunization method.

The origin of the antigen is not limited to a human, and thus, an animal can also be immunized with an antigen derived from a non-human animal such as a mouse or a rat. In this case, an antibody applicable to the disease of a human can be selected by examining the cross-reactivity of the obtained antibody binding to the heterologous antigen with the human antigen.

Furthermore, antibody-producing cells that produce an antibody against the antigen can be fused with myeloma cells according to a known method (e.g., Kohler and Milstein, Nature (1975) 256, 495-497; and Kennet, R. ed., Monoclonal Antibodies, 365-367, Plenum Press, N. Y. (1980)) to establish hybridomas, so as to obtain a monoclonal antibody.

Hereinafter, the method for obtaining an antibody against CDH6 will be specifically described.

### (1) Preparation of Antigen

The antigen can be obtained by allowing host cells to produce a gene encoding the antigen protein according to genetic manipulation. Specifically, a vector capable of expressing the antigen gene is produced, and the vector is then introduced into host cells, so that the gene is expressed therein, and thereafter, the expressed antigen may be purified. The antibody can also be obtained by a method of immunizing an animal with the antigen-expressing cells based on the above-described genetic manipulation, or a cell line expressing the antigen.

Alternatively, the antibody can also be obtained, without the use of the antigen protein, by incorporating cDNA of the antigen protein into an expression vector, then administering the expression vector to an animal to be immunized, and expressing the antigen protein in the body of the animal thus immunized, so that an antibody against the antigen protein is produced therein.

### (2) Production of Anti-CDH6 Monoclonal Antibody

The anti-CDH6 antibody used in the present disclosure is not particularly limited. For example, an antibody specified by an amino acid sequence shown in the sequence listing of the present application can be suitably used. The anti-CDH6 antibody used in the present disclosure is desirably an antibody having the following properties:
(1) an antibody having the following properties:
(a) specifically binding to CDH6, and
(b) having the activity of being internalized into CDH6-expressing cells by binding to CDH6;
(2) the antibody according to the above (1), wherein the CDH6 is human CDH6; or
(3) the antibody according to the above (1) or (2), wherein the antibody specifically recognizes EC3 of human CDH6, and has internalization activity.

The method for obtaining the antibody against CDH6 of the present disclosure is not particularly limited as long as an anti-CDH6 antibody can be obtained. It is preferred to use CDH6 retaining its conformation as an antigen.

One preferred example of the method for obtaining the antibody can include a DNA immunization method. The DNA immunization method is an approach which involves transfecting an animal (e.g., mouse or rat) individual with an antigen expression plasmid, and then expressing the antigen in the individual to induce immunity against the antigen. The transfection approach includes a method of directly injecting the plasmid to the muscle, a method of injecting a transfection reagent such as a liposome or polyethylenimine to the vein, an approach using a viral vector, an approach of injecting gold particles attached with the plasmid using a gene gun, a hydrodynamic method of rapidly injecting a plasmid solution in a large amount to the vein, and the like. With regard to the transfection method of injecting the expression plasmid to the muscle, a technique called in vivo electroporation, which involves applying electroporation to the intramuscular injection site of the plasmid, is known as an approach for improving expression levels (Aihara H, Miyazaki J. Nat Biotechnol. 1998 September; 16 (9): 867-70 or Mir L M, Bureau MF, Gehl J, Rangara R, Rouy D, Caillaud J M, Delaere P, Branellec D, Schwartz B, Scherman D. Proc Natl Acad Sci USA. 1999 Apr. 13; 96 (8): 4262-7). This approach further improves the expression level by treating the muscle with hyaluronidase before the intramuscular injection of the plasmid (McMahon JM1, Signori E, Wells K E, Fazio V M, Wells D J., Gene Ther. 2001 August; 8 (16): 1264-70). Furthermore, the hybridoma production can be performed by a known method, and can also be performed using, for example, a Hybrimune Hybridoma Production System (Cyto Pulse Sciences, Inc.).

Specific examples of obtaining a monoclonal antibody can include the following procedures:
(a) immune response can be induced by incorporating CDH6 CDNA into an expression vector (e.g., pcDNA3.1; Thermo Fisher Scientific Inc.), and directly administering the vector to an animal (e.g., a rat or a mouse) to be immunized by a method such as electroporation or a gene gun, so as to express CDH6 in the body of the animal. The administration of the vector by electroporation or the like may be performed one or more times, preferably a plurality of times, if necessary for enhancing antibody titer;
(b) collection of tissue (e.g., a lymph node) containing antibody-producing cells from the aforementioned animal in which the immune response has been induced;
(c) preparation of myeloma cells (hereinafter, referred to as “myelomas”) (e.g., mouse myeloma SP2/0-ag14 cells); (d) cell fusion between the antibody-producing cells and the myelomas;
(e) selection of a hybridoma group producing an antibody of interest;
(f) division into single cell clones (cloning);
(g) optionally, the culture of hybridomas for the mass production of monoclonal antibodies, or the breeding of animals into which the hybridomas are inoculated; and/or
(h) study of the physiological activity (internalization activity) and binding specificity of the monoclonal antibody thus produced, or examination of the properties of the antibody as a labeling reagent.

Examples of the method for measuring the antibody titer used herein can include, but are not limited to, flow cytometry and Cell-ELISA.

Examples of the hybridoma strain thus established can include anti-CDH6 antibody-producing hybridomas rG019, rG055, rG056 and rG061. It is to be noted that, in the present description, an antibody produced by the anti-CDH6 antibody-producing hybridoma rG019 is referred to as a “rG019 antibody” or simply “rG019”, an antibody produced by the hybridoma rG055 is referred to as a “rG055 antibody” or simply “rG055”, an antibody produced by the hybridoma rG056 is referred to as a “rG056 antibody” or simply “rG056”, and an antibody produced by the hybridoma rG061 is referred to as a “rG061 antibody” or simply “rG061”.

The light chain variable region of the rG019 antibody consists of the amino acid sequence shown in SEQ ID NO: 10. The amino acid sequence of the light chain variable region of the rG019 antibody is encoded by the nucleotide sequence shown in SEQ ID NO: 11. The light chain variable region of the rG019 antibody has CDRL1 consisting of the amino acid sequence shown in SEQ ID NO: 12, CDRL2 consisting of the amino acid sequence shown in SEQ ID NO: 13, and CDRL3 consisting of the amino acid sequence shown in SEQ ID NO: 14. The heavy chain variable region of the rG019 antibody consists of the amino acid sequence shown in SEQ ID NO: 15. The amino acid sequence of the heavy chain variable region of the rG019 antibody is encoded by the nucleotide sequence shown in SEQ ID NO: 16. The heavy chain variable region of the rG019 antibody has CDRH1 consisting of the amino acid sequence shown in SEQ ID NO: 17, CDRH2 consisting of the amino acid sequence shown in SEQ ID NO: 18, and CDRH3 consisting of the amino acid sequence shown in SEQ ID NO: 19. The sequence of the rG019 antibody is shown in Table 1.

The light chain variable region of the rG055 antibody consists of the amino acid sequence shown in SEQ ID NO: 20. The amino acid sequence of the light chain variable region of the rG055 antibody is encoded by the nucleotide sequence shown in SEQ ID NO: 21. The light chain variable region of the rG055 antibody has CDRL1 consisting of the amino acid sequence shown in SEQ ID NO: 22, CDRL2 consisting of the amino acid sequence shown in SEQ ID NO: 23, and CDRL3 consisting of the amino acid sequence shown in SEQ ID NO: 24. The heavy chain variable region of the rG055 antibody consists of the amino acid sequence shown in SEQ ID NO: 25. The amino acid sequence of the heavy chain variable region of the rG055 antibody is encoded by the nucleotide sequence shown in SEQ ID NO: 26. The heavy chain variable region of the rG055 antibody has CDRH1 consisting of the amino acid sequence shown in SEQ ID NO: 27, CDRH2 consisting of the amino acid sequence shown in SEQ ID NO: 28, and CDRH3 consisting of the amino acid sequence shown in SEQ ID NO: 29. The sequence of the rG055 antibody is shown in Table 1.

The light chain variable region of the rG056 antibody consists of the amino acid sequence shown in SEQ ID NO: 30. The amino acid sequence of the light chain variable region of the rG056 antibody is encoded by the nucleotide sequence shown in SEQ ID NO: 31. The light chain variable region of the rG056 antibody has CDRL1 consisting of the amino acid sequence shown in SEQ ID NO: 32, CDRL2 consisting of the amino acid sequence shown in SEQ ID NO: 33, and CDRL3 consisting of the amino acid sequence shown in SEQ ID NO: 34. The heavy chain variable region of the rG056 antibody consists of the amino acid sequence shown in SEQ ID NO: 35. The amino acid sequence of the heavy chain variable region of the rG056 antibody is encoded by the nucleotide sequence shown in SEQ ID NO: 36. The heavy chain variable region of the rG056 antibody has CDRH1 consisting of the amino acid sequence shown in SEQ ID NO: 37, CDRH2 consisting of the amino acid sequence shown in SEQ ID NO: 38, and CDRH3 consisting of the amino acid sequence shown in SEQ ID NO: 39. The sequence of the rG056 antibody is shown in Table 1.

The light chain variable region of the rG061 antibody consists of the amino acid sequence shown in SEQ ID NO: 40. The amino acid sequence of the light chain variable region of the rG061 antibody is encoded by the nucleotide sequence shown in SEQ ID NO: 41. The light chain variable region of the rG061 antibody has CDRL1 consisting of the amino acid sequence shown in SEQ ID NO: 42, CDRL2 consisting of the amino acid sequence shown in SEQ ID NO: 43, and CDRL3 consisting of the amino acid sequence shown in SEQ ID NO: 44. The heavy chain variable region of the rG061 antibody consists of the amino acid sequence shown in SEQ ID NO: 45. The amino acid sequence of the heavy chain variable region of the rG061 antibody is encoded by the nucleotide sequence shown in SEQ ID NO: 46. The heavy chain variable region of the rG061 antibody has CDRH1 consisting of the amino acid sequence shown in SEQ ID NO: 47, CDRH2 consisting of the amino acid sequence shown in SEQ ID NO: 48, and CDRH3 consisting of the amino acid sequence shown in SEQ ID NO: 49. The sequence of the rG061 antibody is shown in Table 1.

Furthermore, in the case where the steps (a) to (h) in the above “2. Production of anti-CDH6 antibody” are carried out again to obtain independently a monoclonal antibody separately and also in the case where a monoclonal antibody is obtained separately by other methods, an antibody having internalization activity equivalent to that of the rG019 antibody, the rG055 antibody, the rG056 antibody or the rG061 antibody can be obtained. One example of such an antibody can include an antibody binding to the same epitope to which the rG019 antibody, the rG055 antibody, the rG056 antibody or the rG061 antibody binds. If a newly prepared monoclonal antibody binds to a partial peptide or a partial three-dimensional structure to which the rG019 antibody, the rG055 antibody, the rG056 antibody or the rG061 antibody binds, it can be determined that the monoclonal antibody binds to the same epitope to which the rG019 antibody, the rG055 antibody, the rG056 antibody or the rG061 antibody binds. Moreover, by confirming that the monoclonal antibody competes with the rG019 antibody, the rG055 antibody, the rG056 antibody or the rG061 antibody in the binding of the antibody to CDH6 (i.e., the monoclonal antibody interferes with the binding of the rG019 antibody, the rG055 antibody, the rG056 antibody or the rG061 antibody to CDH6), it can be determined that the monoclonal antibody binds to the same epitope to which the anti-CDH6 antibody binds, even if the specific sequence or structure of the epitope has not been determined. When it is confirmed that the monoclonal antibody binds to the same epitope to which the rG019 antibody, the rG055 antibody, the rG056 antibody or the rG061 antibody binds, then it is strongly expected that the monoclonal antibody should have antigen-binding ability, biological activity and/or internalization activity equivalent to that of the rG019 antibody, the rG055 antibody, the rG056 antibody or the rG061 antibody.

### (3) Other Antibodies

The antibody of the present disclosure also includes genetically recombinant antibodies that have been artificially modified for the purpose of reducing heterogenetic antigenicity to humans, such as a chimeric antibody, a humanized antibody and a human antibody, as well as the above-described monoclonal antibody against CDH6. These antibodies can be produced by known methods.

Example of the chimeric antibody can include antibodies in which a variable region and a constant region are heterologous to each other, such as a chimeric antibody formed by conjugating the variable region of a mouse- or rat-derived antibody to a human-derived constant region (see Proc. Natl. Acad. Sci. U.S.A., 81, 6851-6855, (1984)).

Examples of the chimeric antibody derived from the rat anti-human CDH6 antibody include an antibody consisting of a light chain comprising the light chain variable region of each rat anti-human CDH6 antibody described in the present description (e.g., the rG019 antibody, the rG055 antibody, the rG056 antibody or the rG061 antibody) and a human-derived constant region, and a heavy chain comprising the heavy chain variable region thereof and a human-derived constant region.

Other examples of the chimeric antibody derived from the rat anti-human CDH6 antibody include an antibody consisting of a light chain comprising a light chain variable region having a substitution of one to several residues, 1 to 3 residues, 1 or 2 residues, preferably 1 residue, of amino acids in the light chain variable region of each rat anti-human CDH6 antibody described in the present description (e.g., the rG019 antibody, the rG055 antibody, the rG056 antibody or the rG061 antibody) with other amino acid residues, and a heavy chain comprising a heavy chain variable region having a substitution of one to several residues, 1 to 3 residues, 1 or 2 residues, preferably 1 residue, of amino acids in the heavy chain variable region thereof with other amino acid residues. This antibody may have any given human-derived constant region.

Other examples of the chimeric antibody derived from the rat anti-human CDH6 antibody include an antibody consisting of a light chain comprising a light chain variable region having a substitution of 1 or 2 residues, preferably 1 residue, of amino acids in any 1 to 3 CDRs in the light chain variable region of each rat anti-human CDH6 antibody described in the present description (e.g., the rG019 antibody, the rG055 antibody, the rG056 antibody or the rG061 antibody) with other amino acid residues, and a heavy chain comprising a heavy chain variable region having a substitution of 1 or 2 residues, preferably 1 residue, of amino acids in any 1 to 3 CDRs in the heavy chain variable region thereof with other amino acid residues. This antibody may have any given human-derived constant region.

Examples of the chimeric antibody derived from the rG019 antibody include an antibody consisting of a light chain comprising a light chain variable region consisting of the amino acid sequence shown in SEQ ID NO: 10, and a heavy chain comprising a heavy chain variable region consisting of the amino acid sequence shown in SEQ ID NO: 15. This antibody may have any given human-derived constant region.

Other examples of the chimeric antibody derived from the rG019 antibody include an antibody consisting of a light chain comprising a light chain variable region having a substitution of one to several residues, 1 to 3 residues, 1 or 2 residues, preferably 1 residue, of amino acids in the light chain variable region consisting of the amino acid sequence shown in SEQ ID NO: 10 with other amino acid residues, and a heavy chain comprising a heavy chain variable region having a substitution of one to several residues, 1 to 3 residues, 1 or 2 residues, preferably 1 residue, of amino acids in the heavy chain variable region consisting of the amino acid sequence shown in SEQ ID NO: 15 with other amino acid residues. This antibody may have any given human-derived constant region.

Other examples of the chimeric antibody derived from the rG019 antibody include an antibody consisting of a light chain comprising a light chain variable region having a substitution of 1 or 2 residues (preferably 1 residue) of amino acids in any 1 to 3 CDRs in the light chain variable region consisting of the amino acid sequence shown in SEQ ID NO: 10 with other amino acid residues, and a heavy chain comprising a heavy chain variable region having a substitution of 1 or 2 residues (preferably 1 residue) of amino acids in any 1 to 3 CDRs in the heavy chain variable region consisting of the amino acid sequence shown in SEQ ID NO: 15 with other amino acid residues. This antibody may have any given human-derived constant region.

Other examples of the chimeric antibody derived from the rG019 antibody include an antibody consisting of a light chain comprising a light chain variable region consisting of the amino acid sequence shown in SEQ ID NO: 10, and a heavy chain comprising a heavy chain variable region consisting of the amino acid sequence shown in SEQ ID NO: 58. This antibody may have any given human-derived constant region. The amino acid sequence shown in SEQ ID NO: 58 is a sequence with a cysteine residue substituted with a proline residue in CDRH2 in the amino acid sequence shown in SEQ ID NO: 15.

Specific examples of the chimeric antibody derived from the rG019 antibody include an antibody consisting of a light chain consisting of the light chain full-length amino acid sequence shown in SEQ ID NO: 53, and a heavy chain consisting of the heavy chain full-length amino acid sequence shown in SEQ ID NO: 56. In the present description, this chimeric anti-human CDH6 antibody is referred to as a “chimeric G019 antibody”, a “chG019 antibody” or “chG019”. The light chain full-length amino acid sequence of the chG019 antibody is encoded by the nucleotide sequence shown in SEQ ID NO: 54, and the heavy chain full-length amino acid sequence of the chG019 antibody is encoded by the nucleotide sequence shown in SEQ ID NO: 57.

The amino acid sequence of the light chain variable region of the chG019 antibody is identical to the amino acid sequence of the light chain variable region of the rG019 antibody, and consists of the amino acid sequence shown in SEQ ID NO: 10. The light chain of the chG019 antibody has CDRL1 consisting of the amino acid sequence shown in SEQ ID NO: 12, CDRL2 consisting of the amino acid sequence shown in SEQ ID NO: 13, and CDRL3 consisting of the amino acid sequence shown in SEQ ID NO: 14, which are identical to the light chain CDRL1, CDRL2 and CDRL3, respectively, of rG019. The amino acid sequence of the light chain variable region of the chG019 antibody is encoded by the nucleotide sequence shown in SEQ ID NO: 55.

The amino acid sequence of the heavy chain variable region of the chG019 antibody consists of the amino acid sequence shown in SEQ ID NO: 58. The heavy chain of the chG019 antibody has CDRH1 consisting of the amino acid sequence shown in SEQ ID NO: 17, CDRH2 consisting of the amino acid sequence shown in SEQ ID NO: 60, and CDRH3 consisting of the amino acid sequence shown in SEQ ID NO: 19. The amino acid sequence shown in SEQ ID NO: 58 is a sequence with a cysteine residue substituted with a proline residue in CDRH2 in the amino acid sequence shown in SEQ ID NO: 15. The CDRH2 consisting of the amino acid sequence shown in SEQ ID NO: 60 is a sequence with a cysteine residue substituted with a proline residue in the rG019 CDRH2 shown in SEQ ID NO: 18. The amino acid sequence of the heavy chain variable region of the chG019 antibody is encoded by the nucleotide sequence shown in SEQ ID NO: 59.

The sequence of the chG019 antibody is shown in Table 1.

Examples of the chimeric antibody derived from the rat anti-human CDH6 antibody rG055 antibody include a chimeric antibody consisting of a light chain comprising a light chain variable region consisting of the amino acid sequence shown in SEQ ID NO: 20, and a heavy chain comprising a heavy chain variable region consisting of the amino acid sequence shown in SEQ ID NO: 25. This antibody may have any given human-derived constant region.

Examples of the chimeric antibody derived from the rat anti-human CDH6 antibody rG056 antibody include a chimeric antibody consisting of a light chain comprising a light chain variable region consisting of the amino acid sequence shown in SEQ ID NO: 30, and a heavy chain comprising a heavy chain variable region consisting of the amino acid sequence shown in SEQ ID NO: 35. This antibody may have any given human-derived constant region.

Examples of the chimeric antibody derived from the rat anti-human CDH6 antibody rG061 antibody include a chimeric antibody consisting of a light chain comprising a light chain variable region consisting of the amino acid sequence shown in SEQ ID NO: 40, and a heavy chain comprising a heavy chain variable region consisting of the amino acid sequence shown in SEQ ID NO: 45. This antibody may have any given human-derived constant region.

Examples of the humanized antibody can include an antibody formed by incorporating only complementarity determining regions (CDRs) into a human-derived antibody (see Nature (1986) 321, p. 522-525), an antibody formed by incorporating the amino acid residues from some frameworks, as well as CDR sequences, into a human antibody according to a CDR grafting method (International Publication No. WO90/07861), and an antibody formed by modifying the amino acid sequences of some CDRs while maintaining antigen-binding ability.

In the present description, the humanized antibody derived from the rG019 antibody, the rG055 antibody, the rG056 antibody, the rG061 antibody or the chG019 antibody is not limited to a specific humanized antibody as long as the humanized antibody retains all 6 CDR sequences unique to the rG019 antibody, the rG055 antibody, the rG056 antibody, the rG061 antibody or the chG019 antibody and has internalization activity. The amino acid sequences of some CDRs of this humanized antibody may be further modified as long as it has internalization activity.

Concrete examples of the humanized antibody of the chG019 antibody can include any given combination of: a light chain comprising a light chain variable region consisting of any one amino acid sequence selected from the group consisting of (1) the amino acid sequence shown in SEQ ID NO: 63 or 67, (2) an amino acid sequence having an identity of at least 95% or more (preferably an amino acid sequence having a sequence identity of at least 95% or more to the sequence of a framework region other than at each CDR sequence) to the above-described amino acid sequence (1), and (3) an amino acid sequence comprising a deletion, substitution or addition of one or several amino acids in the above-described amino acid sequence (1); and a heavy chain comprising a heavy chain variable region consisting of any one amino acid sequence selected from the group consisting of (4) the amino acid sequence shown in SEQ ID NO: 71, 75 or 79, (5) an amino acid sequence having an identity of at least 95% or more (preferably an amino acid sequence having a sequence identity of at least 95% or more to the sequence of a framework region other than at each CDR sequence) to the above-described amino acid sequence (4), and (6) an amino acid sequence comprising a deletion, substitution or addition of one or several amino acids in the above-described amino acid sequence (4).

Alternatively, an antibody having a humanized heavy chain or light chain and the other chain derived from a rat antibody or a chimeric antibody can also be used. Examples of such an antibody can include any given combination of: a light chain comprising a light chain variable region consisting of any one amino acid sequence selected from the group consisting of (1) the amino acid sequence shown in SEQ ID NO: 63 or 67, (2) an amino acid sequence having an identity of at least 95% or more (preferably an amino acid sequence having a sequence identity of at least 95% or more to the sequence of a framework region other than at each CDR sequence) to the above-described amino acid sequence (1), and (3) an amino acid sequence comprising a deletion, substitution or addition of one or several amino acids in the above-described amino acid sequence (1); and a heavy chain comprising a heavy chain variable region consisting of any one amino acid sequence selected from the group consisting of (4) the amino acid sequence shown in SEQ ID NO: 15, 25, 35, 45 or 58, (5) an amino acid sequence having an identity of at least 95% or more (preferably an amino acid sequence having a sequence identity of at least 95% or more to the sequence of a framework region other than at each CDR sequence) to the above-described amino acid sequence (4), and (6) an amino acid sequence comprising a deletion, substitution or addition of one or several amino acids in the above-described amino acid sequence (4). Other examples of such an antibody can include any given combination of: a light chain comprising a light chain variable region consisting of any one amino acid sequence selected from the group consisting of (1) the amino acid sequence shown in SEQ ID NO: 10, 20, 30 or 40, (2) an amino acid sequence having an identity of at least 95% or more (preferably an amino acid sequence having a sequence identity of at least 95% or more to the sequence of a framework region other than at each CDR sequence) to the above-described amino acid sequence (1), and (3) an amino acid sequence comprising a deletion, substitution or addition of one or several amino acids in the above-described amino acid sequence (1); and a heavy chain comprising a heavy chain variable region consisting of any one amino acid sequence selected from the group consisting of (4) the amino acid sequence shown in SEQ ID NO: 71, 75 or 79, (5) an amino acid sequence having an identity of at least 95% or more (preferably an amino acid sequence having a sequence identity of at least 95% or more to the sequence of a framework region other than at each CDR sequence) to the above-described amino acid sequence (4), and (6) an amino acid sequence comprising a deletion, substitution or addition of one or several amino acids in the above-described amino acid sequence (4).

The amino acid substitution in the present description is preferably a conservative amino acid substitution. The conservative amino acid substitution is a substitution occurring within an amino acid group associated with certain amino acid side chains. Preferred amino acid groups are the following: acidic group=aspartic acid and glutamic acid; basic group=lysine, arginine, and histidine; non-polar group=alanine, valine, leucine, isoleucine, proline, phenylalanine, methionine, and tryptophan; and uncharged polar family=glycine, asparagine, glutamine, cysteine, serine, threonine, and tyrosine. Other preferred amino acid groups are the following: aliphatic hydroxy group=serine and threonine; amide-containing group=asparagine and glutamine; aliphatic group=alanine, valine, leucine and isoleucine; and aromatic group=phenylalanine, tryptophan and tyrosine. Such amino acid substitution is preferably carried out without impairing the properties of a substance having the original amino acid sequence.

Examples of the antibody having a preferred combination of the above-described light chains and heavy chains include an antibody consisting of a light chain having the light chain variable region amino acid sequence shown in SEQ ID NO: 63 (in the present description, also referred to as a hL02 light chain variable region amino acid sequence) or a light chain having the light chain variable region amino acid sequence shown in SEQ ID NO: 67 (in the present description, also referred to as a hL03 light chain variable region amino acid sequence), and a heavy chain having the heavy chain variable region amino acid sequence shown in SEQ ID NO: 71 (in the present description, also referred to as a hH01 heavy chain variable region amino acid sequence), a heavy chain having the heavy chain variable region amino acid sequence shown in SEQ ID NO: 75 (in the present description, also referred to as a hH02 heavy chain variable region amino acid sequence) or a heavy chain having the heavy chain variable region amino acid sequence shown in SEQ ID NO: 79 (in the present description, also referred to as a hH04 heavy chain variable region amino acid sequence). Preferred examples thereof include: an antibody consisting of a light chain having the light chain variable region amino acid sequence shown in SEQ ID NO: 63 and a heavy chain having the heavy chain variable region amino acid sequence shown in SEQ ID NO: 71; an antibody consisting of a light chain having the light chain variable region amino acid sequence shown in SEQ ID NO: 63 and a heavy chain having the heavy chain variable region amino acid sequence shown in SEQ ID NO: 75; an antibody consisting of a light chain having the light chain variable region amino acid sequence shown in SEQ ID NO: 63 and a heavy chain having the heavy chain variable region amino acid sequence shown in SEQ ID NO: 79; an antibody consisting of a light chain having the light chain variable region amino acid sequence shown in SEQ ID NO: 67 and a heavy chain having the heavy chain variable region amino acid sequence shown in SEQ ID NO: 71; an antibody consisting of a light chain having the light chain variable region amino acid sequence shown in SEQ ID NO: 67 and a heavy chain having the heavy chain variable region amino acid sequence shown in SEQ ID NO: 75; and an antibody consisting of a light chain having the light chain variable region amino acid sequence shown in SEQ ID NO: 67 and a heavy chain having the heavy chain variable region amino acid sequence shown in SEQ ID NO: 79. More preferred examples thereof include: an antibody consisting of a light chain having the light chain variable region amino acid sequence shown in SEQ ID NO: 63 and a heavy chain having the heavy chain variable region amino acid sequence shown in SEQ ID NO: 71; an antibody consisting of a light chain having the light chain variable region amino acid sequence shown in SEQ ID NO: 63 and a heavy chain having the heavy chain variable region amino acid sequence shown in SEQ ID NO: 75; an antibody consisting of a light chain having the light chain variable region amino acid sequence shown in SEQ ID NO: 63 and a heavy chain having the heavy chain variable region amino acid sequence shown in SEQ ID NO: 79; and an antibody consisting of a light chain having the light chain variable region amino acid sequence shown in SEQ ID NO: 67 and a heavy chain having the heavy chain variable region amino acid sequence shown in SEQ ID NO: 75.

Other examples of the antibody having a preferred combination of the above-described light chains and heavy chains include an antibody consisting of a light chain consisting of the amino acid sequence at positions 21 to 233 in the light chain full-length amino acid sequence shown in SEQ ID NO: 61 (in the present description, also referred to as the hL02 light chain full-length amino acid sequence) or a light chain consisting of the amino acid sequence at positions 21 to 233 in the light chain full-length amino acid sequence shown in SEQ ID NO: 65 (in the present description, also referred to as the hL03 light chain full-length amino acid sequence), and a heavy chain consisting of the amino acid sequence at positions 20 to 471 in the heavy chain full-length amino acid sequence shown in SEQ ID NO: 69 (in the present description, also referred to as the hH01 heavy chain full-length amino acid sequence), a heavy chain consisting of the amino acid sequence at positions 20 to 471 in the heavy chain full-length amino acid sequence shown in SEQ ID NO: 73 (in the present description, also referred to as the hH02 heavy chain full-length amino acid sequence) or a heavy chain consisting of the amino acid sequence at positions 20 to 471 in the heavy chain full-length amino acid sequence shown in SEQ ID NO: 77 (in the present description, also referred to as the hH04 heavy chain full-length amino acid sequence). Preferred examples thereof include: an antibody consisting of a light chain consisting of the amino acid sequence at positions 21 to 233 in the light chain full-length amino acid sequence shown in SEQ ID NO: 61 and a heavy chain consisting of the amino acid sequence at positions 20 to 471 in the heavy chain full-length amino acid sequence shown in SEQ ID NO: 69; an antibody consisting of a light chain consisting of the amino acid sequence at positions 21 to 233 in the light chain full-length amino acid sequence shown in SEQ ID NO: 61 and a heavy chain consisting of the amino acid sequence at positions 20 to 471 in the heavy chain full-length amino acid sequence shown in SEQ ID NO: 73; an antibody consisting of a light chain consisting of the amino acid sequence at positions 21 to 233 in the light chain full-length amino acid sequence shown in SEQ ID NO: 61 and a heavy chain consisting of the amino acid sequence at positions 20 to 471 in the heavy chain full-length amino acid sequence shown in SEQ ID NO: 77; an antibody consisting of a light chain consisting of the amino acid sequence at positions 21 to 233 in the light chain full-length amino acid sequence shown in SEQ ID NO: 65 and a heavy chain consisting of the amino acid sequence at positions 20 to 471 in the heavy chain full-length amino acid sequence shown in SEQ ID NO: 69; an antibody consisting of a light chain consisting of the amino acid sequence at positions 21 to 233 in the light chain full-length amino acid sequence shown in SEQ ID NO: 65 and a heavy chain consisting of the amino acid sequence at positions 20 to 471 in the heavy chain full-length amino acid sequence shown in SEQ ID NO: 73; and an antibody consisting of a light chain consisting of the amino acid sequence at positions 21 to 233 in the light chain full-length amino acid sequence shown in SEQ ID NO: 65 and a heavy chain consisting of the amino acid sequence at positions 20 to 471 in the heavy chain full-length amino acid sequence shown in SEQ ID NO: 77. More preferred examples thereof include: an antibody consisting of a light chain consisting of the amino acid sequence at positions 21 to 233 in the light chain full-length amino acid sequence shown in SEQ ID NO: 61 and a heavy chain consisting of the amino acid sequence at positions 20 to 471 in the heavy chain full-length amino acid sequence shown in SEQ ID NO: 69 (in the present description, also referred to as the “H01L02 antibody” or “H01L02”); an antibody consisting of a light chain consisting of the amino acid sequence at positions 21 to 233 in the light chain full-length amino acid sequence shown in SEQ ID NO: 61 and a heavy chain consisting of the amino acid sequence at positions 20 to 471 in the heavy chain full-length amino acid sequence shown in SEQ ID NO: 73 (in the present description, also referred to as the “H02L02 antibody” or “H02L02”); an antibody consisting of a light chain consisting of the amino acid sequence at positions 21 to 233 in the light chain full-length amino acid sequence shown in SEQ ID NO: 61 and a heavy chain consisting of the amino acid sequence at positions 20 to 471 in the heavy chain full-length amino acid sequence shown in SEQ ID NO: 77 (in the present description, also referred to as the “H04L02 antibody” or “H04L02”); and an antibody consisting of a light chain consisting of the amino acid sequence at positions 21 to 233 in the light chain full-length amino acid sequence shown in SEQ ID NO: 65 and a heavy chain consisting of the amino acid sequence at positions 20 to 471 in the heavy chain full-length amino acid sequence shown in SEQ ID NO: 73 (in the present description, also referred to as the “H02L03 antibody” or “H02L03”). The sequences of the H01L02 antibody, the H02L02 antibody, the H02L03 antibody or the H04L02 antibody are shown in Table 1.

By combining together sequences showing a high identity to the above-described heavy chain amino acid sequences and light chain amino acid sequences, it is possible to select an antibody having a biological activity equivalent to that of each of the above-described antibodies. Such an identity is an identity of generally 80% or more, preferably 90% or more, more preferably 95% or more, and most preferably 99% or more. Moreover, also by combining amino acid sequences of a heavy chain and a light chain comprising a substitution, deletion or addition of one or several amino acid residues thereof with respect to the amino acid sequence of a heavy chain or a light chain, it is possible to select an antibody having a biological activity equivalent to that of each of the above-described antibodies.

The identity between two types of amino acid sequences can be determined by aligning the sequences using the default parameters of Clustal W version 2 (Larkin M A, Blackshields G, Brown N P, Chenna R, McGettigan P A, McWilliam H, Valentin F, Wallace I M, Wilm A, Lopez R, Thompson J D, Gibson T J and Higgins D G (2007), “Clustal W and Clustal X version 2.0”, Bioinformatics. 23 (21): 2947-2948).

It is to be noted that, in the hL02 light chain full-length amino acid sequence shown in SEQ ID NO: 61, the amino acid sequence consisting of the amino acid residues at positions 1 to 20 is the signal sequence, the amino acid sequence consisting of the amino acid residues at positions 21 to 128 is the variable region, and the amino acid sequence consisting of the amino acid residues at positions 129 to 233 is the constant region. In the hL02 light chain full-length nucleotide sequence shown in SEQ ID NO: 62, the nucleotide sequence consisting of the nucleotides at positions 1 to 60 encodes the signal sequence, the nucleotide sequence consisting of the nucleotides at positions 61 to 384 encodes the variable region, and the nucleotide sequence consisting of the nucleotides at positions 385 to 699 encodes the constant region.

In the hL03 light chain full-length amino acid sequence shown in SEQ ID NO: 65, the amino acid sequence consisting of the amino acid residues at positions 1 to 20 is the signal sequence, the amino acid sequence consisting of the amino acid residues at positions 21 to 128 is the variable region, and the amino acid sequence consisting of the amino acid residues at positions 129 to 233 is the constant region. In the hL03 light chain full-length nucleotide sequence shown in SEQ ID NO: 66, the nucleotide sequence consisting of the nucleotides at positions 1 to 60 encodes the signal sequence, the nucleotide sequence consisting of the nucleotides at positions 61 to 384 encodes the variable region, and the nucleotide sequence consisting of the nucleotides at positions 385 to 699 encodes the constant region.

In the hH01 heavy chain full-length amino acid sequence shown in SEQ ID NO: 69, the amino acid sequence consisting of the amino acid residues at positions 1 to 19 is the signal sequence, the amino acid sequence consisting of the amino acid residues at positions 20 to 141 is the variable region, and the amino acid sequence consisting of the amino acid residues at positions 142 to 471 is the constant region. In the hH01 heavy chain full-length nucleotide sequence shown in SEQ ID NO: 70, the nucleotide sequence consisting of the nucleotides at positions 1 to 57 encodes the signal sequence, the nucleotide sequence consisting of the nucleotides at positions 58 to 423 encodes the variable region, and the nucleotide sequence consisting of the nucleotides at positions 424 to 1413 encodes the constant region.

In the hH02 heavy chain full-length amino acid sequence shown in SEQ ID NO: 73, the amino acid sequence consisting of the amino acid residues at positions 1 to 19 is the signal sequence, the amino acid sequence consisting of the amino acid residues at positions 20 to 141 is the variable region, and the amino acid sequence consisting of the amino acid residues at positions 142 to 471 is the constant region. In the hH02 heavy chain full-length nucleotide sequence shown in SEQ ID NO: 74, the nucleotide sequence consisting of the nucleotides at positions 1 to 57 encodes the signal sequence, the nucleotide sequence consisting of the nucleotides at positions 58 to 423 encodes the variable region, and the nucleotide sequence consisting of the nucleotides at positions 424 to 1413 encodes the constant region.

In the hH04 heavy chain full-length amino acid sequence shown in SEQ ID NO: 77, the amino acid sequence consisting of the amino acid residues at positions 1 to 19 is the signal sequence, the amino acid sequence consisting of the amino acid residues at positions 20 to 141 is the variable region, and the amino acid sequence consisting of the amino acid residues at positions 142 to 471 is the constant region. In the hH04 heavy chain full-length nucleotide sequence shown in SEQ ID NO: 78, the nucleotide sequence consisting of the nucleotides at positions 1 to 57 encodes the signal sequence, the nucleotide sequence consisting of the nucleotides at positions 58 to 423 encodes the variable region, and the nucleotide sequence consisting of the nucleotides at positions 424 to 1413 encodes the constant region.

TABLE 1-1

| SEQ | | |
|---|---|---|
| ID NO | | Sequence |
| 1 | Amino acid | MRTYRYFLLLFWVGQPYPTLSTPLSKRTSGFPAKKRALELSGNSKNELNRSKRSWMW |
| | sequence of | NQFFLLEEYTGSDYQYVGKLHSDQDRGDGSLKYILSGDGAGDLFIINENTGDIQATK |
| | human CDH6 | RLDREEKPVYILRAQAINRRTGRPVEPESEFIIKIHDINDNEPIFTKEVYTATVPEM |
| | ORF | SDVGTFVVQVTATDADDPTYGNSAKVVYSILQGQPYFSVESETGIIKTALLNMDREN |
| | | REQYQVVIQAKDMGGQMGGLSGTTTVNITLTDVNDNPPRFPQSTYQFKTPESSPPGT |
| | | PIGRIKASDADVGENAEIEYSITDGEGLDMFDVITDQETQEGIITVKKLLDFEKKKV |
| | | YTLKVEASNPYVEPRFLYLGPFKDSATVRIVVEDVDEPPVFSKLAYILQIREDAQIN |
| | | TTIGSVTAQDPDAARNPVKYSVDRHTDMDRIFNIDSGNGSIFTSKLLDRETLLWHNI |
| | | TVIATEINNPKQSSRVPLYIKVLDVNDNAPEFAEFYETFVCEKAKADQLIQTLHAVD |
| | | KDDPYSGHQFSFSLAPEAASGSNFTIQDNKDNTAGILTRKNGYNRHEMSTYLLPVVI |
| | | SDNDYPVQSSTGTVTVRVCACDHHGNMQSCHAEALIHPTGLSTGALVAILLCIVILL |
| | | VTVVLFAALRRQRKKEPLIISKEDIRDNIVSYNDEGGGEEDTQAFDIGTLRNPEAIE |
| | | DNKLRRDIVPEALFLPRRTPTARDNTDVRDFINQRLKENDTDPTAPPYDSLATYAYE |
| | | GTGSVADSLSSLESVTTDADQDYDYLSDWGPRFKKLADMYGGVDSDKDS |
| 2 | Human CDH6 | SWMWNQFFLLEEYTGSDYQYVGKLHSDQDRGDGSLKYILSGDGAGDLFIINENTGDI |
| | EC1 | QATKRLDREEKPVYILRAQAINRRTGRPVEPESEFIIKIHDINDNEPIF |
| 3 | Human CDH6 | TKEVYTATVPEMSDVGTFVVQVTATDADDPTYGNSAKVVYSILQGQPYFSVESETGI |
| | EC2 | IKTALLNMDRENREQYQVVIQAKDMGGQMGGLSGTTTVNITLTDVNDNPPRF |
| 4 | Human CDH6 | PQSTYQFKTPESSPPGTPIGRIKASDADVGENAEIEYSITDGEGLDMFDVITDQETQ |
| | EC3 | EGIITVKKLLDFEKKKVYTLKVEASNPYVEPRFLYLGPFKDSATVRIVVEDVDEPPV |
| | | F |
| 5 | Human CDH6 | SKLAYILQIREDAQINTTIGSVTAQDPDAARNPVKYSVDRHTDMDRIFNIDSGNGSI |
| | EC4 | FTSKLLDRETLLWHNITVIATEINNPKQSSRVPLYIKVLDVNDNAP |
| 6 | Human CDH6 | EFAEFYETFVCEKAKADQLIQTLHAVDKDDPYSGHQFSFSLAPEAASGSNFTIQDNK |
| | EC5 | DNTAGILTRKNGYNRHEMSTYLLPVVISDNDYPVQSSTGTVTVRVCACDHHGNMQSC |
| | | HAEALIHP |
| 7 | Amino acid | MRTYRYFLLLFWVGQPYPTFSNPLSKRTSGFPAKRKALELSANSRNELSRSKRSWMW |
| | sequence of | NQFFLLEEYTGSDYQYVGKLHSDQDRGDGSLKYILSGDGAGDLFIINENTGDIQATK |
| | mouse CDH6 ORF | RLDREEKPVYILRAQAVNRRTGRPVEPESEFIIKIHDINDNEPIFTKDVYTATVPEM |
| | | ADVGTFVVQVTATDADDPTYGNSAKVVYSILQGQPYFSVESETGIIKTALLNMDREN |
| | | REQYQVVIQAKDMGGQMGGLSGTTTVNITLTDVNDNPPRFPQSTYQFKTPESSPPGT |
| | | PIGRIKASDADVGENAEIEYSITDGEGHEMFDVITDQETQEGIITVKKLLDFEKKKV |
| | | YTLKVEASNPHVEPRFLYLGPFKDSATVRIVVDDVDEPPVFSKLAYILQIREDARIN |
| | | TTIGSVAAQDPDAARNPVKYSVDRHTDMDRIFNIDSGNGSIFTSKLLDRETLLWHNI |
| | | TVIATEINNPKQSSRVPLYIKVLDVNDNAPEFAEFYETFVCEKAKADQLIQTLRAVD |
| | | KDDPYSGHQFSFSLAPEAASSSNFTIQDNKDNTAGILTRKNGYNRHEMSTYLLPVVI |
| | | SDNDYPVQSSTGTVTVRVCACDHHGNMQSCHAEALIHPTGLSTGALVAILLCIVILL |
| | | VTVVLFAALRRQRKKEPLIISKEDIRDNIVSYNDEGGGEEDTQAFDIGTLRNPEAME |
| | | DSKSRRDIVPEALFLPRRTPTARDNTDVRDFINQRLKENDTDPTAPPYDSLATYAYE |
| | | GTGSVADSLSSLESVTTDGDQDYDYLSDWGPRFKKLADMYGGMDSDKDS |

TABLE 1-2

| | | |
|---|---|---|
| 8 | Amino acid | MRTYRYFLLLFWVGQPYPTFSNPLSKRTSGFPAKRRALELSANSRNELSRSKRSWMW |
| | sequence of rat | NQFFLLEEYTGSDYQYVGKLHSDQDRGDGSLKYILSGDGAGDLFIINENTGDIQATK |
| | CDH6 ORF | RLDREEKPVYILRAQAINRRTGRPVEPESEFIIKIHDINDNEPIFTKDVYTATVPEM |
| | | ADVGTFVVQVTATDADDPTYGNSAKVVYSILQGQPYFSVESETGIIKTALLNMDREN |
| | | REQYQVVIQAKDMGGQMGGLSGTTTVNITLTDVNDNPPRFPQSTYQFKTPESSPPGT |
| | | PIGRIKASDADVGENAEIEYSITDGEGHDMFDVITDQETQEGIITVKKLLDFEKKRV |
| | | YTLKVEASNPHIEPRFLYLGPFKDSATVRIVVDDVDEPPVFSKPAYILQIREDAQIN |
| | | TTIGSVAAQDPDAARNPVKYSVDRHTDMDRIFNIDSGNGSIFTSKLLDRETLLWHNI |
| | | TVIATEINNPKQSSRVPLYIKVLDVNDNAPEFAEFYETFVCEKAKADQLIQTLHAVD |
| | | KDDPYSGHQFSFSLAPEAASGSNFTIQDNKDNTAGILTRKNGYNRHEMSTYLLPVVI |
| | | SDNDYPVQSSTGTVTVRVCACDHHGNMQSCHAEALIHPTGLSTGALVAILLCIVILL |
| | | VTVVLFAALRRQRKKEPLIISKEDIRDNIVSYNDEGGGEEDTQAFDIGTLRNPKPWR |
| | | QQSRRDMVPEALFLPRRTPTARDNTDVRDFISQRLRKMNTDPTAPPYDSLATYAYEG |
| | | TGSVADSLSSLESVTTDGDQDYGYLSDWGPRFKKLADMYGGMDSDKDS |
| 9 | Amino acid | MRTYRYFLLLFWVGQPYPTLSTPLSKRTSGFPAKKRALELSGNSKNELNRSKRSWMW |
| | sequence of | NQFFLLEEYTGSDYQYVGKLHSDQDRGDGSLKYILSGDGAGDLFIINENTGDIQATK |
| | cynomolgus | RLDREEKPVYILRAQAINRRTGRPVEPESEFIIKIHDINDNEPIFTKEVYTATVPEM |
| | monkey CDH6 | SDVGTFVVQVTATDADDPTYGNSAKVVYSILQGQPYFSVESETGIIKTALLNMDREN |
| | ORF | REQYQVVIQAKDMGGQMGGLSGTTTVNITLTDVNDNPPRFPQSTYQFKTPESSPPGT |
| | | PIGRIKASDADVGENAEIEYSITDGEGLDMFDVITDQETQEGIITVKKLLDFEKKKV |
| | | YTLKVEASNPHVEPRFLYLGPFKDSATVRIVVEDVDEPPVFSKLAYILQIREDAQIN |
| | | TTIGSVTAQDPDAARNPVKYSVDRHTDMDRIFNIDSGNGSIFTSKLLDRETLLWHNI |
| | | TVIATEINNPKQSSRVPLYIKVLDVNDNAPEFAEFYETFVCEKAKADQLIQTLRAVD |
| | | KDDPYSGHQFSFSLAPEAASGSNFTIQDNKDNTAGILTRKNGYNRHEMSTYLLPVVI |
| | | SDNDYPVQSSTGTVTVRVCACDHHGNMQSCHAEALIHPTGLSTGALVAILLCIVILL |
| | | VTVVLFAALRRQRKKEPLIISKEDIRDNIVSYNDEGGGEEDTQAFDIGTLRNPEAIE |
| | | DNKLRRDIVPEALFLPRRTPTARDNTDVRDFINQRLKENDTDPTAPPYDSLATYAYE |
| | | GTGSVADSLSSLESVTTDGDQDYDYLSDWGPRFKKLADMYGGVDSDKDS |
| 10 | rG019 light chain | DIQMTQSPSLLSASVGDRVTLNCKASQNIYKNLAWYQQKLGEGPKLLIYDANTLQTG |
| | variable region | IPSRFSGSGSGSDFTLTISSLQPEDVATYFCQQYYSGWAFGGVTNLELKRA |
| | amino acid | |
| | sequence | |
| 11 | rG019 light chain | GACATCCAGATGACCCAGTCTCCTTCACTCCTGTCTGCATCTGTGGGAGACAGAGTC |
| | variable region | ACTCTCAACTGCAAAGCAAGTCAGAATATTTATAAGAACTTAGCCTGGTATCAGCAA |
| | nucleotide | AAGCTTGGAGAAGGTCCCAAACTCCTGATTTATGATGCAAACACTTTGCAAACGGGC |
| | sequence | ATCCCATCAAGGTTCAGTGGCAGTGGATCTGGTTCAGATTTCACACTCACCATCAGC |
| | | AGCCTGCAGCCTGAAGATGTTGCCACATATTTCTGCCAGCAGTACTATAGCGGGTGG |
| | | GCGTTCGGTGGAGTCACCAACCTGGAATTGAAACGGGCT |
| 12 | rG019 CDRL1 | KASQNIYKNLA |
| 13 | rG019 CDRL2 | DANTLQT |
| 14 | rG019 CDRL3 | QQYYSGWA |

TABLE 1-3

| | | |
|---|---|---|
| 15 | rG019 heavy | QVQLQQSGAELVKPGSSVKISCKASGYTFTRNFMHWIKQQPGNGLEWIGWIYCGDGE |
| | chain variable | TEYNQKFNGKATLTADRSSSTAYMELSRLTSEDSAVYFCARGVYGGFAGGYFDFWGQ |
| | region amino acid | GVMVTVSS |
| | sequence | |
| 16 | rG019 heavy | CAGGTACAGCTGCAGCAATCTGGGGCTGAACTGGTGAAGCCTGGGTCCTCAGTGAAA |
| | chain variable | ATTTCCTGCAAGGCTTCTGGCTACACCTTCACCAGGAACTTTATGCACTGGATAAAA |
| | region nucleotide | CAGCAGCCTGGAAATGGCCTTGAGTGGATTGGGTGGATTTATTGTGGAGATGGTGAG |
| | sequence | ACAGAGTACAATCAAAAGTTCAATGGGAAGGCAACACTCACTGCGGACAGATCCTCC |
| | | AGCACAGCCTATATGGAGCTCAGCAGACTGACATCTGAGGACTCTGCAGTCTATTTC |
| | | TGTGCAAGAGGGGTTTACGGAGGGTTTGCCGGGGGCTACTTTGATTTCTGGGGCCAA |
| | | GGAGTCATGGTCACAGTCTCCTCA |
| 17 | rG019 CDRH1 | GYTFTRNFMH |
| 18 | rG019 CDRH2 | WIYCGDGETE |
| 19 | rG019 CDRH3 | GVYGGFAGGYFDF |
| 20 | rG055 light chain | DVQMTHSPSYLAASPGESVSISCKTSKNISNYLVWYQQKPGEAYKLLIYSGSTLQSG |
| | variable region | TPSRFSGSGSGTDFTLTIRSLEPEDFGLYFCQQYYEKPFTFGSGTKLEIKRA |
| | amino acid | |
| | sequence | |
| 21 | rG055 light chain | GATGTCCAGATGACCCACTCTCCGTCTTATCTTGCTGCGTCTCCTGGAGAAAGTGTT |
| | variable region | TCCATCAGTTGCAAGACAAGTAAGAACATTAGTAATTATTTAGTCTGGTATCAACAG |
| | nucleotide | AAACCTGGGGAAGCATATAAGCTTCTTATCTATTCTGGGTCAACTTTGCAATCTGGA |
| | sequence | ACTCCATCAAGGTTCAGTGGCAGTGGATCTGGTACAGATTTCACTCTCACTATCAGA |
| | | AGCCTGGAGCCTGAAGATTTTGGACTCTATTTCTGTCAACAGTATTATGAAAAACCA |
| | | TTCACGTTCGGCTCAGGGACGAAGTTGGAAATAAAACGGGCT |
| 22 | rG055 CDRL1 | KTSKNISNYLV |
| 23 | rG055 CDRL2 | SGSTLQS |
| 24 | rG055 CDRL3 | QQYYEKPFT |
| 25 | rG055 heavy | EVQLQESGPGLVRPSQSLSLSCSVTDYSITSNYWGWIRRFPGNKMEWMGYITYSGYT |
| | chain variable | SYNPSLQSRISITRDTSKNQFFLQLNSVTAEDTATYYCARSINHGGYSYVVDAWGPG |
| | region amino acid | ASVTVSS |
| | sequence | |
| 26 | rG055 heavy | GAGGTGCAACTTCAGGAGTCAGGACCTGGCCTTGTGAGACCCTCACAGTCACTCTCC |
| | chain variable | CTCTCCTGTTCTGTCACTGATTACTCCATCACTAGTAATTACTGGGGCTGGATCCGG |
| | region nucleotide | AGGTTCCCAGGAAATAAAATGGAGTGGATGGGATACATAACCTATAGTGGTTACACT |
| | sequence | AGCTACAACCCATCTCTCCAAAGTCGAATCTCCATTACTAGAGACACATCGAAGAAT |
| | | CAGTTCTTCCTGCAGTTGAACTCTGTAACTGCTGAGGACACAGCCACATATTACTGT |
| | | GCAAGATCGATTAACCACGGAGGATATAGTTATGTTGTGGATGCCTGGGGTCCGGGA |
| | | GCTTCAGTCACTGTCTCCTCA |
| 27 | rG055 CDRH1 | DYSITSNYWG |
| 28 | rG055 CDRH2 | YITYSGYTS |
| 29 | rG055 CDRH3 | SINHGGYSYVVDA |

TABLE 1-4

| | | |
|---|---|---|
| 30 | rG056 light chain | DVQMTQSPSSLAASPGESVSISCRATKSIGIYLAWYQQKPGKTFKLLIYSGSTLQSG |
| | variable region | TPSRFSGSGSGTDFTLTIRSLEPEDFGLYFCQQFYENPFTFGSGTKLEIRRA |
| | amino acid | |
| | sequence | |
| 31 | rG056 light chain | GATGTCCAGATGACCCAGTCTCCGTCTTCTCTTGCTGCGTCTCCTGGAGAAAGTGTT |
| | variable region | TCCATCAGTTGCAGGGCAACTAAGAGCATTGGTATTTATTTAGCCTGGTATCAACAG |
| | nucleotide | AAACCTGGGAAAACATTTAAGCTTCTTATCTACTCTGGGTCAACTTTGCAATCTGGA |
| | sequence | ACTCCATCAAGGTTCAGTGGCAGTGGGTCTGGTACAGATTTCACTCTCACCATCAGA |
| | | AGCCTGGAGCCTGAAGATTTTGGACTCTATTTCTGTCAACAGTTTTATGAAAACCCA |
| | | TTCACGTTCGGCTCAGGGACGAAGTTGGAAATAAGACGGGCT |
| 32 | rG056 CDRL1 | RATKSIGIYLA |
| 33 | rG056 CDRL2 | SGSTLQS |
| 34 | rG056 CDRL3 | QQFYENPFT |
| 35 | rG056 heavy | EVQLQESGPGLVKPSQSLSLTCSVTDYSITTYFWGWIRKFPGNKMEWMGYMSYRGGT |
| | chain variable | SYNPSLKSRISITRDTSKNQFFLQLNSVTTEDTATYYCARCPNYGGHSLVFDYWGQG |
| | region amino acid | VMVTVSS |
| | sequence | |
| 36 | rG056 heavy | GAGGTGCAGCTTCAGGAGTCAGGACCTGGCCTTGTGAAACCCTCACAGTCACTCTCC |
| | chain variable | CTCACCTGTTCTGTCACTGATTACTCCATCACTACTTATTTCTGGGGCTGGATCCGG |
| | region nucleotide | AAGTTCCCAGGAAATAAAATGGAGTGGATGGGATACATGAGCTACCGTGGTGGCACT |
| | sequence | TCCTACAACCCATCTCTCAAGAGTCGAATCTCCATTACTAGAGACACATCGAAGAAT |
| | | CAGTTCTTCCTGCAGTTGAACTCTGTAACTACTGAGGACACAGCCACATATTACTGT |
| | | GCAAGATGCCCTAACTACGGAGGGCATTCCCTTGTTTTTGATTACTGGGGCCAAGGA |
| | | GTCATGGTCACAGTGTCCTCA |
| 37 | rG056 CDRH1 | DYSITTYFWG |
| 38 | rG056 CDRH2 | YMSYRGGTS |
| 39 | rG056 CDRH3 | CPNYGGHSLVFDY |
| 40 | rG061 light chain | DVQMTQSPSYLAASPGESVSISCKATKSISNYLAWYQQKPGEAYKVLIYSGSTLQSG |
| | variable region | TPSRFSGSGSGTDFTLTIRSLEPEDFGLYSCQQYYEKPLTFGSGTKLEIKRA |
| | amino acid | |
| | sequence | |
| 41 | rG061 light chain | GATGTCCAGATGACCCAGTCTCCGTCTTATCTTGCTGCGTCTCCTGGAGAAAGTGTT |
| | variable region | TCCATCAGTTGCAAGGCAACTAAGAGCATTAGTAATTATTTAGCCTGGTATCAACAG |
| | nucleotide | AAACCTGGGGAAGCATATAAGGTTCTTATCTATTCTGGGTCAACTTTGCAATCTGGA |
| | sequence | ACTCCATCAAGGTTCAGTGGCAGTGGATCTGGTACAGATTTCACTCTCACCATCAGA |
| | | AGCCTGGAGCCTGAAGATTTTGGACTCTATTCCTGTCAACAGTATTATGAAAAACCG |
| | | CTCACGTTCGGTTCTGGGACCAAGCTGGAGATCAAACGGGCT |
| 42 | rG061 CDRL1 | KATKSISNYLA |
| 43 | rG061 CDRL2 | SGSTLQS |
| 44 | rG061 CDRL3 | QQYYEKPLT |

TABLE 1-5

| | | |
|---|---|---|
| 45 | rG061 heavy | EVQLQESGPGLVKPSQSLSLTCSVTGYSITTYYWGWIRKFPGNKMEWMGYISYSGRT |
| | chain variable | SYNPSLKSRMSITRDASKNQFFLQLNSVTTDDTATYYCARSPINHGGYWYFDFWGPG |
| | region amino acid | TMVTVSS |
| | sequence | |
| 46 | rG061 heavy | GAGGTGCAGCTTCAGGAGTCAGGACCTGGCCTTGTGAAACCCTCACAGTCACTCTCC |
| | chain variable | CTCACCTGTTCTGTCACTGGTTACTCCATCACTACTTATTACTGGGGCTGGATCCGG |
| | region nucleotide | AAGTTCCCAGGAAATAAAATGGAGTGGATGGGGTACATAAGCTACAGTGGTCGCACT |
| | sequence | AGTTATAACCCATCTCTCAAAAGTCGAATGTCCATTACTAGAGACGCATCGAAGAAT |
| | | CAGTTCTTCCTACAGTTGAACTCTGTAACTACTGACGACACAGCCACATATTACTGT |
| | | GCAAGATCCCCAATTAACCACGGAGGGTACTGGTACTTTGACTTCTGGGGCCCAGGA |
| | | ACCATGGTCACCGTGTCCTCA |
| 47 | rG061 CDRH1 | GYSITTYYWG |
| 48 | rG061 CDRH2 | YISYSGRTS |
| 49 | rG061 CDRH3 | SPINHGGYWYFDF |
| 50 | DNAfragment | gcctccggactctagagccaccATGGTGCTGCAGACCCAGGTGTTCATCTCCCTGCT |
| | comprising DNA | GCTGTGGATCTCCGGCGCGTACGGCGATATCGTGATGATTAAACGTACGGTGGCCGC |
| | sequence | CCCCTCCGTGTTCATCTTCCCCCCCTCCGACGAGCAGCTGAAGTCCGGCACCGCCTC |
| | encoding human | CGTGGTGTGCCTGCTGAATAACTTCTACCCCAGAGAGGCCAAGGTGCAGTGGAAGGT |
| | light chain signal | GGACAACGCCCTGCAGTCCGGGAACTCCCAGGAGAGCGTGACCGAGCAGGACAGCAA |
| | sequence and | GGACAGCACCTACAGCCTGAGCAGCACCCTGACCCTGAGCAAAGCCGACTACGAGAA |
| | human k chain | GCACAAGGTGTACGCCTGCGAGGTGACCCACCAGGGCCTGAGCTCCCCCGTCACCAA |
| | constant region | GAGCTTCAACAGGGGGGAGTGTtaggggcccgtttaaacgggggaggcta |

TABLE 1-6

| | | |
|---|---|---|
| 51 | DNA fragment | gcctccggactctagagccaccATGAAACACCTGTGGTTCTTCCTCCTGCTGGTGGC |
| | comprising DNA | AGCTCCCAGATGGGTGCTGAGCCAGGTGCAATTGTGCAGGGGGTTAGCTCAGCCTCC |
| | sequence | ACCAAGGGCCCAAGCGTCTTCCCCCTGGCACCCTCCTCCAAGAGCACCTCTGGCGGC |
| | encoding human | ACAGCCGCCCTGGGCTGCCTGGTCAAGGACTACTTCCCCGAACCCGTGACCGTGAGC |
| | heavy chain signal | TGGAACTCAGGCGCCCTGACCAGCGGCGTGCACACCTTCCCCGCTGTCCTGCAGTCC |
| | sequence and | TCAGGACTCTACTCCCTCAGCAGCGTGGTGACCGTGCCCTCCAGCAGCTTGGGCACC |
| | human IgG1 | CAGACCTACATCTGCAACGTGAATCACAAGCCCAGCAACACCAAGGTGGACAAGAGA |
| | constant region | GTTGAGCCCAAATCTTGTGACAAAACTCACACATGCCCACCCTGCCCAGCACCTGAA |
| | | CTCCTGGGGGGACCCTCAGTCTTCCTCTTCCCCCCAAAACCCAAGGACACCCTCATG |
| | | ATCTCCCGGACCCCTGAGGTCACATGCGTGGTGGTGGACGTGAGCCACGAAGACCCT |
| | | GAGGTCAAGTTCAACTGGTACGTGGACGGCGTGGAGGTGCATAATGCCAAGACAAAG |
| | | CCCCGGGAGGAGCAGTACAACAGCACGTACCGGGTGGTCAGCGTCCTCACCGTCCTG |
| | | CACCAGGACTGGCTGAATGGCAAGGAGTACAAGTGCAAGGTCTCCAACAAAGCCCTC |
| | | CCAGCCCCCATCGAGAAAACCATCTCCAAAGCCAAAGGCCAGCCCCGGGAACCACAG |
| | | GTGTACACCCTGCCCCCATCCCGGGAGGAGATGACCAAGAACCAGGTCAGCCTGACC |
| | | TGCCTGGTCAAAGGCTTCTATCCCAGCGACATCGCCGTGGAGTGGGAGAGCAATGGC |
| | | CAGCCCGAGAACAACTACAAGACCACCCCTCCCGTGCTGGACTCCGACGGCTCCTTC |
| | | TTCCTCTACAGCAAGCTCACCGTGGACAAGAGCAGGTGGCAGCAGGGCAACGTCTTC |
| | | TCATGCTCCGTGATGCATGAGGCTCTGCACAACCACTACACCCAGAAGAGCCTCTCC |
| | | CTGTCTCCCGGCAAAtgagatatcgggcccgtttaaacgggggaggcta |
| 52 | DNA fragment | ccagcctccggactctagagccaccATGGTGCTGCAGACCCAGGTGTTCATCAGCCT |
| | comprising DNA | GCTGCTGTGGATCAGCGGCGCCTACGGCGACATCCAGATGACCCAGAGCCCTAGCCT |
| | sequence | GCTGAGCGCCAGCGTGGGCGATAGAGTGACCCTGAACTGCAAGGCCAGCCAGAACAT |
| | encoding chG019 | CTACAAGAACCTGGCCTGGTATCAGCAGAAGCTGGGCGAGGGCCCCAAGCTGCTGAT |
| | light chain | CTACGACGCCAACACCCTGCAGACCGGCATCCCCAGCAGATTTTCTGGCAGCGGCAG |
| | | CGGCTCCGACTTCACCCTGACAATCAGCAGCCTGCAGCCCGAGGACGTGGCCACCTA |
| | | CTTTTGCCAGCAGTACTACAGCGGCTGGGCCTTCGGGGGCGTGACCAACCTGGAACT |
| | | GAAGAGAGCCGTGGCCGCTCCCTCCGTGTTCATCTTCCCACCTAGCGACGAGCAGCT |
| | | GAAGTCCGGCACAGCCTCTGTCGTGTGCCTGCTGAACAACTTCTACCCCCGCGAGGC |
| | | CAAGGTGCAGTGGAAGGTGGACAATGCCCTGCAGTCTGGCAACAGCCAGGAAAGCGT |
| | | GACCGAGCAGGACAGCAAGGACTCCACCTACAGCCTGAGCAGCACCCTGACCCTGAG |
| | | CAAGGCCGACTACGAGAAGCACAAGGTGTACGCCTGCGAAGTGACCCACCAGGGCCT |
| | | GTCTAGCCCCGTGACCAAGAGCTTCAACCGGGGCGAGTGTtgagtttaaacggggga |
| | | ggctaact |
| 53 | chG019 light chain | MVLQTQVFISLLLWISGAYGDIQMTQSPSLLSASVGDRVTLNCKASQNIYKNLAWYQ |
| | full-length amino | QKLGEGPKLLIYDANTLQTGIPSRFSGSGSGSDFTLTISSLQPEDVATYFCQQYYSG |
| | acid sequence | WAFGGVTNLELKRAVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLSSPVTKSF |
| | | NRGEC |

TABLE 1-7

| | | |
|---|---|---|
| 54 | chG019 light | ATGGTGCTGCAGACCCAGGTGTTCATCAGCCTGCTGCTGTGGATCAGCGGCGCCTAC |
| | chain full-length | GGCGACATCCAGATGACCCAGAGCCCTAGCCTGCTGAGCGCCAGCGTGGGCGATAGA |
| | nucleotide | GTGACCCTGAACTGCAAGGCCAGCCAGAACATCTACAAGAACCTGGCCTGGTATCAG |
| | sequence | CAGAAGCTGGGCGAGGGCCCCAAGCTGCTGATCTACGACGCCAACACCCTGCAGACC |
| | | GGCATCCCCAGCAGATTTTCTGGCAGCGGCAGCGGCTCCGACTTCACCCTGACAATC |
| | | AGCAGCCTGCAGCCCGAGGACGTGGCCACCTACTTTTGCCAGCAGTACTACAGCGGC |
| | | TGGGCCTTCGGCGGCGTGACCAACCTGGAACTGAAGAGAGCCGTGGCCGCTCCCTCC |
| | | GTGTTCATCTTCCCACCTAGCGACGAGCAGCTGAAGTCCGGCACAGCCTCTGTCGTG |
| | | TGCCTGCTGAACAACTTCTACCCCCGCGAGGCCAAGGTGCAGTGGAAGGTGGACAAT |
| | | GCCCTGCAGTCTGGCAACAGCCAGGAAAGCGTGACCGAGCAGGACAGCAAGGACTCC |
| | | ACCTACAGCCTGAGCAGCACCCTGACCCTGAGCAAGGCCGACTACGAGAAGCACAAG |
| | | GTGTACGCCTGCGAAGTGACCCACCAGGGCCTGTCTAGCCCCGTGACCAAGAGCTTC |
| | | AACCGGGGCGAGTGT |
| 10 | chG019 light | DIQMTQSPSLLSASVGDRVTLNCKASQNIYKNLAWYQQKLGEGPKLLIYDANTLQTG |
| | chain variable | IPSRFSGSGSGSDFTLTISSLQPEDVATYFCQQYYSGWAFGGVTNLELKRA |
| | region amino acid | |
| | sequence | |
| 55 | chG019 light | GACATCCAGATGACCCAGAGCCCTAGCCTGCTGAGCGCCAGCGTGGGCGATAGAGTG |
| | chain variable | ACCCTGAACTGCAAGGCCAGCCAGAACATCTACAAGAACCTGGCCTGGTATCAGCAG |
| | region nucleotide | AAGCTGGGCGAGGGCCCCAAGCTGCTGATCTACGACGCCAACACCCTGCAGACCGGC |
| | sequence | ATCCCCAGCAGATTTTCTGGCAGCGGCAGCGGCTCCGACTTCACCCTGACAATCAGC |
| | | AGCCTGCAGCCCGAGGACGTGGCCACCTACTTTTGCCAGCAGTACTACAGGGGCTGG |
| | | GCCTTCGGCGGCGTGACCAACCTGGAACTGAAGAGAGCC |
| 12 | chG019 CDRL1 | KASQNIYKNLA |
| 13 | chG019 CDRL2 | DANTLQT |
| 14 | chG019 CDRL3 | QQYYSGWA |
| 56 | chG019 heavy | MKHLWFFLLLVAAPRWVLSQVQLQQSGAELVKPGSSVKISCKASGYTFTRNFMHWIK |
| | chain full-length | QQPGNGLEWIGWIYPGDGETEYNQKFNGKATLTADRSSSTAYMELSRLTSEDSAVYF |
| | amino acid | CARGVYGGFAGGYFDFWGQGVMVTVSSASTKGPSVFPLAPSSKSTSGGTAALGCLVK |
| | sequence | DYFPEPVTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNVFSCSVMHEAL |
| | | HNHYTQKSLSLSPGK |

TABLE 1-8

| | | |
|---|---|---|
| 57 | chG019 heavy | ATGAAACACCTGTGGTTCTTCCTCCTGCTGGTGGCAGCTCCCAGATGGGTGCTGAGC |
| | chain full-length | CAGGTGCAGCTGCAGCAGTCTGGCGCCGAGCTCGTGAAGCCTGGCAGCAGCGTGAAG |
| | nucleotide | ATCAGCTGCAAGGCCAGCGGCTACACCTTCACCCGGAACTTCATGCACTGGATCAAG |
| | sequence | CAGCAGCCCGGCAACGGCCTGGAATGGATCGGCTGGATCTATCCCGGCGACGGCGAG |
| | | ACAGAGTACAACCAGAAGTTCAACGGCAAGGCCACCCTGACCGCCGACAGAAGCAGC |
| | | TCCACCGCCTACATGGAACTGAGCCGGCTGACCAGCGAGGACAGCGCCGTGTACTTT |
| | | TGCGCCAGAGGCGTGTACGGGGGCTTCGCTGGGGGCTACTTCGATTTTTGGGGCCAG |
| | | GGCGTGATGGTCACCGTCAGCTCAGCCTCCACCAAGGGCCCAAGCGTCTTCCCCCTG |
| | | GCACCCTCCTCCAAGAGCACCTCTGGCGGCACAGCCGCCCTGGGCTGCCTGGTCAAG |
| | | GACTACTTCCCCGAACCCGTGACCGTGAGCTGGAACTCAGGCGCCCTGACCAGCGGC |
| | | GTGCACACCTTCCCCGCTGTCCTGCAGTCCTCAGGACTCTACTCCCTCAGCAGCGTG |
| | | GTGACCGTGCCCTCCAGCAGCTTGGGCACCCAGACCTACATCTGCAACGTGAATCAC |
| | | AAGCCCAGCAACACCAAGGTGGACAAGAGAGTTGAGCCCAAATCTTGTGACAAAACT |
| | | CACACATGCCCACCCTGCCCAGCACCTGAACTCCTGGGGGGACCCTCAGTCTTCCTC |
| | | TTCCCCCCAAAACCCAAGGACACCCTCATGATCTCCCGGACCCCTGAGGTCACATGC |
| | | GTGGTGGTGGACGTGAGCCACGAAGACCCTGAGGTCAAGTTCAACTGGTACGTGGAC |
| | | GGCGTGGAGGTGCATAATGCCAAGACAAAGCCCCGGGAGGAGCAGTACAACAGCACG |
| | | TACCGGGTGGTCAGCGTCCTCACCGTCCTGCACCAGGACTGGCTGAATGGCAAGGAG |
| | | TACAAGTGCAAGGTCTCCAACAAAGCCCTCCCAGCCCCCATCGAGAAAACCATCTCC |
| | | AAAGCCAAAGGCCAGCCCCGGGAACCACAGGTGTACACCCTGCCCCCATCCCGGGAG |
| | | GAGATGACCAAGAACCAGGTCAGCCTGACCTGCCTGGTCAAAGGCTTCTATCCCAGC |
| | | GACATCGCCGTGGAGTGGGAGAGCAATGGCCAGCCCGAGAACAACTACAAGACCACC |
| | | CCTCCCGTGCTGGACTCCGACGGCTCCTTCTTCCTCTACAGCAAGCTCACCGTGGAC |
| | | AAGAGCAGGTGGCAGCAGGGCAACGTCTTCTCATGCTCCGTGATGCATGAGGCTCTG |
| | | CACAACCACTACACCCAGAAGAGCCTCTCCCTGTCTCCCGGCAAA |
| 58 | chG019 heavy | QVQLQQSGAELVKPGSSVKISCKASGYTFTRNFMHWIKQQPGNGLEWIGWIYPGDGE |
| | chain variable | TEYNQKFNGKATLTADRSSSTAYMELSRLTSEDSAVYFCARGVYGGFAGGYFDFWGQ |
| | region amino acid | GVMVTVSS |
| | sequence | |
| 59 | chG019 heavy | CAGGTGCAGCTGCAGCAGTCTGGCGCCGAGCTCGTGAAGCCTGGCAGCAGCGTGAAG |
| | chain variable | ATCAGCTGCAAGGCCAGCGGCTACACCTTCACCCGGAACTTCATGCACTGGATCAAG |
| | region nucleotide | CAGCAGCCCGGCAACGGCCTGGAATGGATCGGCTGGATCTATCCCGGCGACGGCGAG |
| | sequence | ACAGAGTACAACCAGAAGTTCAACGGCAAGGCCACCCTGACCGCCGACAGAAGCAGC |
| | | TCCACCGCCTACATGGAACTGAGCCGGCTGACCAGCGAGGACAGCGCCGTGTACTTT |
| | | TGCGCCAGAGGCGTGTACGGCGGCTTCGCTGGCGGCTACTTCGATTTTTGGGGCCAG |
| | | GGCGTGATGGTCACCGTCAGCTCA |
| 17 | chG019 CDRH1 | GYTFTRNFMH |
| 60 | chG019 CDRH2 | WIYPGDGETE |
| 19 | chG019 CDRH3 | GVYGGFAGGYFDF |

TABLE 1-9

| | | |
|---|---|---|
| 61 | hL02 light chain | MVLQTQVFISLLLWISGAYGDIQMTQSPSSLSASVGDRVTITCKASQNIYKNLAWYQ |
| | full-length amino | QKPGKAPKLLIYDANTLQTGVPSRFSGSGSGSDFTLTISSLQPEDFATYFCQQYYSG |
| | acid sequence | WAFGQGTKVEIKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLSSPVTKSF |
| | | NRGEC |
| 62 | hL02 light chain | ATGGTGCTGCAGACCCAGGTGTTCATCTCCCTGCTGCTGTGGATCTCCGGCGCGTAC |
| | full-length | GGCGACATCCAGATGACCCAGAGCCCTAGCAGCCTGAGCGCCAGCGTGGGCGACAGA |
| | nucleotide | GTGACCATCACATGCAAGGCCAGCCAGAACATCTACAAGAACCTGGCCTGGTATCAG |
| | sequence | CAGAAGCCCGGCAAGGCCCCCAAGCTGCTGATCTACGACGCCAACACCCTGCAGACC |
| | | GGCGTGCCCAGCAGATTTTCTGGCAGCGGCAGCGGCTCCGACTTCACCCTGACAATC |
| | | AGCAGCCTGCAGCCCGAGGACTTCGCCACCTACTTTTGCCAGCAGTACTACAGCGGC |
| | | TGGGCCTTCGGCCAGGGCACCAAGGTGGAAATCAAGCGTACGGTGGCCGCCCCCTCC |
| | | GTGTTCATCTTCCCCCCCTCCGACGAGCAGCTGAAGTCCGGCACCGCCTCCGTGGTG |
| | | TGCCTGCTGAATAACTTCTACCCCAGAGAGGCCAAGGTGCAGTGGAAGGTGGACAAC |
| | | GCCCTGCAGTCCGGGAACTCCCAGGAGAGCGTGACCGAGCAGGACAGCAAGGACAGC |
| | | ACCTACAGCCTGAGCAGCACCCTGACCCTGAGCAAAGCCGACTACGAGAAGCACAAG |
| | | GTGTACGCCTGCGAGGTGACCCACCAGGGCCTGAGCTCCCCCGTCACCAAGAGCTTC |
| | | AACAGGGGGGAGTGT |
| 63 | hL02 light chain | DIQMTQSPSSLSASVGDRVTITCKASQNIYKNLAWYQQKPGKAPKLLIYDANTLQTG |
| | variable region | VPSRFSGSGSGSDFTLTISSLQPEDFATYFCQQYYSGWAFGQGTKVEIKRT |
| | amino acid | |
| | sequence | |
| 64 | hL02 light chain | GACATCCAGATGACCCAGAGCCCTAGCAGCCTGAGCGCCAGCGTGGGCGACAGAGTG |
| | variable region | ACCATCACATGCAAGGCCAGCCAGAACATCTACAAGAACCTGGCCTGGTATCAGCAG |
| | nucleotide | AAGCCCGGCAAGGCCCCCAAGCTGCTGATCTACGACGCCAACACCCTGCAGACCGGC |
| | sequence | GTGCCCAGCAGATTTTCTGGCAGCGGCAGCGGCTCCGACTTCACCCTGACAATCAGC |
| | | AGCCTGCAGCCCGAGGACTTCGCCACCTACTTTTGCCAGCAGTACTACAGCGGCTGG |
| | | GCCTTCGGCCAGGGCACCAAGGTGGAAATCAAGCGTACG |
| 65 | hL03 light chain | MVLQTQVFISLLLWISGAYGDIQMTQSPSSLSASVGDRVTITCKASQNIYKNLAWYQ |
| | full-length amino | QKLGEGPKLLIYDANTLQTGVPSRFSGSGSGTDFTLTISSLQPEDFATYYCQQYYSG |
| | acid sequence | WAFGQGTKVEIKRTVAAPSVFIFPPSDEQLKSGTASVVCLLNNFYPREAKVQWKVDN |
| | | ALQSGNSQESVTEQDSKDSTYSLSSTLTLSKADYEKHKVYACEVTHQGLSSPVTKSF |
| | | NRGEC |

TABLE 1-10

| | | |
|---|---|---|
| 66 | hL03 light chain | ATGGTGCTGCAGACCCAGGTGTTCATCTCCCTGCTGCTGTGGATCTCCGGCGCGTAC |
| | full-length | GGCGACATCCAGATGACCCAGAGCCCTAGCAGCCTGAGCGCCAGCGTGGGCGACAGA |
| | nucleotide | GTGACCATCACATGCAAGGCCAGCCAGAACATCTACAAGAACCTGGCCTGGTATCAG |
| | sequence | CAGAAGCTGGGCGAGGGCCCCAAGCTGCTGATCTACGACGCCAACACCCTGCAGACC |
| | | GGCGTGCCCAGCAGATTTTCTGGCAGCGGCTCCGGCACCGACTTCACCCTGACAATC |
| | | AGCAGCCTGCAGCCCGAGGACTTCGCCACCTACTACTGCCAGCAGTACTACAGCGGC |
| | | TGGGCCTTTGGCCAGGGCACCAAGGTGGAAATCAAGCGTACGGTGGCCGCCCCCTCC |
| | | GTGTTCATCTTCCCCCCCTCCGACGAGCAGCTGAAGTCCGGCACCGCCTCCGTGGTG |
| | | TGCCTGCTGAATAACTTCTACCCCAGAGAGGCCAAGGTGCAGTGGAAGGTGGACAAC |
| | | GCCCTGCAGTCCGGGAACTCCCAGGAGAGCGTGACCGAGCAGGACAGCAAGGACAGC |
| | | ACCTACAGCCTGAGCAGCACCCTGACCCTGAGCAAAGCCGACTACGAGAAGCACAAG |
| | | GTGTACGCCTGCGAGGTGACCCACCAGGGCCTGAGCTCCCCCGTCACCAAGAGCTTC |
| | | AACAGGGGGGAGTGT |
| 67 | hL03 light chain | DIQMTQSPSSLSASVGDRVTITCKASQNIYKNLAWYQQKLGEGPKLLIYDANTLQTG |
| | variable region | VPSRFSGSGSGTDFTLTISSLQPEDFATYYCQQYYSGWAFGQGTKVEIKRT |
| | amino acid | |
| | sequence | |
| 68 | hL03 light chain | GACATCCAGATGACCCAGAGCCCTAGCAGCCTGAGCGCCAGCGTGGGCGACAGAGTG |
| | variable region | ACCATCACATGCAAGGCCAGCCAGAACATCTACAAGAACCTGGCCTGGTATCAGCAG |
| | nucleotide | AAGCTGGGCGAGGGCCCCAAGCTGCTGATCTACGACGCCAACACCCTGCAGACCGGC |
| | sequence | GTGCCCAGCAGATTTTCTGGCAGCGGCTCCGGCACCGACTTCACCCTGACAATCAGC |
| | | AGCCTGCAGCCCGAGGACTTCGCCACCTACTACTGCCAGCAGTACTACAGCGGCTGG |
| | | GCCTTTGGCCAGGGCACCAAGGTGGAAATCAAGCGTACG |
| 69 | hH01 heavy chain | MKHLWFFLLLVAAPRWVLSEVQLVQSGAEVKKPGASVKVSCKASGYTFTRNFMHWVR |
| | full-length amino | QAPGQGLEWMGWIYPGDGETEYAQKFQGRVTITADTSTSTAYMELSSLRSEDTAVYY |
| | acid sequence | CARGVYGGFAGGYFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNVFSCSVMHEAL |
| | | HNHYTQKSLSLSPGK |

TABLE 1-11

| | | |
|---|---|---|
| 70 | hH01 heavy chain | ATGAAACACCTGTGGTTCTTCCTCCTGCTGGTGGCAGCTCCCAGATGGGTGCTGAGC |
| | full-length | GAAGTGCAGCTGGTGCAGTCTGGCGCCGAAGTGAAGAAACCAGGCGCCAGCGTGAAG |
| | nucleotide | GTGTCCTGCAAGGCCAGCGGCTACACCTTTACCCGGAACTTCATGCACTGGGTGCGC |
| | sequence | CAGGCTCCAGGCCAGGGACTGGAATGGATGGGCTGGATCTATCCCGGCGACGGCGAG |
| | | ACAGAGTACGCCCAGAAATTCCAGGGCAGAGTGACCATCACCGCCGACACCAGCACC |
| | | TCCACCGCCTACATGGAACTGAGCAGCCTGCGGAGCGAGGACACCGCCGTGTACTAT |
| | | TGTGCCAGAGGCGTGTACGGCGGCTTCGCTGGCGGCTACTTCGATTTTTGGGGCCAG |
| | | GGCACCCTCGTGACCGTCAGCTCAGCCTCCACCAAGGGCCCAAGCGTCTTCCCCCTG |
| | | GCACCCTCCTCCAAGAGCACCTCTGGGGGCACAGCCGCCCTGGGCTGCCTGGTCAAG |
| | | GACTACTTCCCCGAACCCGTGACCGTGAGCTGGAACTCAGGCGCCCTGACCAGCGGC |
| | | GTGCACACCTTCCCCGCTGTCCTGCAGTCCTCAGGACTCTACTCCCTCAGCAGCGTG |
| | | GTGACCGTGCCCTCCAGCAGCTTGGGCACCCAGACCTACATCTGCAACGTGAATCAC |
| | | AAGCCCAGCAACACCAAGGTGGACAAGAGAGTTGAGCCCAAATCTTGTGACAAAACT |
| | | CACACATGCCCACCCTGCCCAGCACCTGAACTCCTGGGGGGACCCTCAGTCTTCCTC |
| | | TTCCCCCCAAAACCCAAGGACACCCTCATGATCTCCCGGACCCCTGAGGTCACATGC |
| | | GTGGTGGTGGACGTGAGCCACGAAGACCCTGAGGTCAAGTTCAACTGGTACGTGGAC |
| | | GGCGTGGAGGTGCATAATGCCAAGACAAAGCCCCGGGAGGAGCAGTACAACAGCACG |
| | | TACCGGGTGGTCAGCGTCCTCACCGTCCTGCACCAGGACTGGCTGAATGGCAAGGAG |
| | | TACAAGTGCAAGGTCTCCAACAAAGCCCTCCCAGCCCCCATCGAGAAAACCATCTCC |
| | | AAAGCCAAAGGCCAGCCCCGGGAACCACAGGTGTACACCCTGCCCCCATCCCGGGAG |
| | | GAGATGACCAAGAACCAGGTCAGCCTGACCTGCCTGGTCAAAGGCTTCTATCCCAGC |
| | | GACATCGCCGTGGAGTGGGAGAGCAATGGCCAGCCCGAGAACAACTACAAGACCACC |
| | | CCTCCCGTGCTGGACTCCGACGGCTCCTTCTTCCTCTACAGCAAGCTCACCGTGGAC |
| | | AAGAGCAGGTGGCAGCAGGGCAACGTCTTCTCATGCTCCGTGATGCATGAGGCTCTG |
| | | CACAACCACTACACCCAGAAGAGCCTCTCCCTGTCTCCCGGCAAA |
| 71 | hH01 heavy chain | EVQLVQSGAEVKKPGASVKVSCKASGYTFTRNFMHWVRQAPGQGLEWMGWIYPGDGE |
| | variable region | TEYAQKFQGRVTITADTSTSTAYMELSSLRSEDTAVYYCARGVYGGFAGGYFDFWGQ |
| | amino acid | GTLVTVSS |
| | sequence | |
| 72 | hH01 heavy chain | GAAGTGCAGCTGGTGCAGTCTGGCGCCGAAGTGAAGAAACCAGGCGCCAGCGTGAAG |
| | variable region | GTGTCCTGCAAGGCCAGCGGCTACACCTTTACCCGGAACTTCATGCACTGGGTGCGC |
| | nucleotide | CAGGCTCCAGGCCAGGGACTGGAATGGATGGGCTGGATCTATCCCGGCGACGGCGAG |
| | sequence | ACAGAGTACGCCCAGAAATTCCAGGGCAGAGTGACCATCACCGCCGACACCAGCACC |
| | | TCCACCGCCTACATGGAACTGAGCAGCCTGCGGAGCGAGGACACCGCCGTGTACTAT |
| | | TGTGCCAGAGGCGTGTACGGGGGCTTCGCTGGGGGCTACTTCGATTTTTGGGGCCAG |
| | | GGCACCCTCGTGACCGTCAGCTCA |

TABLE 1-12

| | | |
|---|---|---|
| 73 | hH02 heavy chain | MKHLWFFLLLVAAPRWVLSEVQLVQSGAEVKKPGASVKVSCKASGYTFTRNFMHWVR |
| | full-length amino | QAPGQGLEWMGWIYPGDGETEYNQKFQGRVTITADRSTSTAYMELSSLRSEDTAVYF |
| | acid sequence | CARGVYGGFAGGYFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNVFSCSVMHEAL |
| | | HNHYTQKSLSLSPGK |
| 74 | hH02 heavy chain | ATGAAACACCTGTGGTTCTTCCTCCTGCTGGTGGCAGCTCCCAGATGGGTGCTGAGC |
| | full-length | GAAGTGCAGCTGGTGCAGTCTGGCGCCGAAGTGAAGAAACCAGGCGCCAGCGTGAAG |
| | nucleotide | GTGTCCTGCAAGGCCAGCGGCTACACCTTTACCCGGAACTTCATGCACTGGGTGCGC |
| | sequence | CAGGCTCCAGGCCAGGGACTGGAATGGATGGGCTGGATCTATCCCGGCGACGGCGAG |
| | | ACAGAGTACAACCAGAAATTCCAGGGCAGAGTGACCATCACCGCCGACAGAAGCACC |
| | | AGCACCGCCTACATGGAACTGAGCAGCCTGCGGAGCGAGGATACCGCCGTGTACTTC |
| | | TGTGCCAGAGGCGTGTACGGCGGCTTCGCTGGGGGCTACTTCGATTTTTGGGGCCAG |
| | | GGCACCCTCGTGACCGTCAGCTCAGCCTCCACCAAGGGCCCAAGCGTCTTCCCCCTG |
| | | GCACCCTCCTCCAAGAGCACCTCTGGCGGCACAGCCGCCCTGGGCTGCCTGGTCAAG |
| | | GACTACTTCCCCGAACCCGTGACCGTGAGCTGGAACTCAGGCGCCCTGACCAGGGGC |
| | | GTGCACACCTTCCCCGCTGTCCTGCAGTCCTCAGGACTCTACTCCCTCAGCAGCGTG |
| | | GTGACCGTGCCCTCCAGCAGCTTGGGCACCCAGACCTACATCTGCAACGTGAATCAC |
| | | AAGCCCAGCAACACCAAGGTGGACAAGAGAGTTGAGCCCAAATCTTGTGACAAAACT |
| | | CACACATGCCCACCCTGCCCAGCACCTGAACTCCTGGGGGGACCCTCAGTCTTCCTC |
| | | TTCCCCCCAAAACCCAAGGACACCCTCATGATCTCCCGGACCCCTGAGGTCACATGC |
| | | GTGGTGGTGGACGTGAGCCACGAAGACCCTGAGGTCAAGTTCAACTGGTACGTGGAC |
| | | GGCGTGGAGGTGCATAATGCCAAGACAAAGCCCCGGGAGGAGCAGTACAACAGCACG |
| | | TACCGGGTGGTCAGCGTCCTCACCGTCCTGCACCAGGACTGGCTGAATGGCAAGGAG |
| | | TACAAGTGCAAGGTCTCCAACAAAGCCCTCCCAGCCCCCATCGAGAAAACCATCTCC |
| | | AAAGCCAAAGGCCAGCCCCGGGAACCACAGGTGTACACCCTGCCCCCATCCCGGGAG |
| | | GAGATGACCAAGAACCAGGTCAGCCTGACCTGCCTGGTCAAAGGCTTCTATCCCAGC |
| | | GACATCGCCGTGGAGTGGGAGAGCAATGGCCAGCCCGAGAACAACTACAAGACCACC |
| | | CCTCCCGTGCTGGACTCCGACGGCTCCTTCTTCCTCTACAGCAAGCTCACCGTGGAC |
| | | AAGAGCAGGTGGCAGCAGGGCAACGTCTTCTCATGCTCCGTGATGCATGAGGCTCTG |
| | | CACAACCACTACACCCAGAAGAGCCTCTCCCTGTCTCCCGGCAAA |
| 75 | hH02 heavy chain | EVQLVQSGAEVKKPGASVKVSCKASGYTFTRNFMHWVRQAPGQGLEWMGWIYPGDGE |
| | variable region | TEYNQKFQGRVTITADRSTSTAYMELSSLRSEDTAVYFCARGVYGGFAGGYFDFWGQ |
| | amino acid | GTLVTVSS |
| | sequence | |

TABLE 1-13

| | | |
|---|---|---|
| 76 | hH02 heavy chain | GAAGTGCAGCTGGTGCAGTCTGGCGCCGAAGTGAAGAAACCAGGCGCCAGCGTGAAG |
| | variable region | GTGTCCTGCAAGGCCAGCGGCTACACCTTTACCCGGAACTTCATGCACTGGGTGCGC |
| | nucleotide | CAGGCTCCAGGCCAGGGACTGGAATGGATGGGCTGGATCTATCCCGGCGACGGCGAG |
| | sequence | ACAGAGTACAACCAGAAATTCCAGGGCAGAGTGACCATCACCGCCGACAGAAGCACC |
| | | AGCACCGCCTACATGGAACTGAGCAGCCTGCGGAGCGAGGATACCGCCGTGTACTTC |
| | | TGTGCCAGAGGCGTGTACGGCGGCTTCGCTGGGGGCTACTTCGATTTTTGGGGCCAG |
| | | GGCACCCTCGTGACCGTCAGCTCA |
| 77 | hH04 heavy chain | MKHLWFFLLLVAAPRWVLSQVQLVQSGAEVKKPGASVKVSCKASGYTFTRNFMHWIR |
| | full-length amino | QAPGQGLEWMGWIYPGDGETEYAQKFQGRVTLTADRSTSTAYMELSSLRSEDTAVYY |
| | acid sequence | CARGVYGGFAGGYFDFWGQGTLVTVSSASTKGPSVFPLAPSSKSTSGGTAALGCLVK |
| | | DYFPEPVTVSWNSGALTSGVHTFPAVLQSSGLYSLSSVVTVPSSSLGTQTYICNVNH |
| | | KPSNTKVDKRVEPKSCDKTHTCPPCPAPELLGGPSVFLFPPKPKDTLMISRTPEVTC |
| | | VVVDVSHEDPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLVKGFYPS |
| | | DIAVEWESNGQPENNYKTTPPVLDSDGSFFLYSKLTVDKSRWQQGNVFSCSVMHEAL |
| | | HNHYTQKSLSLSPGK |
| 78 | hH04 heavy chain | ATGAAACACCTGTGGTTCTTCCTCCTGCTGGTGGCAGCTCCCAGATGGGTGCTGAGC |
| | full-length | CAGGTGCAGCTGGTGCAGTCTGGCGCCGAAGTGAAGAAACCAGGCGCCAGCGTGAAG |
| | nucleotide | GTGTCCTGCAAGGCCAGCGGCTACACCTTTACCCGGAACTTCATGCACTGGATCCGG |
| | sequence | CAGGCCCCTGGACAGGGCCTGGAATGGATGGGCTGGATCTATCCCGGCGACGGCGAG |
| | | ACAGAGTACGCCCAGAAATTCCAGGGCAGAGTGACCCTGACCGCCGACAGAAGCACC |
| | | AGCACCGCCTACATGGAACTGAGCAGCCTGCGGAGCGAGGACACCGCCGTGTACTAT |
| | | TGTGCCAGAGGCGTGTACGGCGGCTTCGCTGGCGGCTACTTCGATTTTTGGGGCCAG |
| | | GGCACCCTCGTGACCGTCAGCTCAGCCTCCACCAAGGGCCCAAGCGTCTTCCCCCTG |
| | | GCACCCTCCTCCAAGAGCACCTCTGGGGGCACAGCCGCCCTGGGCTGCCTGGTCAAG |
| | | GACTACTTCCCCGAACCCGTGACCGTGAGCTGGAACTCAGGCGCCCTGACCAGCGGC |
| | | GTGCACACCTTCCCCGCTGTCCTGCAGTCCTCAGGACTCTACTCCCTCAGCAGCGTG |
| | | GTGACCGTGCCCTCCAGCAGCTTGGGCACCCAGACCTACATCTGCAACGTGAATCAC |
| | | AAGCCCAGCAACACCAAGGTGGACAAGAGAGTTGAGCCCAAATCTTGTGACAAAACT |
| | | CACACATGCCCACCCTGCCCAGCACCTGAACTCCTGGGGGGACCCTCAGTCTTCCTC |
| | | TTCCCCCCAAAACCCAAGGACACCCTCATGATCTCCCGGACCCCTGAGGTCACATGC |
| | | GTGGTGGTGGACGTGAGCCACGAAGACCCTGAGGTCAAGTTCAACTGGTACGTGGAC |
| | | GGCGTGGAGGTGCATAATGCCAAGACAAAGCCCCGGGAGGAGCAGTACAACAGCACG |
| | | TACCGGGTGGTCAGCGTCCTCACCGTCCTGCACCAGGACTGGCTGAATGGCAAGGAG |
| | | TACAAGTGCAAGGTCTCCAACAAAGCCCTCCCAGCCCCCATCGAGAAAACCATCTCC |
| | | AAAGCCAAAGGCCAGCCCCGGGAACCACAGGTGTACACCCTGCCCCCATCCCGGGAG |
| | | GAGATGACCAAGAACCAGGTCAGCCTGACCTGCCTGGTCAAAGGCTTCTATCCCAGC |
| | | GACATCGCCGTGGAGTGGGAGAGCAATGGCCAGCCCGAGAACAACTACAAGACCACC |
| | | CCTCCCGTGCTGGACTCCGACGGCTCCTTCTTCCTCTACAGCAAGCTCACCGTGGAC |
| | | AAGAGCAGGTGGCAGCAGGGCAACGTCTTCTCATGCTCCGTGATGCATGAGGCTCTG |
| | | CACAACCACTACACCCAGAAGAGCCTCTCCCTGTCTCCCGGCAAA |

TABLE 1-14

| | | |
|---|---|---|
| 79 | hH04 heavy chain | QVQLVQSGAEVKKPGASVKVSCKASGYTFTRNFMHWIRQAPGQGLEWMGWIYPGDGE |
| | variable region | TEYAQKFQGRVTLTADRSTSTAYMELSSLRSEDTAVYYCARGVYGGFAGGYFDFWGQ |
| | amino acid | GTLVTVSS |
| | sequence | |
| 80 | hH04 heavy chain | CAGGTGCAGCTGGTGCAGTCTGGCGCCGAAGTGAAGAAACCAGGCGCCAGCGTGAAG |
| | variable region | GTGTCCTGCAAGGCCAGCGGCTACACCTTTACCCGGAACTTCATGCACTGGATCCGG |
| | nucleotide | CAGGCCCCTGGACAGGGCCTGGAATGGATGGGCTGGATCTATCCCGGCGACGGCGAG |
| | sequence | ACAGAGTACGCCCAGAAATTCCAGGGCAGAGTGACCCTGACCGCCGACAGAAGCACC |
| | | AGCACCGCCTACATGGAACTGAGCAGCCTGCGGAGCGAGGACACCGCCGTGTACTAT |
| | | TGTGCCAGAGGCGTGTACGGGGGCTTCGCTGGGGGCTACTTCGATTTTTGGGGCCAG |
| | | GGCACCCTCGTGACCGTCAGCTCA |
| 81 | NOV0712 light | MVLQTQVFISLLLWISGAYGDIQMTQSPSSLSASVGDRVTITCRASQSISSYLNWYQ |
| | chain full-length | QKPGKAPKLLIYAVSTLQSGVPSRFSGSGSGTDFTLTISSLQPEDFATYYCQQSGTF |
| | amino acid | PPTTFGQGTKVEIKRTVAAPSVFIFPPSDEQLKSGTASVVCLLNNFYPREAKVQWKV |
| | sequence | DNALQSGNSQESVTEQDSKDSTYSLSSTLTLSKADYEKHKVYACEVTHQGLSSPVTK |
| | | SFNRGEC |
| 82 | NOV0712 light | ATGGTGCTGCAGACCCAGGTGTTCATCTCCCTGCTGCTGTGGATCTCCGGCGCGTAC |
| | chain full-length | GGCGACATCCAGATGACCCAGAGCCCTAGCAGCCTGAGCGCCAGCGTGGGCGACAGA |
| | nucleotide | GTGACCATCACCTGTAGAGCCAGCCAGAGCATCAGCAGCTACCTGAACTGGTATCAG |
| | sequence | CAGAAGCCCGGCAAGGCCCCCAAACTGCTGATCTACGCCGTGTCCACACTGCAGAGC |
| | | GGCGTGCCCAGCAGATTTTCTGGCAGCGGCTCCGGCACCGACTTCACCCTGACAATC |
| | | AGCAGCCTGCAGCCCGAGGACTTCGCCACCTACTACTGTCAGCAGTCCGGCACCTTC |
| | | CCCCCCACCACATTTGGCCAGGGCACCAAGGTGGAAATCAAGCGTACGGTGGCCGCC |
| | | CCCTCCGTGTTCATCTTCCCCCCCTCCGACGAGCAGCTGAAGTCCGGCACCGCCTCC |
| | | GTGGTGTGCCTGCTGAATAACTTCTACCCCAGAGAGGCCAAGGTGCAGTGGAAGGTG |
| | | GACAACGCCCTGCAGTCCGGGAACTCCCAGGAGAGCGTGACCGAGCAGGACAGCAAG |
| | | GACAGCACCTACAGCCTGAGCAGCACCCTGACCCTGAGCAAAGCCGACTACGAGAAG |
| | | CACAAGGTGTACGCCTGCGAGGTGACCCACCAGGGCCTGAGCTCCCCCGTCACCAAG |
| | | AGCTTCAACAGGGGGGAGTGT |
| 83 | NOV0712 heavy | MKHLWFFLLLVAAPRWVLSQVQLLESGGGLVQPGGSLRLSCAASGFTFSSHGMHWVR |
| | chain full-length | QAPGKGLEWVSVISGSGSNTGYADSVKGRFTISRDNSKNTLYLQMNSLRAEDTAVYY |
| | amino acid | CARQWGSYAFDSWGQGTLVTVSSASTKGPSVFPLAPSSKSTSGGTAALGCLVKDYFP |
| | sequence | EPVTVSWNSGALTSGVHTFPAVLQSSGLYSLSSVVTVPSSSLGTQTYICNVNHKPSN |
| | | TKVDKRVEPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHEALHNHY |
| | | TQKSLSLSPGK |

TABLE 1-15

| | | |
|---|---|---|
| 84 | NOV0712 heavy | ATGAAACACCTGTGGTTCTTCCTCCTGCTGGTGGCAGCTCCCAGATGGGTGCTGAGC |
| | chain full-length | CAGGTGCAGCTGCTGGAATCTGGCGGAGGACTGGTGCAGCCTGGGGGCTCTCTGAGA |
| | nucleotide | CTGTCTTGTGCCGCCAGGGGCTTCACCTTCAGCAGCCACGGAATGCACTGGGTGCGC |
| | sequence | CAGGCCCCTGGAAAGGGACTGGAATGGGTGTCCGTGATCAGCGGCAGCGGCTCCAAT |
| | | ACCGGCTACGCCGATAGCGTGAAGGGCCGGTTCACCATCAGCCGGGACAACAGCAAG |
| | | AACACCCTGTACCTGCAGATGAACAGCCTGGGGGGGGAGGACACCGCCGTGTACTAT |
| | | TGTGCCAGACAGTGGGGCAGCTACGCCTTCGATTCTTGGGGCCAGGGCACCCTCGTG |
| | | ACCGTCAGCTCAGCCTCCACCAAGGGCCCAAGCGTCTTCCCCCTGGCACCCTCCTCC |
| | | AAGAGCACCTCTGGCGGCACAGCCGCCCTGGGCTGCCTGGTCAAGGACTACTTCCCC |
| | | GAACCCGTGACCGTGAGCTGGAACTCAGGCGCCCTGACCAGCGGCGTGCACACCTTC |
| | | CCCGCTGTCCTGCAGTCCTCAGGACTCTACTCCCTCAGCAGCGTGGTGACCGTGCCC |
| | | TCCAGCAGCTTGGGCACCCAGACCTACATCTGCAACGTGAATCACAAGCCCAGCAAC |
| | | ACCAAGGTGGACAAGAGAGTTGAGCCCAAATCTTGTGACAAAACTCACACATGCCCA |
| | | CCCTGCCCAGCACCTGAACTCCTGGGGGGACCCTCAGTCTTCCTCTTCCCCCCAAAA |
| | | CCCAAGGACACCCTCATGATCTCCCGGACCCCTGAGGTCACATGCGTGGTGGTGGAC |
| | | GTGAGCCACGAAGACCCTGAGGTCAAGTTCAACTGGTACGTGGACGGCGTGGAGGTG |
| | | CATAATGCCAAGACAAAGCCCCGGGAGGAGCAGTACAACAGCACGTACCGGGTGGTC |
| | | AGCGTCCTCACCGTCCTGCACCAGGACTGGCTGAATGGCAAGGAGTACAAGTGCAAG |
| | | GTCTCCAACAAAGCCCTCCCAGCCCCCATCGAGAAAACCATCTCCAAAGCCAAAGGC |
| | | CAGCCCCGGGAACCACAGGTGTACACCCTGCCCCCATCCCGGGAGGAGATGACCAAG |
| | | AACCAGGTCAGCCTGACCTGCCTGGTCAAAGGCTTCTATCCCAGCGACATCGCCGTG |
| | | GAGTGGGAGAGCAATGGCCAGCCCGAGAACAACTACAAGACCACCCCTCCCGTGCTG |
| | | GACTCCGACGGCTCCTTCTTCCTCTACAGCAAGCTCACCGTGGACAAGAGCAGGTGG |
| | | CAGCAGGGCAACGTCTTCTCATGCTCCGTGATGCATGAGGCTCTGCACAACCACTAC |
| | | ACCCAGAAGAGCCTCTCCCTGTCTCCCGGCAAA |

TABLE 1-16

| | | |
|---|---|---|
| 87 | The matured | EVQLVQSGAEVKKPGASVKVSCKASGYTFTRNFMHWVRQAPGQGLEWM |
| | heavy chain | GWIYPGDGETEYAQKFQGRVTITADTSTSTAYMELSSLRSEDTAVYYCAR |
| | amino acid | GVYGGFAGGYFDFWGQGTLVTVSSASTKGPSVFPLAPSSKSTSGGTAAL |
| | sequesnce of | GCLVKDYFPEPVTVSWNSGALTSGVHTFPAVLQSSGLYSLSSVVTVPSSS |
| | hH01 heavy | LGTQTYICNVNHKPSNTKVDKRVEPKSCDKTHTCPPCPAPELLGGPSVFLF |
| | chain, without a | PPKPKDTLMISRTPEVTCVVVDVSHEDPEVKFNWYVDGVEVHNAKTKPRE |
| | signal sequence | EQYNSTYRVVSVLTVLHQDWLNGKEYKCKVSNKALPAPIEKTISKAKGQPR |
| | | EPQVYTLPPSREEMTKNQVSLTCLVKGFYPSDIAVEWESNGQPENNYKTT |
| | | PPVLDSDGSFFLYSKLTVDKSRWQQGNVFSCSVMHEALHNHYTQKSLSLS |
| | | PGK |
| 88 | The matured light | DIQMTQSPSSLSASVGDRVTITCKASQNIYKNLAWYQQKPGKAPKLLIYDA |
| | chain amino acid | NTLQTGVPSRFSGSGSGSDFTLTISSLQPEDFATYFCQQYYSGWAFGQGT |
| | sequesnce of | KVEIKRTVAAPSVFIFPPSDEQLKSGTASVVCLLNNFYPREAKVQWKVDNA |
| | hL02 light chain, | LQSGNSQESVTEQDSKDSTYSLSSTLTLSKADYEKHKVYACEVTHQGLSS |
| | without a signal | PVTKSFNRGEC |
| | sequence | |

In the present description, Tables 1-1 to 1-16 are also collectively referred to as Table 1.

Further examples of the antibody of the present disclosure can include a human antibody binding to CDH6. The anti-CDH6 human antibody means a human antibody having only the gene sequence of an antibody derived from human chromosomes. The anti-CDH6 human antibody can be obtained by a method using a human antibody-producing mouse having a human chromosomal fragment comprising the heavy chain and light chain genes of a human antibody (see Tomizuka, K. et al., Nature Genetics (1997) 16, p. 133-143; Kuroiwa, Y. et al., Nucl. Acids Res. (1998) 26, p. 3447-3448; Yoshida, H. et al., Animal Cell Technology: Basic and Applied Aspects vol. 10, p. 69-73 (Kitagawa, Y., Matsuda, T. and Iijima, S. eds.), Kluwer Academic Publishers, 1999; Tomizuka, K. et al., Proc. Natl. Acad. Sci. USA (2000) 97, p. 722-727; etc.).

Such a human antibody-producing mouse can be specifically produced by using a genetically modified animal, the gene loci of endogenous immunoglobulin heavy chain and light chain of which have been disrupted and instead the gene loci of human immunoglobulin heavy chain and light chain have been then introduced using a yeast artificial chromosome (YAC) vector or the like, then producing a knock-out animal and a transgenic animal from such a genetically modified animal, and then breeding such animals with one another.

Otherwise, the anti-CDH6 human antibody can also be obtained by transforming eukaryotic cells with cDNA encoding each of the heavy chain and light chain of such a human antibody, or preferably with a vector comprising the cDNA, according to genetic recombination techniques, and then culturing the transformed cells producing a genetically modified human monoclonal antibody, so that the antibody can be obtained from the culture supernatant.

In this context, eukaryotic cells, and preferably, mammalian cells such as CHO cells, lymphocytes or myelomas can, for example, be used as a host.

Furthermore, a method of obtaining a phage display-derived human antibody that has been selected from a human antibody library (see Wormstone, I. M. et al., Investigative Ophthalmology & Visual Science. (2002) 43 (7), p. 2301-2308; Carmen, S. et al., Briefings in Functional Genomics and Proteomics (2002), 1 (2), p. 189-203; Siriwardena, D. et al., Ophthalmology (2002) 109 (3), p. 427-431; etc.) is also known.

For example, a phage display method, which comprises allowing the variable regions of a human antibody to express as a single chain antibody (scFv) on the surface of phages, and then selecting a phage binding to an antigen, can be applied (Nature Biotechnology (2005), 23, (9), p. 1105-1116).

By analyzing the phage gene that has been selected because of its binding ability to the antigen, DNA sequences encoding the variable regions of a human antibody binding to the antigen can be determined.

Once the DNA sequence of scFv binding to the antigen is determined, an expression vector having the aforementioned sequence is produced, and the produced expression vector is then introduced into an appropriate host and can be allowed to express therein, thereby obtaining a human antibody (International Publication Nos. WO92/01047, WO92/20791, WO93/06213, WO93/11236, WO93/19172, WO95/01438, and WO95/15388, Annu. Rev. Immunol (1994) 12, p. 433-455, Nature Biotechnology (2005) 23 (9), p. 1105-1116).

If a newly produced human antibody binds to a partial peptide or a partial three-dimensional structure to which any one rat anti-human CDH6 antibody, chimeric anti-human CDH6 antibody or humanized anti-human CDH6 antibody described in the present description (e.g., the rG019 antibody, the rG055 antibody, the rG056 antibody, the rG061 antibody, the chG019 antibody, the H01L02 antibody, the H02L02 antibody, the H02L03 antibody or the H04L02 antibody) binds, it can be determined that the human antibody binds to the same epitope to which the rat anti-human CDH6 antibody, the chimeric anti-human CDH6 antibody or the humanized anti-human CDH6 antibody binds. Alternatively, by confirming that the human antibody competes with the rat anti-human CDH6 antibody, the chimeric anti-human CDH6 antibody or the humanized anti-human CDH6 antibody described in the present description (e.g., the rG019 antibody, the rG055 antibody, the rG056 antibody, the rG061 antibody, the chG019 antibody, the H01L02 antibody, the H02L02 antibody, the H02L03 antibody or the H04L02 antibody) in the binding of the antibody to CDH6 (e.g., the human antibody interferes with the binding of the rG019 antibody, the rG055 antibody, the rG056 antibody, the rG061 antibody, the chG019 antibody, the H01L02 antibody, the H02L02 antibody, the H02L03 antibody or the H04L02 antibody to CDH6, preferably EC3 of CDH6), it can be determined that the human antibody binds to the same epitope to which the rat anti-human CDH6 antibody, the chimeric anti-human CDH6 antibody or the humanized anti-human CDH6 antibody described in the present description binds, even if the specific sequence or structure of the epitope has not been determined. In the present description, when it is determined by at least one of these determination methods that the human antibody “binds to the same epitope”, it is concluded that the newly prepared human antibody “binds to the same epitope” as that for the rat anti-human CDH6 antibody, the chimeric anti-human CDH6 antibody or the humanized anti-human CDH6 antibody described in the present description. When it is confirmed that the human antibody binds to the same epitope, then it is expected that the human antibody should have a biological activity equivalent to that of the rat anti-human CDH6 antibody, the chimeric anti-human CDH6 antibody or the humanized anti-human CDH6 antibody (e.g., the rG019 antibody, the rG055 antibody, the rG056 antibody, the rG061 antibody, the chG019 antibody, the H01L02 antibody, the H02L02 antibody, the H02L03 antibody or the H04L02 antibody).

The chimeric antibodies, the humanized antibodies, or the human antibodies obtained by the above-described methods are evaluated for their binding activity against the antigen according to a known method, etc., so that a preferred antibody can be selected.

One example of another indicator for comparison of the properties of antibodies can include the stability of an antibody. A differential scanning calorimeter (DSC) is an apparatus capable of promptly and exactly measuring a thermal denaturation midpoint (Tm) serving as a good indicator for the relative structural stability of a protein. By using DSC to measure Tm values and making a comparison regarding the obtained values, differences in thermal stability can be compared. It is known that the preservation stability of an antibody has a certain correlation with the thermal stability of the antibody (Lori Burton, et al., Pharmaceutical Development and Technology (2007) 12, p. 265-273), and thus, a preferred antibody can be selected using thermal stability as an indicator. Other examples of the indicator for selection of an antibody can include high yield in suitable host cells and low agglutination in an aqueous solution. For example, since an antibody with the highest yield does not always exhibit the highest thermal stability, it is necessary to select an antibody most suitable for administration to a human by comprehensively determining it based on the aforementioned indicators.

The antibody of the present disclosure also includes a modification of an antibody. The modification is used to mean the antibody of the present disclosure, which is chemically or biologically modified. Examples of such a chemical modification include the binding of a chemical moiety to an amino acid skeleton, and the chemical modification of an N-linked or O-linked carbohydrate chain. Examples of such a biological modification include antibodies which have undergone a posttranslational modification (e.g., N-linked or O-linked glycosylation, N-terminal or C-terminal processing, deamidation, isomerization of aspartic acid, oxidation of methionine, and conversion of N-terminal glutamine or N-terminal glutamic acid to pyroglutamic acid), and antibodies, to the N-terminus of which a methionine residue is added as a result of having been allowed to be expressed using prokaryote host cells. In addition, such a modification is also meant to include labeled antibodies for enabling detection or isolation of the antibody of the present disclosure or an antigen, for example, an enzymatically labeled antibody, a fluorescently labeled antibody, and an affinity-labeled antibody. Such a modification of the antibody of the present disclosure is useful for the improvement of the stability and retention in blood of an antibody; a reduction in antigenicity; detection or isolation of an antibody or an antigen; etc.

Moreover, by regulating a sugar chain modification (glycosylation, de-fucosylation, etc.) that binds to the antibody of the present disclosure, antibody-dependent cellular cytotoxic activity can be enhanced. As techniques of regulating the sugar chain modification of an antibody, those described in International Publication Nos. WO1999/54342, WO2000/61739, and WO2002/31140, etc. are known, though the techniques are not limited thereto. The antibody of the present disclosure also includes antibodies in respect of which the aforementioned sugar chain modification has been regulated.

Once an antibody gene is isolated, the gene can be introduced into an appropriate host to produce an antibody, using an appropriate combination of a host and an expression vector. A specific example of the antibody gene can be a combination of a gene encoding the heavy chain sequence of the antibody described in the present description and a gene encoding the light chain sequence of the antibody described therein. Upon transformation of host cells, such a heavy chain sequence gene and a light chain sequence gene may be inserted into a single expression vector, or these genes may instead each be inserted into different expression vectors.

When eukaryotic cells are used as hosts, animal cells, plant cells or eukaryotic microorganisms can be used. In particular, examples of the animal cells can include mammalian cells such as COS cells which are monkey cells (Gluzman, Y., Cell (1981) 23, p. 175-182, ATCC CRL-1650), mouse fibroblasts NIH3T3 (ATCC No. CRL-1658), a dihydrofolate reductase-deficient cell line of Chinese hamster ovary cells (CHO cells, ATCC CCL-61) (Urlaub, G. and Chasin, L. A. Proc. Natl. Acad. Sci. U.S.A. (1980) 77, p. 4126-4220), and FreeStyle 293F cells (Invitrogen Corp.).

When prokaryotic cells are used as hosts, Escherichia coli or Bacillus subtilis can be used, for example.

An antibody gene of interest is introduced into these cells for transformation, and the transformed cells are then cultured in vitro to obtain an antibody. In the aforementioned culture, there are cases where yield is different depending on the sequence of the antibody, and thus, it is possible to select an antibody, which is produced as a medicament, from antibodies having equivalent binding activity, using the yield as an indicator. Accordingly, the antibody of the present disclosure also includes an antibody obtained by the above-described method for producing an antibody, which comprises a step of culturing the transformed host cells and a step of collecting an antibody of interest or a functional fragment of the antibody from the culture obtained in the aforementioned step.

It is known that the lysine residue at the carboxyl terminus of the heavy chain of an antibody produced in cultured mammalian cells is deleted (Journal of Chromatography A, 705:129-134 (1995)), and also, it is known that the two amino acid residues at the heavy chain carboxyl terminus, glycine and lysine, are deleted, and that the proline residue newly positioned at the carboxyl terminus is amidated (Analytical Biochemistry, 360:75-83 (2007)). However, such deletion and modification of these heavy chain sequences does not have an influence on the antigen-binding activity and effector function (activation of complement, antibody-dependent cellular cytotoxicity, etc.) of an antibody. Accordingly, the antibody according to the present disclosure also includes an antibody that has undergone the aforementioned modification, and a functional fragment of the antibody, and specific examples of such an antibody include a deletion mutant comprising a deletion of 1 or 2 amino acids at the heavy chain carboxyl terminus, and a deletion mutant formed by amidating the aforementioned deletion mutant (e.g., a heavy chain in which the proline residue at the carboxyl-terminal site is amidated). However, deletion mutants involving a deletion at the carboxyl terminus of the heavy chain of the antibody according to the present disclosure are not limited to the above-described deletion mutants, as long as they retain antigen-binding activity and effector function. Two heavy chains constituting the antibody according to the present disclosure may be any one type of heavy chain selected from the group consisting of a full-length antibody and the above-described deletion mutants, or may be a combination of any two types selected from the aforementioned group. The ratio of individual deletion mutants can be influenced by the types of cultured mammalian cells that produce the antibody according to the present disclosure, and the culture conditions. Examples of the main ingredient of the antibody according to the present disclosure can include antibodies where one amino acid residue is deleted at each of the carboxyl termini of the two heavy chains.

Examples of the isotype of the antibody of the present disclosure can include IgG (IgG1, IgG2, IgG3, and IgG4). Among others, IgG1 and IgG4 are preferable.

Examples of the biological activity of an antibody can generally include antigen-binding activity, activity of being internalized into cells expressing an antigen by binding to the antigen, activity of neutralizing the activity of an antigen, activity of enhancing the activity of an antigen, antibody-dependent cellular cytotoxic (ADCC) activity, complement-dependent cytotoxic (CDC) activity, and antibody-dependent cellular phagocytosis (ADCP). The function of the antibody according to the present disclosure is binding activity against CDH6 and is preferably the activity of being internalized into CDH6-expressing cells by binding to CDH6. Moreover, the antibody of the present disclosure may have ADCC activity, CDC activity and/or ADCP activity, as well as cellular internalization activity.

The obtained antibody can be purified to a homogenous state. For separation and purification of the antibody, separation and purification methods used for ordinary proteins may be used. For example, column chromatography, filtration, ultrafiltration, salting-out, dialysis, preparative polyacrylamide gel electrophoresis, and isoelectric focusing are appropriately selected and combined with one another, so that the antibody can be separated and purified (Strategies for Protein Purification and Characterization: A Laboratory Course Manual, Daniel R. Marshak et al. eds., Cold Spring Harbor Laboratory Press (1996); and Antibodies: A Laboratory Manual. Ed Harlow and David Lane, Cold Spring Harbor Laboratory (1988)), though examples of the separation and purification methods are not limited thereto.

Examples of the chromatography can include affinity chromatography, ion exchange chromatography, hydrophobic chromatography, gel filtration chromatography, reverse phase chromatography, and absorption chromatography.

These chromatographic techniques can be carried out using liquid chromatography such as HPLC or FPLC.

Examples of the column used in the affinity chromatography can include a Protein A column and a Protein G column. Examples of the column involving the use of Protein A can include Hyper D, POROS, and Sepharose F. F. (Pharmacia).

Also, using an antigen-immobilized carrier, the antibody can be purified by utilizing the binding activity of the antibody to the antigen.

### 3. Anti-CDH6 Antibody-Drug Conjugate

### (1) Drug

The anti-CDH6 antibody obtained in the above “2. Production of anti-CDH6 antibody” can be conjugated to a drug via a linker structure moiety to prepare an anti-CDH6 antibody-drug conjugate. The drug is not particularly limited as long as it has a substituent or a partial structure that can be connected to a linker structure. The anti-CDH6 antibody-drug conjugate can be used for various purposes according to the conjugated drug. Examples of such a drug can include substances having antitumor activity, substances effective for blood diseases, substances effective for autoimmune diseases, anti-inflammatory substances, antimicrobial substances, antifungal substances, antiparasitic substances, antiviral substances, and anti-anesthetic substances.

### (1)-1 Antitumor Compound

An example using an antitumor compound as a compound to be conjugated in the anti-CDH6 antibody-drug conjugate of the present disclosure will be described below. The antitumor compound is not particularly limited as long as the compound has an antitumor effect and has a substituent or a partial structure that can be connected to a linker structure. Upon cleavage of a part or the whole of the linker in tumor cells, the antitumor compound moiety is released so that the antitumor compound exhibits an antitumor effect. As the linker is cleaved at a connecting position with the drug, the antitumor compound is released in its original structure to exert its original antitumor effect.

The anti-CDH6 antibody obtained in the above “2. Production of anti-CDH6 antibody” can be conjugated to the antitumor compound via a linker structure moiety to prepare an anti-CDH6 antibody-drug conjugate.

As one example of the antitumor compound used in the present disclosure, exatecan, a camptothecin derivative ((1S,9S)-1-amino-9-ethyl-5-fluoro-2,3-dihydro-9-hydroxy-4-methyl-1H, 12H-benzo[de]pyrano[3′,4′: 6,7]indolizino[1,2-b]quinoline-10, 13 (9H, 15H)-dione represented by the following formula) can preferably be used.

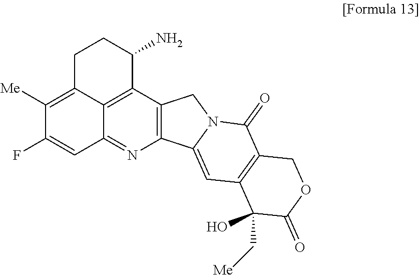 [SMILES: CC[C@@]1(O)C(=O)OCc2c1cc1n(c2=O)Cc2c-1nc1cc(F)c(C)c3c1c2[C@@H](N)CC3]

The compound can be obtained by, for example, a method described in U.S. Patent Publication No. US2016/0297890 or other known methods, and the amino group at position 1 can be preferably used as a connecting position to the linker structure. Further, exatecan may be released in tumor cells while a part of the linker is still attached thereto. However, the compound exerts an excellent antitumor effect even in such a state.

Since exatecan has a camptothecin structure, it is known that the equilibrium shifts to a structure with a formed lactone ring (closed ring) in an acidic aqueous medium (e.g., of the order of pH 3) whereas the equilibrium shifts to a structure with an opened lactone ring (open ring) in a basic aqueous medium (e.g., of the order of pH 10). A drug conjugate into which exatecan residues corresponding to such a closed ring structure and an open ring structure have been introduced is also expected to have an equivalent antitumor effect, and it is needless to say that any of such drug conjugate is included within the scope of the present disclosure.

Other examples of the antitumor compound can include antitumor compounds described in the literature (Pharmacological Reviews, 68, p. 3-19, 2016). Specific examples thereof can include doxorubicin, calicheamicin, dolastatin 10, auristatins such as monomethyl auristatin E (MMAE) and monomethyl auristatin F (MMAF), maytansinoids such as DM1 and DM4, a pyrrolobenzodiazepine dimer SG2000 (SJG-136), a camptothecin derivative SN-38, duocarmycins such as CC-1065, amanitin, daunorubicin, mitomycin C, bleomycin, cyclocytidine, vincristine, vinblastine, methotrexate, platinum-based antitumor agents (cisplatin and derivatives thereof), and Taxol and derivatives thereof.

In the antibody-drug conjugate, the number of conjugated drug molecules per antibody molecule is a key factor having an influence on the efficacy and safety thereof. The production of the antibody-drug conjugate is carried out by specifying reaction conditions such as the amounts of starting materials and reagents used for reaction, so as to attain a constant number of conjugated drug molecules. Unlike the chemical reaction of a low-molecular-weight compound, a mixture containing different numbers of conjugated drug molecules is usually obtained. The number of conjugated drug molecules per antibody molecule is defined and indicated as an average value, i.e., the average number of conjugated drug molecules. Unless otherwise specified, i.e., except in the case of representing an antibody-drug conjugate having a specific number of conjugated drug molecules that is included in an antibody-drug conjugate mixture having different numbers of conjugated drug molecules, the number of conjugated drug molecules according to the present disclosure also means an average value as a rule. The number of exatecan molecules conjugated to an antibody molecule is controllable, and as an average number of conjugated drug molecules per antibody, approximately 1 to 10 exatecan molecules can be conjugated. The number of exatecan molecules is preferably 2 to 8, 3 to 8, 4 to 8, 5 to 8, 6 to 8, or 7 to 8, more preferably 5 to 8, further preferably 7 to 8, still further preferably 8. It is to be noted that a person skilled in the art can design a reaction for conjugating a required number of drug molecules to an antibody molecule based on the description of Examples of the present application, and can obtain an antibody-drug conjugate with a controlled number of conjugated exatecan molecules.

### (2) Linker Structure

The linker structure which conjugates the drug to the anti-CDH6 antibody in the anti-CDH6 antibody-drug conjugate of the present disclosure will be described.

In the antibody-drug conjugate of the present application, the linker structure which conjugates the anti-CDH6 antibody to the drug is not particularly limited as long as the resulting antibody-drug conjugate can be used. The linker structure may be appropriately selected and used according to the purpose of use. One example of the linker structure can include a linker described in known literature (Pharmacol Rev 68:3-19, January 2016, Protein Cell DOI 10.1007/s13238-016-0323-0, etc.). Further specific examples thereof can include VC (valine-citrulline), MC (maleimidocaproyl), SMCC (succinimidyl 4-(N-maleimidomethyl) cyclohexane-1-carboxylate), SPP (N-succinimidyl 4-(2-pyridyldithio) pentanoate, SS (disulfide), SPDB (N-succinimidyl 4-(2-pyridyldithio) butyrate, SS/hydrazone, hydrazone and carbonate.

Another example can include a linker structure described in U.S. Patent Publication No. US2016/0297890 (as one example, those described in paragraphs to thereof). Any linker structure given below can preferably be used. It is to be noted that the left terminus of the structure is a connecting position to the antibody, and the right terminus thereof is a connecting position to the drug. Furthermore, GGFG (SEQ ID NO: 89) in the linker structures given below represents an amino acid sequence consisting of glycine-glycine-phenylalanine-glycine (GGFG) (SEQ ID NO: 89) linked through peptide bonds.

-(Succinimid-3-yl-N)—CH₂CH₂—C(═O)-GGFG-NH—CH₂CH₂CH₂—C(═O)—,

-(Succinimid-3-yl-N)—CH₂CH₂CH₂CH₂CH₂—C(═O)-GGFG-NH—CH₂CH₂CH₂—C(═O)—,

-(Succinimid-3-yl-N)—CH₂CH₂CH₂CH₂CH₂—C(═O)-GGFG-NH—CH₂—O—CH₂—C(═O)—,

-(Succinimid-3-yl-N)—CH₂CH₂CH₂CH₂CH₂—C(═O)-GGFG-NH—CH₂CH₂—O—CH₂—C(═O)—,

-(Succinimid-3-yl-N)—CH₂CH₂—C(═O)—NH—CH₂CH₂O—CH₂CH₂O—CH₂CH₂—C(═O)-GGFG-NH—CH₂CH₂CH₂—C(═O)—, and

-(Succinimid-3-yl-N)—CH₂CH₂—C(═O)—NH—CH₂CH₂O—CH₂CH₂O—CH₂CH₂O—CH₂CH₂O—CH₂CH₂—C(═O)-GGFG-NH—CH₂CH₂CH₂—C(═O)—.

More preferred are the following:

-(Succinimid-3-yl-N)—CH₂CH₂CH₂CH₂CH₂—C(═O)-GGFG-NH—CH₂—O—CH₂—C(═O)—,

-(Succinimid-3-yl-N)—CH₂CH₂CH₂CH₂CH₂—C(═O)-GGFG-NH—CH₂CH₂—O—CH₂—C(═O)—, and

-(Succinimid-3-yl-N)—CH₂CH₂—C(═O)—NH—CH₂CH₂O—CH₂CH₂O—CH₂CH₂—C(═O)-GGFG-NH—CH₂CH₂CH₂—C(═O)—.

Still more preferred are the following:

-(Succinimid-3-yl-N)—CH₂CH₂CH₂CH₂CH₂—C(═O)-GGFG-NH—CH₂—O—CH₂—C(═O)—, and

-(Succinimid-3-yl-N)—CH₂CH₂—C(═O)—NH—CH₂CH₂O—CH₂CH₂O—CH₂CH₂—C(═O)-GGFG-NH—CH₂CH₂CH₂—C(═O)—.

The antibody is connected to the terminus of -(Succinimid-3-yl-N) (e.g., a terminus opposite (left terminus) to the terminus to which —CH₂CH₂CH₂CH₂CH₂— is connected in “-(Succinimid-3-yl-N)—CH₂CH₂CH₂CH₂CH₂—C(═O)-GGFG-NH—CH₂—O—CH₂—C(═O)—”), and the antitumor compound is connected to a terminus (the carbonyl group of CH₂—O—CH₂—C(═O)— at the right terminus in the above-described example) opposite to the terminus to which the antibody is connected to -(Succinimid-3-yl-N). “-(Succinimid-3-yl-N)—” has a structure represented by the following formula:

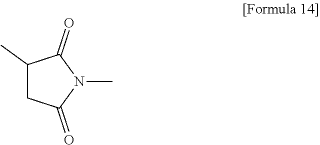

Position 3 of this partial structure is the connecting position to the anti-CDH6 antibody. This connection to the antibody at position 3 is characterized by forming a thioether bond. The nitrogen atom at position 1 of this structure moiety is connected to the carbon atom of methylene which is present within the linker including the structure.

In the antibody-drug conjugate of the present disclosure having exatecan as the drug, a drug-linker structure moiety having any structure given below is preferred for conjugation to the antibody. For these drug-linker structure moieties, the average number conjugated per antibody may be 1 to 10 and is preferably 2 to 8, more preferably 5 to 8, further preferably 7 to 8, and still further preferably 8.

-(Succinimid-3-yl-N)—CH₂CH₂—C(═O)-GGFG-NH—CH₂CH₂CH₂—C(═O)—(NH-DX),

-(Succinimid-3-yl-N)—CH₂CH₂CH₂CH₂CH₂—C(═O)-GGFG-NH—CH₂CH₂CH₂—C(═O)—(NH-DX),

-(Succinimid-3-yl-N)—CH₂CH₂CH₂CH₂CH₂—C(═O)-GGFG-NH—CH₂—O—CH₂—C(═O)—(NH-DX),

-(Succinimid-3-yl-N)—CH₂CH₂CH₂CH₂CH₂—C(═O)-GGFG-NH—CH₂CH₂—O—CH₂—C(═O)—(NH-DX),

-(Succinimid-3-yl-N)—CH₂CH₂—C(═O)—NH—CH₂CH₂O—CH₂CH₂O—CH₂CH₂—C(═O)-GGFG-NH—CH₂CH₂CH₂—C(═O)—(NH-DX), and

-(Succinimid-3-yl-N)—CH₂CH₂—C(═O)—NH—CH₂CH₂O—CH₂CH₂O—CH₂CH₂O—CH₂CH₂O—CH₂CH₂—C(═O)-GGFG-NH—CH₂CH₂CH₂—C(═O)—(NH-DX).

More preferred are the following:

-(Succinimid-3-yl-N)—CH₂CH₂CH₂CH₂CH₂—C(═O)-GGFG-NH—CH₂—O—CH₂—C(═O)—(NH-DX),

-(Succinimid-3-yl-N)—CH₂CH₂CH₂CH₂CH₂—C(═O)-GGFG-NH—CH₂CH₂—O—CH₂—C(═O)—(NH-DX), and

-(Succinimid-3-yl-N)—CH₂CH₂—C(═O)—NH—CH₂CH₂O—CH₂CH₂O—CH₂CH₂—C(═O)-GGFG-NH—CH₂CH₂CH₂—C(═O)—(NH-DX).

Still more preferred are the following:

-(Succinimid-3-yl-N)—CH₂CH₂CH₂CH₂CH₂—C(═O)-GGFG-NH—CH₂—O—CH₂—C(═O)—(NH-DX), and

-(Succinimid-3-yl-N)—CH₂CH₂—C(═O)—NH—CH₂CH₂O—CH₂CH₂O—CH₂CH₂—C(═O)-GGFG-NH—CH₂CH₂CH₂—C(═O)—(NH-DX).

—(NH-DX) has a structure represented by the following formula:

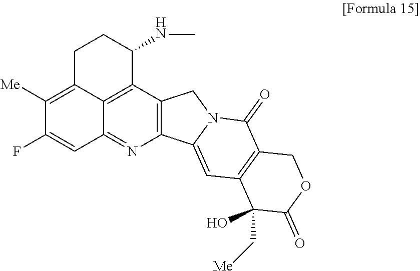 [SMILES: CC[C@@]1(O)C(=O)OCc2c1cc1n(c2=O)Cc2c-1nc1cc(F)c(C)c3c1c2[C@@H](NC)CC3]

and it represents a group that is derived by removing one hydrogen atom from the amino group at position 1 of exatecan.

### (3) Method for Producing Antibody-Drug Conjugate

The antibody that can be used in the antibody-drug conjugate of the present disclosure is not particularly limited as long as it is an anti-CDH6 antibody having internalization activity or a functional fragment of the antibody, as described in the above section “2. Production of anti-CDH6 antibody” and the Examples.

Next, a typical method for producing the antibody-drug conjugate of the present disclosure will be described. It is to be noted that, in the description below, “compound No.” shown in each reaction scheme is used to represent a compound. Specifically, each compound is referred to as a “compound of formula (1)”, “compound (1)”, or the like. The same holds true for the other compound Nos.

### (3)-1 Production Method 1

The antibody-drug conjugate represented by formula (1) given below in which the anti-CDH6 antibody is connected to the linker structure via a thioether can be produced by reacting an antibody having a sulfhydryl group converted from a disulfide bond by the reduction of the anti-CDH6 antibody, with the compound (2), the compound (2) being obtainable by a known method (e.g., obtainable by a method described in the patent publication literature US2016/297890 (e.g., a method described in the paragraphs to [0374])). This antibody-drug conjugate can be produced by the following method, for example.

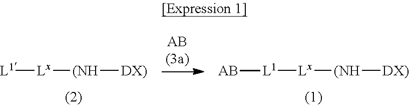

wherein AB represents an antibody with a sulfhydryl group, wherein
L¹ has a structure represented by -(Succinimid-3-yl-N)—, and L¹′ represents a maleimidyl group represented by the following formula.

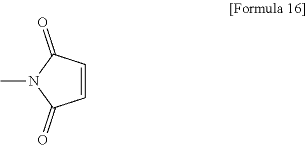

-L¹-L^{X} has a structure represented by any of the following formulas:

-(Succinimid-3-yl-N)—CH₂CH₂—C(═O)-GGFG-NH—CH₂CH₂CH₂—C(═O)—,

-(Succinimid-3-yl-N)—CH₂CH₂CH₂CH₂CH₂—C(═O)-GGFG-NH—CH₂CH₂CH₂—C(═O)—,

-(Succinimid-3-yl-N)—CH₂CH₂CH₂CH₂CH₂—C(═O)-GGFG-NH—CH₂—O—CH₂—C(═O)—,

-(Succinimid-3-yl-N)—CH₂CH₂CH₂CH₂CH₂—C(═O)-GGFG-NH—CH₂CH₂—O—CH₂—C(═O)—,

-(Succinimid-3-yl-N)—CH₂CH₂—C(═O)—NH—CH₂CH₂O—CH₂CH₂O—CH₂CH₂—C(═O)-GGFG-NH—CH₂CH₂CH₂—C(═O)—, and

-(Succinimid-3-yl-N)—CH₂CH₂—C(═O)—NH—CH₂CH₂O—CH₂CH₂O—CH₂CH₂O—CH₂CH₂O—CH₂CH₂—C(═O)-GGFG-NH—CH₂CH₂CH₂—C(═O)—.

Among them, more preferred are the following:

-(Succinimid-3-yl-N)—CH₂CH₂CH₂CH₂CH₂—C(═O)-GGFG-NH—CH₂—O—CH₂—C(═O)—,

-(Succinimid-3-yl-N)—CH₂CH₂CH₂CH₂CH₂—C(═O)-GGFG-NH—CH₂CH₂—O—CH₂—C(═O)—, and

-(Succinimid-3-yl-N)—CH₂CH₂—C(═O)—NH—CH₂CH₂O—CH₂CH₂O—CH₂CH₂—C(═O)-GGFG-NH—CH₂CH₂CH₂—C(═O)—.

Further preferred are the following:

-(Succinimid-3-yl-N)—CH₂CH₂CH₂CH₂CH₂—C(═O)-GGFG-NH—CH₂—O—CH₂—C(═O)—, and

-(Succinimid-3-yl-N)—CH₂CH₂—C(═O)—NH—CH₂CH₂O—CH₂CH₂O—CH₂CH₂—C(═O)-GGFG-NH—CH₂CH₂CH₂—C(═O)—.

In the above-described reaction scheme, the antibody-drug conjugate (1) can be understood as having a structure in which one structure moiety from the drug to the linker terminus is connected to one antibody. However, this description is given for the sake of convenience, and there are actually many cases in which a plurality of the aforementioned structure moieties is connected to one antibody molecule. The same holds true for the explanation of the production method described below.

Specifically, the antibody-drug conjugate (1) can be produced by reacting the compound (2) obtainable by a known method (e.g., obtainable by a method described in the patent publication literature US2016/297890 (e.g., obtainable by a method described in the paragraphs to [0374])), with the antibody (3a) having a sulfhydryl group.

The antibody (3a) having a sulfhydryl group can be obtained by a method well known to a person skilled in the art (Hermanson, G. T, Bioconjugate Techniques, pp. 56-136, pp. 456-493, Academic Press (1996)). Examples of the method can include, but are not limited to: Traut's reagent being reacted with the amino group of the antibody; N-succinimidyl S-acetylthioalkanoates being reacted with the amino group of the antibody followed by reaction with hydroxylamine; N-succinimidyl 3-(pyridyldithio) propionate being reacted with the antibody, followed by reaction with a reducing agent; the antibody being reacted with a reducing agent such as dithiothreitol, 2-mercaptoethanol, or tris(2-carboxyethyl) phosphine hydrochloride (TCEP) to reduce the interchain disulfide bond in the antibody, so as to form a sulfhydryl group.

Specifically, an antibody with interchain disulfide bonds partially or completely reduced can be obtained by using 0.3 to 3 molar equivalents of TCEP as a reducing agent per interchain disulfide bond in the antibody, and reacting the reducing agent with the antibody in a buffer solution containing a chelating agent. Examples of the chelating agent can include ethylenediaminetetraacetic acid (EDTA) and diethylenetriaminepentaacetic acid (DTPA). The chelating agent can be used at a concentration of 1 mM to 20 mM. A solution of sodium phosphate, sodium borate, sodium acetate, or the like can be used as the buffer solution. As a specific example, the antibody (3a) having partially or completely reduced sulfhydryl groups can be obtained by reacting the antibody with TCEP at 4° C. to 37° C. for 1 to 4 hours.

It is to be noted that by carrying out an addition reaction of a sulfhydryl group to a drug-linker moiety, the drug-linker moiety can be conjugated by a thioether bond.

Then, using 2 to 20 molar equivalents of the compound (2) per antibody (3a) having a sulfhydryl group, the antibody-drug conjugate (1) in which 2 to 8 drug molecules are conjugated per antibody can be produced. Specifically, a solution containing the compound (2) dissolved therein may be added to a buffer solution containing the antibody (3a) having a sulfhydryl group for the reaction. In this context, a sodium acetate solution, sodium phosphate, sodium borate, or the like can be used as the buffer solution. pH for the reaction is 5 to 9, and more preferably, the reaction may be performed near pH 7. An organic solvent such as dimethyl sulfoxide (DMSO), dimethylformamide (DMF), dimethylacetamide (DMA), or N-methyl-2-pyrrolidone (NMP) can be used as a solvent for dissolving the compound (2). The reaction may be performed by adding the solution containing the compound (2) dissolved in the organic solvent at 1 to 20% v/v to a buffer solution containing the antibody (3a) having a sulfhydryl group. The reaction temperature is 0 to 37° C., more preferably 10 to 25° C., and the reaction time is 0.5 to 2 hours. The reaction can be terminated by deactivating the reactivity of unreacted compound (2) with a thiol-containing reagent. The thiol-containing reagent is, for example, cysteine or N-acetyl-L-cysteine (NAC). More specifically, the reaction can be terminated by adding 1 to 2 molar equivalents of NAC to the compound (2) used, and incubating the obtained mixture at room temperature for 10 to 30 minutes.

### (4) Identification of Antibody-Drug Conjugate

The produced antibody-drug conjugate (1) can be subjected to concentration, buffer exchange, purification, and measurement of antibody concentration and the average number of conjugated drug molecules per antibody molecule according to common procedures described below, to identify the antibody-drug conjugate (1).

### (4)-1 Common Procedure A: Concentration of Aqueous Solution of Antibody or Antibody-Drug Conjugate

To an Amicon Ultra (50,000 MWCO, Millipore Corporation) container, a solution of an antibody or an antibody-drug conjugate was added, and the solution of the antibody or the antibody-drug conjugate was concentrated by centrifugation (centrifugation at 2000 G to 3800 G for 5 to 20 minutes) using a centrifuge (Allegra X-15R, Beckman Coulter, Inc.)

### (4)-2 Common Procedure B: Measurement of Antibody Concentration

Using a UV detector (Nanodrop 1000, Thermo Fisher Scientific Inc.), measurement of the antibody concentration was carried out according to the method defined by the manufacturer. In this respect, 280 nm absorption coefficient differing among antibodies (1.3 mLmg⁻¹ cm⁻¹ to 1.8 mLmg⁻¹ cm⁻¹) was used.

### (4)-3 Common Procedure C: Buffer Exchange for Antibody

A NAP-25 column (Cat. No. 17-0852-02, GE Healthcare Japan Corporation) using Sephadex G-25 carrier was equilibrated with a phosphate buffer (50 mM, pH 6.0) (referred to as PBS6.0/EDTA in the present description) containing sodium chloride (50 mM) and EDTA (2 mM) according to the method defined by the manufacturer. An aqueous solution of the antibody was applied in an amount of 2.5 mL per NAP-25 column, and thereafter, a fraction (3.5 mL) eluted with 3.5 mL of PBS6.0/EDTA was collected. This fraction was concentrated by common procedure A. After measurement of the concentration of the antibody using common procedure B, the antibody concentration was adjusted to 20 mg/mL using PBS6.0/EDTA.

### (4)-4 Common Procedure D: Purification of Antibody-Drug Conjugate

A NAP-25 column was equilibrated with any commercially available buffer solution such as an acetate buffer containing sorbitol (5%) (10 mM, pH 5.5; referred to as ABS in the present description). An aqueous reaction solution of the antibody-drug conjugate (approximately 2.5 mL) was applied to the NAP-25 column, and thereafter, elution was carried out with the buffer solution in an amount defined by the manufacturer, so as to collect an antibody fraction. A gel filtration purification process, in which the collected fraction was applied again to the NAP-25 column, and elution was carried out with the buffer solution, was repeated a total of 2 or 3 times to obtain the antibody-drug conjugate excluding non-conjugated drug linker and low-molecular-weight compounds (tris(2-carboxyethyl) phosphine hydrochloride (TCEP), N-acetyl-L-cysteine (NAC), and dimethyl sulfoxide).

### (4)-5 Common Procedure E: Measurement of Antibody Concentration in Antibody-Drug Conjugate and Average Number of Conjugated Drug Molecules Per Antibody Molecule

The conjugated drug concentration in the antibody-drug conjugate can be calculated by measuring UV absorbance of an aqueous solution of the antibody-drug conjugate at two wavelengths of 280 nm and 370 nm, and thereafter performing the calculation shown below.

The total absorbance at any given wavelength is equal to the sum of the absorbance of all light-absorbing chemical species that are present in a system [additivity of absorbance]. Therefore, based on the hypothesis that the molar absorption coefficients of the antibody and the drug do not vary between before and after conjugation between the antibody and the drug, the antibody concentration and the drug concentration in the antibody-drug conjugate are represented by the following equations.

$${A}_{280} = {A}_{D , 280} + {A}_{A , 280} = {ε}_{D , 280} {C}_{D} + {ε}_{A , 280} {C}_{A} \qquad Equation ( 1 )$$

$${A}_{370} = {A}_{D , 370} + {A}_{A , 370} = {ε}_{D , 370} {C}_{D} + {ε}_{A , 370} {C}_{A} \qquad Equation ( 2 )$$

In this context, A₂₈₀ represents the absorbance of an aqueous solution of the antibody-drug conjugate at 280 nm, A₃₇₀ represents the absorbance of an aqueous solution of the antibody-drug conjugate at 370 nm, A_{A,280} represents the absorbance of the antibody at 280 nm, A_{A,370} represents the absorbance of the antibody at 370 nm, A_{D,280} represents the absorbance of a conjugate precursor at 280 nm, A_{D,370} represents the absorbance of a conjugate precursor at 370 nm, ε_{A,280} represents the molar absorption coefficient of the antibody at 280 nm, ε_{A,370} represents the molar absorption coefficient of the antibody at 370 nm, ε_{D,280} represents the molar absorption coefficient of a conjugate precursor at 280 nm, ED, 370 represents the molar absorption coefficient of a conjugate precursor at 370 nm, CA represents the antibody concentration in the antibody-drug conjugate, and CD represent the drug concentration in the antibody-drug conjugate.

In this context, with regard to ε_{A,280}, ε_{A,370}, ε_{D,280}, and ε_{D,370}, preliminarily prepared values (estimated values based on calculation or measurement values obtained by UV measurement of the compound) are used. For example, ε_{A,280} can be estimated from the amino acid sequence of the antibody by a known calculation method (Protein Science, 1995, vol. 4, 2411-2423). ε_{A,370} is generally zero. ε_{D,280} and ε_{D,370} can be obtained according to Lambert-Beer's law (Absorbance=Molar concentration×Molar absorption coefficient×Cell path length) by measuring the absorbance of a solution in which the conjugate precursor used is dissolved at a certain molar concentration. C_{A} and C_{D} can be determined by measuring A₂₈₀ and A₃₇₀ of an aqueous solution of the antibody-drug conjugate, and then solving the simultaneous equations (1) and (2) by substitution of these values. Further, by dividing CD by CA, the average number of conjugated drug molecules per antibody can be determined.

### (4)-6 Common Procedure F: Measurement of Average Number of Conjugated Drug Molecules Per Antibody Molecule in Antibody-Drug Conjugate—(2)

The average number of conjugated drug molecules per antibody molecule in the antibody-drug conjugate can also be determined by high-performance liquid chromatography (HPLC) analysis using the following method, in addition to the aforementioned “(4)-5 Common procedure E”. Hereinafter, the method for measuring the average number of conjugated drug molecules by HPLC when the antibody is conjugated to the drug linker by a disulfide bond will be described. A person skilled in the art is capable of appropriately measuring the average number of conjugated drug molecules by HPLC, depending on the connecting manner between the antibody and the drug linker, with reference to this method.

### F-1. Preparation of Sample for HPLC Analysis (Reduction of Antibody-Drug Conjugate)

An antibody-drug conjugate solution (approximately 1 mg/mL, 60 μL) is mixed with an aqueous solution of dithiothreitol (DTT) (100 mM, 15 μL). By incubating the mixture at 37° C. for 30 minutes, the disulfide bond between the light chain and heavy chain of the antibody-drug conjugate is cleaved. The resulting sample is used in HPLC analysis.

### F-2. HPLC Analysis

The HPLC analysis is carried out under the following measurement conditions.

HPLC system: Agilent 1290 HPLC system (Agilent Technologies, Inc.)
Detector: Ultraviolet absorption spectrometer (measurement wavelength: 280 nm)
Column: ACQUITY UPLC BEH Phenyl (2.1×50 mm, 1.7 μm, 130 angstroms; Waters Corp., P/N 186002884)
Column temperature: 80° C.
Mobile phase A: Aqueous solution containing 0.10% trifluoroacetic acid (TFA) and 15% 2-propanol
Mobile phase B: Acetonitrile solution containing 0.075% TFA and 15% 2-propanol
Gradient program: 14%-36% (0 min-15 min), 36%-80% (15 min-17 min), 80%-14% (17 min-17.01 min.), and 14% (17.01 min-25 min)
Sample injection: 10 μL

### F-3. Data Analysis

F-3-1. Compared with non-conjugated antibody light (L0) and heavy (H0) chains, a light chain bound to drug molecule(s) (light chain bound to i drug molecule(s): Lᵢ) and a heavy chain bound to drug molecule(s) (heavy chain bound to i drug molecule(s): Hᵢ) exhibit higher hydrophobicity in proportion to the number of conjugated drug molecules and thus have a larger retention time. These chains are therefore eluted in the order of, for example, L0 and L1 or H0, H1, H2, and H3. Detection peaks can be assigned to any of L0, L1, H0, H1, H2, and H3 by the comparison of retention times with L0 and H0. The number of conjugated drug molecules can be defined by a person skilled in the art, but is preferably L0, L1, H0, H1, H2, and H3.

F-3-2. Since the drug linker has UV absorption, peak area values are corrected in response to the number of conjugated drug linker molecules according to the following expression using the molar absorption coefficients of the light chain or heavy chain and the drug linker.

$$[ Expression 2 ]$$

$$Corrected value of peak area of light chain bound to i drug molecule ( s ) ( {A}_{Li} ) = Peak area \times \frac{Molar absorption coefficient of light chain}{\begin{matrix}Molar absorption coefficient of light chain + \\ The number of conjugated drug molecules ( i ) \times \\ Molar absorption coefficient of drug linker\end{matrix}}$$

$$[ Expression 3 ]$$

$$Corrected value of peak area of heavy chain bound to i drug molecule ( s ) ( {A}_{Hi} ) = Peak area \times \frac{Molar absorption coefficient of heavy chain}{\begin{matrix}Molar absorption coefficient of heavy chain + \\ The number of conjugated drug molecules ( i ) \times \\ Molar absorption coefficient of drug linker\end{matrix}}$$

In this context, a value estimated from the amino acid sequence of the light chain or heavy chain of each antibody by a known calculation method (Protein Science, 1995, vol. 4, 2411-2423) can be used as the molar absorption coefficient (280 nm) of the light chain or heavy chain of the antibody. In the case of H01L02, a molar absorption coefficient of 31710 and a molar absorption coefficient of 79990 were used as estimated values for the light chain and heavy chain, respectively, according to the amino acid sequence of the antibody. The actually measured molar absorption coefficient (280 nm) of a compound in which the maleimide group has been converted to succinimide thioether by the reaction of each drug linker with mercaptoethanol or N-acetylcysteine was used as the molar absorption coefficient (280 nm) of the drug linker. The wavelength for absorbance measurement can be appropriately set by a person skilled in the art, but is preferably a wavelength at which the peak of the antibody can be measured, and more preferably 280 nm.

F-3-3. The peak area ratio (%) of each chain is calculated for the total of the corrected values of peak areas according to the following expression.

$$[ Expression 4 ]$$

$$Peak area ratio of light chain bound to i drug molecule ( s ) = \frac{{A}_{Li}}{{A}_{L 0} + {A}_{L 1}} \times 100$$

$$Peak area ratio of heavy chain bound to i drug molecule ( s ) = \frac{{A}_{Hi}}{{A}_{H 0} + {A}_{H 1} + {A}_{H 2} + {A}_{H 3}} \times 100$$

$${A}_{Li} and {A}_{Hi} : Corrected values of peak areas of {L}_{i} and {H}_{i} , respectively$$

F-3-4. The average number of conjugated drug molecules per antibody molecule in the antibody-drug conjugate is calculated according to the following expression.

$$Average number of conjugated drug molecules = ( {L}_{0} peak area ratio \times 0 + {L}_{1} peak area ratio \times 1 + {H}_{0} peak area ratio \times 0 + {H}_{1} peak area ratio \times 1 + {H}_{2} peak area ratio \times 2 + {H}_{3} peak area ratio \times 3 ) / 100 \times 2$$

It is to be noted that, in order to secure the amount of the antibody-drug conjugate, a plurality of antibody-drug conjugates having almost the same average number of conjugated drug molecules (e.g., on the order of ±1), which have been produced under similar conditions, can be mixed to prepare a new lot. In this case, the average number of drug molecules of the new lot falls between the average numbers of drug molecules before the mixing.

One specific example of the antibody-drug conjugate of the present disclosure can include an antibody-drug conjugate having a structure represented by the following formula:

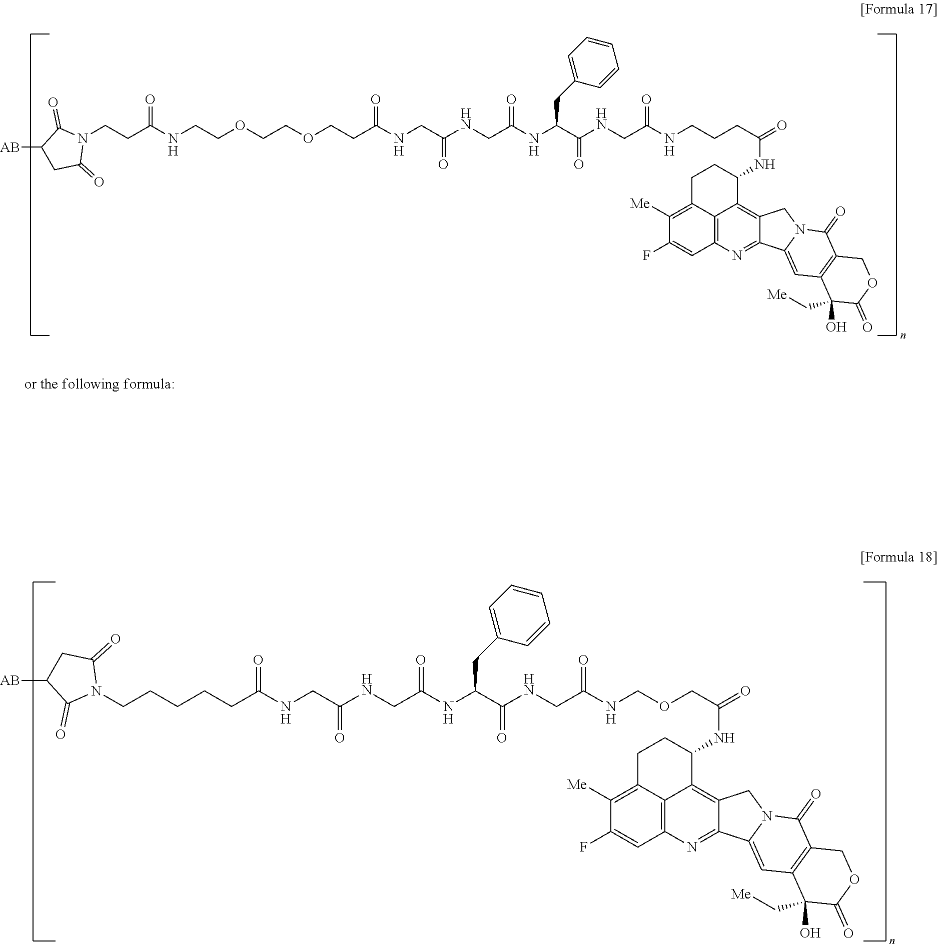

In this context, AB represents the anti-CDH6 antibody disclosed in the present description, and the antibody is conjugated to the drug linker via a sulfhydryl group stemming from the antibody. In this context, n has the same meaning as that of the so-called DAR (drug- to -antibody Ratio), and represents a drug- to -antibody ratio per antibody. Specifically, n represents the number of conjugated drug molecules per antibody molecule, which is a numeric value defined and indicated as an average value, i.e., the average number of conjugated drug molecules. In the case of the antibody-drug conjugate represented by [Formula 17] or [Formula 18] of the present disclosure, n can be 2 to 8 and is preferably 5 to 8, more preferably 7 to 8, and still more preferably 8, in measurement by common procedure F.

One example of the antibody-drug conjugate of the present disclosure can include an antibody-drug conjugate having a structure represented by the above-described formula [Formula 17] or [Formula 18] wherein the antibody represented by AB comprises any one antibody selected from the group consisting of the following antibodies (a) to (g), or a functional fragment of the antibody, or a pharmacologically acceptable salt of the antibody-drug conjugate:
(a) an antibody consisting of a light chain consisting of the amino acid sequence at positions 21 to 233 in the light chain full-length amino acid sequence shown in SEQ ID NO: 61 and a heavy chain consisting of the amino acid sequence at positions 20 to 471 in the heavy chain full-length amino acid sequence shown in SEQ ID NO: 69;
(b) an antibody consisting of a light chain consisting of the amino acid sequence at positions 21 to 233 in the light chain full-length amino acid sequence shown in SEQ ID NO: 61 and a heavy chain consisting of the amino acid sequence at positions 20 to 471 in the heavy chain full-length amino acid sequence shown in SEQ ID NO: 73;
(c) an antibody consisting of a light chain consisting of the amino acid sequence at positions 21 to 233 in the light chain full-length amino acid sequence shown in SEQ ID NO: 61 and a heavy chain consisting of the amino acid sequence at positions 20 to 471 in the heavy chain full-length amino acid sequence shown in SEQ ID NO: 77;
(d) an antibody consisting of a light chain consisting of the amino acid sequence at positions 21 to 233 in the light chain full-length amino acid sequence shown in SEQ ID NO: 65 and a heavy chain consisting of the amino acid sequence at positions 20 to 471 in the heavy chain full-length amino acid sequence shown in SEQ ID NO: 69;
(e) an antibody consisting of a light chain consisting of the amino acid sequence at positions 21 to 233 in the light chain full-length amino acid sequence shown in SEQ ID NO: 65 and a heavy chain consisting of the amino acid sequence at positions 20 to 471 in the heavy chain full-length amino acid sequence shown in SEQ ID NO: 73;
(f) an antibody consisting of a light chain consisting of the amino acid sequence at positions 21 to 233 in the light chain full-length amino acid sequence shown in SEQ ID NO: 65 and a heavy chain consisting of the amino acid sequence at positions 20 to 471 in the heavy chain full-length amino acid sequence shown in SEQ ID NO: 77; and
(g) any one antibody selected from the group consisting of the antibodies (a) to (f), wherein the heavy chain or the light chain comprises one or two or more modifications selected from the group consisting of posttranslational modifications typified by N-linked glycosylation, O-linked glycosylation, N-terminal processing, C-terminal processing, deamidation, isomerization of aspartic acid, oxidation of methionine, addition of a methionine residue to the N-terminus, amidation of a proline residue, and conversion of N-terminal glutamine or N-terminal glutamic acid to pyroglutamic acid, and a deletion of one or two amino acids at the carboxyl terminus

### 4. Medicament

Since the anti-CDH6 antibody of the present disclosure or the functional fragment of the antibody described in the above section “2. Production of anti-CDH6 antibody” and the Examples binds to CDH6 on the surface of tumor cells and has internalization activity, it can be used as a medicament, and in particular, as a therapeutic agent for cancer such as renal cell tumor or ovarian tumor, for example, renal cell carcinoma, renal clear cell carcinoma, papillary renal cell carcinoma, ovarian cancer, ovarian serous adenocarcinoma, thyroid cancer, bile duct cancer, lung cancer (e.g., small-cell lung cancer or non-small cell lung cancer), glioblastoma, mesothelioma, uterine cancer, pancreatic cancer, Wilms' tumor or neuroblastoma, either alone or in combination with an additional drug.

Furthermore, the anti-CDH6 antibody of the present disclosure or the functional fragment of the antibody can be used in the detection of cells expressing CDH6.

Moreover, since the anti-CDH6 antibody of the present disclosure or the functional fragment of the antibody has internalization activity, it can be applied as the antibody in an antibody-drug conjugate.

When a drug having antitumor activity such as cytotoxic activity is used as the drug, the anti-CDH6 antibody-drug conjugate of the present disclosure described in the above section “3. Anti-CDH6 antibody-drug conjugate” and the Examples is a conjugate of the anti-CDH6 antibody and/or the functional fragment of the antibody having internalization activity, and the drug having antitumor activity such as cytotoxic activity. Since this anti-CDH6 antibody-drug conjugate exhibits antitumor activity against cancer cells expressing CDH6, it can be used as a medicament, and in particular, as a therapeutic agent and/or a prophylactic agent for cancer.

The anti-CDH6 antibody-drug conjugate of the present disclosure may absorb moisture or have adsorption water, for example, to turn into a hydrate when it is left in air or subjected to recrystallization or purification procedures. Such a compound or a pharmacologically acceptable salt containing water is also included in the present disclosure.

When the anti-CDH6 antibody-drug conjugate of the present disclosure has a basic group such as an amino group, it can form a pharmacologically acceptable acid-addition salt, if desired. Examples of such an acid-addition salt can include: hydrohalides such as hydrofluoride, hydrochloride, hydrobromide, and hydroiodide; inorganic acid salts such as nitrate, perchlorate, sulfate, and phosphate; lower alkanesulfonates such as methanesulfonate, trifluoromethanesulfonate, and ethanesulfonate; arylsulfonates such as benzenesulfonate and p-toluenesulfonate; organic acid salts such as formate, acetate, trifluoroacetate, malate, fumarate, succinate, citrate, tartrate, oxalate, and maleate; and amino acid salts such as ornithine salt, glutamate, and aspartate.

When the anti-CDH6 antibody-drug conjugate of the present disclosure has an acidic group such as a carboxy group, it can form a pharmacologically acceptable base-addition salt, if desired. Examples of such a base-addition salt can include: alkali metal salts such as a sodium salt, a potassium salt, and lithium salt; alkaline earth metal salts such as a calcium salt and a magnesium salt; inorganic salts such as an ammonium salt; and organic amine salts such as a dibenzylamine salt, a morpholine salt, a phenylglycine alkyl ester salt, an ethylenediamine salt, an N-methylglucamine salt, a diethylamine salt, a triethylamine salt, a cyclohexylamine salt, a dicyclohexylamine salt, an N,N′-dibenzylethylenediamine salt, a diethanolamine salt, an N-benzyl-N-(2-phenylethoxy)amine salt, a piperazine salt, tetramethylammonium salt, and a tris(hydroxymethyl)aminomethane salt.

The present disclosure can also include an anti-CDH6 antibody-drug conjugate in which one or more atoms constituting the antibody-drug conjugate are replaced with isotopes of the atoms. There exist two types of isotopes: radioisotopes and stable isotopes. Examples of the isotope can include isotypes of hydrogen (2H and 3H), isotopes of carbon (11C, 13C and 14C), isotopes of nitrogen (13N and 15N), isotopes of oxygen (150, 170 and 180), and isotopes of fluorine (18F). A composition comprising the antibody-drug conjugate labeled with such an isotope is useful as, for example, a therapeutic agent, a prophylactic agent, a research reagent, an assay reagent, a diagnostic agent, and an in vivo diagnostic imaging agent. Each and every antibody-drug conjugate labeled with an isotope, and mixtures of antibody-drug conjugates labeled with an isotope at any given ratio are included in the present disclosure. The antibody-drug conjugate labeled with an isotope can be produced, for example, by using a starting material labeled with an isotope, instead of a starting material for the production method of the present disclosure mentioned later, according to a method known in the art.

In vitro cytotoxicity can be measured based on the activity of suppressing the proliferative responses of cells, for example. For example, a cancer cell line overexpressing CDH6 is cultured, and the anti-CDH6 antibody-drug conjugate is added at different concentrations to the culture system. Thereafter, its suppressive activity against focus formation, colony formation and spheroid growth can be measured. In this context, for example, by using a renal cell tumor- or ovarian tumor-derived cancer cell line, cell growth inhibition activity against renal cell tumor or ovarian tumor can be examined.

In vivo therapeutic effects on cancer in an experimental animal can be measured, for example, by administering the anti-CDH6 antibody-drug conjugate to a nude mouse into which a tumor cell line highly expressing CDH6 has been inoculated, and then measuring a change in the cancer cells. In this context, for example, by using an animal model derived from an immunodeficient mouse by the inoculation of renal cell carcinoma-, renal clear cell carcinoma-, papillary renal cell carcinoma-, ovarian cancer-, ovarian serous adenocarcinoma- or thyroid cancer-derived cells, therapeutic effects on renal cell carcinoma, renal clear cell carcinoma, papillary renal cell carcinoma, ovarian cancer, ovarian serous adenocarcinoma or thyroid cancer can be measured.

The type of cancer to which the anti-CDH6 antibody-drug conjugate of the present disclosure is applied is not particularly limited as long as the cancer expresses CDH6 in cancer cells to be treated. Examples thereof can include renal cell carcinoma (e.g., renal clear cell carcinoma or papillary renal cell carcinoma), ovarian cancer, ovarian serous adenocarcinoma, thyroid cancer, bile duct cancer, lung cancer (e.g., small-cell lung cancer or non-small cell lung cancer), glioblastoma, mesothelioma, uterine cancer, pancreatic cancer, Wilms' tumor and neuroblastoma, though the cancer is not limited thereto as long as the cancer expresses CDH6. Examples thereof can also include renal cell carcinoma, renal clear cell carcinoma, papillary renal cell carcinoma, ovarian cancer, ovarian serous adenocarcinoma, clear cell carcinoma of ovary, endometrioid carcinoma of ovary, ovarian mucinous tumor, thyroid cancer, bile duct cancer, lung cancer, non-small cell lung cancer, cervix cancer, brain tumor, head and neck cancer, sarcoma, osteosarcoma, small cell lung cancer, glioblastoma, mesothelioma, uterine cancer, pancreatic cancer, Wilms' tumor, neuroblastoma, colorectal cancer, gastric cancer, endometrial cancer, nasopharyngeal cancer, prostate cancer or cancers associated with von Hippel-Lindau disease. Preferred examples thereof can include renal cell carcinoma, renal clear cell carcinoma, papillary renal cell carcinoma, ovarian cancer, ovarian serous adenocarcinoma, clear cell carcinoma of ovary, endometrioid carcinoma of ovary, ovarian mucinous tumor, thyroid cancer, bile duct cancer, lung cancer, non-small cell lung cancer, cervix cancer, brain tumor, head and neck cancer, sarcoma, osteosarcoma, small cell lung cancer, glioblastoma, mesothelioma, uterine cancer, pancreatic cancer, Wilms' tumor and neuroblastoma. More preferred examples of the cancer can include renal cell carcinoma (e.g., renal clear cell carcinoma and papillary renal cell carcinoma) and ovarian cancer. Furthermore preferred examples of the cancer can include ovarian cancer (e.g., epithelial ovarian cancer, fallopian tube cancer, and primary peritoneal cancer). The cancer may be metastatic.

The anti-CDH6 antibody-drug conjugate of the present disclosure can preferably be administered to a mammal, and more preferably to a human.

### Pharmaceutical Compositions and Modes of Administration

A substance used in a pharmaceutical composition comprising the anti-CDH6 antibody-drug conjugate of the present disclosure can be appropriately selected from pharmaceutical additives and others usually used in this field, in terms of the applied dose or the applied concentration, and then used.

The anti-CDH6 antibody-drug conjugate of the present disclosure can be administered as a pharmaceutical composition comprising one or more pharmaceutically compatible components. For example, the pharmaceutical composition typically comprises one or more pharmaceutical carriers (e.g., sterilized liquids (e.g., water and oil (including petroleum oil and oil of animal origin, plant origin, or synthetic origin (e.g., peanut oil, soybean oil, mineral oil, and sesame oil))). Water is a more typical carrier when the pharmaceutical composition is intravenously administered. An aqueous saline solution, an aqueous dextrose solution, and an aqueous glycerol solution can also be used as a liquid carrier, in particular, for an injection solution. Suitable pharmaceutical vehicles are known in the art. If desired, the composition may also comprise a trace amount of a moisturizing agent, an emulsifying agent, or a pH buffering agent. Examples of suitable pharmaceutical carriers are disclosed in “Remington's Pharmaceutical Sciences” by E. W. Martin. The prescription corresponds to an administration mode.

In some aspects, the antibody-drug conjugate (ADC) comprises formula 3. In some aspects, the ADC comprising formula 3 is administered to patients exhibiting resistance to platinum based cancer-treatments. In some aspects, the ADC comprising formula 3 is administered to platinum-resistant cancer patients having a recurrence of cancer within 2, 3, 4, 5, 6, 7, 8, 9, 10, 11, or 12 months of completion of a platinum-based cancer treatment. In some aspects, the ADC comprising formula 3 is administered for platinum-resistant cancer patients having a disease recurrence within at least 2, 3, 4, 5, 6, 7, 8, 9, 10, 11, or 12 months of completion of a platinum-based cancer treatment. In some aspects, the ADC comprising formula 3 is administered for platinum-resistant cancer patients having a disease recurrence within at least 2, 3, 4, 5, 6, 7, 8, 9, 10, 11, or 12 months of completion of a carboplatin and/or paclitaxel regimen.

In some aspects, the ADC comprising formula 3 is administered after treatment with a platinum/taxane chemotherapy, such as carboplatin/docetaxel, cisplatin/paclitaxel, and carboplatin/paclitaxel/bevacizumab regimens.

In some aspects, the ADC comprising formula 3 is administered after treatment with one or more of the following platinum-based chemotherapies, such as carboplatin/liposomal doxorubicin, carboplatin/gemcitabine, cisplatin/gemcitabine, carboplatin/ifosfamide, cisplatin/ifosfamide, oxaliplatin/5-FU/Luecovorin, and oxaliplatin/capecitabine regimens.

In some aspects, the ADC comprising formula 3 is administered to a patient in need thereof for treatment of platinum-resistant, preferably, having a disease recurrence of, ovarian cancer, non-small cell lung cancer (NSCLC), breast cancer, bladder cancer, endometrial cancer, castrate-resistant prostate cancer (CRPC), and other cancers which express CDH6. In some aspects, ovarian cancer includes epithelial ovarian cancer, fallopian tube cancer, and primary peritoneal cancer.

In some aspects, the ADC comprising formula 3 is indicated for the treatment of patients with ovarian cancer whose disease has progressed or recurred-after platinum-based chemotherapy. In some aspects, the ADC comprising formula 3 is indicated for the treatment of advanced ovarian cancer in women who have failed a first-line platinum-based chemotherapy regimen. In some aspects, the ADC comprising formula 3 is indicated for treating ovarian cancer after disease progression after the chemotherapy.

In some aspects, the ADC comprising formula 3 is indicated for the treatment of patients with metastatic ovarian cancer after disease progression on or after initial or subsequent chemotherapy, as a single agent. In some aspects, the ADC comprising formula 3 is indicated for the treatment of patients with metastatic carcinoma of the ovary after failure of first-line or subsequent therapy.

Various delivery systems are known, and they can be used for administering the anti-CDH6 antibody-drug conjugate of the present disclosure. Examples of the administration route can include, but are not limited to, intradermal, intramuscular, intraperitoneal, intravenous, and subcutaneous routes. The administration can be made by injection or bolus injection, for example. According to a specific preferred embodiment, the administration of the above-described antibody-drug conjugate is performed by injection. Parenteral administration is a preferred administration route.

According to a representative embodiment, the pharmaceutical composition is prescribed, as a pharmaceutical composition suitable for intravenous administration to a human, according to conventional procedures. The composition for intravenous administration is typically a solution in a sterile and isotonic aqueous buffer solution. If necessary, the medicament may also contain a solubilizing agent and a local anesthetic to alleviate pain at an injection area (e.g., lignocaine). In general, the above-described ingredients are provided, either separately or together in a mixture in unit dosage form, as a freeze-dried powder or an anhydrous concentrate contained in a container which is obtained by sealing in, for example, an ampoule or a sachet indicating the amount of the active agent. When the medicament is to be administered by injection, it may be administered using, for example, an injection bottle containing water or saline of sterile pharmaceutical grade. When the medicament is to be administered by injection, an ampoule of sterile water or saline for injection may be provided such that the above-described ingredients are admixed with one another before administration.

The pharmaceutical composition of the present disclosure may be a pharmaceutical composition comprising only the anti-CDH6 antibody-drug conjugate of the present application, or may be a pharmaceutical composition comprising the anti-CDH6 antibody-drug conjugate and at least one other therapeutic agent for cancer. The anti-CDH6 antibody-drug conjugate of the present disclosure can also be administered together with an additional therapeutic agent for cancer, and can thereby enhance an anticancer effect. The additional anticancer agent used for such a purpose may be administered to an individual, simultaneously, separately, or continuously, together with the antibody-drug conjugate. Otherwise, the additional anticancer agent and the anti-CDH6 antibody-drug conjugate may each be administered to the subject at different administration intervals. In the present disclosure, the phrase “second drug” means a therapeutic agent other than the anti-CDH6 antibody-drug conjugate of the present disclosure. Additional anticancer agents can be “second drugs”. In the present disclosure, however, a “second drug” doesn't have to be a drug used for so-called “second line therapies”. Examples of such a therapeutic agent or a second drug for cancer, or an additional anticancer agent can include tyrosine kinase inhibitors including imatinib, sunitinib, and regorafenib, CDK4/6 inhibitors including palbociclib, HSP90 inhibitors including TAS-116, MEK inhibitors including MEK162, and immune checkpoint inhibitors including nivolumab, pembrolizumab, and ipilimumab, though the therapeutic agent for cancer is not limited thereto as long as the drug has antitumor activity. In the case that a cancer is resistant to a particular anticancer agent, the second drug may be selected from anticancer agents other than the particular therapeutic agent.

Such a pharmaceutical composition can be prepared as a formulation having a selected composition and a necessary purity in the form of a freeze-dried formulation or a liquid formulation. The pharmaceutical composition prepared as a freeze-dried formulation may be a formulation containing an appropriate pharmaceutical additive used in this field. Likewise, the liquid formulation can be prepared such that the liquid formulation contains various pharmaceutical additives used in this field.

The composition and concentration of the pharmaceutical composition also vary depending on the administration method. With regard to the affinity of the anti-CDH6 antibody-drug conjugate comprised in the pharmaceutical composition of the present disclosure for the antigen, i.e., the dissociation constant (Kd value) of the anti-CDH6 antibody-drug conjugate to the antigen, as the affinity increases (i.e., the Kd value is low), the pharmaceutical composition can exert medicinal effects, even if the applied dose thereof is decreased. Accordingly, the applied dose of the antibody-drug conjugate can also be determined by setting the applied dose based on the status of the affinity of the antibody-drug conjugate for the antigen. When the antibody-drug conjugate of the present disclosure is administered to a human, it may be administered at a dose of, for example, from approximately 0.001 to 100 mg/kg once or a plurality of times at intervals of 1 to 180 days.

### Methods of Treatment and Uses

The present disclosure provides methods of treating cancer comprising administering an anti-CDH6 antibody-drug conjugate as disclosed herein. Also provided herein are any of the disclosed anti-CDH6 ADC for use in treating a cancer.

In some embodiments, the cancer is a CDH6-expressing cancer. CDH6-expressing cancers may include, but are not limited to, renal cell carcinoma, renal clear cell carcinoma, papillary renal cell carcinoma, ovarian cancer, ovarian serous adenocarcinoma, thyroid cancer, bile duct cancer, lung cancer, small-cell lung cancer, glioblastoma, mesothelioma, uterine cancer, pancreatic cancer, Wilms' tumor and neuroblastoma.

For the purposes of the present disclosure, the term “CDH6-overexpressing cancer” is not particularly limited as long as it is recognized as CDH6-overexpressing cancer by those skilled in the art. Preferred examples of the CDH6-overexpressing cancer can include cancer given a high score for the expression of CDH6 in an immunohistochemical method (IHC) or an in situ hybridization method (ISH) or a next generation sequencing method (NGS). The in situ hybridization method of the present disclosure includes a fluorescence in situ hybridization method (FISH) and a dual color in situ hybridization method (DISH).

The method for scoring the degree of CDH6 expression by the immunohistochemical method, or the method for determining positivity or negativity to CDH6 expression by the in situ hybridization method is not particularly limited as long as it is recognized by those skilled in the art.

In some embodiments, the ADC and the treatment methods and uses of the present disclosure can also be used as a pharmaceutical composition for treatment of cancer comprising the antibody-drug conjugate used in the present disclosure, a salt thereof, or a hydrate thereof as an active component, and a pharmaceutically acceptable formulation component.

In some embodiments, the ADC and the treatment methods and uses of the present disclosure exhibit excellent antitumor activity against cancer that exhibits resistance to an existing anticancer drug (i.e., resistant cancer), particularly, cancer that has acquired resistance to an existing anticancer drug (i.e., secondary resistant cancer). Thus, the ADC for treatment of the present disclosure exerts a remarkable antitumor effect when applied to a patient group with cancer having resistance to an existing anticancer drug (patients having a history of treatment with an existing anticancer drug) among cancer patients. In particular, the cancer being treated may be resistant to or refractory from treatment with a platinum-based chemotherapeutics (i.e., cisplatin, carboplatin), a chemotherapeutic (i.e., docetaxel, gemcitabine, liposomal doxorubicin, paclitaxel, topotecan), a PARP inhibitor treatment (i.e., niraparib, olaparib, rucaparib, talazoparib), an immune checkpoint inhibitor treatment (i.e., nivolumab, pembrolizumab, atezolizumab, avelumab, ipilimumab, durvalumab, tislelizumab, sintilimab, cemiplimab), an angiogenic inhibitor treatment (i.e., bevacizumab, everolimus) and/or a VEGFR-TKI treatment (i.e., pazopanib, sunitinib, sorafenib, cabozantinib, Lenvatinib, axitinib).

The ADC and the treatment methods and uses of the present disclosure can be preferably used for the treatment of inoperable or recurrent cancer.

The ADC and methods or uses for treatment of the present disclosure can delay development of cancer cells, inhibit growth thereof, and further kill cancer cells. These effects can allow cancer patients to be free from symptoms caused by cancer or achieve improvement in quality of life (QOL) of cancer patients and attain a therapeutic effect by sustaining the lives of the cancer patients. Even if the anti-CDH6 antibody-drug conjugate of the present disclosure does not accomplish killing cancer cells, it can provide higher QOL of cancer patients while achieving longer-term survival, by inhibiting or controlling the growth of cancer cells.

In some embodiments of the methods and uses, a subject with cancer (e.g., a CDH6-expressing cancer) may be administered about 0.1 to about 15 mg/kg, about 0.5 to about 12 mg/kg, about 1.0 to about 10 mg/kg, or about 4 to about 8 mg/kg. In some embodiments, the dose may be about 1.0 mg/kg to about 12 mg/kg, about 1.6 mg/kg to about 3.2 mg/kg, about 1.6 mg/kg to about 4.8 mg/kg, about 1.6 mg/kg to about 5.4 mg/kg, about 1.6 mg/kg to about 5.6 mg/kg, about 1.6 mg/kg to about 6.4 mg/kg, about 1.6 mg/kg to about 8.0 mg/kg, about 1.6 mg/kg to about 9.6 mg/kg, about 3.2 mg/kg to about 4.8 mg/kg, about 3.2 mg/kg to about 5.4 mg/kg, about 3.2 mg/kg to about 5.6 mg/kg, about 3.2 mg/kg to about 6.4 mg/kg, about 3.2 mg/kg to about 8.0 mg/kg, about 3.2 mg/kg to about 9.6 mg/kg, about 4.8 mg/kg to about 5.4 mg/kg, about 4.8 mg/kg to about 5.6 mg/kg, about 4.8 mg/kg to about 6.4 mg/kg, about 4.8 mg/kg to about 8.0 mg/kg, about 4.8 mg/kg to about 9.6 mg/kg, about 5.4 mg/kg to about 5.6 mg/kg, about 5.4 mg/kg to about 6.4 mg/kg, about 5.4 mg/kg to about 8.0 mg/kg, about 5.4 mg/kg to about 9.6 mg/kg, about 5.6 mg/kg to about 6.4 mg/kg, about 5.6 mg/kg to about 8.0 mg/kg, about 5.6 mg/kg to about 9.6 mg/kg, about 6.4 mg/kg to about 9.6 mg/kg, about 6.4 mg/kg to about 8.0 mg/kg, or about 8.0 mg/kg to about 9.6 mg/kg. In some embodiments the dose of the ADC administered to the subject may be about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9 or about 12.0 mg/kg or more. In preferred embodiments, the dose may be about 1.6 mg/kg, about 3.2 mg/kg, about 4.8 mg/kg, about 5.4 mg/kg, about 5.6 mg/kg, about 6.4 mg/kg, about 8.0 mg/kg or about 9.6 mg/kg, but more preferably about 3.2 mg/kg, about 4.8 mg/kg, about 5.4 mg/kg, about 5.6 mg/kg, about 6.4 mg/kg, about 8.0 mg/kg or about 9.6 mg/kg, but further preferably about 4.8 mg/kg, about 5.4 mg/kg, about 5.6 mg/kg, about 6.4 mg/kg or about 8.0 mg/kg, but furthermore preferably about 4.8 mg/kg, about 5.6 mg/kg, about 6.4 mg/kg or about 8.0 mg/kg.

In preferred embodiments, the dose may be about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5.0 mg/kg, about 5.1 mg/kg, about 5.2 mg/kg, about 5.3 mg/kg, about 5.4 mg/kg, about 5.5 mg/kg, about 5.6 mg/kg, about 5.7 mg/kg, about 5.8 mg/kg, about 5.9 mg/kg, about 6.0 mg/kg, about 6.1 mg/kg, about 6.2 mg/kg, about 6.3 mg/kg or about 6.4 mg/kg, but more preferably about 4.8 mg/kg, about 4.9 mg/kg, about 5.0 mg/kg, about 5.1 mg/kg, about 5.2 mg/kg, about 5.3 mg/kg, about 5.4 mg/kg, about 5.5 mg/kg, about 5.6 mg/kg, about 5.7 mg/kg, about 5.8 mg/kg, about 5.9 mg/kg, about 6.0 mg/kg, about 6.1 mg/kg, about 6.2 mg/kg, about 6.3 mg/kg or about 6.4 mg/kg.

In some embodiments of the methods and uses, a subject with cancer (e.g., a CDH6-expressing cancer) may be administered 0.1 to 15 mg/kg, 0.5 to 12 mg/kg, 1.0 to 10 mg/kg, or 4 to 8 mg/kg. In some embodiments, the dose may be 1.0 mg/kg to 12 mg/kg, 1.6 mg/kg to 3.2 mg/kg, 1.6 mg/kg to 4.8 mg/kg, 1.6 mg/kg to 5.4 mg/kg, 1.6 mg/kg to 5.6 mg/kg, 1.6 mg/kg to 6.4 mg/kg, 1.6 mg/kg to 8.0 mg/kg, 1.6 mg/kg to 9.6 mg/kg, 3.2 mg/kg to 4.8 mg/kg, 3.2 mg/kg to 5.4 mg/kg, 3.2 mg/kg to 5.6 mg/kg, 3.2 mg/kg to 6.4 mg/kg, 3.2 mg/kg to 8.0 mg/kg, 3.2 mg/kg to 9.6 mg/kg, 4.8 mg/kg to 5.4 mg/kg, 4.8 mg/kg to 5.6 mg/kg, 4.8 mg/kg to 6.4 mg/kg, 4.8 mg/kg to 8.0 mg/kg, 4.8 mg/kg to 9.6 mg/kg, 5.4 mg/kg to 5.6 mg/kg, 5.4 mg/kg to 6.4 mg/kg, 5.4 mg/kg to 8.0 mg/kg, 5.4 mg/kg to 9.6 mg/kg, 5.6 mg/kg to 6.4 mg/kg, 5.6 mg/kg to 8.0 mg/kg, 5.6 mg/kg to 9.6 mg/kg, 6.4 mg/kg to 9.6 mg/kg, 6.4 mg/kg to 8.0 mg/kg, or 8.0 mg/kg to 9.6 mg/kg. In some embodiments, the dose of the ADC administered to the subject may be 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9 or 12.0 mg/kg or more. In preferred embodiments, the dose may be 1.6 mg/kg, 3.2 mg/kg, 4.8 mg/kg, 5.4 mg/kg, 5.6 mg/kg, 6.4 mg/kg, 8.0 mg/kg, 9.6 mg/kg, but more preferably 3.2 mg/kg, 4.8 mg/kg, 5.4 mg/kg, 5.6 mg/kg, 6.4 mg/kg, 8.0 mg/kg or 9.6 mg/kg, but further preferably 4.8 mg/kg, 5.4 mg/kg, 5.6 mg/kg, 6.4 mg/kg or 8.0 mg/kg, but furthermore preferably 4.8 mg/kg, 5.6 mg/kg, 6.4 mg/kg or 8.0 mg/kg.

In preferred embodiments, the dose may be 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg or 6.4 mg/kg, but more preferably 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg or 6.4 mg/kg.

In some embodiments of the methods and uses, a subject with cancer (e.g., a CDH6-expressing cancer) may be administered about 5 to 3000 mg, about 5 to 2500 mg, about 5 to 2000 mg, about 5 to about 1500 mg, about 5 to about 1000 mg, about 10 to about 3000 mg, about 10 to about 2500 mg, about 10 to about 2000 mg, about 10 to about 1500 mg, about 10 to about 1000 mg, about 25 to about 2500 mg, about 25 to about 2000 mg, about 25 to about 1500 mg, about 25 to about 1000 mg, about 50 to about 2500 mg, about 50 to about 2000 mg, about 50 to about 1500 mg, about 50 to about 1000 mg, about 50 to about 950 mg, about 50 to about 900 mg, about 50 to about 850 mg, about 50 to about 800 mg, about 50 to about 750 mg, about 50 to about 700 mg, about 50 to about 650 mg, about 50 to about 600 mg, about 50 to about 550 mg, about 50 to about 500 mg, about 50 to about 450 mg, about 50 to about 400 mg, about 50 to about 350 mg, about 50 to about 300 mg, about 50 to about 250 mg, about 100 to about 2500 mg, about 100 to about 2000 mg, about 100 to about 1500 mg, about 100 to about 1000 mg, about 100 to about 950 mg, about 100 to about 900 mg, about 100 to about 850 mg, about 100 to about 800 mg, about 100 to about 750 mg, about 100 to about 700 mg, about 100 to about 650 mg, about 100 to about 600 mg, about 100 to about 550 mg, about 100 to about 500 mg, about 100 to about 450 mg, about 100 to about 400 mg, about 100 to about 350 mg, about 100 to about 300 mg, about 100 to about 250 mg, about 150 to about 2000 mg, about 150 to about 1500 mg, about 150 to about 1000 mg, about 150 to about 950 mg, about 150 to about 900 mg, about 150 to about 850 mg, about 150 to about 800 mg, about 150 to about 750 mg, about 150 to about 700 mg, about 150 to about 650 mg, about 150 to about 600 mg, about 150 to about 550 mg, about 150 to about 500 mg, about 150 to about 450 mg, about 150 to about 400 mg, about 150 to about 350 mg, about 150 to about 300 mg, about 150 to about 250 mg, about 200 to about 1000 mg, about 200 to about 950 mg, about 200 to about 900 mg, about 200 to about 850 mg, about 200 to about 800 mg, about 200 to about 750 mg, about 200 to about 700 mg, about 200 to about 650 mg, about 200 to about 600 mg, about 200 to about 550 mg, about 200 to about 500 mg, about 200 to about 450 mg, about 200 to about 400 mg, about 200 to about 350 mg, about 200 to about 300 mg, about 200 to about 250 mg, about 250 to about 1000 mg, about 250 to about 950 mg, about 250 to about 900 mg, about 250 to about 850 mg, about 250 to about 800 mg, about 250 to about 750 mg, about 250 to about 700 mg, about 250 to about 650 mg, about 250 to about 600 mg, about 250 to about 550 mg, about 250 to about 500 mg, about 250 to about 450 mg, about 250 to about 400 mg, about 250 to about 350 mg, about 250 to about 300 mg, about 300 to about 1000 mg, about 300 to about 950 mg, about 300 to about 900 mg, about 300 to about 850 mg, about 300 to about 800 mg, about 300 to about 750 mg, about 300 to about 700 mg, about 300 to about 650 mg, about 300 to about 600 mg, about 300 to about 550 mg, about 300 to about 500 mg, about 300 to about 450 mg, about 300 to about 400 mg, about 300 to about 350 mg, about 350 to about 1000 mg, about 350 to about 950 mg, about 350 to about 900 mg, about 350 to about 850 mg, about 350 to about 800 mg, about 350 to about 750 mg, about 350 to about 700 mg, about 350 to about 650 mg, about 350 to about 600 mg, about 350 to about 550 mg, about 350 to about 500 mg, about 350 to about 450 mg, about 350 to about 400 mg, about 400 to about 1000 mg, about 400 to about 950 mg, about 400 to about 900 mg, about 400 to about 850 mg, about 400 to about 800 mg, about 400 to about 750 mg, about 400 to about 700 mg, about 400 to about 650 mg, about 400 to about 600 mg, about 400 to about 550 mg, about 400 to about 500 mg, about 400 to about 450 mg, about 450 to about 1000 mg, about 450 to about 950 mg, about 450 to about 900 mg, about 450 to about 850 mg, about 450 to about 800 mg, about 450 to about 750 mg, about 450 to about 700 mg, about 450 to about 650 mg, about 450 to about 600 mg, about 450 to about 550 mg, about 450 to about 500 mg, about 500 to about 1000 mg, about 500 to about 950 mg, about 500 to about 900 mg, about 500 to about 850 mg, about 500 to about 800 mg, about 500 to about 750 mg, about 500 to about 700 mg, about 500 to about 650 mg, about 500 to about 600 mg, or about 500 to about 550 mg. In other words, in some embodiments the dose of the ADC administered to the subject may be about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, or about 2000 mg, or any value in between.

In preferred embodiments, the dose of the ADC administered to the subject may be about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg

In some embodiments of the methods and uses, a subject with cancer (e.g., a CDH6-expressing cancer) may be administered 5 to 3000 mg, 5 to 2500 mg, 5 to 2000 mg, 5 to 1500 mg, 5 to 1000 mg, 10 to 3000 mg, 10 to 2500 mg, 10 to 2000 mg, 10 to 1500 mg, 10 to 1000 mg, 25 to 2500 mg, 25 to 2000 mg, 25 to 1500 mg, 25 to 1000 mg, 50 to 2500 mg, 50 to 2000 mg, 50 to 1500 mg, 50 to 1000 mg, 50 to 950 mg, 50 to 900 mg, 50 to 850 mg, 50 to 800 mg, 50 to 750 mg, 50 to 700 mg, 50 to 650 mg, 50 to 600 mg, 50 to 550 mg, 50 to 500 mg, 50 to 450 mg, 50 to 400 mg, 50 to 350 mg, 50 to 300 mg, 50 to 250 mg, 100 to 2500 mg, 100 to 2000 mg, 100 to 1500 mg, 100 to 1000 mg, 100 to 950 mg, 100 to 900 mg, 100 to 850 mg, 100 to 800 mg, 100 to 750 mg, 100 to 700 mg, 100 to 650 mg, 100 to 600 mg, 100 to 550 mg, 100 to 500 mg, 100 to 450 mg, 100 to 400 mg, 100 to 350 mg, 100 to 300 mg, 100 to 250 mg, 150 to 2000 mg, 150 to 1500 mg, 150 to 1000 mg, 150 to 950 mg, 150 to 900 mg, 150 to 850 mg, 150 to 800 mg, 150 to 750 mg, 150 to 700 mg, 150 to 650 mg, 150 to 600 mg, 150 to 550 mg, 150 to 500 mg, 150 to 450 mg, 150 to 400 mg, 150 to 350 mg, 150 to 300 mg, 150 to 250 mg, 200 to 1000 mg, 200 to 950 mg, 200 to 900 mg, 200 to 850 mg, 200 to 800 mg, 200 to 750 mg, 200 to 700 mg, 200 to 650 mg, 200 to 600 mg, 200 to 550 mg, 200 to 500 mg, 200 to 450 mg, 200 to 400 mg, 200 to 350 mg, 200 to 300 mg, 200 to 250 mg, 250 to 1000 mg, 250 to 950 mg, 250 to 900 mg, 250 to 850 mg, 250 to 800 mg, 250 to 750 mg, 250 to 700 mg, 250 to 650 mg, 250 to 600 mg, 250 to 550 mg, 250 to 500 mg, 250 to 450 mg, 250 to 400 mg, 250 to 350 mg, 250 to 300 mg, 300 to 1000 mg, 300 to 950 mg, 300 to 900 mg, 300 to 850 mg, 300 to 800 mg, 300 to 750 mg, 300 to 700 mg, 300 to 650 mg, 300 to 600 mg, 300 to 550 mg, 300 to 500 mg, 300 to 450 mg, 300 to 400 mg, 300 to 350 mg, 350 to 1000 mg, 350 to 950 mg, 350 to 900 mg, 350 to 850 mg, 350 to 800 mg, 350 to 750 mg, 350 to 700 mg, 350 to 650 mg, 350 to 600 mg, 350 to 550 mg, 350 to 500 mg, 350 to 450 mg, 350 to 400 mg, 400 to 1000 mg, 400 to 950 mg, 400 to 900 mg, 400 to 850 mg, 400 to 800 mg, 400 to 750 mg, 400 to 700 mg, 400 to 650 mg, 400 to 600 mg, 400 to 550 mg, 400 to 500 mg, 400 to 450 mg, 450 to 1000 mg, 450 to 950 mg, 450 to 900 mg, 450 to 850 mg, 450 to 800 mg, 450 to 750 mg, 450 to 700 mg, 450 to 650 mg, 450 to 600 mg, 450 to 550 mg, 450 to 500 mg, 500 to 1000 mg, 500 to 950 mg, 500 to 900 mg, 500 to 850 mg, 500 to 800 mg, 500 to 750 mg, 500 to 700 mg, 500 to 650 mg, 500 to 600 mg, or 500 to 550 mg. In other words, in some embodiments the dose of the ADC administered to the subject may be 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg, 1025 mg, 1050 mg, 1075 mg, 1100 mg, 1125 mg, 1150 mg, 1175 mg, 1200 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, or 2000 mg, or any value in between.

In preferred embodiments, the dose of the ADC administered to the subject may be 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg.

In some embodiments of the methods and uses, the anti-CDH6 ADC or a pharmaceutical composition thereof is administered to a subject with cancer via parenteral administration. Preferred parenteral routes of administering include, but are not limited to, injections, such as intravenous, intramuscular, and subcutaneous injections. The anti-CDH6 antibody-drug conjugate used in the present disclosure can be expected to exert a therapeutic effect by application as systemic therapy to patients, and additionally, by local application to cancer tissues.

The timing or regimen of administration may be once every 1 week (q1w), once every 2 weeks (q2w), once every 3 weeks (q3w), once every 4 weeks (q4w), once every 5 weeks (q5w), once every 6 weeks (q6w), once every 7 weeks (q7w), once every 8 week (q8w), once every 9 weeks (q9w), or once every 10 weeks (q10w), but is preferably once every 3 or 4 weeks, but is more preferably once every 3 weeks.

Dosage regimens may be adjusted to provide the optimum desired response (e.g., a therapeutic response like tumor regression or remission). The below dosages are shown for once every three weeks, but could also be provided q1w, q2w, q4w, q5w, q6w, q7w, q8w, q9w, or q10w. For example, in some embodiments, dosage regimen may be 0.1 mg/kg once every 3 weeks (q3w), 0.2 mg/kg once every 3 weeks (q3w), 0.3 mg/kg once every 3 weeks (q3w), 0.4 mg/kg once every 3 weeks (q3w), 0.5 mg/kg once every 3 weeks (q3w), 0.6 mg/kg once every 3 weeks (q3w), 0.7 mg/kg once every 3 weeks (q3w), 0.8 mg/kg once every 3 weeks (q3w), 0.9 mg/kg once every 3 weeks (q3w), 1.0 mg/kg once every 3 weeks (q3w), 1.1 mg/kg once every 3 weeks (q3w), 1.2 mg/kg once every 3 weeks (q3w), 1.3 mg/kg once every 3 weeks (q3w), 1.4 mg/kg once every 3 weeks (q3w), 1.5 mg/kg once every 3 weeks (q3w), 1.6 mg/kg once every 3 weeks (q3w), 1.7 mg/kg once every 3 weeks (q3w), 1.8 mg/kg once every 3 weeks (q3w), 1.9 mg/kg once every 3 weeks (q3w), 2.0 mg/kg once every 3 weeks (q3w), 2.1 mg/kg once every 3 weeks (q3w), 2.2 mg/kg once every 3 weeks (q3w), 2.3 mg/kg once every 3 weeks (q3w), 2.4 mg/kg once every 3 weeks (q3w), 2.5 mg/kg once every 3 weeks (q3w), 2.6 mg/kg once every 3 weeks (q3w), 2.7 mg/kg once every 3 weeks (q3w), 2.8 mg/kg once every 3 weeks (q3w), 2.9 mg/kg once every 3 weeks (q3w), 3.0 mg/kg once every 3 weeks (q3w), 3.1 mg/kg once every 3 weeks (q3w), 3.2 mg/kg once every 3 weeks (q3w), 3.3 mg/kg once every 3 weeks (q3w), 3.4 mg/kg once every 3 weeks (q3w), 3.5 mg/kg once every 3 weeks (q3w), 3.6 mg/kg once every 3 weeks (q3w), 3.7 mg/kg once every 3 weeks (q3w), 3.8 mg/kg once every 3 weeks (q3w), 3.9 mg/kg once every 3 weeks (q3w), 4.0 mg/kg once every 3 weeks (q3w), 4.1 mg/kg once every 3 weeks (q3w), 4.2 mg/kg once every 3 weeks (q3w), 4.3 mg/kg once every 3 weeks (q3w), 4.4 mg/kg once every 3 weeks (q3w), 4.5 mg/kg once every 3 weeks (q3w), 4.6 mg/kg once every 3 weeks (q3w), 4.7 mg/kg once every 3 weeks (q3w), 4.8 mg/kg once every 3 weeks (q3w), 4.9 mg/kg once every 3 weeks (q3w), 5.0 mg/kg once every 3 weeks (q3w), 5.1 mg/kg once every 3 weeks (q3w), 5.2 mg/kg once every 3 weeks (q3w), 5.3 mg/kg once every 3 weeks (q3w), 5.4 mg/kg once every 3 weeks (q3w), 5.5 mg/kg once every 3 weeks (q3w), 5.6 mg/kg once every 3 weeks (q3w), 5.7 mg/kg once every 3 weeks (q3w), 5.8 mg/kg once every 3 weeks (q3w), 5.9 mg/kg once every 3 weeks (q3w), 6.0 mg/kg once every 3 weeks (q3w), 6.1 mg/kg once every 3 weeks (q3w), 6.2 mg/kg once every 3 weeks (q3w), 6.3 mg/kg once every 3 weeks (q3w), 6.4 mg/kg once every 3 weeks (q3w), 6.5 mg/kg once every 3 weeks (q3w), 6.6 mg/kg once every 3 weeks (q3w), 6.7 mg/kg once every 3 weeks (q3w), 6.8 mg/kg once every 3 weeks (q3w), 6.9 mg/kg once every 3 weeks (q3w), 7.0 mg/kg once every 3 weeks (q3w), 7.1 mg/kg once every 3 weeks (q3w), 7.2 mg/kg once every 3 weeks (q3w), 7.3 mg/kg once every 3 weeks (q3w), 7.4 mg/kg once every 3 weeks (q3w), 7.5 mg/kg once every 3 weeks (q3w), 7.6 mg/kg once every 3 weeks (q3w), 7.7 mg/kg once every 3 weeks (q3w), 7.8 mg/kg once every 3 weeks (q3w), 7.9 mg/kg once every 3 weeks (q3w), 8.0 mg/kg once every 3 weeks (q3w), 8.1 mg/kg once every 3 weeks (q3w), 8.2 mg/kg once every 3 weeks (q3w), 8.3 mg/kg once every 3 weeks (q3w), 8.4 mg/kg once every 3 weeks (q3w), 8.5 mg/kg once every 3 weeks (q3w), 8.6 mg/kg once every 3 weeks (q3w), 8.7 mg/kg once every 3 weeks (q3w), 8.8 mg/kg once every 3 weeks (q3w), 8.9 mg/kg once every 3 weeks (q3w), 9.0 mg/kg once every 3 weeks (q3w), 9.1 mg/kg once every 3 weeks (q3w), 9.2 mg/kg once every 3 weeks (q3w), 9.3 mg/kg once every 3 weeks (q3w), 9.4 mg/kg once every 3 weeks (q3w), 9.5 mg/kg once every 3 weeks (q3w), 9.6 mg/kg once every 3 weeks (q3w), 9.7 mg/kg once every 3 weeks (q3w), 9.8 mg/kg once every 3 weeks (q3w), 9.9 mg/kg once every 3 weeks (q3w), 10.0 mg/kg once every 3 weeks (q3w), 10.1 mg/kg once every 3 weeks (q3w), 10.2 mg/kg once every 3 weeks (q3w), 10.3 mg/kg once every 3 weeks (q3w), 10.4 mg/kg once every 3 weeks (q3w), 10.5 mg/kg once every 3 weeks (q3w), 10.6 mg/kg once every 3 weeks (q3w), 10.7 mg/kg once every 3 weeks (q3w), 10.8 mg/kg once every 3 weeks (q3w), 10.9 mg/kg once every 3 weeks (q3w), 11.0 mg/kg once every 3 weeks (q3w), 11.1 mg/kg once every 3 weeks (q3w), 11.2 mg/kg once every 3 weeks (q3w), 11.3 mg/kg once every 3 weeks (q3w), 11.4 mg/kg once every 3 weeks (q3w), 11.5 mg/kg once every 3 weeks (q3w), 11.6 mg/kg once every 3 weeks (q3w), 11.7 mg/kg once every 3 weeks (q3w), 11.8 mg/kg once every 3 weeks (q3w), 11.9 mg/kg once every 3 weeks (q3w) or 12.0 mg/kg once every 3 weeks (q3w). In preferred embodiments, dosage regimen may be 1.6 mg/kg once every 3 weeks (q3w), 3.2 mg/kg once every 3 weeks (q3w), 4.8 mg/kg once every 3 weeks (q3w), 6.4 mg/kg once every 3 weeks (q3w), 8.0 mg/kg once every 3 weeks (q3w), 9.6 mg/kg once every 3 weeks (q3w), but more preferably 3.2 mg/kg once every 3 weeks (q3w), 4.8 mg/kg once every 3 weeks (q3w), 6.4 mg/kg once every 3 weeks (q3w), 8.0 mg/kg once every 3 weeks (q3w), or 9.6 mg/kg once every 3 weeks (q3w), but further preferably 6.4 mg/kg once every 3 weeks (q3w) or 8.0 mg/kg once every 3 weeks (q3w), but furthermore preferably 8.0 mg/kg once every 3 weeks (q3w).

In preferred embodiments, dosage regimen may be 3.2 mg/kg once every 3 weeks (q3w), 3.3 mg/kg once every 3 weeks (q3w), 3.4 mg/kg once every 3 weeks (q3w), 3.5 mg/kg once every 3 weeks (q3w), 3.6 mg/kg once every 3 weeks (q3w), 3.7 mg/kg once every 3 weeks (q3w), 3.8 mg/kg once every 3 weeks (q3w), 3.9 mg/kg once every 3 weeks (q3w), 4.0 mg/kg once every 3 weeks (q3w), 4.1 mg/kg once every 3 weeks (q3w), 4.2 mg/kg once every 3 weeks (q3w), 4.3 mg/kg once every 3 weeks (q3w), 4.4 mg/kg once every 3 weeks (q3w), 4.5 mg/kg once every 3 weeks (q3w), 4.6 mg/kg once every 3 weeks (q3w), 4.7 mg/kg once every 3 weeks (q3w), 4.8 mg/kg once every 3 weeks (q3w), 4.9 mg/kg once every 3 weeks (q3w), 5.0 mg/kg once every 3 weeks (q3w), 5.1 mg/kg once every 3 weeks (q3w), 5.2 mg/kg once every 3 weeks (q3w), 5.3 mg/kg once every 3 weeks (q3w), 5.4 mg/kg once every 3 weeks (q3w), 5.5 mg/kg once every 3 weeks (q3w), 5.6 mg/kg once every 3 weeks (q3w), 5.7 mg/kg once every 3 weeks (q3w), 5.8 mg/kg once every 3 weeks (q3w), 5.9 mg/kg once every 3 weeks (q3w), 6.0 mg/kg once every 3 weeks (q3w), 6.1 mg/kg once every 3 weeks (q3w), 6.2 mg/kg once every 3 weeks (q3w), 6.3 mg/kg once every 3 weeks (q3w) or 6.4 mg/kg once every 3 weeks (q3w), but more preferably 4.8 mg/kg once every 3 weeks (q3w), 4.9 mg/kg once every 3 weeks (q3w), 5.0 mg/kg once every 3 weeks (q3w), 5.1 mg/kg once every 3 weeks (q3w), 5.2 mg/kg once every 3 weeks (q3w), 5.3 mg/kg once every 3 weeks (q3w), 5.4 mg/kg once every 3 weeks (q3w), 5.5 mg/kg once every 3 weeks (q3w), 5.6 mg/kg once every 3 weeks (q3w), 5.7 mg/kg once every 3 weeks (q3w), 5.8 mg/kg once every 3 weeks (q3w), 5.9 mg/kg once every 3 weeks (q3w), 6.0 mg/kg once every 3 weeks (q3w), 6.1 mg/kg once every 3 weeks (q3w), 6.2 mg/kg once every 3 weeks (q3w), 6.3 mg/kg once every 3 weeks (q3w) or 6.4 mg/kg once every 3 weeks (q3w).

In some embodiments, dosage regimen may be 5 mg once every 3 weeks (q3w), 10 mg once every 3 weeks (q3w), 15 mg once every 3 weeks (q3w), 20 mg once every 3 weeks (q3w), 25 mg once every 3 weeks (q3w), 30 mg once every 3 weeks (q3w), 35 mg once every 3 weeks (q3w), 40 mg once every 3 weeks (q3w), 45 mg once every 3 weeks (q3w), 50 mg once every 3 weeks (q3w), 55 mg once every 3 weeks (q3w), 60 mg once every 3 weeks (q3w), 65 mg once every 3 weeks (q3w), 70 mg once every 3 weeks (q3w), 75 mg once every 3 weeks (q3w), 80 mg once every 3 weeks (q3w), 85 mg once every 3 weeks (q3w), 90 mg once every 3 weeks (q3w), 95 mg once every 3 weeks (q3w), 100 mg once every 3 weeks (q3w), 125 mg once every 3 weeks (q3w), 150 mg once every 3 weeks (q3w), 175 mg once every 3 weeks (q3w), 200 mg once every 3 weeks (q3w), 205 mg once every 3 weeks (q3w), 210 mg once every 3 weeks (q3w), 215 mg once every 3 weeks (q3w), 220 mg once every 3 weeks (q3w), 225 mg once every 3 weeks (q3w), 230 mg once every 3 weeks (q3w), 235 mg once every 3 weeks (q3w), 240 mg once every 3 weeks (q3w), 245 mg once every 3 weeks (q3w), 250 mg once every 3 weeks (q3w), 255 mg once every 3 weeks (q3w), 260 mg once every 3 weeks (q3w), 265 mg once every 3 weeks (q3w), 270 mg once every 3 weeks (q3w), 275 mg once every 3 weeks (q3w), 280 mg once every 3 weeks (q3w), 285 mg once every 3 weeks (q3w), 290 mg once every 3 weeks (q3w), 295 mg once every 3 weeks (q3w), 300 mg once every 3 weeks (q3w), 305 mg once every 3 weeks (q3w), 310 mg once every 3 weeks (q3w), 315 mg once every 3 weeks (q3w), 320 mg once every 3 weeks (q3w), 325 mg once every 3 weeks (q3w), 330 mg once every 3 weeks (q3w), 335 mg once every 3 weeks (q3w), 340 mg once every 3 weeks (q3w), 345 mg once every 3 weeks (q3w), 350 mg once every 3 weeks (q3w), 355 mg once every 3 weeks (q3w), 360 mg once every 3 weeks (q3w), 365 mg once every 3 weeks (q3w), 370 mg once every 3 weeks (q3w), 375 mg once every 3 weeks (q3w), 380 mg once every 3 weeks (q3w), 385 mg once every 3 weeks (q3w), 390 mg once every 3 weeks (q3w), 395 mg once every 3 weeks (q3w), 400 mg once every 3 weeks (q3w), 405 mg once every 3 weeks (q3w), 410 mg once every 3 weeks (q3w), 415 mg once every 3 weeks (q3w), 420 mg once every 3 weeks (q3w), 425 mg once every 3 weeks (q3w), 430 mg once every 3 weeks (q3w), 435 mg once every 3 weeks (q3w), 440 mg once every 3 weeks (q3w), 445 mg once every 3 weeks (q3w), 450 mg once every 3 weeks (q3w), 455 mg once every 3 weeks (q3w), 460 mg once every 3 weeks (q3w), 465 mg once every 3 weeks (q3w), 470 mg once every 3 weeks (q3w), 475 mg once every 3 weeks (q3w), 480 mg once every 3 weeks (q3w), 485 mg once every 3 weeks (q3w), 490 mg once every 3 weeks (q3w), 495 mg once every 3 weeks (q3w), 500 mg once every 3 weeks (q3w), 505 mg once every 3 weeks (q3w), 510 mg once every 3 weeks (q3w), 515 mg once every 3 weeks (q3w), 520 mg once every 3 weeks (q3w), 525 mg once every 3 weeks (q3w), 530 mg once every 3 weeks (q3w), 535 mg once every 3 weeks (q3w), 540 mg once every 3 weeks (q3w), 545 mg once every 3 weeks (q3w), 550 mg once every 3 weeks (q3w), 555 mg once every 3 weeks (q3w), 560 mg once every 3 weeks (q3w), 565 mg once every 3 weeks (q3w), 570 mg once every 3 weeks (q3w), 575 mg once every 3 weeks (q3w), 580 mg once every 3 weeks (q3w), 585 mg once every 3 weeks (q3w), 590 mg once every 3 weeks (q3w), 595 mg once every 3 weeks (q3w), 600 mg once every 3 weeks (q3w), 605 mg once every 3 weeks (q3w), 610 mg once every 3 weeks (q3w), 615 mg once every 3 weeks (q3w), 620 mg once every 3 weeks (q3w), 625 mg once every 3 weeks (q3w), 630 mg once every 3 weeks (q3w), 635 mg once every 3 weeks (q3w), 640 mg once every 3 weeks (q3w), 645 mg once every 3 weeks (q3w), 650 mg once every 3 weeks (q3w), 655 mg once every 3 weeks (q3w), 660 mg once every 3 weeks (q3w), 665 mg once every 3 weeks (q3w), 670 mg once every 3 weeks (q3w), 675 mg once every 3 weeks (q3w), 680 mg once every 3 weeks (q3w), 685 mg once every 3 weeks (q3w), 690 mg once every 3 weeks (q3w), 695 mg once every 3 weeks (q3w), 700 mg once every 3 weeks (q3w), 705 mg once every 3 weeks (q3w), 710 mg once every 3 weeks (q3w), 715 mg once every 3 weeks (q3w), 720 mg once every 3 weeks (q3w), 725 mg once every 3 weeks (q3w), 730 mg once every 3 weeks (q3w), 735 mg once every 3 weeks (q3w), 740 mg once every 3 weeks (q3w), 745 mg once every 3 weeks (q3w), 750 mg once every 3 weeks (q3w), 755 mg once every 3 weeks (q3w), 760 mg once every 3 weeks (q3w), 765 mg once every 3 weeks (q3w), 770 mg once every 3 weeks (q3w), 775 mg once every 3 weeks (q3w), 780 mg once every 3 weeks (q3w), 785 mg once every 3 weeks (q3w), 790 mg once every 3 weeks (q3w), 795 mg once every 3 weeks (q3w), 800 mg once every 3 weeks (q3w), 805 mg once every 3 weeks (q3w), 810 mg once every 3 weeks (q3w), 815 mg once every 3 weeks (q3w), 820 mg once every 3 weeks (q3w), 825 mg once every 3 weeks (q3w), 830 mg once every 3 weeks (q3w), 835 mg once every 3 weeks (q3w), 840 mg once every 3 weeks (q3w), 845 mg once every 3 weeks (q3w), 850 mg once every 3 weeks (q3w), 855 mg once every 3 weeks (q3w), 860 mg once every 3 weeks (q3w), 865 mg once every 3 weeks (q3w), 870 mg once every 3 weeks (q3w), 875 mg once every 3 weeks (q3w), 880 mg once every 3 weeks (q3w), 885 mg once every 3 weeks (q3w), 890 mg once every 3 weeks (q3w), 895 mg once every 3 weeks (q3w), 900 mg once every 3 weeks (q3w), 905 mg once every 3 weeks (q3w), 910 mg once every 3 weeks (q3w), 915 mg once every 3 weeks (q3w), 920 mg once every 3 weeks (q3w), 925 mg once every 3 weeks (q3w), 930 mg once every 3 weeks (q3w), 935 mg once every 3 weeks (q3w), 940 mg once every 3 weeks (q3w), 945 mg once every 3 weeks (q3w), 950 mg once every 3 weeks (q3w), 955 mg once every 3 weeks (q3w), 960 mg once every 3 weeks (q3w), 965 mg once every 3 weeks (q3w), 970 mg once every 3 weeks (q3w), 975 mg once every 3 weeks (q3w), 980 mg once every 3 weeks (q3w), 985 mg once every 3 weeks (q3w), 990 mg once every 3 weeks (q3w), 995 mg once every 3 weeks (q3w), 1000 mg once every 3 weeks (q3w), 1025 mg once every 3 weeks (q3w), 1050 mg once every 3 weeks (q3w), 1075 mg once every 3 weeks (q3w), 1100 mg once every 3 weeks (q3w), 1125 mg once every 3 weeks (q3w), 1150 mg once every 3 weeks (q3w), 1175 mg once every 3 weeks (q3w), 1200 mg once every 3 weeks (q3w), 1225 mg once every 3 weeks (q3w), 1250 mg once every 3 weeks (q3w), 1275 mg once every 3 weeks (q3w), 1300 mg once every 3 weeks (q3w), 1325 mg once every 3 weeks (q3w), 1350 mg once every 3 weeks (q3w), 1375 mg once every 3 weeks (q3w), 1400 mg once every 3 weeks (q3w), 1425 mg once every 3 weeks (q3w), 1450 mg once every 3 weeks (q3w), 1475 mg once every 3 weeks (q3w), 1500 mg once every 3 weeks (q3w), 1525 mg once every 3 weeks (q3w), 1550 mg once every 3 weeks (q3w), 1575 mg once every 3 weeks (q3w), 1600 mg once every 3 weeks (q3w), 1625 mg once every 3 weeks (q3w), 1650 mg once every 3 weeks (q3w), 1675 mg once every 3 weeks (q3w), 1700 mg once every 3 weeks (q3w), 1725 mg once every 3 weeks (q3w), 1750 mg once every 3 weeks (q3w), 1775 mg once every 3 weeks (q3w), 1800 mg once every 3 weeks (q3w), 1825 mg once every 3 weeks (q3w), 1850 mg once every 3 weeks (q3w), 1875 mg once every 3 weeks (q3w), 1900 mg once every 3 weeks (q3w), 1925 mg once every 3 weeks (q3w), 1950 mg once every 3 weeks (q3w), 1975 mg once every 3 weeks (q3w), or 2000 mg once every 3 weeks (q3w).

In preferred embodiments, dose regimen may be 200 mg once every 3 weeks (q3w), 205 mg once every 3 weeks (q3w), 210 mg once every 3 weeks (q3w), 215 mg once every 3 weeks (q3w), 220 mg once every 3 weeks (q3w), 225 mg once every 3 weeks (q3w), 230 mg once every 3 weeks (q3w), 235 mg once every 3 weeks (q3w), 240 mg once every 3 weeks (q3w), 245 mg once every 3 weeks (q3w), 250 mg once every 3 weeks (q3w), 255 mg once every 3 weeks (q3w), 260 mg once every 3 weeks (q3w), 265 mg once every 3 weeks (q3w), 270 mg once every 3 weeks (q3w), 275 mg once every 3 weeks (q3w), 280 mg once every 3 weeks (q3w), 285 mg once every 3 weeks (q3w), 290 mg once every 3 weeks (q3w), 295 mg once every 3 weeks (q3w), 300 mg once every 3 weeks (q3w), 305 mg once every 3 weeks (q3w), 310 mg once every 3 weeks (q3w), 315 mg once every 3 weeks (q3w), 320 mg once every 3 weeks (q3w), 325 mg once every 3 weeks (q3w), 330 mg once every 3 weeks (q3w), 335 mg once every 3 weeks (q3w), 340 mg once every 3 weeks (q3w), 345 mg once every 3 weeks (q3w), 350 mg once every 3 weeks (q3w), 355 mg once every 3 weeks (q3w), 360 mg once every 3 weeks (q3w), 365 mg once every 3 weeks (q3w), 370 mg once every 3 weeks (q3w), 375 mg once every 3 weeks (q3w), 380 mg once every 3 weeks (q3w), 385 mg once every 3 weeks (q3w), 390 mg once every 3 weeks (q3w), 395 mg once every 3 weeks (q3w), 400 mg once every 3 weeks (q3w), 405 mg once every 3 weeks (q3w), 410 mg once every 3 weeks (q3w), 415 mg once every 3 weeks (q3w), 420 mg once every 3 weeks (q3w), 425 mg once every 3 weeks (q3w), 430 mg once every 3 weeks (q3w), 435 mg once every 3 weeks (q3w), 440 mg once every 3 weeks (q3w), 445 mg once every 3 weeks (q3w), 450 mg once every 3 weeks (q3w), 455 mg once every 3 weeks (q3w), 460 mg once every 3 weeks (q3w), 465 mg once every 3 weeks (q3w), 470 mg once every 3 weeks (q3w), 475 mg once every 3 weeks (q3w), 480 mg once every 3 weeks (q3w), 485 mg once every 3 weeks (q3w), 490 mg once every 3 weeks (q3w), 495 mg once every 3 weeks (q3w), 500 mg once every 3 weeks (q3w), 505 mg once every 3 weeks (q3w), 510 mg once every 3 weeks (q3w), 515 mg once every 3 weeks (q3w), 520 mg once every 3 weeks (q3w), 525 mg once every 3 weeks (q3w), 530 mg once every 3 weeks (q3w), 535 mg once every 3 weeks (q3w), 540 mg once every 3 weeks (q3w), 545 mg once every 3 weeks (q3w), 550 mg once every 3 weeks (q3w), 555 mg once every 3 weeks (q3w), 560 mg once every 3 weeks (q3w), 565 mg once every 3 weeks (q3w), 570 mg once every 3 weeks (q3w), 575 mg once every 3 weeks (q3w), 580 mg once every 3 weeks (q3w), 585 mg once every 3 weeks (q3w), 590 mg once every 3 weeks (q3w), 595 mg once every 3 weeks (q3w), 600 mg once every 3 weeks (q3w), 605 mg once every 3 weeks (q3w), 610 mg once every 3 weeks (q3w), 615 mg once every 3 weeks (q3w), 620 mg once every 3 weeks (q3w), 625 mg once every 3 weeks (q3w), 630 mg once every 3 weeks (q3w), 635 mg once every 3 weeks (q3w), 640 mg once every 3 weeks (q3w), 645 mg once every 3 weeks (q3w), 650 mg once every 3 weeks (q3w), 655 mg once every 3 weeks (q3w), 660 mg once every 3 weeks (q3w), 665 mg once every 3 weeks (q3w), 670 mg once every 3 weeks (q3w), 675 mg once every 3 weeks (q3w), 680 mg once every 3 weeks (q3w), 685 mg once every 3 weeks (q3w), 690 mg once every 3 weeks (q3w), 695 mg once every 3 weeks (q3w), 700 mg once every 3 weeks (q3w), 705 mg once every 3 weeks (q3w), 710 mg once every 3 weeks (q3w), 715 mg once every 3 weeks (q3w), 720 mg once every 3 weeks (q3w), 725 mg once every 3 weeks (q3w), 730 mg once every 3 weeks (q3w), 735 mg once every 3 weeks (q3w), 740 mg once every 3 weeks (q3w), 745 mg once every 3 weeks (q3w), 750 mg once every 3 weeks (q3w), 755 mg once every 3 weeks (q3w), 760 mg once every 3 weeks (q3w), 765 mg once every 3 weeks (q3w), 770 mg once every 3 weeks (q3w), 775 mg once every 3 weeks (q3w), 780 mg once every 3 weeks (q3w), 785 mg once every 3 weeks (q3w), 790 mg once every 3 weeks (q3w), 795 mg once every 3 weeks (q3w), 800 mg once every 3 weeks (q3w), 805 mg once every 3 weeks (q3w), 810 mg once every 3 weeks (q3w), 815 mg once every 3 weeks (q3w), 820 mg once every 3 weeks (q3w), 825 mg once every 3 weeks (q3w), 830 mg once every 3 weeks (q3w), 835 mg once every 3 weeks (q3w), 840 mg once every 3 weeks (q3w), 845 mg once every 3 weeks (q3w), 850 mg once every 3 weeks (q3w), 855 mg once every 3 weeks (q3w), 860 mg once every 3 weeks (q3w), 865 mg once every 3 weeks (q3w), 870 mg once every 3 weeks (q3w), 875 mg once every 3 weeks (q3w), 880 mg once every 3 weeks (q3w), 885 mg once every 3 weeks (q3w), 890 mg once every 3 weeks (q3w), 895 mg once every 3 weeks (q3w), 900 mg once every 3 weeks (q3w), 905 mg once every 3 weeks (q3w), 910 mg once every 3 weeks (q3w), 915 mg once every 3 weeks (q3w), 920 mg once every 3 weeks (q3w), 925 mg once every 3 weeks (q3w), 930 mg once every 3 weeks (q3w), 935 mg once every 3 weeks (q3w), 940 mg once every 3 weeks (q3w), 945 mg once every 3 weeks (q3w), 950 mg once every 3 weeks (q3w), 955 mg once every 3 weeks (q3w), 960 mg once every 3 weeks (q3w), 965 mg once every 3 weeks (q3w), 970 mg once every 3 weeks (q3w), 975 mg once every 3 weeks (q3w), 980 mg once every 3 weeks (q3w), 985 mg once every 3 weeks (q3w), 990 mg once every 3 weeks (q3w), 995 mg once every 3 weeks (q3w), 1000 mg once every 3 weeks (q3w). And in some embodiments, a single bolus may be administered, while in some embodiments, several divided doses may be administered over time or the dose may be proportionally reduced or increased as indicated by the situation.

Furthermore, while the subject of the methods and uses is generally a cancer patient, the age of the patient is not limited. The disclosed methods and uses are useful for treating cancer, malignant disease, or cancer cell proliferation with various recurrence and prognostic outcomes across all age groups and cohorts. Thus, in some embodiments, the subject may be a pediatric subject, while in other embodiments, the subject may be an adult subject.

In some embodiments, the objective response rate of a subject administered the ADC of the present disclosure is at least about 10%, about 20%, about 30%, about 40%, about 50%, about 60%, about 70% or about 80%. In preferred embodiments, the objective response rate of a subject administered the ADC of the present disclosure is at least about 20%. In further preferred embodiments, the objective response rate of a subject administered the ADC of the present disclosure is at least about 30%. In further preferred embodiments, the objective response rate of a subject administered the ADC of the present disclosure is at least about 40%. In further preferred embodiments, the objective response rate of a subject administered the ADC of the present disclosure is at least about 50%. In further preferred embodiments, the objective response rate of a subject administered the ADC of the present disclosure is at least about 60%. In further preferred embodiments, the objective response rate of a subject administered the ADC of the present disclosure is at least about 70%. In further preferred embodiments, the objective response rate of a subject administered the ADC of the present disclosure is at least about 80%.

In some embodiments, the progression free survival of a subject administered the ADC of the present disclosure is at least 3.0, 3.5, 4.0, 4.5, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.5, 15.0 months. In preferred embodiments, the progression free survival of a subject administered the ADC of the present disclosure is at least 4.0 months. In further preferred embodiments, the progression free survival of a subject administered the ADC of the present disclosure is at least 4.5 months. In further preferred embodiments, the progression free survival of a subject administered the ADC of the present disclosure is at least 5.0 months. In further preferred embodiments, the progression free survival of a subject administered the ADC of the present disclosure is at least 5.5 months. In further preferred embodiments, the progression free survival of a subject administered the ADC of the present disclosure is at least 6.0 months. In further preferred embodiments, the progression free survival of a subject administered the ADC of the present disclosure is at least 6.5 months. In further preferred embodiments, the progression free survival of a subject administered the ADC of the present disclosure is at least 7.0 months. In further preferred embodiments, the progression free survival of a subject administered the ADC of the present disclosure is at least 7.5 months. In further preferred embodiments, the progression free survival of a subject administered the ADC of the present disclosure is at least 8.0 months. In further preferred embodiments, the progression free survival of a subject administered the ADC of the present disclosure is at least 8.5 months. In further preferred embodiments, the progression free survival of a subject administered the ADC of the present disclosure is at least 9.0 months. In further preferred embodiments, the progression free survival of a subject administered the ADC of the present disclosure is at least 9.5 months. In further preferred embodiments, the progression free survival of a subject administered the ADC of the present disclosure is at least 10.0 months. In further preferred embodiments, the progression free survival of a subject administered the ADC of the present disclosure is at least 5.6, 5.8, 7.9 or 13.9 months.

In some embodiments, the median progression free survival of a subject administered the ADC of the present disclosure is at least 3.0, 3.5, 4.0, 4.5, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.5, 15.0 months. In preferred embodiments, the median progression free survival of a subject administered the ADC of the present disclosure is at least 4.0 months. In further preferred embodiments, the median progression free survival of a subject administered the ADC of the present disclosure is at least 4.5 months. In further preferred embodiments, the median progression free survival of a subject administered the ADC of the present disclosure is at least 5.0 months. In further preferred embodiments, the median progression free survival of a subject administered the ADC of the present disclosure is at least 5.5 months. In further preferred embodiments, the median progression free survival of a subject administered the ADC of the present disclosure is at least 6.0 months. In further preferred embodiments, the median progression free survival of a subject administered the ADC of the present disclosure is at least 6.5 months. In further preferred embodiments, the median progression free survival of a subject administered the ADC of the present disclosure is at least 7.0 months. In further preferred embodiments, the median progression free survival of a subject administered the ADC of the present disclosure is at least 7.5 months. In further preferred embodiments, the median progression free survival of a subject administered the ADC of the present disclosure is at least 8.0 months. In further preferred embodiments, the median progression free survival of a subject administered the ADC of the present disclosure is at least 8.5 months. In further preferred embodiments, the median progression free survival of a subject administered the ADC of the present disclosure is at least 9.0 months. In further preferred embodiments, the median progression free survival of a subject administered the ADC of the present disclosure is at least 9.5 months. In further preferred embodiments, the median progression free survival of a subject administered the ADC of the present disclosure is at least 10.0 months. In further preferred embodiments, the median progression free survival of a subject administered the ADC of the present disclosure is at least 5.6, 5.8, 7.9 or 13.9 months.

In some embodiments, the ADC of the present disclosure may be administered as a therapy in combination with a surgical procedure. In some aspects, the ADC of the present disclosure may be administered for the purpose of diminishing the size of a tumor before surgical procedure (referred to as pre-operative adjuvant therapy or neoadjuvant therapy), or may be used after a surgical procedure for the purpose of preventing the recurrence of a tumor (referred to as post-operative adjuvant therapy or adjuvant therapy).

In some embodiments, the ADC of the present disclosure may be administered as a maintenance therapy. In some embodiments, the ADC of the present disclosure may be administered for the purpose of preventing the recurrence of a tumor after initial chemotherapy.

In some embodiments for adjuvant therapy, neoadjuvant therapy or maintenance therapy, the dose regimen may be 1.6 mg/kg once every 1 week (q1w), 1.7 mg/kg once every 1 week (q1w), 1.8 mg/kg once every 1 week (q1w), 1.9 mg/kg once every 1 week (q1w), 2.0 mg/kg once every 1 week (q1w), 2.1 mg/kg once every 1 week (q1w), 2.2 mg/kg once every 1 week (q1w), 2.3 mg/kg once every 1 week (q1w), 2.4 mg/kg once every 1 week (q1w), 2.5 mg/kg once every 1 week (q1w), 2.6 mg/kg once every 1 week (q1w), 2.7 mg/kg once every 1 week (q1w), 2.8 mg/kg once every 1 week (q1w), 2.9 mg/kg once every 1 week (q1w), 3.0 mg/kg once every 1 week (q1w), 3.1 mg/kg once every 1 week (q1w), 3.2 mg/kg once every 1 week (q1w), 3.3 mg/kg once every 1 week (q1w), 3.4 mg/kg once every 1 week (q1w), 3.5 mg/kg once every 1 week (q1w), 3.6 mg/kg once every 1 week (q1w), 3.7 mg/kg once every 1 week (q1w), 3.8 mg/kg once every 1 week (q1w), 3.9 mg/kg once every 1 week (q1w), 4.0 mg/kg once every 1 week (q1w), 4.1 mg/kg once every 1 week (q1w), 4.2 mg/kg once every 1 week (q1w), 4.3 mg/kg once every 1 week (q1w), 4.4 mg/kg once every 1 week (q1w), 4.5 mg/kg once every 1 week (q1w), 4.6 mg/kg once every 1 week (q1w), 4.7 mg/kg once every 1 week (q1w), 4.8 mg/kg once every 1 week (q1w), 4.9 mg/kg once every 1 week (q1w), 5.0 mg/kg once every 1 week (q1w), 5.1 mg/kg once every 1 week (q1w), 5.2 mg/kg once every 1 week (q1w), 5.3 mg/kg once every 1 week (q1w), 5.4 mg/kg once every 1 week (q1w), 5.5 mg/kg once every 1 week (q1w), 5.6 mg/kg once every 1 week (q1w), 5.7 mg/kg once every 1 week (q1w), 5.8 mg/kg once every 1 week (q1w), 5.9 mg/kg once every 1 week (q1w), 6.0 mg/kg once every 1 week (q1w), 6.1 mg/kg once every 1 week (q1w), 6.2 mg/kg once every 1 week (q1w), 6.3 mg/kg once every 1 week (q1w), 6.4 mg/kg once every 1 week (q1w), 6.5 mg/kg once every 1 week (q1w), 6.6 mg/kg once every 1 week (q1w), 6.7 mg/kg once every 1 week (q1w), 6.8 mg/kg once every 1 week (q1w), 6.9 mg/kg once every 1 week (q1w), 7.0 mg/kg once every 1 week (q1w), 7.1 mg/kg once every 1 week (q1w), 7.2 mg/kg once every 1 week (q1w), 7.3 mg/kg once every 1 week (q1w), 7.4 mg/kg once every 1 week (q1w), 7.5 mg/kg once every 1 week (q1w), 7.6 mg/kg once every 1 week (q1w), 7.7 mg/kg once every 1 week (q1w), 7.8 mg/kg once every 1 week (q1w), 7.9 mg/kg once every 1 week (q1w), 8.0 mg/kg once every 1 week (q1w), 8.1 mg/kg once every 1 week (q1w), 8.2 mg/kg once every 1 week (q1w), 8.3 mg/kg once every 1 week (q1w), 8.4 mg/kg once every 1 week (q1w), 8.5 mg/kg once every 1 week (q1w), 8.6 mg/kg once every 1 week (q1w), 8.7 mg/kg once every 1 week (q1w), 8.8 mg/kg once every 1 week (q1w), 8.9 mg/kg once every 1 week (q1w), 9.0 mg/kg once every 1 week (q1w), 9.1 mg/kg once every 1 week (q1w), 9.2 mg/kg once every 1 week (q1w), 9.3 mg/kg once every 1 week (q1w), 9.4 mg/kg once every 1 week (q1w), 9.5 mg/kg once every 1 week (q1w), 9.6 mg/kg once every 1 week (q1w), 1.6 mg/kg once every 2 weeks (q2w), 1.7 mg/kg once every 2 weeks (q2w), 1.8 mg/kg once every 2 weeks (q2w), 1.9 mg/kg once every 2 weeks (q2w), 2.0 mg/kg once every 2 weeks (q2w), 2.1 mg/kg once every 2 weeks (q2w), 2.2 mg/kg once every 2 weeks (q2w), 2.3 mg/kg once every 2 weeks (q2w), 2.4 mg/kg once every 2 weeks (q2w), 2.5 mg/kg once every 2 weeks (q2w), 2.6 mg/kg once every 2 weeks (q2w), 2.7 mg/kg once every 2 weeks (q2w), 2.8 mg/kg once every 2 weeks (q2w), 2.9 mg/kg once every 2 weeks (q2w), 3.0 mg/kg once every 2 weeks (q2w), 3.1 mg/kg once every 2 weeks (q2w), 3.2 mg/kg once every 2 weeks (q2w), 3.3 mg/kg once every 2 weeks (q2w), 3.4 mg/kg once every 2 weeks (q2w), 3.5 mg/kg once every 2 weeks (q2w), 3.6 mg/kg once every 2 weeks (q2w), 3.7 mg/kg once every 2 weeks (q2w), 3.8 mg/kg once every 2 weeks (q2w), 3.9 mg/kg once every 2 weeks (q2w), 4.0 mg/kg once every 2 weeks (q2w), 4.1 mg/kg once every 2 weeks (q2w), 4.2 mg/kg once every 2 weeks (q2w), 4.3 mg/kg once every 2 weeks (q2w), 4.4 mg/kg once every 2 weeks (q2w), 4.5 mg/kg once every 2 weeks (q2w), 4.6 mg/kg once every 2 weeks (q2w), 4.7 mg/kg once every 2 weeks (q2w), 4.8 mg/kg once every 2 weeks (q2w), 4.9 mg/kg once every 2 weeks (q2w), 5.0 mg/kg once every 2 weeks (q2w), 5.1 mg/kg once every 2 weeks (q2w), 5.2 mg/kg once every 2 weeks (q2w), 5.3 mg/kg once every 2 weeks (q2w), 5.4 mg/kg once every 2 weeks (q2w), 5.5 mg/kg once every 2 weeks (q2w), 5.6 mg/kg once every 2 weeks (q2w), 5.7 mg/kg once every 2 weeks (q2w), 5.8 mg/kg once every 2 weeks (q2w), 5.9 mg/kg once every 2 weeks (q2w), 6.0 mg/kg once every 2 weeks (q2w), 6.1 mg/kg once every 2 weeks (q2w), 6.2 mg/kg once every 2 weeks (q2w), 6.3 mg/kg once every 2 weeks (q2w), 6.4 mg/kg once every 2 weeks (q2w), 6.5 mg/kg once every 2 weeks (q2w), 6.6 mg/kg once every 2 weeks (q2w), 6.7 mg/kg once every 2 weeks (q2w), 6.8 mg/kg once every 2 weeks (q2w), 6.9 mg/kg once every 2 weeks (q2w), 7.0 mg/kg once every 2 weeks (q2w), 7.1 mg/kg once every 2 weeks (q2w), 7.2 mg/kg once every 2 weeks (q2w), 7.3 mg/kg once every 2 weeks (q2w), 7.4 mg/kg once every 2 weeks (q2w), 7.5 mg/kg once every 2 weeks (q2w), 7.6 mg/kg once every 2 weeks (q2w), 7.7 mg/kg once every 2 weeks (q2w), 7.8 mg/kg once every 2 weeks (q2w), 7.9 mg/kg once every 2 weeks (q2w), 8.0 mg/kg once every 2 weeks (q2w), 8.1 mg/kg once every 2 weeks (q2w), 8.2 mg/kg once every 2 weeks (q2w), 8.3 mg/kg once every 2 weeks (q2w), 8.4 mg/kg once every 2 weeks (q2w), 8.5 mg/kg once every 2 weeks (q2w), 8.6 mg/kg once every 2 weeks (q2w), 8.7 mg/kg once every 2 weeks (q2w), 8.8 mg/kg once every 2 weeks (q2w), 8.9 mg/kg once every 2 weeks (q2w), 9.0 mg/kg once every 2 weeks (q2w), 9.1 mg/kg once every 2 weeks (q2w), 9.2 mg/kg once every 2 weeks (q2w), 9.3 mg/kg once every 2 weeks (q2w), 9.4 mg/kg once every 2 weeks (q2w), 9.5 mg/kg once every 2 weeks (q2w), 9.6 mg/kg once every 2 weeks (q2w), 1.6 mg/kg once every 3 weeks (q3w), 1.7 mg/kg once every 3 weeks (q3w), 1.8 mg/kg once every 3 weeks (q3w), 1.9 mg/kg once every 3 weeks (q3w), 2.0 mg/kg once every 3 weeks (q3w), 2.1 mg/kg once every 3 weeks (q3w), 2.2 mg/kg once every 3 weeks (q3w), 2.3 mg/kg once every 3 weeks (q3w), 2.4 mg/kg once every 3 weeks (q3w), 2.5 mg/kg once every 3 weeks (q3w), 2.6 mg/kg once every 3 weeks (q3w), 2.7 mg/kg once every 3 weeks (q3w), 2.8 mg/kg once every 3 weeks (q3w), 2.9 mg/kg once every 3 weeks (q3w), 3.0 mg/kg once every 3 weeks (q3w), 3.1 mg/kg once every 3 weeks (q3w), 3.2 mg/kg once every 3 weeks (q3w), 3.3 mg/kg once every 3 weeks (q3w), 3.4 mg/kg once every 3 weeks (q3w), 3.5 mg/kg once every 3 weeks (q3w), 3.6 mg/kg once every 3 weeks (q3w), 3.7 mg/kg once every 3 weeks (q3w), 3.8 mg/kg once every 3 weeks (q3w), 3.9 mg/kg once every 3 weeks (q3w), 4.0 mg/kg once every 3 weeks (q3w), 4.1 mg/kg once every 3 weeks (q3w), 4.2 mg/kg once every 3 weeks (q3w), 4.3 mg/kg once every 3 weeks (q3w), 4.4 mg/kg once every 3 weeks (q3w), 4.5 mg/kg once every 3 weeks (q3w), 4.6 mg/kg once every 3 weeks (q3w), 4.7 mg/kg once every 3 weeks (q3w), 4.8 mg/kg once every 3 weeks (q3w), 4.9 mg/kg once every 3 weeks (q3w), 5.0 mg/kg once every 3 weeks (q3w), 5.1 mg/kg once every 3 weeks (q3w), 5.2 mg/kg once every 3 weeks (q3w), 5.3 mg/kg once every 3 weeks (q3w), 5.4 mg/kg once every 3 weeks (q3w), 5.5 mg/kg once every 3 weeks (q3w), 5.6 mg/kg once every 3 weeks (q3w), 5.7 mg/kg once every 3 weeks (q3w), 5.8 mg/kg once every 3 weeks (q3w), 5.9 mg/kg once every 3 weeks (q3w), 6.0 mg/kg once every 3 weeks (q3w), 6.1 mg/kg once every 3 weeks (q3w), 6.2 mg/kg once every 3 weeks (q3w), 6.3 mg/kg once every 3 weeks (q3w), 6.4 mg/kg once every 3 weeks (q3w), 6.5 mg/kg once every 3 weeks (q3w), 6.6 mg/kg once every 3 weeks (q3w), 6.7 mg/kg once every 3 weeks (q3w), 6.8 mg/kg once every 3 weeks (q3w), 6.9 mg/kg once every 3 weeks (q3w), 7.0 mg/kg once every 3 weeks (q3w), 7.1 mg/kg once every 3 weeks (q3w), 7.2 mg/kg once every 3 weeks (q3w), 7.3 mg/kg once every 3 weeks (q3w), 7.4 mg/kg once every 3 weeks (q3w), 7.5 mg/kg once every 3 weeks (q3w), 7.6 mg/kg once every 3 weeks (q3w), 7.7 mg/kg once every 3 weeks (q3w), 7.8 mg/kg once every 3 weeks (q3w), 7.9 mg/kg once every 3 weeks (q3w), 8.0 mg/kg once every 3 weeks (q3w), 8.1 mg/kg once every 3 weeks (q3w), 8.2 mg/kg once every 3 weeks (q3w), 8.3 mg/kg once every 3 weeks (q3w), 8.4 mg/kg once every 3 weeks (q3w), 8.5 mg/kg once every 3 weeks (q3w), 8.6 mg/kg once every 3 weeks (q3w), 8.7 mg/kg once every 3 weeks (q3w), 8.8 mg/kg once every 3 weeks (q3w), 8.9 mg/kg once every 3 weeks (q3w), 9.0 mg/kg once every 3 weeks (q3w), 9.1 mg/kg once every 3 weeks (q3w), 9.2 mg/kg once every 3 weeks (q3w), 9.3 mg/kg once every 3 weeks (q3w), 9.4 mg/kg once every 3 weeks (q3w), 9.5 mg/kg once every 3 weeks (q3w) or 9.6 mg/kg once every 3 weeks (q3w), but more preferably 3.2 mg/kg once every 1 week (q1w), 3.3 mg/kg once every 1 week (q1w), 3.4 mg/kg once every 1 week (q1w), 3.5 mg/kg once every 1 week (q1w), 3.6 mg/kg once every 1 week (q1w), 3.7 mg/kg once every 1 week (q1w), 3.8 mg/kg once every 1 week (q1w), 3.9 mg/kg once every 1 week (q1w), 4.0 mg/kg once every 1 week (q1w), 4.1 mg/kg once every 1 week (q1w), 4.2 mg/kg once every 1 week (q1w), 4.3 mg/kg once every 1 week (q1w), 4.4 mg/kg once every 1 week (q1w), 4.5 mg/kg once every 1 week (q1w), 4.6 mg/kg once every 1 week (q1w), 4.7 mg/kg once every 1 week (q1w), 4.8 mg/kg once every 1 week (q1w), 4.9 mg/kg once every 1 week (q1w), 5.0 mg/kg once every 1 week (q1w), 5.1 mg/kg once every 1 week (q1w), 5.2 mg/kg once every 1 week (q1w), 5.3 mg/kg once every 1 week (q1w), 5.4 mg/kg once every 1 week (q1w), 5.5 mg/kg once every 1 week (q1w), 5.6 mg/kg once every 1 week (q1w), 5.7 mg/kg once every 1 week (q1w), 5.8 mg/kg once every 1 week (q1w), 5.9 mg/kg once every 1 week (q1w), 6.0 mg/kg once every 1 week (q1w), 6.1 mg/kg once every 1 week (q1w), 6.2 mg/kg once every 1 week (q1w), 6.3 mg/kg once every 1 week (q1w), 6.4 mg/kg once every 1 week (q1w), 3.2 mg/kg once every 2 weeks (q2w), 3.3 mg/kg once every 2 weeks (q2w), 3.4 mg/kg once every 2 weeks (q2w), 3.5 mg/kg once every 2 weeks (q2w), 3.6 mg/kg once every 2 weeks (q2w), 3.7 mg/kg once every 2 weeks (q2w), 3.8 mg/kg once every 2 weeks (q2w), 3.9 mg/kg once every 2 weeks (q2w), 4.0 mg/kg once every 2 weeks (q2w), 4.1 mg/kg once every 2 weeks (q2w), 4.2 mg/kg once every 2 weeks (q2w), 4.3 mg/kg once every 2 weeks (q2w), 4.4 mg/kg once every 2 weeks (q2w), 4.5 mg/kg once every 2 weeks (q2w), 4.6 mg/kg once every 2 weeks (q2w), 4.7 mg/kg once every 2 weeks (q2w), 4.8 mg/kg once every 2 weeks (q2w), 4.9 mg/kg once every 2 weeks (q2w), 5.0 mg/kg once every 2 weeks (q2w), 5.1 mg/kg once every 2 weeks (q2w), 5.2 mg/kg once every 2 weeks (q2w), 5.3 mg/kg once every 2 weeks (q2w), 5.4 mg/kg once every 2 weeks (q2w), 5.5 mg/kg once every 2 weeks (q2w), 5.6 mg/kg once every 2 weeks (q2w), 5.7 mg/kg once every 2 weeks (q2w), 5.8 mg/kg once every 2 weeks (q2w), 5.9 mg/kg once every 2 weeks (q2w), 6.0 mg/kg once every 2 weeks (q2w), 6.1 mg/kg once every 2 weeks (q2w), 6.2 mg/kg once every 2 weeks (q2w), 6.3 mg/kg once every 2 weeks (q2w), 6.4 mg/kg once every 2 weeks (q2w), 3.2 mg/kg once every 3 weeks (q3w), 3.3 mg/kg once every 3 weeks (q3w), 3.4 mg/kg once every 3 weeks (q3w), 3.5 mg/kg once every 3 weeks (q3w), 3.6 mg/kg once every 3 weeks (q3w), 3.7 mg/kg once every 3 weeks (q3w), 3.8 mg/kg once every 3 weeks (q3w), 3.9 mg/kg once every 3 weeks (q3w), 4.0 mg/kg once every 3 weeks (q3w), 4.1 mg/kg once every 3 weeks (q3w), 4.2 mg/kg once every 3 weeks (q3w), 4.3 mg/kg once every 3 weeks (q3w), 4.4 mg/kg once every 3 weeks (q3w), 4.5 mg/kg once every 3 weeks (q3w), 4.6 mg/kg once every 3 weeks (q3w), 4.7 mg/kg once every 3 weeks (q3w), 4.8 mg/kg once every 3 weeks (q3w), 4.9 mg/kg once every 3 weeks (q3w), 5.0 mg/kg once every 3 weeks (q3w), 5.1 mg/kg once every 3 weeks (q3w), 5.2 mg/kg once every 3 weeks (q3w), 5.3 mg/kg once every 3 weeks (q3w), 5.4 mg/kg once every 3 weeks (q3w), 5.5 mg/kg once every 3 weeks (q3w), 5.6 mg/kg once every 3 weeks (q3w), 5.7 mg/kg once every 3 weeks (q3w), 5.8 mg/kg once every 3 weeks (q3w), 5.9 mg/kg once every 3 weeks (q3w), 6.0 mg/kg once every 3 weeks (q3w), 6.1 mg/kg once every 3 weeks (q3w), 6.2 mg/kg once every 3 weeks (q3w), 6.3 mg/kg once every 3 weeks (q3w) or 6.4 mg/kg once every 3 weeks (q3w), but furthermore preferably 4.8 mg/kg once every 1 week (q1w), 4.9 mg/kg once every 1 week (q1w), 5.0 mg/kg once every 1 week (q1w), 5.1 mg/kg once every 1 week (q1w), 5.2 mg/kg once every 1 week (q1w), 5.3 mg/kg once every 1 week (q1w), 5.4 mg/kg once every 1 week (q1w), 5.5 mg/kg once every 1 week (q1w), 5.6 mg/kg once every 1 week (q1w), 5.7 mg/kg once every 1 week (q1w), 5.8 mg/kg once every 1 week (q1w), 5.9 mg/kg once every 1 week (q1w), 6.0 mg/kg once every 1 week (q1w), 6.1 mg/kg once every 1 week (q1w), 6.2 mg/kg once every 1 week (q1w), 6.3 mg/kg once every 1 week (q1w), 6.4 mg/kg once every 1 week (q1w), 4.8 mg/kg once every 3 weeks (q3w), 4.9 mg/kg once every 3 weeks (q3w), 5.0 mg/kg once every 3 weeks (q3w), 5.1 mg/kg once every 3 weeks (q3w), 5.2 mg/kg once every 3 weeks (q3w), 5.3 mg/kg once every 3 weeks (q3w), 5.4 mg/kg once every 3 weeks (q3w), 5.5 mg/kg once every 3 weeks (q3w), 5.6 mg/kg once every 3 weeks (q3w), 5.7 mg/kg once every 3 weeks (q3w), 5.8 mg/kg once every 3 weeks (q3w), 5.9 mg/kg once every 3 weeks (q3w), 6.0 mg/kg once every 3 weeks (q3w), 6.1 mg/kg once every 3 weeks (q3w), 6.2 mg/kg once every 3 weeks (q3w), 6.3 mg/kg once every 3 weeks (q3w) or 6.4 mg/kg once every 3 weeks (q3w).

The following examples are given to illustrate the present disclosure. It should be understood, however, that the disclosure is not to be limited to the specific conditions or details described in these examples.

### EXAMPLES

Hereinafter, the present disclosure will be specifically described in the following examples. However, these examples are not intended to limit the scope of the present disclosure. Furthermore, these examples should not be construed in a limited manner by any means. It is to be noted that, in the following examples, unless otherwise specified, individual operations regarding genetic manipulation have been carried out according to the method described in “Molecular Cloning” (Sambrook, J., Fritsch, E. F. and Maniatis, T., published by Cold Spring Harbor Laboratory Press in 1989) or other methods described in experimental manuals used by persons skilled in the art, or when commercially available reagents or kits have been used, the examples have been carried out in accordance with the instructions included in the commercially available products. In the present description, reagents, solvents and starting materials are readily available from commercially available sources, unless otherwise specified.

### Example 1: Production of Antibody Drug Conjugates

According to production methods disclosed in International Publication No. WO2018/212136, an anti-CDH6 antibody (an antibody comprising a heavy chain consisting of the amino acid sequence at amino acid positions 20 to 471 in SEQ ID NO: 69 and a light chain consisting of the amino acid sequence at amino acid positions 21 to 233 in SEQ ID NO: 61) was used to produce an antibody-drug conjugate (H1L2-Dxd) (hereinafter also referred to as the “DS-6000a”) in which the anti-CDH6 antibody is bound, via a thioether bond, to a drug linker represented by the following formula:

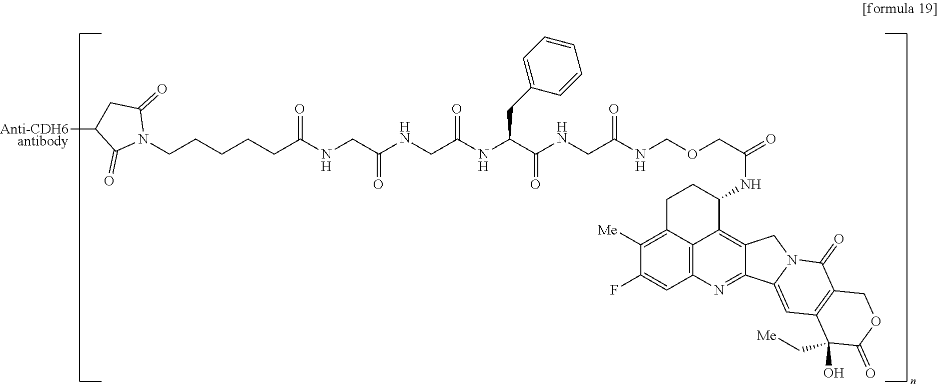

wherein n represents an average drug- to -antibody ratio (DAR) per single antibody molecule; and the value of n of the antibody-drug conjugate (H1L2-Dxd) falls within the range of 7 to 8.

### Example 2: Initial Phase 1 Clinical Study

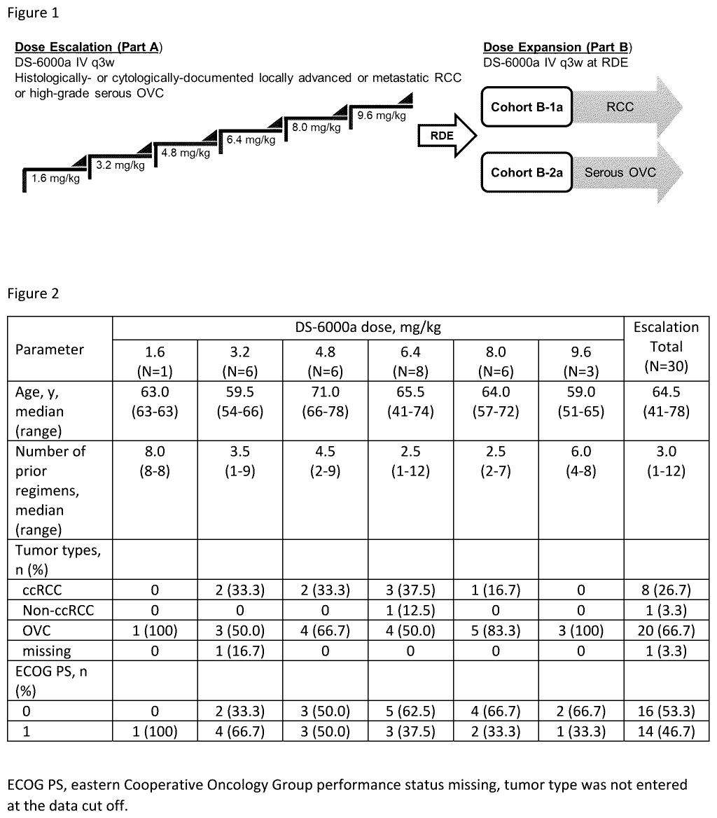

The Phase 1 study included both a dose escalation part and a dose expansion part, as shown in FIG. 1.

### Example 2-1 the Dose Escalation Part of the Phase 1 Study

The dose escalation part of the Phase 1 study was a multicenter, open-label, multiple-dose, first-in-human study of DS-6000a, which enrolled subjects in the United States (see clinicaltrials.gov identifier NCT 04707248). DS-6000a was administered intravenously every 3 weeks. In the dose escalation part, RCC and OVC subjects were enrolled and dose-limiting toxicity (DLT) was assessed during Cycle 1.

The primary objective of the dose escalation part was to investigate the safety and tolerability and determine the maximum-tolerated dose (MTD) and/or recommended dose for expansion (RDE).

Secondary objectives included characterizing the pharmacokinetic (PK) properties of DS-6000a, total anti-CDH6 antibody, drug component and assessing the immunogenicity. Exploratory objectives included evaluating biomarker that correlate with efficacy and disease assessment in OVC using Gynecologic Cancer Intergroup (GCIG) criteria.

Inclusion criteria included: patients aged 218 years; Eastern Cooperative Oncology Group performance status 0 or 1; has adequate organ function; locally advanced or metastatic RCC (clear cell [CC] or non-CC); advanced high grade serous OVC including fallopian tube and primary peritoneal carcinoma and had prior treatment with platinum+taxane therapy; and available tumor tissue for the measurement of CDH6 levels by immunohistochemistry.

Exclusion criteria included: has had prior treatment with other CDH6 targeted agents; has had prior treatment with an ADC that consists of an exatecan derivative; has multiple primary malignancies; or has a history of interstitial lung disease (ILD)/pneumonitis that required corticosteroids.

Patient assessments included echocardiogram or multigated acquisition scan, 12-lead electrocardiogram, AEs, PK, human anti-human antibodies, biomarkers, and tumor assessments at prespecified visits. The demographic and baseline characteristics of the patient enrolled in this dose escalation part of the Phase 1 study are shown in FIG. 2.

For this dose escalation part of the Phase 1 study, Bayesian optimal interval (BOIN) design was used to estimate the MTD. Accelerated titration, treat a single subject at a dose level, was performed until any DLT or ≥Grade 2 AE possibly related to DS-6000a (excluding infusion-related reactions) is observed during Cycle 1. Objective response rate (ORR), clinical benefit rate (CBR), and disease control rate (DCR) were summarized with 95% confidence intervals (CI) using the Clopper-Pearson method; duration of response (DOR) and progression-free survival (PFS) were summarized using the Kaplan-Meier method. Safety endpoints, PK parameters and plasma anti-drug antibodies were summarized using descriptive statistics.

### Results

Thirty patients were enrolled at data cut-off among six DS-6000a dosing groups. The patients (N=30) were exposed to a median (range) of 4.0 (1-18) treatment cycles with DS-6000a, over a median (range) duration of 12.07 (3.0-54.1) weeks.

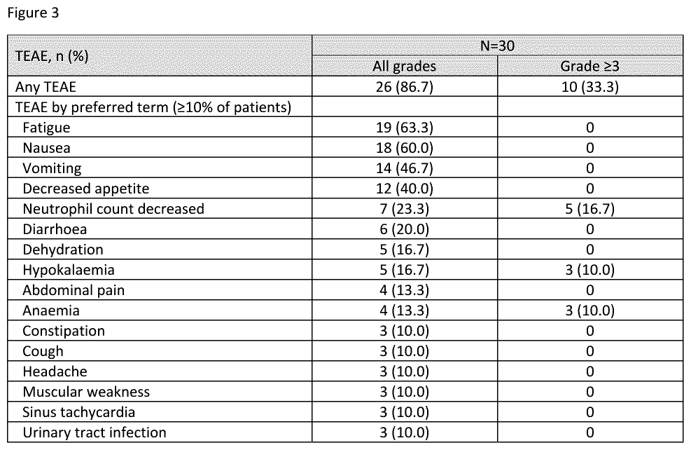

Overall, 86.7% (26/30) of patients reported 21 TEAE (FIG. 3). The most frequent TEAEs (220%) were fatigue (63.3%), nausea (60.0%), vomiting (46.7%), decreased appetite (40.0%), neutrophil count decreased (23.3%) and diarrhoea (20.0%). 33.3% (10/30) of patients reported Grade 23 TEAE, the most frequent Grade 23 TEAEs (210%) were neutrophil count decreased (16.7%), anaemia (10.0%) and hypokalemia (10.0%).

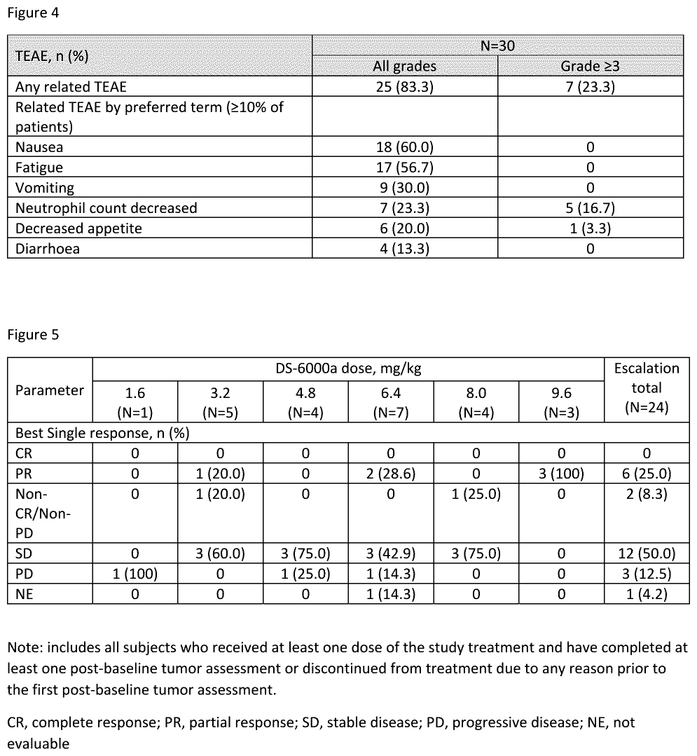

Drug-related TEAEs were reported in 25 patients (83.3%) as shown in FIG. 4. The most frequent related TEAEs (220%) were nausea (60.0%), fatigue (56.7%), vomiting (30.0%), neutrophil count decreased (23.3%) and decreased appetite (20.0%). 23.3% (7/30) of patients reported Grade 23 related TEAE, the most frequent Grade 23 TEAEs (210%) were neutrophil count decreased (16.7%).

Nine patients required DS-6000a dose interruption (n=3 [6.4 mg/kg]; n=2 each [9.6 mg/kg and 4.8 mg/kg]; n=1 each [8.0 mg/kg and 3.2 mg/kg]), and three patients required DS-6000a dose reduction (n=2 [9.6 mg/kg]; n=1 [8.0 mg/kg]). Overall, 13 (43.3%) patients discontinued from DS-6000a treatment. The primary reason for discontinuation was PD per RECIST V1.1 in 5 patients (n=2 each [6.4 and 8.0 mg/kg]; n=1 [1.6 mg/kg]). Four patients discontinued due to clinical progression (n=1 each [3.2, 4.8, 6.4 and 8.0 mg/kg]). One patient was discontinued due to adverse event (9.6 mg/kg), death (3.2 mg/kg), informed consent withdrawn (6.4 mg/kg) and other (4.8 mg/kg), respectively.

Serious TEAEs were reported in 4/30 (13.3%) patients; all events are Grade 3. Two serious TEAEs were considered drug-related (Grade 3 anaemia and Grade 3 febrile neutropenia at 9.6 mg/kg).

Two DLTs occurred at 9.6 mg/kg; Gr3 febrile neutropenia and Gr4 platelet count decreased.

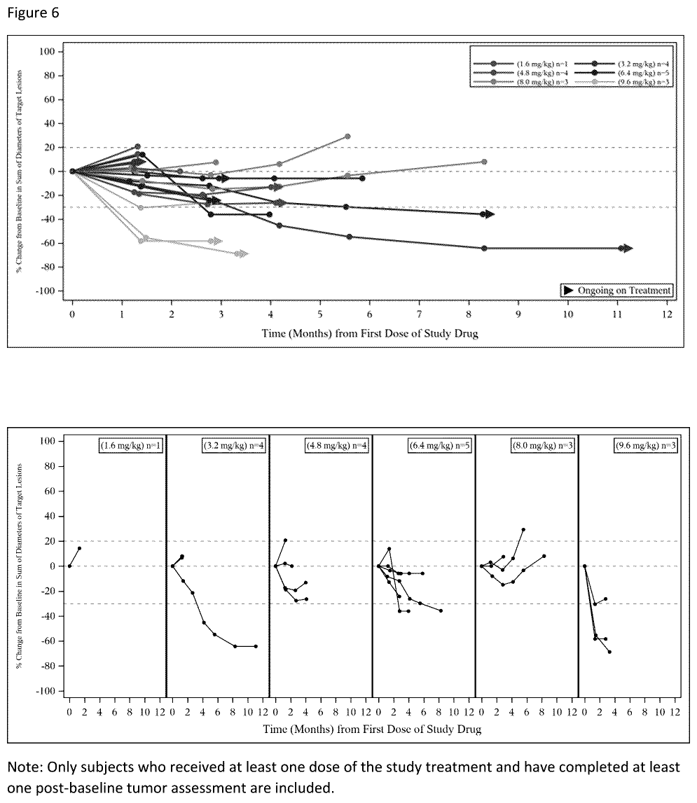

Among 24 efficacy evaluable patients, 6 PRs (including single point PRs, not yet confirmed responses) were observed based on RECIST V1.1 as shown in FIG. 5; one RCC and five platinum-resistant OVC. The responses were durable as shown in FIG. 6. In addition, 12 stable diseases (SDs) and 2 Non-CR/Non-PDs were observed (FIG. 5) and one patient with SD had stayed in treatment more than 180 days at 8.0 mg/kg.

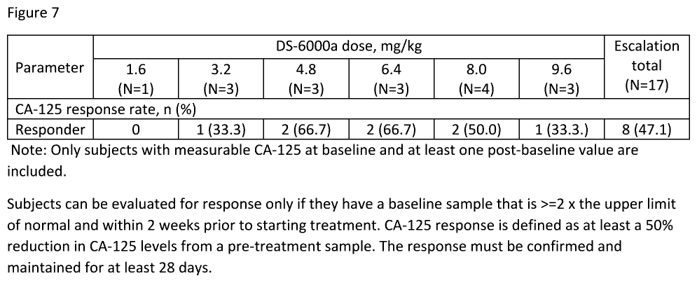

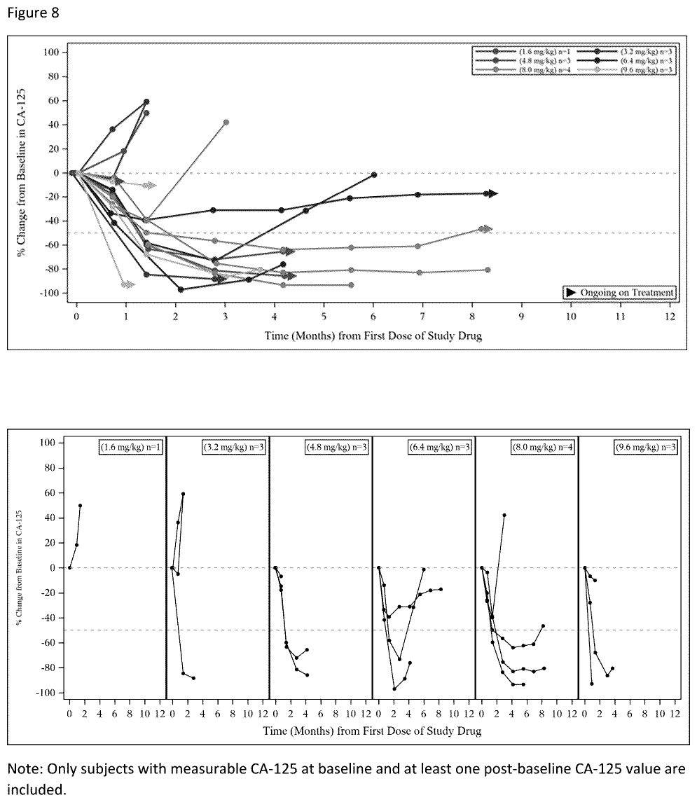

Among 17 efficacy evaluable OVC patients, 8 patients showed CA-125 responder (defined as CA-125 baseline is ≤2× the upper limit of normal within 2 weeks prior to starting treatment and at least 50% reduction in CA-125 levels from baseline. The response must be confirmed and maintained for at least 28 days) as shown in FIG. 7. Higher CA-125 response rates were observed at the dose level of 4.8 to 9.6 mg/kg. The responses were durable as shown in FIG. 8.

It has been reported that RECIST response was preceded by a favorable predictive CA-125 decrease (Gynecologic Oncology, 350-355, 122, 2011).

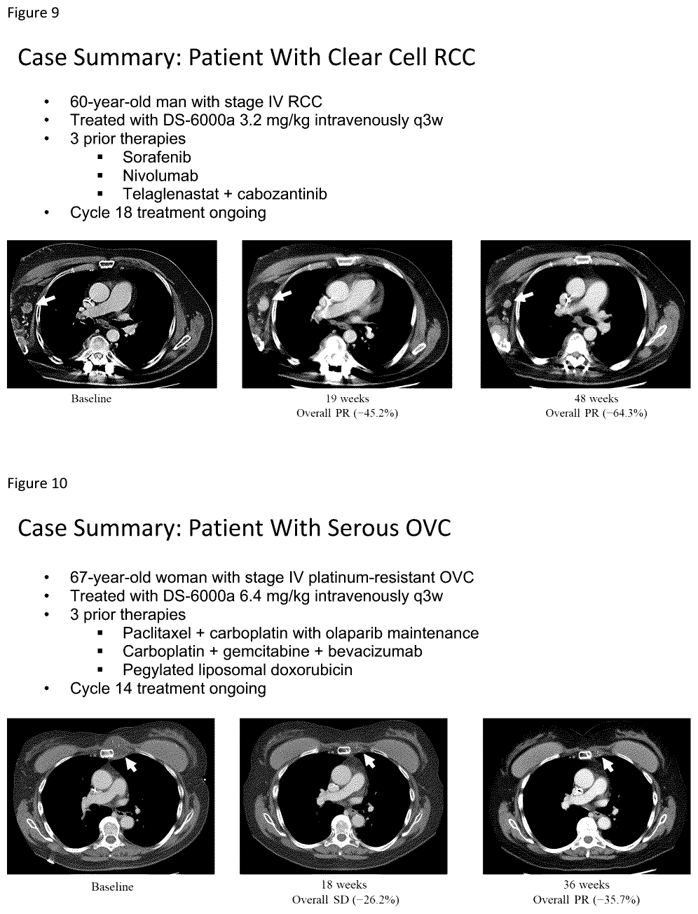

Computed tomography scans were taken in two patients. One clear cell RCC patients who received 3 prior therapies including ICI and VEGFR-TKI at 3.2 mg/kg group demonstrated a maximum 64.3% decrease in tumor size 51.4 weeks following initiation of treatment with DS-6000a (FIG. 9). Another platinum-resistant OVC patient who received 3 prior therapies including bevacizumab and PARP inhibitor at 6.4 mg/kg group demonstrated a maximum 35.7% decrease in tumor size 40.4 weeks following initiation of treatment with DS-6000a (FIG. 10).

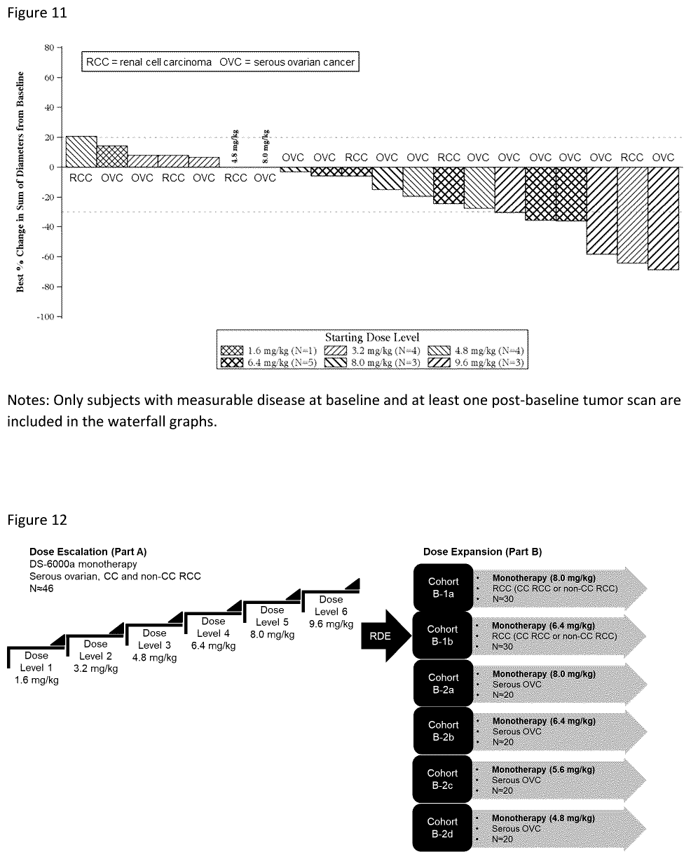

The best percent change in sum of longest dimensions from baseline in target lesion is illustrated in FIG. 11. Tumor shrinkages were observed at the 3.2, 4.8, 6.4, 8.0 and 9.6 mg/kg dose levels and the best percent change (68.8% tumor reduction) was observed in a patient in the 9.6 mg/kg dosing group.

### Summary

As of data cut off, DS-6000a was generally well tolerated and RDE was determined as 8.0 mg/kg. DS-6000a demonstrated early clinical signals in heavily pretreated patients with advanced RCC and OVC.

### Example 2-2 the Dose Expansion Part of the Phase 1 Study

The dose expansion part of the Phase 1 study was a multicenter, open-label, multiple-dose, first-in-human study of DS-6000a, which enrolled subjects in the United States (see clinicaltrials.gov identifier NCT 04707248). DS-6000a was administered intravenously every 3 weeks. The dose expansion part included administering to RCC and OVC subjects at RDEs. Initially, dose of 8.0 mg/kg was expanded, subsequently, the 6.4, 5.6 and 4.8 mg/kg doses were added as RDEs (FIG. 12).

The primary objective of the dose expansion part was to evaluate the safety, tolerability, and efficacy at the RDEs.

Secondary objectives included characterizing the pharmacokinetic (PK) properties of DS-6000a, total anti-CDH6 antibody, drug component and assessing the immunogenicity. Exploratory objectives included evaluating biomarker that correlate with efficacy and disease assessment in OVC using Gynecologic Cancer Intergroup (GCIG) criteria.

Inclusion criteria included: patients aged 218 years; Eastern Cooperative Oncology Group performance status 0 or 1; has adequate organ function; locally advanced or metastatic RCC (clear cell [CC] or non-CC); advanced high grade serous OVC including fallopian tube and primary peritoneal carcinoma and had prior treatment with platinum+taxane therapy; and available tumor tissue for the measurement of CDH6 levels by immunohistochemistry.

Exclusion criteria included: has had prior treatment with other CDH6 targeted agents; has had prior treatment with an ADC that consists of an exatecan derivative; has multiple primary malignancies; or has a history of interstitial lung disease (ILD)/pneumonitis that required corticosteroids.

Patient assessments included echocardiogram or multigated acquisition scan, 12-lead electrocardiogram, AEs, PK, human anti-human antibodies, biomarkers, and tumor assessments at prespecified visits.

Objective response rate (ORR), clinical benefit rate (CBR), and disease control rate (DCR) were summarized with 95% confidence intervals (CI) using the Clopper-Pearson method; duration of response (DOR) and progression-free survival (PFS) were summarized using the Kaplan-Meier method. Safety endpoints, PK parameters and plasma anti-drug antibodies were summarized using descriptive statistics.

### Example 2-3 Phase 1 Clinical Study as of New Cut-Off Date

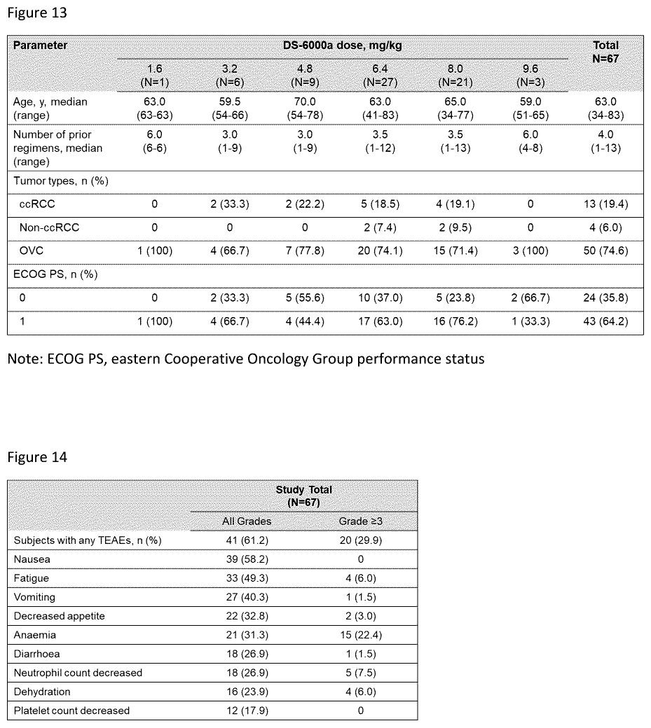

After the initial data-cut, additional patients were enrolled in Phase 1 (both escalation and expansion), bringing the overall number of participants to sixty-seven (N=67). The demographic and baseline characteristics of the patient enrolled in dose escalation and expansion parts of the Phase 1 study as of new cut-off date are shown in FIG. 13. Subjects ranged in age from 34 to 83 years with a median age of 63 years. A median of 4 prior lines of therapy was administered. The prior lines of therapy comprise a platinum-based chemotherapeutic, a chemotherapeutic, a PARP inhibitor, an immune checkpoint inhibitor, an angiogenic inhibitor and a VEGFR-TKI. The subjects were 25.4% RCC, 74.6% OVC and 64.2% had Eastern Cooperative Oncology Group performance status (ECOG PS) of 1.

Subjects had received DS-6000a as monotherapy with a median treatment duration of 15.4 weeks (range 3.0 to 93.9 weeks). The number of patients in the dose escalation and expansion parts of the Phase 1 study as of new cut-off date with TEAE, which occurred in 215% of patients, regardless of causality, is shown in FIG. 14. The most frequently reported TEAEs were gastrointestinal events (nausea, vomiting and diarrhea), fatigue, decreased appetite and hematological events (anaemia, neutrophil count decreased and platelet count decreased). The most frequently reported Grade 23 events were hematological events; the majority of the gastrointestinal events were Grade 1 or 2 in severity.

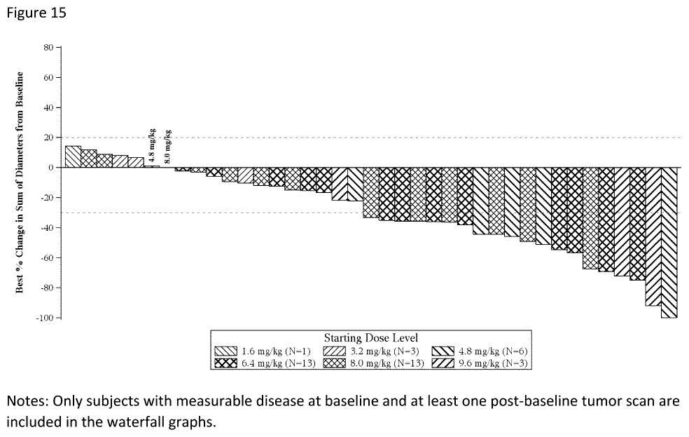

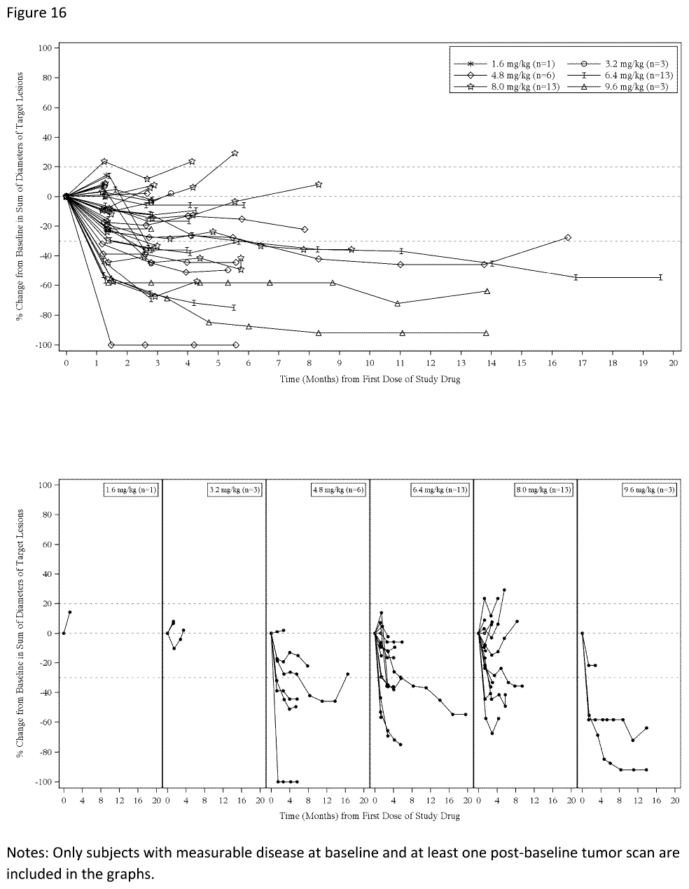

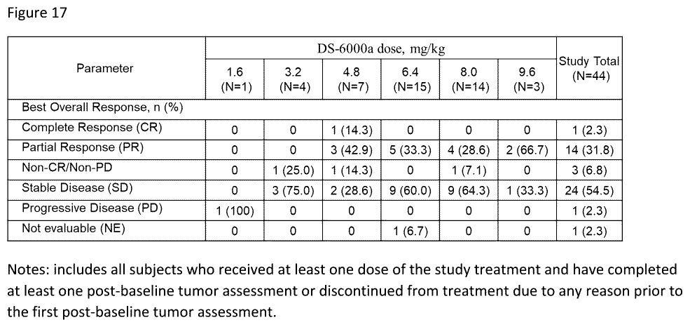

FIG. 15 shows the best percentage change in sum of longest dimension measures from baseline in target lesions of OVC subjects in the dose escalation and expansion parts of the Phase 1 study as of new cut-off date; FIG. 16 shows a dose-effect on efficacy for OVC subjects in the dose escalation and expansion parts of the Phase 1 study as of new cut-off date, as those patients in the higher dosing group tend to show consistent and pronounced reduction in tumor size. One confirmed CR and 14 confirmed PR were observed among 44 evaluable OVC subjects at doses ranging from 1.6 to 9.6 mg/kg. SD was reported for 24 subjects (FIG. 17).

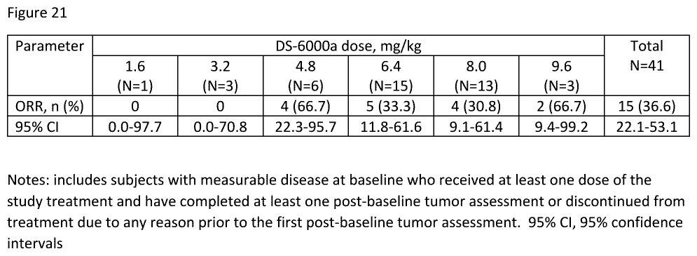

As shown in FIG. 21, ORR was 36.6% among 41 evaluable OVC subjects with measurable disease at baseline and who received doses ranging from 1.6 to 9.6 mg/kg. Higher ORR was observed compared to the current standard of care in platinum resistant ovarian cancer which are single-agent chemotherapies (Pujade-Lauraine E, et al., J. Clin. Oncol. (2014) 32 (13), 1302-1308).

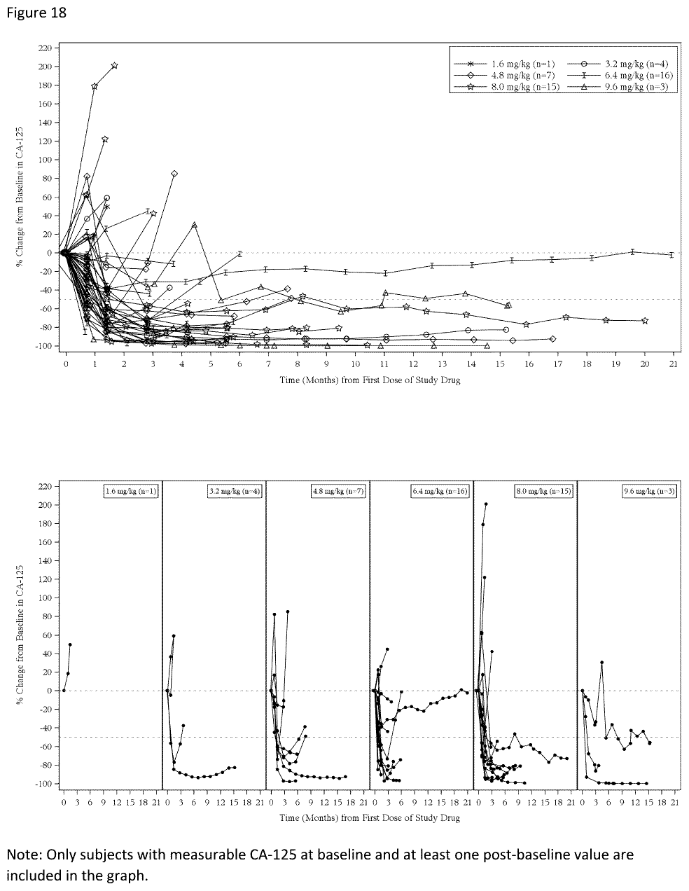

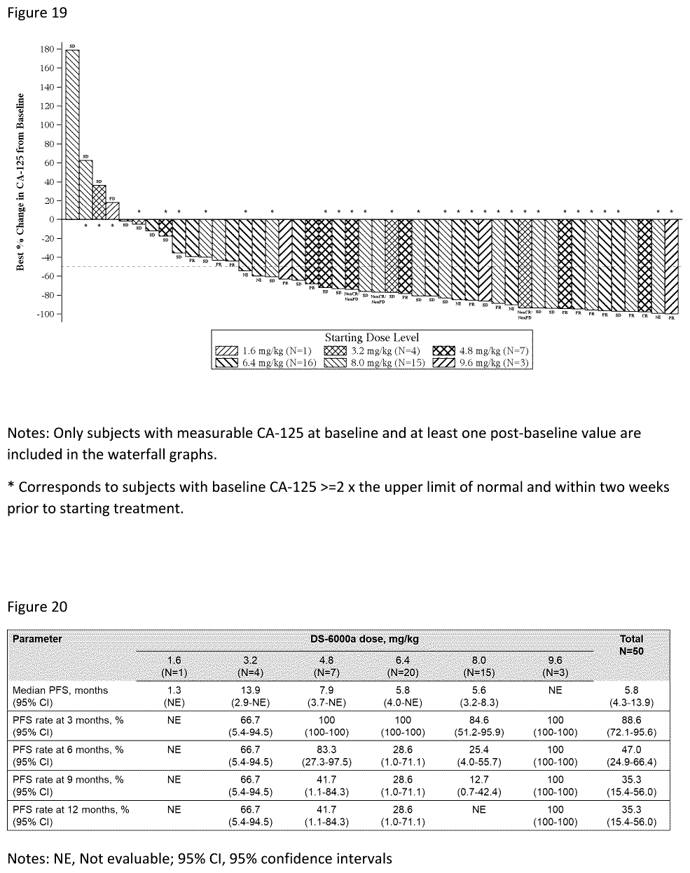

The CA-125 reductions were deep and durable as shown in FIG. 18. FIG. 19 shows the best percentage change in CA-125 from baseline of subjects. Among 28 efficacy evaluable OVC patients, 15 patients showed CA-125 responder (defined as CA-125 baseline is ≤2× the upper limit of normal within 2 weeks prior to starting treatment and at least 50% reduction in CA-125 levels from baseline. The response must be confirmed and maintained for at least 28 days). High CA-125 response rates were observed among dose levels.

The median PFS was 5.8 months in OVC subjects at doses ranging from 1.6 to 9.6 mg/kg; 13.9 months, 7.9 months, 5.8 months and 5.6 months at the 3.2, 4.8, 6.4 and 8.0 mg/kg dose levels, respectively (FIG. 20).

Longer PFS was observed compared to the current standard of care in platinum resistant ovarian cancer which are single-agent chemotherapies (Pujade-Lauraine E, et al., J. Clin. Oncol. (2014) 32 (13), 1302-1308).

In summary, DS-6000a was well tolerated in doses up to 6.4 mg/kg. 8.0 mg/kg was not tolerated with 3 fatal cases and frequent dose reduction/interruption compared to lower doses. Although the sample size is small, 4.8 and 6.4 mg/kg showed better preliminary efficacy signals compared to 8.0 mg/kg. The results are superior to single-agent chemotherapies (i.e., paclitaxel, PLD, gemcitabine, or topotecan) as a standard of care in platinum-resistant ovarian cancer (Pujade-Lauraine E, et al., J. Clin. Oncol. (2014) 32 (13), 1302-1308). Taken together, DS-6000a demonstrated acceptable safety and encouraging efficacy in heavily pretreated OVC subjects.

The safety and efficacy of 4.8, 5.6, 6.4 and 8.0 mg/kg are continuously being evaluated in a phase I study.

What is claimed:

1. An anti-CDH6 antibody-drug conjugate for use in treating or preventing cancer, the antibody-drug conjugate comprising an anti-CDH6 antibody and an antitumor compound connected by a linker, wherein the linker and the antitumor compound are represented by the following formula:
-(Succinimid-3-yl-N)—CH₂CH₂CH₂CH₂CH₂—C(═O)-GGFG-NH—CH₂—O—CH₂—C(═O)—(NH-DX)
wherein -(Succinimid-3-yl-N)— has a structure represented by the following formula: 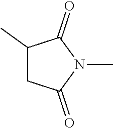
which is connected to the antibody at position 3 thereof and is connected to a methylene group in the linker structure containing this structure on the nitrogen atom at position 1, and (NH-DX) represents a group represented by the following formula: 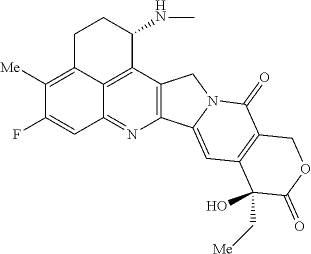 [SMILES: CC[C@@]1(O)C(=O)OCc2c1cc1n(c2=O)Cc2c-1nc1cc(F)c(C)c3c1c2[C@@H](NC)CC3]
wherein the nitrogen atom of the amino group at position 1 is the connecting position, wherein the anti-CDH6 antibody comprises CDRH1 consisting of the amino acid sequence of SEQ ID NO: 17, CDRH2 consisting of the amino acid sequence of SEQ ID NO: 60 and CDRH3 consisting of the amino acid sequence of SEQ ID NO: 19 in its heavy chain variable region and CDRL1 consisting of the amino acid sequence of SEQ ID NO: 12, CDRL2 consisting of the amino acid sequence of SEQ ID NO: 13 and CDRL3 consisting of the amino acid sequence of SEQ ID NO: 14 in its light chain variable region.

2. The antibody-drug conjugate according to claim 1, wherein the antibody comprises a heavy chain variable region comprising amino acids sequence of SEQ ID NO: 71 and a light chain variable region comprising amino acids sequence of SEQ ID NO: 63.

3. The antibody-drug conjugate according to claim 1 or 2, which has a structure represented by the following formula: 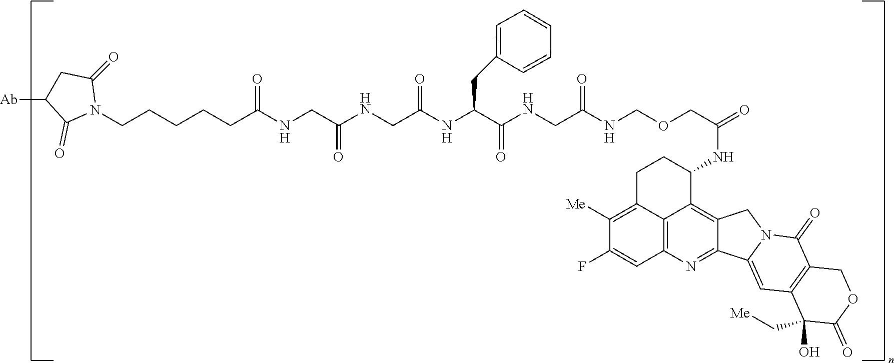
wherein Ab is an antibody comprising a heavy chain variable region comprising SEQ ID NO: 71 and a light chain variable region comprising SEQ ID NO: 63, and n represents the average number of units of the drug-linker structure conjugated to the antibody per antibody, wherein the average number of units of the selected drug-linker structure conjugated per antibody is in the range of from 1 to 10.

4. The antibody-drug conjugate according to any one of claims 1 to 3, wherein the antibody comprises a heavy chain comprising the amino acid sequence at positions 20 to 471 in SEQ ID NO: 69 and a light chain comprising the amino acid sequence at positions 21 to 233 in SEQ ID NO: 61.

5. The antibody-drug conjugate according to any one of claims 1 to 4, wherein the heavy chain or the light chain has undergone one or two or more modifications selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, conversion of N-terminal glutamine or N-terminal glutamic acid to pyroglutamic acid, and a deletion of one or two amino acids from the carboxyl terminus.

6. The antibody-drug conjugate according to any one of claims 1 to 5, wherein the anti-CDH6 antibody lacks a lysine residue at the carboxyl terminus of the heavy chain.

7. The antibody-drug conjugate according to any one of claims 1 to 6, wherein an average number of units of the antitumor compound conjugated per antibody is in a range of from 2 to 8.

8. The antibody-drug conjugate according to any one of claims 1 to 7, wherein an average number of units of the antitumor compound conjugated per antibody is 7 to 8.

9. The antibody-drug conjugate according to any one of claims 1 to 8, wherein a dose of the antibody-drug conjugate in a range of 1.6 mg/kg to 9.6 mg/kg is administered to a subject with cancer.

10. The antibody-drug conjugate according to any one of claims 1 to 8, wherein a dose of the antibody-drug conjugate in a range of 3.2 mg/kg to 9.6 mg/kg is administered to a subject with cancer.

11. The antibody-drug conjugate according to any one of claims 1 to 8, wherein a dose of the antibody-drug conjugate in a range of 4.8 mg/kg to 9.6 mg/kg is administered to a subject with cancer.

12. The antibody-drug conjugate according to any one of claims 1 to 8, wherein a dose of the antibody-drug conjugate in a range of 3.2 mg/kg to 8.0 mg/kg is administered to a subject with cancer.

13. The antibody-drug conjugate according to any one of claims 1 to 8, wherein a dose of the antibody-drug conjugate in a range of 3.2 mg/kg to 6.4 mg/kg is administered to a subject with cancer.

14. The antibody-drug conjugate according to any one of claims 1 to 8, wherein a dose of the antibody-drug conjugate in a range of 4.8 mg/kg to 8.0 mg/kg is administered to a subject with cancer.

15. The antibody-drug conjugate according to any one of claims 1 to 8, wherein a dose of the antibody-drug conjugate in a range of 4.8 mg/kg to 6.4 mg/kg is administered to a subject with cancer.

16. The antibody-drug conjugate according to any one of claims 1 to 8, wherein a dose of the antibody-drug conjugate of about 3.2 mg/kg, about 4.8 mg/kg, about 5.4 mg/kg, about 5.6 mg/kg, about 6.4 mg/kg or about 8.0 mg/kg is administered to a subject with cancer.

17. The antibody-drug conjugate according to any one of claims 1 to 8, wherein a dose of the antibody-drug conjugate of about 3.2 mg/kg is administered to a subject with cancer.

18. The antibody-drug conjugate according to any one of claims 1 to 8, wherein a dose of the antibody-drug conjugate of about 4.8 mg/kg is administered to a subject with cancer.

19. The antibody-drug conjugate according to any one of claims 1 to 8, wherein a dose of the antibody-drug conjugate of about 5.4 mg/kg is administered to a subject with cancer.

20. The antibody-drug conjugate according to any one of claims 1 to 8, wherein a dose of the antibody-drug conjugate of about 5.6 mg/kg is administered to a subject with cancer.

21. The antibody-drug conjugate according to any one of claims 1 to 8, wherein a dose of the antibody-drug conjugate of about 6.4 mg/kg is administered to a subject with cancer.

22. The antibody-drug conjugate according to any one of claims 1 to 8, wherein a dose of the antibody-drug conjugate of about 8.0 mg/kg is administered to a subject with cancer.

23. The antibody-drug conjugate according to any one of claims 1 to 8, wherein a dose of the antibody-drug conjugate of 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg or 6.4 mg/kg is administered to a subject with cancer.

24. The antibody-drug conjugate according to any one of claims 1 to 23, wherein the antibody-drug conjugate is administered by intravenous administration.

25. The antibody-drug conjugate according to any one of claims 1 to 24, wherein the antibody-drug conjugate is administered once every 3 weeks.

26. The antibody-drug conjugate according to any one of claims 1 to 25, wherein the cancer is selected from the group consisting of renal cell carcinoma, renal clear cell carcinoma, papillary renal cell carcinoma, ovarian cancer, ovarian serous adenocarcinoma, clear cell carcinoma of ovary, endometrioid carcinoma of ovary, ovarian mucinous tumor, thyroid cancer, bile duct cancer, lung cancer, non-small cell lung cancer, cervix cancer, brain tumor, head and neck cancer, sarcoma, osteosarcoma, small cell lung cancer, glioblastoma, mesothelioma, uterine cancer, pancreatic cancer, Wilms' tumor, neuroblastoma, colorectal cancer, gastric cancer, endometrial cancer, nasopharyngeal cancer, prostate cancer or cancers associated with von Hippel-Lindau disease.

27. The antibody-drug conjugate according to claim 26, wherein the cancer is renal cell carcinoma, renal clear cell carcinoma or papillary renal cell carcinoma.

28. The antibody-drug conjugate according to claim 26, wherein the cancer is ovarian cancer.

29. The antibody-drug conjugate according to claim 28, wherein the ovarian cancer is selected from the group consisting of epithelial ovarian cancer, fallopian tube cancer, or primary peritoneal cancer.

30. The antibody-drug conjugate according to any one of claims 1 to 29, wherein the cancer is metastatic.

31. The antibody-drug conjugate according to any one of claims 1 to 30, wherein the cancer is resistant or refractory.

32. The antibody-drug conjugate according to claim 31, wherein the resistance or refractoriness is resistance or refractoriness acquired by the cancer due to treatment with an anticancer drug.

33. The antibody-drug conjugate according to claim 32, wherein the anticancer drug is a platinum-based chemotherapeutic, a chemotherapeutic, a PARP inhibitor, an immune checkpoint inhibitor, an angiogenic inhibitor or a VEGFR-TKI.

34. The antibody-drug conjugate according to any one of claims 1 to 33, wherein the subject has a history of treatment with one or two or more anticancer drugs selected from the group consisting of a platinum-based chemotherapeutic, a chemotherapeutic, a PARP inhibitor, an immune checkpoint inhibitor, an angiogenic inhibitor and a VEGFR-TKI.

35. The antibody-drug conjugate according to claim 32, wherein the anticancer drug is a platinum-based chemotherapeutic.

36. The antibody-drug conjugate according to claim 35, wherein the platinum-based chemotherapeutic comprises a platinum-based drug and a taxane.

37. The antibody-drug conjugate according to any one of claims 1 to 36, wherein the cancer is a CDH6-expressing cancer.

38. The antibody-drug conjugate according to claim 37, wherein the CDH6-expressing cancer is CDH6-overexpressing cancer.

39. The antibody-drug conjugate according to any one of claims 1 to 38, wherein the cancer is an inoperable or recurrent cancer.

40. The antibody-drug conjugate according to any one of claims 1 to 39, wherein the subject exhibits a recurrence of the cancer prior to administration of the antibody-drug conjugate.

41. The antibody-drug conjugate according to claim 40, wherein the recurrence of the cancer occurs in less than or within about six months of completion of a chemotherapy regimen comprising a platinum-based drug.

42. The antibody-drug conjugate according to claim 40, wherein the recurrence of the cancer occurs in less than or within about six months of completion of a chemotherapy regimen comprising a platinum-based drug and a taxane.

43. The antibody-drug conjugate according to claim 40, wherein the recurrence of the cancer occurs in or after about six months of completion of a chemotherapy regimen comprising a platinum-based drug.

44. The antibody-drug conjugate according to claim 40, wherein the recurrence of the cancer occurs in or after about six months of completion of a chemotherapy regimen comprising a platinum-based drug and a taxane.

45. The antibody-drug conjugate according to any one of claims 1 to 44, wherein the objective response rate of a subject is at least about 20%.

46. The antibody-drug conjugate according to any one of claims 1 to 44, wherein the objective response rate of a subject is at least about 30%.

47. The antibody-drug conjugate according to any one of claims 1 to 46, wherein the progression free survival of a subject is at least 5 months after administration of the antibody-drug conjugate.

48. The antibody-drug conjugate according to any one of claims 1 to 46, wherein the progression free survival of a subject is at least 5.5 months after administration of the antibody-drug conjugate.

49. The antibody-drug conjugate according to any one of claims 1 to 48, wherein the antibody-drug conjugate is administered as monotherapy.

50. The antibody-drug conjugate according to any one of claims 1 to 48, wherein a subject is administered the ADC with a second drug.

51. The antibody-drug conjugate according to claim 50, wherein the antibody-drug conjugate is administered prior to, after, or concurrently with the second drug.

52. The antibody-drug conjugate according to any one of claims 1 to 44, wherein the antibody-drug conjugate is administered as maintenance therapy.

53. The antibody-drug conjugate according to any one of claims 1 to 44, wherein the antibody-drug conjugate is administered as adjuvant therapy.

54. The antibody-drug conjugate according to any one of claims 1 to 44, wherein the antibody-drug conjugate is administered after surgical resection of the tumor.

55. The antibody-drug conjugate according to any one of claims 1 to 44, wherein the antibody-drug conjugate is administered as neoadjuvant therapy.

56. The antibody-drug conjugate according to any one of claims 1 to 44, wherein the antibody-drug conjugate is administered prior to surgical resection of the tumor.

57. An anti-CDH6 antibody-drug conjugate for use in treating or preventing cancer, the anti-CDH6 antibody-drug conjugate having the structure represented by the following formula: 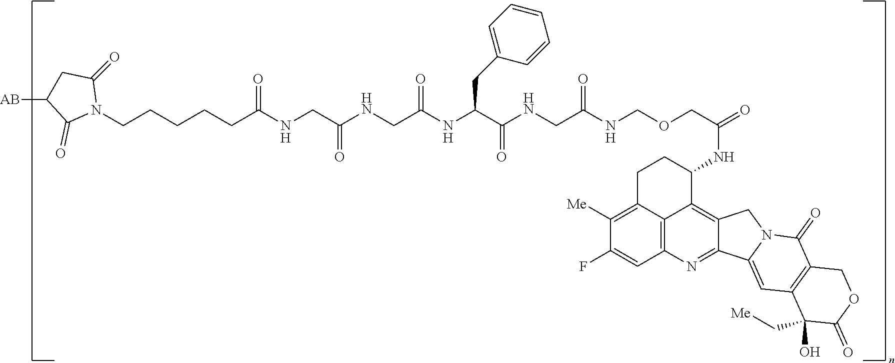
wherein AB represents an anti-CDH6 antibody or the functional fragment of the antibody, n represents the average number of units of the drug-linker structure conjugated to the antibody per antibody, and the antibody is connected to the linker via a sulfhydryl group derived from the antibody; and wherein the anti-CDH6 antibody-drug conjugate is a salt thereof or a hydrate of the anti-CDH6 antibody-drug conjugate or the salt, wherein the average number of units of the drug-linker structure conjugated per antibody is 7 to 8, wherein the antibody comprises: the heavy chain amino acid sequence represented by SEQ ID NO: 87 or an amino acid sequence derived from the amino acid sequence represented by SEQ ID NO: 87 in which one or two amino acids are deleted from the carboxyl terminus thereof; and the light chain amino acid sequence represented by SEQ ID NO: 88, wherein a dose of the antibody-drug conjugate is in a range of 1.6 mg/kg to 9.6 mg/kg, wherein the antibody-drug conjugate is administered by intravenous administration, wherein the antibody-drug conjugate is administered once every 3 weeks.

58. A pharmaceutical composition containing the antibody-drug conjugate according to any one of claims 1 to 57 or a salt thereof as an active component, and a pharmaceutically acceptable formulation component.

59. A method of treating or preventing cancer in a subject, comprising administering to a subject with cancer an anti-CDH6 antibody-drug conjugate comprising an anti-CDH6 antibody and an antitumor compound connected by a linker, wherein the linker and the antitumor compound are represented by the following formula:
-(Succinimid-3-yl-N)—CH₂CH₂CH₂CH₂CH₂—C(═O)-GGFG-NH—CH₂—O—CH₂—C(═O)—(NH-DX)
wherein -(Succinimid-3-yl-N)— has a structure represented by the following formula: 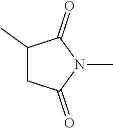
which is connected to the antibody at position 3 thereof and is connected to a methylene group in the linker structure containing this structure on the nitrogen atom at position 1, and (NH-DX) represents a group represented by the following formula: 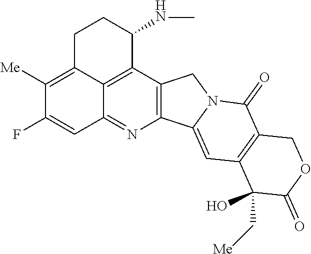 [SMILES: CC[C@@]1(O)C(=O)OCc2c1cc1n(c2=O)Cc2c-1nc1cc(F)c(C)c3c1c2[C@@H](NC)CC3]
wherein the nitrogen atom of the amino group at position 1 is the connecting position, wherein the anti-CDH6 antibody comprises CDRH1 consisting of the amino acid sequence of SEQ ID NO: 17, CDRH2 consisting of the amino acid sequence of SEQ ID NO: 60 and CDRH3 consisting of the amino acid sequence of SEQ ID NO: 19 in its heavy chain variable region and CDRL1 consisting of the amino acid sequence of SEQ ID NO: 12, CDRL2 consisting of the amino acid sequence of SEQ ID NO: 13 and CDRL3 consisting of the amino acid sequence of SEQ ID NO: 14 in its light chain variable region.

60. The method according to claim 59, wherein the antibody comprises a heavy chain variable region comprising amino acids sequence of SEQ ID NO: 71 and a light chain variable region comprising amino acids sequence of SEQ ID NO: 63.

61. The method according to claim 59 or 60, wherein the antibody-drug conjugate has a structure represented by the following formula: 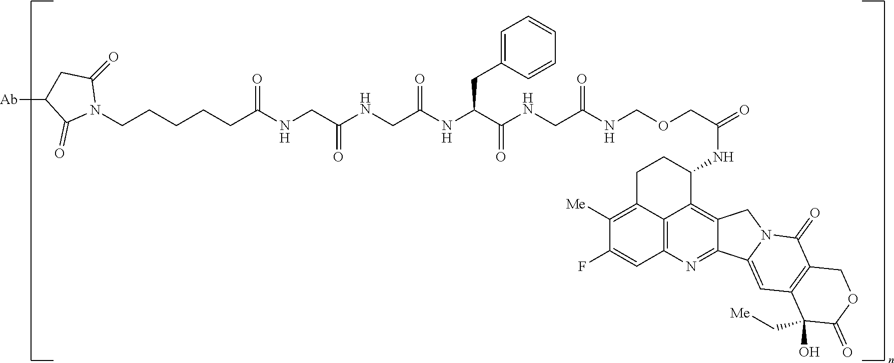
wherein Ab is an antibody comprising a heavy chain variable region comprising SEQ ID NO: 71 and a light chain variable region comprising SEQ ID NO: 63, and n represents the average number of units of the drug-linker structure conjugated to the antibody per antibody, wherein the average number of units of the selected drug-linker structure conjugated per antibody is in the range of from 1 to 10.

62. The method according to any one of claims 59 to 61, wherein the antibody comprises a heavy chain comprising the amino acid sequence at positions 20 to 471 in SEQ ID NO: 69 and a light chain comprising the amino acid sequence at positions 21 to 233 in SEQ ID NO: 61.

63. The method according to any one of claims 59 to 62, wherein the heavy chain or the light chain has undergone one or two or more modifications selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, conversion of N-terminal glutamine or N-terminal glutamic acid to pyroglutamic acid, and a deletion of one or two amino acids from the carboxyl terminus.

64. The method according to any one of claims 59 to 63, wherein the anti-CDH6 antibody lacks a lysine residue at the carboxyl terminus of the heavy chain.

65. The method according to any one of claims 59 to 64, wherein an average number of units of the antitumor compound conjugated per antibody is in a range of from 2 to 8.

66. The method according to any one of claims 59 to 65, wherein an average number of units of the antitumor compound conjugated per antibody is 7 to 8.

67. The method according to any one of claims 59 to 66, wherein a dose of the antibody-drug conjugate in a range of 1.6 mg/kg to 9.6 mg/kg is administered to a subject with cancer.

68. The method according to any one of claims 59 to 66, wherein a dose of the antibody-drug conjugate in a range of 3.2 mg/kg to 9.6 mg/kg is administered to a subject with cancer.

69. The method according to any one of claims 59 to 66, wherein a dose of the antibody-drug conjugate in a range of 4.8 mg/kg to 9.6 mg/kg is administered to a subject with cancer.

70. The method according to any one of claims 59 to 66, wherein a dose of the antibody-drug conjugate in a range of 3.2 mg/kg to 8.0 mg/kg is administered to a subject with cancer.

71. The method according to any one of claims 59 to 66, wherein a dose of the antibody-drug conjugate in a range of 3.2 mg/kg to 6.4 mg/kg is administered to a subject with cancer.

72. The method according to any one of claims 59 to 66, wherein a dose of the antibody-drug conjugate in a range of 4.8 mg/kg to 8.0 mg/kg is administered to a subject with cancer.

73. The method according to any one of claims 59 to 66, wherein a dose of the antibody-drug conjugate in a range of 4.8 mg/kg to 6.4 mg/kg is administered to a subject with cancer.

74. The method according to any one of claims 59 to 66, wherein a dose of the antibody-drug conjugate of about 3.2 mg/kg, about 4.8 mg/kg, about 5.4 mg/kg, about 5.6 mg/kg, about 6.4 mg/kg or about 8.0 mg/kg is administered to a subject with cancer.

75. The method according to any one of claims 59 to 66, wherein a dose of the antibody-drug conjugate of about 3.2 mg/kg is administered to a subject with cancer.

76. The method according to any one of claims 59 to 66, wherein a dose of the antibody-drug conjugate of about 4.8 mg/kg is administered to a subject with cancer.

77. The method according to any one of claims 59 to 66, wherein a dose of the antibody-drug conjugate of about 5.4 mg/kg is administered to a subject with cancer.

78. The method according to any one of claims 59 to 66, wherein a dose of the antibody-drug conjugate of about 5.6 mg/kg is administered to a subject with cancer.

79. The method according to any one of claims 59 to 66, wherein a dose of the antibody-drug conjugate of about 6.4 mg/kg is administered to a subject with cancer.

80. The method according to any one of claims 59 to 66, wherein a dose of the antibody-drug conjugate of about 8.0 mg/kg is administered to a subject with cancer.

81. The method according to any one of claims 59 to 66, wherein a dose of the antibody-drug conjugate of 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg or 6.4 mg/kg is administered to a subject with cancer.

82. The method according to any one of claims 59 to 81, wherein the antibody-drug conjugate is administered by intravenous administration.

83. The method according to any one of claims 59 to 82, wherein the antibody-drug conjugate is administered once every 3 weeks.

84. The method according to any one of claims 59 to 83, wherein the cancer is selected from the group consisting of renal cell carcinoma, renal clear cell carcinoma, papillary renal cell carcinoma, ovarian cancer, ovarian serous adenocarcinoma, clear cell carcinoma of ovary, endometrioid carcinoma of ovary, ovarian mucinous tumor, thyroid cancer, bile duct cancer, lung cancer, non-small cell lung cancer, cervix cancer, brain tumor, head and neck cancer, sarcoma, osteosarcoma, small cell lung cancer, glioblastoma, mesothelioma, uterine cancer, pancreatic cancer, Wilms' tumor, neuroblastoma, colorectal cancer, gastric cancer, endometrial cancer, nasopharyngeal cancer, prostate cancer or cancers associated with von Hippel-Lindau disease.

85. The method according to claim 84, wherein the cancer is renal cell carcinoma, renal clear cell carcinoma or papillary renal cell carcinoma.

86. The method according to claim 84, wherein the cancer is ovarian cancer.

87. The method according to claim 86, wherein the ovarian cancer is selected from the group consisting of epithelial ovarian cancer, fallopian tube cancer, or primary peritoneal cancer.

88. The antibody-drug conjugate according to any one of claims 59 to 87, wherein the cancer is metastatic.

89. The method according to any one of claims 59 to 88, wherein the cancer is resistant or refractory.

90. The method according to claim 89, wherein the resistance or refractoriness is resistance or refractoriness acquired by the cancer due to treatment with an anticancer drug.

91. The method according to claim 90, wherein the anticancer drug is a platinum-based chemotherapeutic, a chemotherapeutic, a PARP inhibitor, an immune checkpoint inhibitor, an angiogenic inhibitor or a VEGFR-TKI.

92. The method according to any one of claims 59 to 91, wherein the subject has a history of treatment with one or two or more anticancer drugs selected from the group consisting of a platinum-based chemotherapeutic, a chemotherapeutic, a PARP inhibitor, an immune checkpoint inhibitor, an angiogenic inhibitor and a VEGFR-TKI.

93. The method according to claim 90, wherein the anticancer drug is a platinum-based chemotherapeutic.

94. The method according to claim 93, wherein the platinum-based chemotherapeutic comprises a platinum-based drug and a taxane.

95. The method according to any one of claims 59 to 94, wherein the cancer is a CDH6-expressing caner.

96. The method according to claim 95, wherein the CDH6-expressing cancer is CDH6-overexpressing cancer.

97. The method according to any one of claims 59 to 96, wherein the cancer is an inoperable or recurrent cancer.

98. The method according to any one of claims 59 to 97, wherein the subject exhibits a recurrence of the cancer prior to administration of the antibody-drug conjugate.

99. The method according to claim 98, wherein the recurrence of the cancer occurs in less than or within about six months of completion of a chemotherapy regimen comprising a platinum-based drug.

100. The method according to claim 98, wherein the recurrence of the cancer occurs in less than or within about six months of completion of a chemotherapy regimen comprising a platinum-based drug and a taxane.

101. The method according to claim 98, wherein the recurrence of the cancer occurs in or after about six months of completion of a chemotherapy regimen comprising a platinum-based drug.

102. The method according to claim 98, wherein the recurrence of the cancer occurs in or after about six months of completion of a chemotherapy regimen comprising a platinum-based drug and a taxane.

103. The method according to any one of claims 59 to 102, wherein the objective response rate of a subject is at least about 20%.

104. The method according to any one of claims 59 to 102, wherein the objective response rate of a subject is at least about 30%.

105. The method according to any one of claims 59 to 104, wherein the progression free survival of a subject is at least 5 months after administration of the antibody-drug conjugate.

106. The method according to any one of claims 59 to 104, wherein the progression free survival of a subject is at least 5.5 months after administration of the antibody-drug conjugate.

107. The method according to any one of claims 59 to 106, wherein the antibody-drug conjugate is administered as monotherapy.

108. The method according to any one of claims 59 to 106, wherein a subject is administered the ADC with a second drug.

109. The method according to claim 108, wherein the antibody-drug conjugate is administered prior to, after, or concurrently with the second drug.

110. The method according to any one of claims 59 to 102, wherein the antibody-drug conjugate is administered as maintenance therapy.

111. The method according to any one of claims 59 to 102, wherein the antibody-drug conjugate is administered as adjuvant therapy.

112. The method according to any one of claims 59 to 102, wherein the antibody-drug conjugate is administered after surgical resection of the tumor.

113. The method according to any one of claims 59 to 102, wherein the antibody-drug conjugate is administered as neoadjuvant therapy.

114. The method according to any one of claims 59 to 102, wherein the antibody-drug conjugate is administered prior to surgical resection of the tumor.

115. A method of treating or preventing cancer in a subject, comprising administering to a subject with cancer an anti-CDH6 antibody-drug conjugate having the structure represented by the following formula: 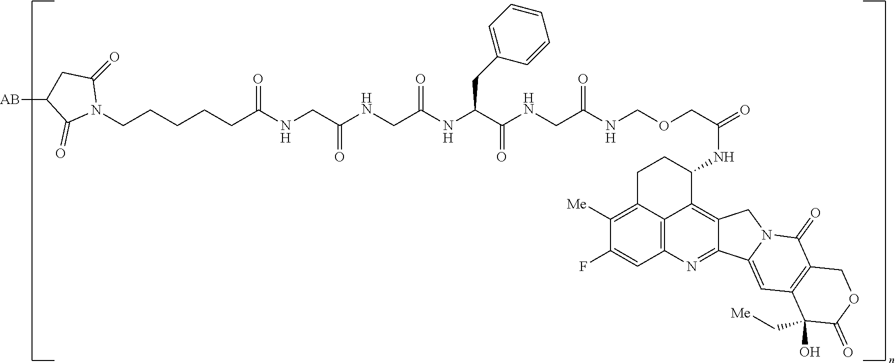
wherein AB represents an anti-CDH6 antibody or the functional fragment of the antibody, n represents the average number of units of the drug-linker structure conjugated to the antibody per antibody, and the antibody is connected to the linker via a sulfhydryl group derived from the antibody; and wherein the anti-CDH6 antibody-drug conjugate is a salt thereof or a hydrate of the anti-CDH6 antibody-drug conjugate or the salt, wherein the average number of units of the drug-linker structure conjugated per antibody is 7 to 8, wherein the antibody comprises: the heavy chain amino acid sequence represented by SEQ ID NO: 87 or an amino acid sequence derived from the amino acid sequence represented by SEQ ID NO: 87 in which one or two amino acids are deleted from the carboxyl terminus thereof; and the light chain amino acid sequence represented by SEQ ID NO: 88, wherein a dose of the antibody-drug conjugate is in a range of 1.6 mg/kg to 9.6 mg/kg, wherein the antibody-drug conjugate is administered by intravenous administration, wherein the antibody-drug conjugate is administered once every 3 weeks.

116. The method according to any one of claims 59 to 115, wherein the antibody-drug conjugate is administered in a pharmaceutical composition comprising at least one pharmaceutically acceptable formulation component.


SEQUENCE LISTING (89 sequences)

SEQ ID NO: 1 - protein, 790 aa, Homo sapiens
MRTYRYFLLLFWVGQPYPTLSTPLSKRTSGFPAKKRALELSGNSKNELNRSKRSWMWNQFFLLEEYTGSDYQYVGKLHSDQDRGDGSLKYILSGDGAGDLFIINENTGDIQATKRLDREEKPVYILRAQAINRRTGRPVEPESEFIIKIHDINDNEPIFTKEVYTATVPEMSDVGTFVVQVTATDADDPTYGNSAKVVYSILQGQPYFSVESETGIIKTALLNMDRENREQYQVVIQAKDMGGQMGGLSGTTTVNITLTDVNDNPPRFPQSTYQFKTPESSPPGTPIGRIKASDADVGENAEIEYSITDGEGLDMFDVITDQETQEGIITVKKLLDFEKKKVYTLKVEASNPYVEPRFLYLGPFKDSATVRIVVEDVDEPPVFSKLAYILQIREDAQINTTIGSVTAQDPDAARNPVKYSVDRHTDMDRIFNIDSGNGSIFTSKLLDRETLLWHNITVIATEINNPKQSSRVPLYIKVLDVNDNAPEFAEFYETFVCEKAKADQLIQTLHAVDKDDPYSGHQFSFSLAPEAASGSNFTIQDNKDNTAGILTRKNGYNRHEMSTYLLPVVISDNDYPVQSSTGTVTVRVCACDHHGNMQSCHAEALIHPTGLSTGALVAILLCIVILLVTVVLFAALRRQRKKEPLIISKEDIRDNIVSYNDEGGGEEDTQAFDIGTLRNPEAIEDNKLRRDIVPEALFLPRRTPTARDNTDVRDFINQRLKENDTDPTAPPYDSLATYAYEGTGSVADSLSSLESVTTDADQDYDYLSDWGPRFKKLADMYGGVDSDKDS

SEQ ID NO: 2 - protein, 106 aa, Homo sapiens
SWMWNQFFLLEEYTGSDYQYVGKLHSDQDRGDGSLKYILSGDGAGDLFIINENTGDIQATKRLDREEKPVYILRAQAINRRTGRPVEPESEFIIKIHDINDNEPIF

SEQ ID NO: 3 - protein, 109 aa, Homo sapiens
TKEVYTATVPEMSDVGTFVVQVTATDADDPTYGNSAKVVYSILQGQPYFSVESETGIIKTALLNMDRENREQYQVVIQAKDMGGQMGGLSGTTTVNITLTDVNDNPPRF

SEQ ID NO: 4 - protein, 115 aa, Homo sapiens
PQSTYQFKTPESSPPGTPIGRIKASDADVGENAEIEYSITDGEGLDMFDVITDQETQEGIITVKKLLDFEKKKVYTLKVEASNPYVEPRFLYLGPFKDSATVRIVVEDVDEPPVF

SEQ ID NO: 5 - protein, 103 aa, Homo sapiens
SKLAYILQIREDAQINTTIGSVTAQDPDAARNPVKYSVDRHTDMDRIFNIDSGNGSIFTSKLLDRETLLWHNITVIATEINNPKQSSRVPLYIKVLDVNDNAP

SEQ ID NO: 6 - protein, 122 aa, Homo sapiens
EFAEFYETFVCEKAKADQLIQTLHAVDKDDPYSGHQFSFSLAPEAASGSNFTIQDNKDNTAGILTRKNGYNRHEMSTYLLPVVISDNDYPVQSSTGTVTVRVCACDHHGNMQSCHAEALIHP

SEQ ID NO: 7 - protein, 790 aa, Mus musculus
MRTYRYFLLLFWVGQPYPTFSNPLSKRTSGFPAKRKALELSANSRNELSRSKRSWMWNQFFLLEEYTGSDYQYVGKLHSDQDRGDGSLKYILSGDGAGDLFIINENTGDIQATKRLDREEKPVYILRAQAVNRRTGRPVEPESEFIIKIHDINDNEPIFTKDVYTATVPEMADVGTFVVQVTATDADDPTYGNSAKVVYSILQGQPYFSVESETGIIKTALLNMDRENREQYQVVIQAKDMGGQMGGLSGTTTVNITLTDVNDNPPRFPQSTYQFKTPESSPPGTPIGRIKASDADVGENAEIEYSITDGEGHEMFDVITDQETQEGIITVKKLLDFEKKKVYTLKVEASNPHVEPRFLYLGPFKDSATVRIVVDDVDEPPVFSKLAYILQIREDARINTTIGSVAAQDPDAARNPVKYSVDRHTDMDRIFNIDSGNGSIFTSKLLDRETLLWHNITVIATEINNPKQSSRVPLYIKVLDVNDNAPEFAEFYETFVCEKAKADQLIQTLRAVDKDDPYSGHQFSFSLAPEAASSSNFTIQDNKDNTAGILTRKNGYNRHEMSTYLLPVVISDNDYPVQSSTGTVTVRVCACDHHGNMQSCHAEALIHPTGLSTGALVAILLCIVILLVTVVLFAALRRQRKKEPLIISKEDIRDNIVSYNDEGGGEEDTQAFDIGTLRNPEAMEDSKSRRDIVPEALFLPRRTPTARDNTDVRDFINQRLKENDTDPTAPPYDSLATYAYEGTGSVADSLSSLESVTTDGDQDYDYLSDWGPRFKKLADMYGGMDSDKDS

SEQ ID NO: 8 - protein, 789 aa, Rattus norvegicus
MRTYRYFLLLFWVGQPYPTFSNPLSKRTSGFPAKRRALELSANSRNELSRSKRSWMWNQFFLLEEYTGSDYQYVGKLHSDQDRGDGSLKYILSGDGAGDLFIINENTGDIQATKRLDREEKPVYILRAQAINRRTGRPVEPESEFIIKIHDINDNEPIFTKDVYTATVPEMADVGTFVVQVTATDADDPTYGNSAKVVYSILQGQPYFSVESETGIIKTALLNMDRENREQYQVVIQAKDMGGQMGGLSGTTTVNITLTDVNDNPPRFPQSTYQFKTPESSPPGTPIGRIKASDADVGENAEIEYSITDGEGHDMFDVITDQETQEGIITVKKLLDFEKKRVYTLKVEASNPHIEPRFLYLGPFKDSATVRIVVDDVDEPPVFSKPAYILQIREDAQINTTIGSVAAQDPDAARNPVKYSVDRHTDMDRIFNIDSGNGSIFTSKLLDRETLLWHNITVIATEINNPKQSSRVPLYIKVLDVNDNAPEFAEFYETFVCEKAKADQLIQTLHAVDKDDPYSGHQFSFSLAPEAASGSNFTIQDNKDNTAGILTRKNGYNRHEMSTYLLPVVISDNDYPVQSSTGTVTVRVCACDHHGNMQSCHAEALIHPTGLSTGALVAILLCIVILLVTVVLFAALRRQRKKEPLIISKEDIRDNIVSYNDEGGGEEDTQAFDIGTLRNPKPWRQQSRRDMVPEALFLPRRTPTARDNTDVRDFISQRLRKMNTDPTAPPYDSLATYAYEGTGSVADSLSSLESVTTDGDQDYGYLSDWGPRFKKLADMYGGMDSDKDS

SEQ ID NO: 9 - protein, 790 aa, Macaca fascicularis
MRTYRYFLLLFWVGQPYPTLSTPLSKRTSGFPAKKRALELSGNSKNELNRSKRSWMWNQFFLLEEYTGSDYQYVGKLHSDQDRGDGSLKYILSGDGAGDLFIINENTGDIQATKRLDREEKPVYILRAQAINRRTGRPVEPESEFIIKIHDINDNEPIFTKEVYTATVPEMSDVGTFVVQVTATDADDPTYGNSAKVVYSILQGQPYFSVESETGIIKTALLNMDRENREQYQVVIQAKDMGGQMGGLSGTTTVNITLTDVNDNPPRFPQSTYQFKTPESSPPGTPIGRIKASDADVGENAEIEYSITDGEGLDMFDVITDQETQEGIITVKKLLDFEKKKVYTLKVEASNPHVEPRFLYLGPFKDSATVRIVVEDVDEPPVFSKLAYILQIREDAQINTTIGSVTAQDPDAARNPVKYSVDRHTDMDRIFNIDSGNGSIFTSKLLDRETLLWHNITVIATEINNPKQSSRVPLYIKVLDVNDNAPEFAEFYETFVCEKAKADQLIQTLRAVDKDDPYSGHQFSFSLAPEAASGSNFTIQDNKDNTAGILTRKNGYNRHEMSTYLLPVVISDNDYPVQSSTGTVTVRVCACDHHGNMQSCHAEALIHPTGLSTGALVAILLCIVILLVTVVLFAALRRQRKKEPLIISKEDIRDNIVSYNDEGGGEEDTQAFDIGTLRNPEAIEDNKLRRDIVPEALFLPRRTPTARDNTDVRDFINQRLKENDTDPTAPPYDSLATYAYEGTGSVADSLSSLESVTTDGDQDYDYLSDWGPRFKKLADMYGGVDSDKDS

SEQ ID NO: 10 - protein, 108 aa, Rattus norvegicus
DIQMTQSPSLLSASVGDRVTLNCKASQNIYKNLAWYQQKLGEGPKLLIYDANTLQTGIPSRFSGSGSGSDFTLTISSLQPEDVATYFCQQYYSGWAFGGVTNLELKRA

SEQ ID NO: 11 - DNA, 324 bp, Rattus norvegicus
  source 1..324: mol_type = other DNA
gacatccagatgacccagtctccttcactcctgtctgcatctgtgggagacagagtcactctcaactgcaaagcaagtcagaatatttataagaacttagcctggtatcagcaaaagcttggagaaggtcccaaactcctgatttatgatgcaaacactttgcaaacgggcatcccatcaaggttcagtggcagtggatctggttcagatttcacactcaccatcagcagcctgcagcctgaagatgttgccacatatttctgccagcagtactatagcgggtgggcgttcggtggagtcaccaacctggaattgaaacgggct

SEQ ID NO: 12 - protein, 11 aa, Rattus norvegicus
KASQNIYKNLA

SEQ ID NO: 13 - protein, 7 aa, Rattus norvegicus
DANTLQT

SEQ ID NO: 14 - protein, 8 aa, Rattus norvegicus
QQYYSGWA

SEQ ID NO: 15 - protein, 122 aa, Rattus norvegicus
QVQLQQSGAELVKPGSSVKISCKASGYTFTRNFMHWIKQQPGNGLEWIGWIYCGDGETEYNQKFNGKATLTADRSSSTAYMELSRLTSEDSAVYFCARGVYGGFAGGYFDFWGQGVMVTVSS

SEQ ID NO: 16 - DNA, 366 bp, Rattus norvegicus
  source 1..366: mol_type = other DNA
caggtacagctgcagcaatctggggctgaactggtgaagcctgggtcctcagtgaaaatttcctgcaaggcttctggctacaccttcaccaggaactttatgcactggataaaacagcagcctggaaatggccttgagtggattgggtggatttattgtggagatggtgagacagagtacaatcaaaagttcaatgggaaggcaacactcactgcggacagatcctccagcacagcctatatggagctcagcagactgacatctgaggactctgcagtctatttctgtgcaagaggggtttacggagggtttgccgggggctactttgatttctggggccaaggagtcatggtcacagtctcctca

SEQ ID NO: 17 - protein, 10 aa, Rattus norvegicus
GYTFTRNFMH

SEQ ID NO: 18 - protein, 10 aa, Rattus norvegicus
WIYCGDGETE

SEQ ID NO: 19 - protein, 13 aa, Rattus norvegicus
GVYGGFAGGYFDF

SEQ ID NO: 20 - protein, 109 aa, Rattus norvegicus
DVQMTHSPSYLAASPGESVSISCKTSKNISNYLVWYQQKPGEAYKLLIYSGSTLQSGTPSRFSGSGSGTDFTLTIRSLEPEDFGLYFCQQYYEKPFTFGSGTKLEIKRA

SEQ ID NO: 21 - DNA, 327 bp, Rattus norvegicus
  source 1..327: mol_type = other DNA
gatgtccagatgacccactctccgtcttatcttgctgcgtctcctggagaaagtgtttccatcagttgcaagacaagtaagaacattagtaattatttagtctggtatcaacagaaacctggggaagcatataagcttcttatctattctgggtcaactttgcaatctggaactccatcaaggttcagtggcagtggatctggtacagatttcactctcactatcagaagcctggagcctgaagattttggactctatttctgtcaacagtattatgaaaaaccattcacgttcggctcagggacgaagttggaaataaaacgggct

SEQ ID NO: 22 - protein, 11 aa, Rattus norvegicus
KTSKNISNYLV

SEQ ID NO: 23 - protein, 7 aa, Rattus norvegicus
SGSTLQS

SEQ ID NO: 24 - protein, 9 aa, Rattus norvegicus
QQYYEKPFT

SEQ ID NO: 25 - protein, 121 aa, Rattus norvegicus
EVQLQESGPGLVRPSQSLSLSCSVTDYSITSNYWGWIRRFPGNKMEWMGYITYSGYTSYNPSLQSRISITRDTSKNQFFLQLNSVTAEDTATYYCARSINHGGYSYVVDAWGPGASVTVSS

SEQ ID NO: 26 - DNA, 363 bp, Rattus norvegicus
  source 1..363: mol_type = other DNA
gaggtgcaacttcaggagtcaggacctggccttgtgagaccctcacagtcactctccctctcctgttctgtcactgattactccatcactagtaattactggggctggatccggaggttcccaggaaataaaatggagtggatgggatacataacctatagtggttacactagctacaacccatctctccaaagtcgaatctccattactagagacacatcgaagaatcagttcttcctgcagttgaactctgtaactgctgaggacacagccacatattactgtgcaagatcgattaaccacggaggatatagttatgttgtggatgcctggggtccgggagcttcagtcactgtctcctca

SEQ ID NO: 27 - protein, 10 aa, Rattus norvegicus
DYSITSNYWG

SEQ ID NO: 28 - protein, 9 aa, Rattus norvegicus
YITYSGYTS

SEQ ID NO: 29 - protein, 13 aa, Rattus norvegicus
SINHGGYSYVVDA

SEQ ID NO: 30 - protein, 109 aa, Rattus norvegicus
DVQMTQSPSSLAASPGESVSISCRATKSIGIYLAWYQQKPGKTFKLLIYSGSTLQSGTPSRFSGSGSGTDFTLTIRSLEPEDFGLYFCQQFYENPFTFGSGTKLEIRRA

SEQ ID NO: 31 - DNA, 327 bp, Rattus norvegicus
  source 1..327: mol_type = other DNA
gatgtccagatgacccagtctccgtcttctcttgctgcgtctcctggagaaagtgtttccatcagttgcagggcaactaagagcattggtatttatttagcctggtatcaacagaaacctgggaaaacatttaagcttcttatctactctgggtcaactttgcaatctggaactccatcaaggttcagtggcagtgggtctggtacagatttcactctcaccatcagaagcctggagcctgaagattttggactctatttctgtcaacagttttatgaaaacccattcacgttcggctcagggacgaagttggaaataagacgggct

SEQ ID NO: 32 - protein, 11 aa, Rattus norvegicus
RATKSIGIYLA

SEQ ID NO: 33 - protein, 7 aa, Rattus norvegicus
SGSTLQS

SEQ ID NO: 34 - protein, 9 aa, Rattus norvegicus
QQFYENPFT

SEQ ID NO: 35 - protein, 121 aa, Rattus norvegicus
EVQLQESGPGLVKPSQSLSLTCSVTDYSITTYFWGWIRKFPGNKMEWMGYMSYRGGTSYNPSLKSRISITRDTSKNQFFLQLNSVTTEDTATYYCARCPNYGGHSLVFDYWGQGVMVTVSS

SEQ ID NO: 36 - DNA, 363 bp, Rattus norvegicus
  source 1..363: mol_type = other DNA
gaggtgcagcttcaggagtcaggacctggccttgtgaaaccctcacagtcactctccctcacctgttctgtcactgattactccatcactacttatttctggggctggatccggaagttcccaggaaataaaatggagtggatgggatacatgagctaccgtggtggcacttcctacaacccatctctcaagagtcgaatctccattactagagacacatcgaagaatcagttcttcctgcagttgaactctgtaactactgaggacacagccacatattactgtgcaagatgccctaactacggagggcattcccttgtttttgattactggggccaaggagtcatggtcacagtgtcctca

SEQ ID NO: 37 - protein, 10 aa, Rattus norvegicus
DYSITTYFWG

SEQ ID NO: 38 - protein, 9 aa, Rattus norvegicus
YMSYRGGTS

SEQ ID NO: 39 - protein, 13 aa, Rattus norvegicus
CPNYGGHSLVFDY

SEQ ID NO: 40 - protein, 109 aa, Rattus norvegicus
DVQMTQSPSYLAASPGESVSISCKATKSISNYLAWYQQKPGEAYKVLIYSGSTLQSGTPSRFSGSGSGTDFTLTIRSLEPEDFGLYSCQQYYEKPLTFGSGTKLEIKRA

SEQ ID NO: 41 - DNA, 327 bp, Rattus norvegicus
  source 1..327: mol_type = other DNA
gatgtccagatgacccagtctccgtcttatcttgctgcgtctcctggagaaagtgtttccatcagttgcaaggcaactaagagcattagtaattatttagcctggtatcaacagaaacctggggaagcatataaggttcttatctattctgggtcaactttgcaatctggaactccatcaaggttcagtggcagtggatctggtacagatttcactctcaccatcagaagcctggagcctgaagattttggactctattcctgtcaacagtattatgaaaaaccgctcacgttcggttctgggaccaagctggagatcaaacgggct

SEQ ID NO: 42 - protein, 11 aa, Rattus norvegicus
KATKSISNYLA

SEQ ID NO: 43 - protein, 7 aa, Rattus norvegicus
SGSTLQS

SEQ ID NO: 44 - protein, 9 aa, Rattus norvegicus
QQYYEKPLT

SEQ ID NO: 45 - protein, 121 aa, Rattus norvegicus
EVQLQESGPGLVKPSQSLSLTCSVTGYSITTYYWGWIRKFPGNKMEWMGYISYSGRTSYNPSLKSRMSITRDASKNQFFLQLNSVTTDDTATYYCARSPINHGGYWYFDFWGPGTMVTVSS

SEQ ID NO: 46 - DNA, 363 bp, Rattus norvegicus
  source 1..363: mol_type = other DNA
gaggtgcagcttcaggagtcaggacctggccttgtgaaaccctcacagtcactctccctcacctgttctgtcactggttactccatcactacttattactggggctggatccggaagttcccaggaaataaaatggagtggatggggtacataagctacagtggtcgcactagttataacccatctctcaaaagtcgaatgtccattactagagacgcatcgaagaatcagttcttcctacagttgaactctgtaactactgacgacacagccacatattactgtgcaagatccccaattaaccacggagggtactggtactttgacttctggggcccaggaaccatggtcaccgtgtcctca

SEQ ID NO: 47 - protein, 10 aa, Rattus norvegicus
GYSITTYYWG

SEQ ID NO: 48 - protein, 9 aa, Rattus norvegicus
YISYSGRTS

SEQ ID NO: 49 - protein, 13 aa, Rattus norvegicus
SPINHGGYWYFDF

SEQ ID NO: 50 - DNA, 449 bp, synthetic construct
  misc_feature 1..449: note = DNA comprising DNA sequence coding for human light chain signal sequence and kappa chain constant region
  source 1..449: mol_type = other DNA
gcctccggactctagagccaccatggtgctgcagacccaggtgttcatctccctgctgctgtggatctccggcgcgtacggcgatatcgtgatgattaaacgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgttaggggcccgtttaaacgggggaggcta

SEQ ID NO: 51 - DNA, 1132 bp, synthetic construct
  misc_feature 1..1132: note = DNA comprising DNA sequence coding for human heavy chain signal sequence and IgG1 constant region
  source 1..1132: mol_type = other DNA
gcctccggactctagagccaccatgaaacacctgtggttcttcctcctgctggtggcagctcccagatgggtgctgagccaggtgcaattgtgcaggcggttagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaatgagatatcgggcccgtttaaacgggggaggcta

SEQ ID NO: 52 - DNA, 749 bp, synthetic construct
  misc_feature 1..749: note = DNA comprising DNA sequence coding for chG019 light chain
  source 1..749: mol_type = other DNA
ccagcctccggactctagagccaccatggtgctgcagacccaggtgttcatcagcctgctgctgtggatcagcggcgcctacggcgacatccagatgacccagagccctagcctgctgagcgccagcgtgggcgatagagtgaccctgaactgcaaggccagccagaacatctacaagaacctggcctggtatcagcagaagctgggcgagggccccaagctgctgatctacgacgccaacaccctgcagaccggcatccccagcagattttctggcagcggcagcggctccgacttcaccctgacaatcagcagcctgcagcccgaggacgtggccacctacttttgccagcagtactacagcggctgggccttcggcggcgtgaccaacctggaactgaagagagccgtggccgctccctccgtgttcatcttcccacctagcgacgagcagctgaagtccggcacagcctctgtcgtgtgcctgctgaacaacttctacccccgcgaggccaaggtgcagtggaaggtggacaatgccctgcagtctggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgttgagtttaaacgggggaggctaact

SEQ ID NO: 53 - protein, 233 aa, synthetic construct
  REGION 1..233: note = amino acid sequence of chG019 light chain full-length
MVLQTQVFISLLLWISGAYGDIQMTQSPSLLSASVGDRVTLNCKASQNIYKNLAWYQQKLGEGPKLLIYDANTLQTGIPSRFSGSGSGSDFTLTISSLQPEDVATYFCQQYYSGWAFGGVTNLELKRAVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - DNA, 699 bp, synthetic construct
  misc_feature 1..699: note = nucleotide sequence of chG019 light chain full-length
  source 1..699: mol_type = other DNA
atggtgctgcagacccaggtgttcatcagcctgctgctgtggatcagcggcgcctacggcgacatccagatgacccagagccctagcctgctgagcgccagcgtgggcgatagagtgaccctgaactgcaaggccagccagaacatctacaagaacctggcctggtatcagcagaagctgggcgagggccccaagctgctgatctacgacgccaacaccctgcagaccggcatccccagcagattttctggcagcggcagcggctccgacttcaccctgacaatcagcagcctgcagcccgaggacgtggccacctacttttgccagcagtactacagcggctgggccttcggcggcgtgaccaacctggaactgaagagagccgtggccgctccctccgtgttcatcttcccacctagcgacgagcagctgaagtccggcacagcctctgtcgtgtgcctgctgaacaacttctacccccgcgaggccaaggtgcagtggaaggtggacaatgccctgcagtctggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 55 - DNA, 324 bp, synthetic construct
  misc_feature 1..324: note = nucleotide sequence of chG019 light chain variable region
  source 1..324: mol_type = other DNA
gacatccagatgacccagagccctagcctgctgagcgccagcgtgggcgatagagtgaccctgaactgcaaggccagccagaacatctacaagaacctggcctggtatcagcagaagctgggcgagggccccaagctgctgatctacgacgccaacaccctgcagaccggcatccccagcagattttctggcagcggcagcggctccgacttcaccctgacaatcagcagcctgcagcccgaggacgtggccacctacttttgccagcagtactacagcggctgggccttcggcggcgtgaccaacctggaactgaagagagcc

SEQ ID NO: 56 - protein, 471 aa, synthetic construct
  REGION 1..471: note = amino acid sequence of chG019 heavy chain full-length
MKHLWFFLLLVAAPRWVLSQVQLQQSGAELVKPGSSVKISCKASGYTFTRNFMHWIKQQPGNGLEWIGWIYPGDGETEYNQKFNGKATLTADRSSSTAYMELSRLTSEDSAVYFCARGVYGGFAGGYFDFWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - DNA, 1413 bp, synthetic construct
  misc_feature 1..1413: note = nucleotide sequence of chG019 heavy chain full-length
  source 1..1413: mol_type = other DNA
atgaaacacctgtggttcttcctcctgctggtggcagctcccagatgggtgctgagccaggtgcagctgcagcagtctggcgccgagctcgtgaagcctggcagcagcgtgaagatcagctgcaaggccagcggctacaccttcacccggaacttcatgcactggatcaagcagcagcccggcaacggcctggaatggatcggctggatctatcccggcgacggcgagacagagtacaaccagaagttcaacggcaaggccaccctgaccgccgacagaagcagctccaccgcctacatggaactgagccggctgaccagcgaggacagcgccgtgtacttttgcgccagaggcgtgtacggcggcttcgctggcggctacttcgatttttggggccagggcgtgatggtcaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 58 - protein, 122 aa, synthetic construct
  REGION 1..122: note = amino acid sequence of chG019 heavy chain variable region
QVQLQQSGAELVKPGSSVKISCKASGYTFTRNFMHWIKQQPGNGLEWIGWIYPGDGETEYNQKFNGKATLTADRSSSTAYMELSRLTSEDSAVYFCARGVYGGFAGGYFDFWGQGVMVTVSS

SEQ ID NO: 59 - DNA, 366 bp, synthetic construct
  misc_feature 1..366: note = nucleotide sequence of chG019 heavy chain variable region
  source 1..366: mol_type = other DNA
caggtgcagctgcagcagtctggcgccgagctcgtgaagcctggcagcagcgtgaagatcagctgcaaggccagcggctacaccttcacccggaacttcatgcactggatcaagcagcagcccggcaacggcctggaatggatcggctggatctatcccggcgacggcgagacagagtacaaccagaagttcaacggcaaggccaccctgaccgccgacagaagcagctccaccgcctacatggaactgagccggctgaccagcgaggacagcgccgtgtacttttgcgccagaggcgtgtacggcggcttcgctggcggctacttcgatttttggggccagggcgtgatggtcaccgtcagctca

SEQ ID NO: 60 - protein, 10 aa, synthetic construct
  REGION 1..10: note = amino acid sequence of chG019 CDRH2
WIYPGDGETE

SEQ ID NO: 61 - protein, 233 aa, synthetic construct
  REGION 1..233: note = amino acid sequence of hL02 light chain full-length
MVLQTQVFISLLLWISGAYGDIQMTQSPSSLSASVGDRVTITCKASQNIYKNLAWYQQKPGKAPKLLIYDANTLQTGVPSRFSGSGSGSDFTLTISSLQPEDFATYFCQQYYSGWAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - DNA, 699 bp, synthetic construct
  misc_feature 1..699: note = nucleotide sequence of hL02 light chain full-length
  source 1..699: mol_type = other DNA
atggtgctgcagacccaggtgttcatctccctgctgctgtggatctccggcgcgtacggcgacatccagatgacccagagccctagcagcctgagcgccagcgtgggcgacagagtgaccatcacatgcaaggccagccagaacatctacaagaacctggcctggtatcagcagaagcccggcaaggcccccaagctgctgatctacgacgccaacaccctgcagaccggcgtgcccagcagattttctggcagcggcagcggctccgacttcaccctgacaatcagcagcctgcagcccgaggacttcgccacctacttttgccagcagtactacagcggctgggccttcggccagggcaccaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 63 - protein, 108 aa, synthetic construct
  REGION 1..108: note = amino acid sequence of hL02 light chain variable region
DIQMTQSPSSLSASVGDRVTITCKASQNIYKNLAWYQQKPGKAPKLLIYDANTLQTGVPSRFSGSGSGSDFTLTISSLQPEDFATYFCQQYYSGWAFGQGTKVEIKRT

SEQ ID NO: 64 - DNA, 324 bp, synthetic construct
  misc_feature 1..324: note = nucleotide sequence of hL02 light chain variable region
  source 1..324: mol_type = other DNA
gacatccagatgacccagagccctagcagcctgagcgccagcgtgggcgacagagtgaccatcacatgcaaggccagccagaacatctacaagaacctggcctggtatcagcagaagcccggcaaggcccccaagctgctgatctacgacgccaacaccctgcagaccggcgtgcccagcagattttctggcagcggcagcggctccgacttcaccctgacaatcagcagcctgcagcccgaggacttcgccacctacttttgccagcagtactacagcggctgggccttcggccagggcaccaaggtggaaatcaagcgtacg

SEQ ID NO: 65 - protein, 233 aa, synthetic construct
  REGION 1..233: note = amino acid sequence of hL03 light chain full-length
MVLQTQVFISLLLWISGAYGDIQMTQSPSSLSASVGDRVTITCKASQNIYKNLAWYQQKLGEGPKLLIYDANTLQTGVPSRFSGSGSGTDFTLTISSLQPEDFATYYCQQYYSGWAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - DNA, 699 bp, synthetic construct
  misc_feature 1..699: note = nucleotide sequence of hL03 light chain full-length
  source 1..699: mol_type = other DNA
atggtgctgcagacccaggtgttcatctccctgctgctgtggatctccggcgcgtacggcgacatccagatgacccagagccctagcagcctgagcgccagcgtgggcgacagagtgaccatcacatgcaaggccagccagaacatctacaagaacctggcctggtatcagcagaagctgggcgagggccccaagctgctgatctacgacgccaacaccctgcagaccggcgtgcccagcagattttctggcagcggctccggcaccgacttcaccctgacaatcagcagcctgcagcccgaggacttcgccacctactactgccagcagtactacagcggctgggcctttggccagggcaccaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 67 - protein, 108 aa, synthetic construct
  REGION 1..108: note = amino acid sequence of hL03 light chain variable region
DIQMTQSPSSLSASVGDRVTITCKASQNIYKNLAWYQQKLGEGPKLLIYDANTLQTGVPSRFSGSGSGTDFTLTISSLQPEDFATYYCQQYYSGWAFGQGTKVEIKRT

SEQ ID NO: 68 - DNA, 324 bp, synthetic construct
  misc_feature 1..324: note = nucleotide sequence of hL03 light chain variable region
  source 1..324: mol_type = other DNA
gacatccagatgacccagagccctagcagcctgagcgccagcgtgggcgacagagtgaccatcacatgcaaggccagccagaacatctacaagaacctggcctggtatcagcagaagctgggcgagggccccaagctgctgatctacgacgccaacaccctgcagaccggcgtgcccagcagattttctggcagcggctccggcaccgacttcaccctgacaatcagcagcctgcagcccgaggacttcgccacctactactgccagcagtactacagcggctgggcctttggccagggcaccaaggtggaaatcaagcgtacg

SEQ ID NO: 69 - protein, 471 aa, synthetic construct
  REGION 1..471: note = amino acid sequence of hH01 heavy chain full-length
MKHLWFFLLLVAAPRWVLSEVQLVQSGAEVKKPGASVKVSCKASGYTFTRNFMHWVRQAPGQGLEWMGWIYPGDGETEYAQKFQGRVTITADTSTSTAYMELSSLRSEDTAVYYCARGVYGGFAGGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 1413 bp, synthetic construct
  misc_feature 1..1413: note = nucleotide sequence of hH01 heavy chain full-length
  source 1..1413: mol_type = other DNA
atgaaacacctgtggttcttcctcctgctggtggcagctcccagatgggtgctgagcgaagtgcagctggtgcagtctggcgccgaagtgaagaaaccaggcgccagcgtgaaggtgtcctgcaaggccagcggctacacctttacccggaacttcatgcactgggtgcgccaggctccaggccagggactggaatggatgggctggatctatcccggcgacggcgagacagagtacgcccagaaattccagggcagagtgaccatcaccgccgacaccagcacctccaccgcctacatggaactgagcagcctgcggagcgaggacaccgccgtgtactattgtgccagaggcgtgtacggcggcttcgctggcggctacttcgatttttggggccagggcaccctcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 71 - protein, 122 aa, synthetic construct
  REGION 1..122: note = amino acid sequence of hH01 heavy chain variable region
EVQLVQSGAEVKKPGASVKVSCKASGYTFTRNFMHWVRQAPGQGLEWMGWIYPGDGETEYAQKFQGRVTITADTSTSTAYMELSSLRSEDTAVYYCARGVYGGFAGGYFDFWGQGTLVTVSS

SEQ ID NO: 72 - DNA, 366 bp, synthetic construct
  misc_feature 1..366: note = nucleotide sequence of hH01 heavy chain variable region
  source 1..366: mol_type = other DNA
gaagtgcagctggtgcagtctggcgccgaagtgaagaaaccaggcgccagcgtgaaggtgtcctgcaaggccagcggctacacctttacccggaacttcatgcactgggtgcgccaggctccaggccagggactggaatggatgggctggatctatcccggcgacggcgagacagagtacgcccagaaattccagggcagagtgaccatcaccgccgacaccagcacctccaccgcctacatggaactgagcagcctgcggagcgaggacaccgccgtgtactattgtgccagaggcgtgtacggcggcttcgctggcggctacttcgatttttggggccagggcaccctcgtgaccgtcagctca

SEQ ID NO: 73 - protein, 471 aa, synthetic construct
  REGION 1..471: note = amino acid sequence of hH02 heavy chain full-length
MKHLWFFLLLVAAPRWVLSEVQLVQSGAEVKKPGASVKVSCKASGYTFTRNFMHWVRQAPGQGLEWMGWIYPGDGETEYNQKFQGRVTITADRSTSTAYMELSSLRSEDTAVYFCARGVYGGFAGGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - DNA, 1413 bp, synthetic construct
  misc_feature 1..1413: note = nucleotide sequence of hH02 heavy chain full-length
  source 1..1413: mol_type = other DNA
atgaaacacctgtggttcttcctcctgctggtggcagctcccagatgggtgctgagcgaagtgcagctggtgcagtctggcgccgaagtgaagaaaccaggcgccagcgtgaaggtgtcctgcaaggccagcggctacacctttacccggaacttcatgcactgggtgcgccaggctccaggccagggactggaatggatgggctggatctatcccggcgacggcgagacagagtacaaccagaaattccagggcagagtgaccatcaccgccgacagaagcaccagcaccgcctacatggaactgagcagcctgcggagcgaggataccgccgtgtacttctgtgccagaggcgtgtacggcggcttcgctggcggctacttcgatttttggggccagggcaccctcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 75 - protein, 122 aa, synthetic construct
  REGION 1..122: note = amino acid sequence of hH02 heavy chain variable region
EVQLVQSGAEVKKPGASVKVSCKASGYTFTRNFMHWVRQAPGQGLEWMGWIYPGDGETEYNQKFQGRVTITADRSTSTAYMELSSLRSEDTAVYFCARGVYGGFAGGYFDFWGQGTLVTVSS

SEQ ID NO: 76 - DNA, 366 bp, synthetic construct
  misc_feature 1..366: note = nucleotide sequence of hH02 heavy chain variable region
  source 1..366: mol_type = other DNA
gaagtgcagctggtgcagtctggcgccgaagtgaagaaaccaggcgccagcgtgaaggtgtcctgcaaggccagcggctacacctttacccggaacttcatgcactgggtgcgccaggctccaggccagggactggaatggatgggctggatctatcccggcgacggcgagacagagtacaaccagaaattccagggcagagtgaccatcaccgccgacagaagcaccagcaccgcctacatggaactgagcagcctgcggagcgaggataccgccgtgtacttctgtgccagaggcgtgtacggcggcttcgctggcggctacttcgatttttggggccagggcaccctcgtgaccgtcagctca

SEQ ID NO: 77 - protein, 471 aa, synthetic construct
  REGION 1..471: note = amino acid sequence of hH04 heavy chain full-length
MKHLWFFLLLVAAPRWVLSQVQLVQSGAEVKKPGASVKVSCKASGYTFTRNFMHWIRQAPGQGLEWMGWIYPGDGETEYAQKFQGRVTLTADRSTSTAYMELSSLRSEDTAVYYCARGVYGGFAGGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - DNA, 1413 bp, synthetic construct
  misc_feature 1..1413: note = nucleotide sequence of hH04 heavy chain full-length
  source 1..1413: mol_type = other DNA
atgaaacacctgtggttcttcctcctgctggtggcagctcccagatgggtgctgagccaggtgcagctggtgcagtctggcgccgaagtgaagaaaccaggcgccagcgtgaaggtgtcctgcaaggccagcggctacacctttacccggaacttcatgcactggatccggcaggcccctggacagggcctggaatggatgggctggatctatcccggcgacggcgagacagagtacgcccagaaattccagggcagagtgaccctgaccgccgacagaagcaccagcaccgcctacatggaactgagcagcctgcggagcgaggacaccgccgtgtactattgtgccagaggcgtgtacggcggcttcgctggcggctacttcgatttttggggccagggcaccctcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 79 - protein, 122 aa, synthetic construct
  REGION 1..122: note = amino acid sequence of hH04 heavy chain variable region
QVQLVQSGAEVKKPGASVKVSCKASGYTFTRNFMHWIRQAPGQGLEWMGWIYPGDGETEYAQKFQGRVTLTADRSTSTAYMELSSLRSEDTAVYYCARGVYGGFAGGYFDFWGQGTLVTVSS

SEQ ID NO: 80 - DNA, 366 bp, synthetic construct
  misc_feature 1..366: note = nucleotide sequence of hH04 heavy chain variable region
  source 1..366: mol_type = other DNA
caggtgcagctggtgcagtctggcgccgaagtgaagaaaccaggcgccagcgtgaaggtgtcctgcaaggccagcggctacacctttacccggaacttcatgcactggatccggcaggcccctggacagggcctggaatggatgggctggatctatcccggcgacggcgagacagagtacgcccagaaattccagggcagagtgaccctgaccgccgacagaagcaccagcaccgcctacatggaactgagcagcctgcggagcgaggacaccgccgtgtactattgtgccagaggcgtgtacggcggcttcgctggcggctacttcgatttttggggccagggcaccctcgtgaccgtcagctca

SEQ ID NO: 81 - protein, 235 aa, synthetic construct
  REGION 1..235: note = amino acid sequence of NOV0712 light chain full-length
MVLQTQVFISLLLWISGAYGDIQMTQSPSSLSASVGDRVTITCRASQSISSYLNWYQQKPGKAPKLLIYAVSTLQSGVPSRFSGSGSGTDFTLTISSLQPEDFATYYCQQSGTFPPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - DNA, 705 bp, synthetic construct
  misc_feature 1..705: note = nucleotide sequence coding for amino acid sequence of sequence number 81
  source 1..705: mol_type = other DNA
atggtgctgcagacccaggtgttcatctccctgctgctgtggatctccggcgcgtacggcgacatccagatgacccagagccctagcagcctgagcgccagcgtgggcgacagagtgaccatcacctgtagagccagccagagcatcagcagctacctgaactggtatcagcagaagcccggcaaggcccccaaactgctgatctacgccgtgtccacactgcagagcggcgtgcccagcagattttctggcagcggctccggcaccgacttcaccctgacaatcagcagcctgcagcccgaggacttcgccacctactactgtcagcagtccggcaccttcccccccaccacatttggccagggcaccaaggtggaaatcaagcgtacggtggccgccccctccgtgttcatcttccccccctccgacgagcagctgaagtccggcaccgcctccgtggtgtgcctgctgaataacttctaccccagagaggccaaggtgcagtggaaggtggacaacgccctgcagtccgggaactcccaggagagcgtgaccgagcaggacagcaaggacagcacctacagcctgagcagcaccctgaccctgagcaaagccgactacgagaagcacaaggtgtacgcctgcgaggtgacccaccagggcctgagctcccccgtcaccaagagcttcaacaggggggagtgt

SEQ ID NO: 83 - protein, 467 aa, synthetic construct
  REGION 1..467: note = amino acid sequence of NOV0712 heavy chain full-length
MKHLWFFLLLVAAPRWVLSQVQLLESGGGLVQPGGSLRLSCAASGFTFSSHGMHWVRQAPGKGLEWVSVISGSGSNTGYADSVKGRFTISRDNSKNTLYLQMNSLRAEDTAVYYCARQWGSYAFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - DNA, 1401 bp, synthetic construct
  misc_feature 1..1401: note = nucleotide sequence coding for amino acid sequence of sequence number 83
  source 1..1401: mol_type = other DNA
atgaaacacctgtggttcttcctcctgctggtggcagctcccagatgggtgctgagccaggtgcagctgctggaatctggcggaggactggtgcagcctggcggctctctgagactgtcttgtgccgccagcggcttcaccttcagcagccacggaatgcactgggtgcgccaggcccctggaaagggactggaatgggtgtccgtgatcagcggcagcggctccaataccggctacgccgatagcgtgaagggccggttcaccatcagccgggacaacagcaagaacaccctgtacctgcagatgaacagcctgcgggccgaggacaccgccgtgtactattgtgccagacagtggggcagctacgccttcgattcttggggccagggcaccctcgtgaccgtcagctcagcctccaccaagggcccaagcgtcttccccctggcaccctcctccaagagcacctctggcggcacagccgccctgggctgcctggtcaaggactacttccccgaacccgtgaccgtgagctggaactcaggcgccctgaccagcggcgtgcacaccttccccgctgtcctgcagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccccgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggccagccccgggaaccacaggtgtacaccctgcccccatcccgggaggagatgaccaagaaccaggtcagcctgacctgcctggtcaaaggcttctatcccagcgacatcgccgtggagtgggagagcaatggccagcccgagaacaactacaagaccacccctcccgtgctggactccgacggctccttcttcctctacagcaagctcaccgtggacaagagcaggtggcagcagggcaacgtcttctcatgctccgtgatgcatgaggctctgcacaaccactacacccagaagagcctctccctgtctcccggcaaa

SEQ ID NO: 85 - DNA, 34 bp, synthetic construct
  misc_feature 1..34: note = primer
  source 1..34: mol_type = other DNA
caccatgagaacttaccgctacttcttgctgctc

SEQ ID NO: 86 - DNA, 30 bp, synthetic construct
  misc_feature 1..30: note = primer
  source 1..30: mol_type = other DNA
ttaggagtctttgtcactgtccactcctcc

SEQ ID NO: 87 - protein, 452 aa, synthetic construct
  REGION 1..452: note = Matured heavy chain amino acid sequence of hH01 heavy chain, without a signal sequence
EVQLVQSGAEVKKPGASVKVSCKASGYTFTRNFMHWVRQAPGQGLEWMGWIYPGDGETEYAQKFQGRVTITADTSTSTAYMELSSLRSEDTAVYYCARGVYGGFAGGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 213 aa, synthetic construct
  REGION 1..213: note = Matured light chain amino acid sequence of hL02 light chain, without a signal sequence
DIQMTQSPSSLSASVGDRVTITCKASQNIYKNLAWYQQKPGKAPKLLIYDANTLQTGVPSRFSGSGSGSDFTLTISSLQPEDFATYFCQQYYSGWAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 4 aa, synthetic construct
GGFG